(19) United States
(12) Patent Application Publication
(10) Pub. No.: US 2022/0380784 A1
(43) Pub. Date: Dec. 1, 2022

(54) UNIVERSAL DNA ASSEMBLY

(71) Applicant: Oxford University Innovation Limited, Oxford (Oxfordshire) (GB)

(72) Inventors: Da LIN, Oxford (Oxfordshire) (GB); Katherine ROBINS, Oxford (Oxfordshire) (GB); Chris O'CALLAGHAN, Oxford (Oxfordshire) (GB)

(21) Appl. No.: 17/599,616

(22) PCT Filed: Apr. 2, 2020
(86) PCT No.: PCT/EP2020/059420

§ 371 Date: Sep. 29, 2021

(30) Foreign Application Priority Data
Apr. 2, 2019 (GB) 1904653.1

(51) Int. Cl. C12N 15/64; C12N 15/11; C12N 9/22; C12N 9/10; C12N 15/66

(52) U.S. Cl. CPC C12N 15/64; C12N 15/11; C12N 9/22; C12N 9/1007; C12Y 201/01072; C12N 15/66; C12N 2800/10; C12N 2310/20; C12N 2800/80; C12N 2830/46

(57) ABSTRACT

The invention relates to a nucleic acid comprising at least one methylation-protectable restriction element, the methylation-protectable restriction element comprising: (i) a type IIS restriction enzyme recognition sequence, or a partial type IIS restriction enzyme recognition sequence, that is recognised by a type IIS restriction enzyme that cleaves outside of the recognition sequence; (ii) a DNA methylase recognition sequence that is recognised and methylated by a DNA methylase, wherein the DNA methylase recognition sequence is identical to, or is encompassed within, the type IIS restriction recognition sequence, such that methylation of the nucleic acid by the DNA methylase methylates the type IIS restriction enzyme recognition sequence and protects the nucleic acid from cleavage by the type IIS restriction enzyme; and (iii) a recognition sequence for a sequence-specific DNA-binding protein, wherein the recognition sequence is positioned such that the binding of the sequence-specific DNA-binding protein overlaps with the DNA methylase recognition sequence such that binding of the sequence-specific DNA-binding protein is capable of preventing methylation of the type IIS restriction enzyme recognition sequence by the DNA methylase such that it is not protected from cleavage by the type IIS restriction enzyme. The invention further relates to associated methods of nucleic acid assembly.

This invention relates to DNA assembly methods, and related nucleic acid material.

Advances in biology and biotechnology have led to development of a variety of methods for assembly of large DNA constructs. Existing methods could be largely divided into two groups: homology-based methods that utilize long overlapping sequence between fragments to specify the order of assembly, such as Gibson assembly and yeast- or B. subtilis-based in vivo homologous recombination approaches, and restriction enzyme/recombinase-based methods that utilize defined enzyme-specific sequence to specify the order of assembly, such as Moclo (Reviewed in Nat Rev Mol Cell Biol. 2015 September; 16(9):568-76. doi: 10.1038/nrm4014 (PMID 26081612) and Crit Rev Biotechnol. 2017 May; 37(3):277-286. doi: 10.3109/07388551.2016.1141394 (PMID 26863154). An ongoing struggle is to achieve a balance between reducing sequence design constraints such as forbidden sequence in assembled DNA and unwanted scar sequence introduced during the assembly process, and improving assembly modularity and part reusability.

The initial type IIS restriction enzyme-based DNA assembly system was named Golden Gate assembly. In Golden Gate assembly system, insert DNA fragments to be assembled within insert plasmids are flanked by recognition sites for a type IIS restriction enzyme that cuts outside the recognition sequence and generates arbitrary adhesive ends with sequences independent of the enzyme recognition sequence. The insert plasmids for Golden Gate assembly contain inserts flanked by type IIS restriction sites within the insert vector backbone facing the inserts, so that once the inserts are released from the plasmid by the type IIS restriction enzyme, they do not contain the restriction sites. The assembly vector for Golden Gate assembly contains a negative selection marker flanked by type IIS restriction enzyme sites located in the selection marker facing the vector, so that once the selection marker is released from the vector, the vector backbone does not contain the type IIS restriction sites. These specially designed plasmids allow simple one-pot assembly of multiple DNA fragments by mixing insert plasmids and vectors together with a type IIS restriction enzyme and DNA ligase. In the Golden Gate assembly reaction, restriction digest of inserts and vector, and ligation between inserts and vector occurs in the same tube. A mixture of ligation products will be generated during the one-pot reaction when inserts and assembly vector backbone have compatible adhesive ends. This includes favorable ligation products among inserts and assembly vector backbone, as well as unfavorable ligation products such as between the negative selection marker and assembly vector backbone, between the negative selection marker and insert vector backbone, and between insert and insert vector backbone. Because the type IIS restriction sites are located within the negative selection marker and the insert vector backbone, all the unfavorable ligation products are susceptible to digestion by the type IIS restriction enzyme, while the favorable ligation products are refractory to digestion. As a result the reaction favors generation of correctly assembled ligation products among inserts and assembly vector backbone in the long term. Followed by selection using positive antibiotic selection markers within the assembly vector backbone and negative selection marker within the assembly vector insert, plasmids containing correctly assembled inserts could be selected with high efficiency.

Moclo-based systems were developed from the Golden Gate assembly system by adding a second pair type IIS restriction sites different from the enzyme used for assembly to the assembly vector in order to release the assembled DNA for next stage assembly. In Golden Gate assembly, the assembled DNA could not be released by the same restriction enzyme used for assembly, because the inserts and assembly plasmid vector backbone lack recognition sequence of the type IIS restriction enzyme used. By adding a different pair of type IIS restriction sites to release the assembled fragment, the process of Golden Gate assembly could be repeated using this second restriction enzyme and a different vector carrying a different antibiotic selection marker. Alternative sequential uses of two different type IIS restriction enzymes and antibiotic selection markers enable hierarchical assembly of large DNA fragments by one-pot restriction/ligation reaction.

A major drawback of type IIS enzyme-based DNA assembly is the necessity to remove internal type IIS restriction sites within the DNA parts to be used for assembly. A minimum of two enzymes are required for basic hierarchical assembly, and three enzymes are required for other schemes such as multi-step linear addition of DNA parts. Because most of the known type IIS restriction enzymes recognize <=6 bp sequence, most Moclo-based systems use 6 bp cutters. This requires that the DNA part must be free of two 6 bp asymmetric sequences, which occur at a frequency of ˜1 in 1 kb for a random DNA sequence with 50% GC content. A potential improvement of the current Moclo system would be to use type IIS restriction enzymes with 7 bp or more specificity. However, this option is limited by the availability of commercial type IIS restriction enzymes. Only two types of 7 bp type IIS restriction enzymes are currently commercially available: LguI/SapI which recognizes GCTCTTC and leaves 3 bp sticky end, and AarI which recognizes CACCTGC and leaves 4 bp sticky end. AarI also requires addition of oligonucleotides for complete digestion, which is undesirable for DNA assembly.

Existing type IIS restriction enzyme-based DNA assembly systems are also designed for modular DNA assembly which leave unwanted scar sequences in the final assembled DNA, and cannot be used to assemble arbitrary DNA sequence without making custom assembly vectors.

US20160002644 describes a system of DNA assembly. It uses two restriction enzymes LguI and EarI, and a strain with inducible expression of M.TaqI to block overlapping restriction sites in the assembly vector. The M.TaqI site overlaps with the 3 bp adhesive end sequence generated by LguI/EarI, which leaves severe constraints on adaptor sequence design.

A previously developed method, named MetClo, is described in WO2018203056, which is herein incorporated by reference. This system allows the use of just a single type IIS restriction enzyme for all stages in any hierarchical DNA assembly. The use of a single type IIS restriction enzyme is made possible through the creation of a type IIS restriction enzyme recognition site that can be switched on and off using site-specific enzymatic methylation. The use of just one type IIS restriction enzyme reduces the sequence constraints, but also greatly enhances the range and flexibility of the assembly schemes that can be undertaken. With MetClo however, the problem remains that the DNA fragment to be assembled needs to be free of restriction sites for the enzyme used.

What is required is an improved DNA assembly method which provides one or more of a greater freedom to choose and design adapter sequences, assembly with minimal scarring, less reaction steps or complexity, and the ability to assemble larger sequences of DNA. Therefore, an aim of the present invention is to provide an improved DNA assembly method and associated material.

According to a first aspect of the invention, there is provided a nucleic acid comprising at least one methylation-protectable restriction element, the methylation-protectable restriction element comprising:
(i) a type IIS restriction enzyme recognition sequence, or a partial type IIS restriction enzyme recognition sequence, that is recognised by a type IIS restriction enzyme that cleaves outside of the recognition sequence;
(ii) a DNA methylase recognition sequence that is recognised and methylated by a DNA methylase,
wherein the DNA methylase recognition sequence is identical to, or is encompassed within, the type IIS restriction recognition sequence, such that methylation of the nucleic acid by the DNA methylase methylates the type IIS restriction enzyme recognition sequence and protects the nucleic acid from cleavage by the type IIS restriction enzyme; and
(iii) a recognition sequence for a sequence-specific DNA-binding protein, wherein the recognition sequence is positioned such that the binding of the sequence-specific DNA-binding protein overlaps with the DNA methylase recognition sequence such that binding of the sequence-specific DNA-binding protein is capable of preventing methylation of the type IIS restriction enzyme recognition sequence by the DNA methylase such that it is not protected from cleavage by the type IIS restriction enzyme.

Advantageously, the presence of the sequence-specific DNA-binding protein protects the type IIS restriction enzyme recognition sequence from methylation, and effectively switches ON the type IIS restriction recognition sequence such that it can be cut by the type IIS restriction enzyme. In an embodiment where the sequence-specific DNA-binding protein is not present, the DNA methylase may bind to and methylate the type IIS restriction recognition sequence, such that it is protected by methylation and effectively switches OFF the type IIS restriction recognition sequence such that the type IIS restriction enzyme cannot cut the nucleic acid.

Described herein is a new method for type IIS restriction enzyme-based DNA assembly that overcomes the problem of sequence constraint and so eliminates the requirement to remove internal type IIS restriction sites from DNA parts to assemble. The method, herein termed “universal assembly”, is based on the methylation protection approach, whereby a DNA methylase is used to methylate and so block any internal restriction sites for the type IIS restriction enzyme in any DNA fragment to be assembled. In parallel, a sequence-specific DNA binding protein, such as a deactivated and programmable CRISPR Cas9, or other sequence-specific DNA binding protein, can be used to bind near to and prevent methylation of particular type IIS restriction sites of methylation-protectable restriction elements, which can be positioned in DNA vectors to flank DNA inserts or fragments to be excised. This advantageously provides control of the restriction digestion and release of the DNA insert/fragment during the assembly process.

The Type IIS Restriction Enzyme and its Recognition Sequence

In one embodiment, the type IIS restriction enzyme recognition sequence of the methylation-protectable restriction element comprises or consists of the sequence GGTCTC (i.e. a BsaI recognition sequence). In one embodiment, the DNA methylase recognition sequence comprises or consists of the sequence GGTCTC (M2.Eco31l recognition sequence). In particular, the type IIS restriction enzyme recognition sequence and the first DNA methylase of the methylation-controlled restriction enzyme recognition element may comprise or consist of the same sequence of GGTCTC (i.e. both a BsaI recognition sequence and M2.Eco31l recognition sequence).

A type IIS restriction enzyme may be a high-fidelity type IIS restriction enzyme, for example where a BsaI restriction enzyme is used it may be BsaI-HFv2, which is a high-fidelity version of BsaI.

The restriction enzyme that recognizes the restriction enzyme recognition sequence of the methylation-protectable restriction element, may be capable of cutting nucleic acid to leave at least a 2 bp overhang/sticky end. Alternatively, the restriction enzyme that recognizes the restriction enzyme recognition sequence of the methylation-protectable restriction element may be capable of cutting nucleic acid to leave at least a 3 bp overhang/sticky end. In another embodiment, the restriction enzyme that recognizes the restriction enzyme recognition sequence of the methylation-protectable restriction element may be capable of cutting nucleic acid to leave a 4 bp overhang/sticky end. In another embodiment, the restriction enzyme that recognizes the restriction enzyme recognition sequence of the methylation-protectable restriction element may be capable of cutting nucleic acid to leave a 5 bp overhang/sticky end. In another embodiment, the restriction enzyme that recognizes the restriction enzyme recognition sequence of the methylation-protectable restriction element may be capable of cutting nucleic acid to leave a 6 bp overhang/sticky end.

In one embodiment, the restriction enzyme comprises or consists of any one type IIS restriction enzyme identified herein. In another embodiment, the type IIS restriction enzyme comprises or consists of BsaI.

The Sequence-Specific DNA Binding Protein

In one embodiment, the recognition sequence for the sequence-specific DNA-binding protein overlaps with the DNA methylase recognition sequence. In one embodiment, the sequence specific DNA binding protein is a nucleic acid-guided DNA binding protein. In an alternative embodiment, the sequence specific DNA binding protein may be a second methylase, such that the recognition sequence of the DNA binding protein is a second DNA methylase recognition sequence relative to the first DNA methylase recognition sequence of ii). In one embodiment, methylation by the second methylase does not block the type IIS restriction enzyme recognition sequence. For example, the type IIS restriction enzyme may still recognise and cut the nucleic acid in the presence of the second DNA methylase or in the event of methylation of the nucleic acid by the second DNA methylase. Alternatively, the sequence specific DNA binding protein may not be a methylase.

Advantageously, the invention provides different methods for blocking the methylation of the type IIS restriction enzyme recognition sequence of the methylation-protectable restriction element via the design of the recognition sequence of the sequence specific DNA binding protein. In particular, the sequence specific DNA binding protein can be a nucleic acid-guided DNA binding protein, which would engage with the recognition sequence of the sequence specific DNA binding protein within the methylation-protectable restriction element and block the DNA methylase from methylating the type IIS restriction enzyme recognition sequence. In an alternative embodiment, the sequence specific DNA binding protein can be a second DNA methylase, which would engage with the second DNA methylase recognition sequence of the methylase-protectable restriction element and block the first DNA methylase from methylating the type IIS restriction enzyme recognition sequence. In another embodiment, a similar effect can be achieved by a sequence that is recognised by an alternative sequence specific DNA binding protein.

Without being bound by theory, it is understood that the blocking by the sequence specific DNA binding protein may rely on steric hindrance to block the DNA methylase (of part ii) from appropriately docking and methylating the type IIS restriction enzyme recognition sequence. Once the type IIS restriction enzyme recognition sequence is free from methylation (due to the blocking of the DNA methylase), it is free to be cut by the type IIS restriction enzyme. In contrast any non-protectable or non-switchable type IIS restriction enzyme recognition sequences that are present in the nucleic acid molecule (for example by random chance) would be methylated by the same DNA methylase that recognises such a sequence, and thereby protected from cutting, because there would be no sequence specific DNA binding protein recognition sequence provided to control the methylation.

The provision of a sequence specific DNA binding protein, such as a nucleic acid-guided DNA binding protein, can be controlled by cloning the nucleic acid in strains that express the sequence specific DNA binding protein (and any associated guide nucleic acid if required). Alternatively, if the type IIS restriction enzyme recognition sequence is to be protected/blocked from cutting then the strain used to clone the nucleic acid may express the DNA methylase (i.e. the first DNA methylase) and not express the sequence specific DNA binding protein and/or not express any guide nucleic acid for such a sequence specific DNA binding protein. The strain used to clone the nucleic acid may express an alternative sequence specific DNA binding protein and/or guide nucleic acid for such a sequence specific DNA binding protein, which may be directed to target an alternative sequence/methylation-protectable restriction element.

Alternatively, the provision of a sequence specific DNA binding protein, such as a nucleic acid-guided DNA binding protein, can be controlled in vitro by adding the the sequence specific DNA binding protein (and any associated guide nucleic acid if required) to the nucleic acid, e.g. in vitro. Alternatively, if the type IIS restriction enzyme recognition sequence is to be protected/blocked from cutting then the DNA methylase (i.e. the first DNA methylase) may be provided in vitro and the sequence specific DNA binding protein (and any associated guide nucleic acid if required) may not be provided.

In one embodiment of the invention, the nucleic acid is complexed with (i.e. bound by) the DNA methylase (i.e. the first DNA methylase) and/or the sequence specific DNA binding protein (and any associated guide nucleic acid if required). Therefore, according to another aspect of the invention, there is provided a nucleic acid-protein complex comprising the nucleic acid according to the invention and a sequence specific DNA binding protein that is arranged to bind to the associated sequence specific DNA binding protein recognition sequence in the nucleic acid.

Where the sequence specific DNA binding protein requires a guide nucleic acid, such a guide nucleic acid may also be provided in the complex.

The complex may further be complexed with the DNA methylase of part ii (i.e. the first DNA methylase). The complex may be in vitro (e.g. not within a cell, such as a bacterium). The complex may be in a buffer solution. Alternatively, the complex may be in vivo (e.g. within a cell, such as a bacterial strain described herein).

The complex may further be complexed with the type IIS restriction enzyme that recognises the type IIS restriction enzyme recognition sequence of part (i) of the nucleic acid.

The recognition sequence for the sequence-specific DNA-binding protein may be a predetermined sequence. The recognition sequence for the sequence-specific DNA-binding protein may be a sequence designed to be complementary to the guide nucleic acid of a nucleic acid-guided DNA binding protein, such as dCas9. The guide nucleic acid may be pre-designed/pre-determined. In an alternative embodiment, the recognition sequence for the sequence-specific DNA-binding protein may be a sequence designed to be recognised by a particular sequence-specific DNA-binding protein, such as a methylase.

In one embodiment, the sequence specific DNA binding protein may comprise or consist of a Transcription activator-like effector (TALE or otherwise known as TAL effector). The sequence specific DNA binding protein recognition sequence may be a TALE recognition sequence. The TALE recognition sequence may be wild-type (e.g. a sequence that wild-type TALE recognises) or an engineered sequence to correspond with an engineered TALE sequence specificity.

The skilled person will recognise that the simple correspondence between amino acids in TAL effectors and DNA bases in their target sites makes them useful for protein engineering applications. Numerous groups have designed artificial TAL effectors capable of recognizing new DNA sequences in a variety of experimental systems (Zhang et al. 2011. Nature Biotechnology. 29 (2): 149-53. doi:10.1038/nbt.1775. PMC 3084533. PMID 21248753).

In another embodiment, the sequence specific DNA binding protein may comprise or consist of a deactivated endonuclease. The sequence specific DNA binding protein may comprise or consist of a deactivated endodeoxyribonuclease, such as a deactivated meganuclease or a deactivated restriction enzyme. The sequence specific DNA binding protein recognition sequence may be a meganuclease recognition sequence. The meganuclease recognition sequence may be wild-type (e.g. a sequence that a wild-type meganuclease recognises) or an engineered sequence to correspond with an engineered meganuclease sequence specificity. The deactivation of the endonuclease may refer to the deactivation of the cleavage activity (i.e. such that it does not cut). The ability to deactivate such enzymes is now routine, and the skilled person will be familiar with standard techniques to modify (e.g. mutate) an endonuclease such that it retains sequence specific binding, but loses its cleaving activity.

In another embodiment, the sequence specific DNA binding protein may comprise or consist of a sequence specific zinc finger protein. The sequence specific DNA binding protein recognition sequence may be a zinc finger protein recognition sequence. The zinc finger protein recognition sequence may be wild-type (e.g. a sequence that a wild-type zinc finger protein recognises) or an engineered sequence to correspond with an engineered zinc finger protein sequence specificity.

The skilled person will recognise that the basic principle of the method of the invention is to use a sequence specific DNA binding protein to block the methylation of DNA by a DNA methylase at specific sites. Therefore, any suitable DNA binding protein with enough high affinity specific DNA binding activity that is enough to compete with methylase DNA binding affinity may be used.

In one embodiment, the sequence specific DNA binding protein may be a sequence-specific deactivated restriction enzyme (e.g. capable of sequence specific binding, but not cutting).

In one embodiment, the sequence specific DNA binding protein is a methylase. In one embodiment, the recognition sequence of the sequence specific DNA binding protein is a second DNA methylase recognition sequence. The second DNA methylase recognition sequence may be different from the first DNA methylase recognition sequence. The second DNA methylase recognition sequence may be recognised by a second DNA methylase. The second DNA methylase may be different to the first DNA methylase. In an embodiment wherein the sequence specific DNA binding protein is a methylase, the methylase may be a deactivated methylase (i.e. does not methylate) or an active methylase).

In one embodiment, the second DNA methylase recognition sequence is recognised by M.Csp205I. In one embodiment, the second DNA methylase is M.Csp205I. The second DNA methylase recognition sequence may comprise or consist of the sequence TTCANNNNNNNNCTC (SEQ ID NO: 1).

In one embodiment, the methylation-protectable restriction element comprises the sequence TTCANNNNNGGTCTCNNNNN (M.Csp205I/BsaI/M2.Eco31I—SEQ ID NO: 2).

The Nucleic Acid-Guided DNA Binding Protein

In one embodiment, the sequence specific DNA binding protein is a nucleic acid-guided DNA binding protein. For example a nucleic acid-guided DNA binding protein recognition sequence could be provided, such as a dCas9 recognition sequence. The sequence specific DNA binding protein would engage with the sequence specific DNA binding protein recognition sequence of the methylation-protectable restriction element and block the DNA methylase from methylating the type IIS restriction enzyme recognition sequence.

The nucleic acid-guided DNA binding protein may be a RNA-guided DNA binding protein. The nucleic acid-guided DNA binding protein may be a nucleic acid-guided DNA endonuclease enzyme, such as a RNA-guided DNA endonuclease enzyme. Preferably the endonuclease enzyme activity is deactivated, for example by mutation. In one embodiment, the nucleic acid-guided DNA binding protein comprises Cas9 (CRISPR associated protein 9). The Cas9 may be deactivated, such that the Cas9 may bind to the DNA, but does not function to cut the DNA. The Cas9 may be a mutation-deactivated Cas9 (herein also termed dCas9). The mutations for dCas9 that inactivate Cas9 nuclease activity are described in PMID 22745249 (Science. 2012 Aug. 17; 337(6096):816-21. doi: 10.1126/science.1225829. Epub 2012 Jun. 28), which is incorporated herein by reference. The use of Cas9 with double mutations (D10A and H840A) as a tool for interfering with transcription was first described in PMID 23452860. (Cell. 2013 Feb. 28; 152(5):1173-83. doi: 10.1016/j.cell.2013.02.022), which is incorporated herein by reference. In one embodiment, the Cas9 mutations may be D10 and/or H840, such as D10A and/or H840A.

The skilled person will understand that the recognition sequence of the nucleic acid-guided DNA binding protein may be designed to be any appropriate sequence. For example, the nucleic acid sequence of the nucleic acid-guided DNA binding protein may be of any appropriate sequence, and the nucleic acid-guided DNA binding protein recognition sequence may be designed to be complimentary to the guiding nucleic acid.

Advantageously, the use of a nucleic acid-guided DNA binding protein allows several methylation protection systems to be designed with different guide nucleic acid (such as RNA) sequences, which effectively eliminates the need to remove internal forbidden sequences and so allows DNA assembly without sequence constraint.

The DNA binding sequence of deactivated Cas9 comprises a PAM sequence (NGG) at 3′ of the programmable guide RNA recognition sequence (e.g. any 20 base pair sequence). The DNA binding specificity guided by the guide RNA may be the last 5 base pair of the guide RNA recognition sequence. For example, if the guide RNA recognition sequence is NNNNNNNNNNNNNNNabcde, then the DNA binding sequence may be NNNNNNNNNNNNNNNabcdeNGG, and the DNA binding specificity is determined by 7 underlined bases.

The dCas9 may be derived from Cas9 from Streptococcus pyogenes (SpCas9). The nucleic acid-guided DNA binding protein may be from other families of RNA-guided CRISPR molecules. The nucleic acid-guided DNA binding protein may comprise or consist of Cas9 from the Type II CRISPR system.

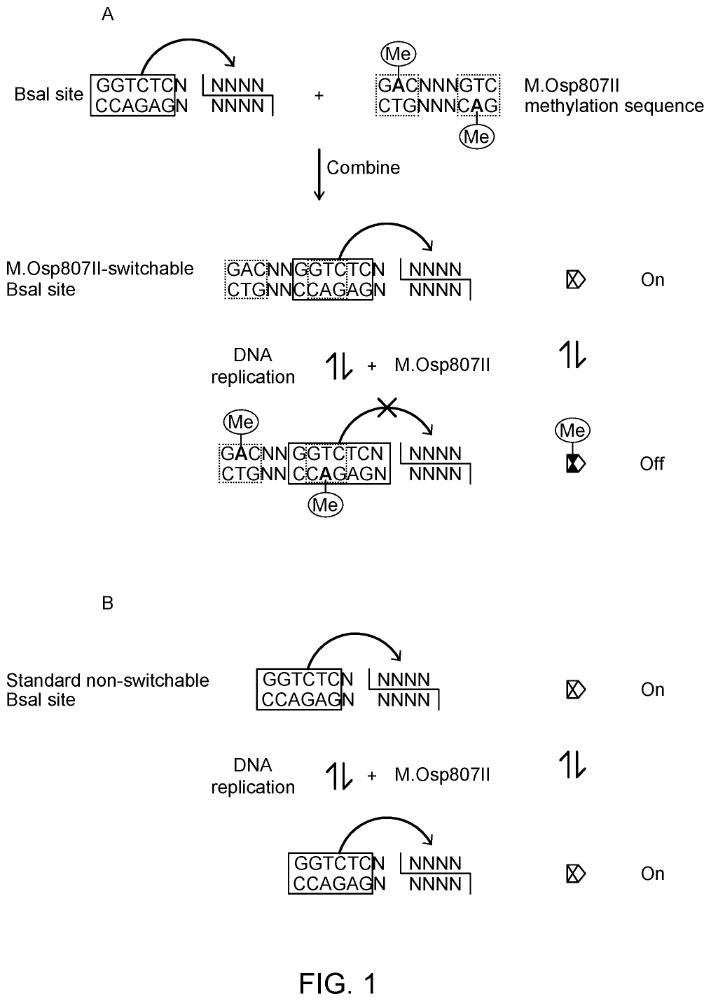

In one embodiment, the nucleic acid-guided DNA binding protein may comprise or consist of a Cas9 molecule selected from Spy (e.g. from S. pyogenes); Sth3 (e.g. from S. thermophilus—CRISPR3); Sau (e.g. from S. aureus); Sth1 (e.g. from S. thermophilus—CRISPR1); Spa (e.g. from S. pasteurianus); Cla (e.g. from C. lari); Cje (e.g. from C. jejuni); Nme (e.g. from N. meningitidis); Pmu (e.g. P. multocida); Pla (e.g. from P. lavamentivorans) and Cdi (e.g. from C. diphtheria), (e.g. any of Cas9 molecules listed in Rock J M et al. Nat Microbiol. 2017 Feb. 6; 2:16274. doi: 10.1038/nmicrobiol.2016.274 PMID 28165460 FIG. 1b); or a variant or homologue thereof. A variant may be a deactivated variant of such Cas9 molecules.

In another embodiment, the nucleic acid-guided DNA binding protein may comprise or consist of Cas12a/Cpf1, or a variant or homologue thereof (e.g. as described in Zetsche et al., 2015. Cell.; 163(3):759-71. doi: 10.1016/j.cell.2015.09.038 (PMID 26422227)). A variant may be a deactivated variant of such Cas12a/Cpf1.

In another embodiment, the nucleic acid-guided DNA binding protein may comprise or consist of the cascade complex, for example from the Type I-E CRISPR-Cas system in E. coli(Luo et al., 2015. Nucleic Acids Res. 43(1): 674-681 (PMID 25326321)).

In another embodiment, the nucleic acid-guided DNA binding protein may comprise or consist of CasX, or a variant or homologue thereof (e.g. as described in Liu et al., Nature. 2019 February; 566(7743):218-223, which is herein incorporated by reference).

In another embodiment, the nucleic acid-guided DNA binding protein may comprise or consist of dST1Cas9 from the Streptococcus thermophiles CRISPR1-Cas9 system. dST1Cas9 (also known as dCas9_{Sth1}) is a variant of Cas9 from the CRISPR1 locus of Streptococcus thermophiles, carrying deactivating point mutations (D9A, H599A). In another embodiment, the he nucleic acid-guided DNA binding protein may comprise or consist of Cas9_{Sth1}, which may be modified to inactivate the nuclease activity, for example by one or more point mutations. The one or more point mutations may comprise or consist of D9A and/or H599A.

In one embodiment, one or more bases of the PAM motif (protospacer adjacent motif) in the nucleic acid-guided DNA binding protein (e.g. Cas9 or variants thereof) recognition sequence overlaps with one or more base pairs of the restriction enzyme restriction site, to form a methylation protectable restriction enzyme site. In another embodiment, the last base of the PAM motif in the nucleic acid-guided DNA binding protein (e.g. Cas9 or variants thereof) recognition sequence overlaps with the first base pair of the restriction enzyme restriction site, to form a methylation protectable restriction enzyme site.

The skilled person will recognise that any CRISPR mechanisms that can be used for CRISPR interference to block transcription can also be used for CRISPR interference of DNA methylation.

In one embodiment, the methylation-protectable restriction element comprises the sequence

(SEQ ID NO: 3)
NNNNNNNNNNNNNNNNNNNNNGGTCTCNNNNN

(dCas9/BsaI/M2.Eco31I). The underlined nucleotides (including the double underlined nucleotides) may correspond to the guide nucleic acid sequence, for example of dCas9. The double underlined nucleotides may be the 5 bp seed sequence that is critical for specificity of dCas9.

In another embodiment, the methylation-protectable restriction element comprises the sequence

(SEQ ID NO: 107)
NNNNNNNNNNNNNNNNNNNN NNAGAAGGTCTC.

(dST1Cas9/BsaI/M2.Eco31I). The underlined nucleotides may correspond to the guide nucleic acid sequence, for example of dST1Cas9.

In another embodiment, the methylation-protectable restriction element comprises the sequence

(SEQ ID NO: 108)
GTCTAGTATGCGGATGCCAGTGAGAAGGTCTC.

In another embodiment, the methylation-protectable restriction element comprises the sequence

(SEQ ID NO: 109)
GAGACCNNNNNNNNNNNNNNNNAGAAG.

(dST1Cas9/BsaI/M2.Eco31I). The underlined nucleotides may correspond to the guide nucleic acid sequence, for example of dST1Cas9.

In another embodiment, the methylation-protectable restriction element comprises the sequence CTTCTNNNNNNNNNNNNNNNNGGTCTC (SEQ ID NO: 110). In another embodiment, the methylation-protectable restriction element comprises the sequence CTTCTCAAGCCCCAGAATGGTGGTCTC (SEQ ID NO: 111).

The skilled person will recognise that the methylation-protectable restriction element may alternatively comprise or consist of the reverse compliment sequence of the methylation-protectable restriction element sequences described herein.

A Methylase-Switch Element

The methylation-protectable restriction element may further comprise a methylase-switch element, wherein the methylase-switch element comprises a recognition sequence for a DNA methylase, herein termed a “switch DNA methylase”. The type IIS restriction enzyme recognition sequence (i) of the methylation-protectable restriction element may also be part of the methylase-switch element. The recognition sequence for the switch DNA methylase of the methylation switch element may be 5′ upstream of, and partially overlap with, the recognition sequence of the type IIS restriction enzyme of the methylation-protectable restriction element.

In one embodiment, the type IIS restriction enzyme recognition sequence and the switch DNA methylase recognition sequence of the methylase-switch element overlap such that the base modified by the switch DNA methylase of the methylase-switch element lies within the type IIS restriction enzyme recognition sequence such that methylation or binding by the switch DNA methylase may block/impair the overlapping type IIS restriction enzyme recognition site.

The switch DNA methylase recognition sequence of the methylase-switch element may not be identical to, or enclosed by, the type IIS restriction enzyme recognition sequence. Additionally, the switch DNA methylase recognition sequence of the methylase-switch element may not overlap with the sequence that would form the overhang end sequence generated by the type IIS restriction enzyme.

Advantageously, the methylase-switch element introduces a further switchable control element that is capable of switching ON and OFF the ability of the type IIS restriction enzyme to cut the nucleic acid by methylation of the type IIS restriction enzyme recognition sequence.

Therefore, in one embodiment, the methylation-protectable restriction element may comprise:
(i) a type IIS restriction enzyme recognition sequence that is recognised by a type IIS restriction enzyme that cleaves outside of the recognition sequence;
(ii) a first DNA methylase recognition sequence that is recognised and methylated by a first DNA methylase,
wherein the first DNA methylase recognition sequence is identical to, or is encompassed within, the type IIS restriction recognition sequence, such that methylation of the nucleic acid by the first DNA methylase methylates the type IIS restriction enzyme recognition sequence and protects the nucleic acid from cleavage by the type IIS restriction enzyme;
(iii) a recognition sequence for a sequence-specific DNA-binding protein, wherein the recognition sequence is 5′ upstream of the first DNA methylase recognition sequence (ii) such that binding of the sequence-specific DNA-binding protein is capable of preventing methylation of the type IIS restriction enzyme recognition sequence of (i) by the first DNA methylase of (ii) such that it is not protected from cleavage by the type IIS restriction enzyme of (i); and
(iv) a methylase-switch element, wherein the methylase-switch element comprises a recognition sequence for a switch DNA methylase,
wherein the type IIS restriction enzyme recognition sequence of (i) and the switch DNA methylase recognition sequence of the methylase-switch element overlap such that the base modified by the switch DNA methylase of the methylase-switch element lies within the type IIS restriction enzyme recognition sequence of (i) such that methylation by the switch DNA methylase of the methylase-switch element blocks/impairs the overlapping type IIS restriction enzyme recognition sequence (i),
   wherein the switch DNA methylase recognition sequence of the methylase-switch element is not be identical to, or enclosed by, the type IIS restriction enzyme recognition sequence of (i), and
   wherein the switch DNA methylase recognition sequence of the methylase-switch element does not overlap with the sequence that would form the overhang end sequence generated by the type IIS restriction enzyme of (i).

The switch DNA methylase recognition sequence of the methylase-switch element may be 5′ upstream of the type IIS restriction enzyme recognition sequence, and partially overlap therewith.

In one embodiment, the switch DNA methylase of the methylase-switch element comprises a type II DNA methylase. In another embodiment, the switch DNA methylase of the methylase-switch element comprises a type I DNA methylase. In one embodiment, the DNA methylase of the methylase-switch element comprises a single domain DNA methylase without restriction enzyme activity, or a modified DNA methylase having a non-functional restriction enzyme activity. For example, the modification may comprise modifying the N6 position of methyladenine. In one embodiment, the switch DNA methylase recognition sequence of the methylase-switch element comprises at least 4 base pairs. The DNA methylase of the methylase-switch element may be active, such as optimally active, at about 37° C.

The type IIS restriction enzyme recognition sequence of the methylation-protectable restriction element may be methylated, for example by the switch DNA methylase of the methylase-switch element that recognises the switch DNA methylase recognition sequence of the methylase-switch element. The switch DNA methylase of the methylase-switch element that recognises the switch DNA methylase recognition sequence of the methylase-switch element may be arranged to methylate a nucleotide within the sequence of the restriction enzyme recognition sequence of the methylation-protectable restriction element. The methylation may be capable of blocking the cutting of the DNA by the type IIS restriction enzyme that recognises the type IIS restriction enzyme recognition sequence of the methylation-protectable restriction element. In one embodiment, the type IIS restriction enzyme recognition sequence of the methylation-protectable restriction element may become non-functional as a result of methylation of at least one of the nucleotides in the sequence. In one embodiment, the methylated nucleotide may be an adenine, or the nucleotide arranged to be methylated may be an adenine. In another embodiment, the methylated nucleotide may be a cytosine, or the nucleotide arranged to be methylated may be a cytosine. The methylation may be any one of methylation types selected from N4-methylcytosine (m4C), C5-methylcytosine (m5C) or N6-methyladenine (m6A). The skilled person will recognize that the type of methylation provided should block/impair the overlapping restriction site function. Additionally, for in vivo methylation, the strain used to express the switch DNA methylase of the methylase-switch element may be deficient for modification-dependent restriction enzymes that may recognize the methylated bases, such as Mcr/Mrr family restriction enzymes. In one embodiment, the methylation type may comprise N6-methyladenine (m6A).

In one embodiment, the DNA methylase of the methylase-switch element may or may not methylate the outer-most bases of the restriction enzyme recognition sequence. For example in a restriction enzyme recognition sequence of 6 bp, the bases 1 or 6 may not be methylated. In one embodiment, the switch DNA methylase of the methylase-switch element may or may not methylate the 2″ base of the restriction enzyme recognition sequence. In one embodiment, the methylase may methylate position 3, 4, or 5 for 6 bp restriction enzyme recognition sites. In one embodiment, the switch DNA methylase of the methylase-switch element may methylate position 3, 4, 5, or 6 for restriction enzyme recognition sites.

In one embodiment, the switch DNA methylase for the methylase-switch element comprises or consists of any one type II DNA methylase identified herein. In another embodiment, the switch DNA methylase for the methylase-switch element comprises or consists of M.Osp807II. In another embodiment, the switch DNA methylase for the methylase-switch element comprises or consists of M.Sen0738I.

In one embodiment, the restriction enzyme and the switch DNA methylase for the methylase-switch element comprise or consist of any one pairing of type IIS restriction enzymes and the type II DNA methylases identified herein. In one embodiment, the restriction enzyme recognition sequence and the switch DNA methylase recognition sequence of the methylase-switch element comprise or consist of any one pairing of type IIS restriction enzyme recognition sequences and the type II DNA methylase recognition sequences identified herein. In one embodiment, the type IIS restriction enzyme and the switch DNA methylase for the methylase-switch element comprise or consist of the pairings BsaI/M.Osp807II. In one embodiment, the type IIS restriction enzyme and the switch DNA methylase for the methylase-switch element comprise or consist of the pairings BsaI/M.Sen0738I.

In one embodiment, the switch DNA methylase recognition sequence of the methylase-switch element comprises or consists of the sequence GACNNNGTC (M.Osp807II).

In one embodiment, the methylation-protectable restriction element comprises the sequence GACNNGGTCTCNNNNN (BsaI/M.Osp807II—SEQ ID NO: 4). In one embodiment, the methylation-protectable restriction element comprises or consists of the sequence TTCAGACNNGGTCTCNNNNN (M.Csp205I/BsaI/M2.Eco31I/M.Osp807II—SEQ ID NO: 5).

In another embodiment, the switch DNA methylase recognition sequence of the methylase-switch element comprises or consists of the sequence CCAGNNNNNNNNTCT (M.Sen0738I—SEQ ID NO: 6) (the complement thereof is AGANNNNNNNNCTGG (SEQ ID NO: 7)).

In one embodiment, the methylation-protectable restriction element comprises the sequence CCAGNNNNNNGGTCTC (BsaI/M.Sen0738I—SEQ ID NO: 8).

In one embodiment, the methylation-protectable restriction element comprises the sequence

(SEQ ID NO: 9)
NNNNNNNNNNNNNNNNGACNNGGTCTCNNNNN

(dCas9/BsaI/M2.Eco31I/M.Osp807II). In one embodiment, the methylation-protectable restriction element comprises the sequence

(SEQ ID NO: 10)
NNNNNNNNNNNCCAGNNNNNNGGTCTCNNNNN

(dCas9/BsaI/M2.Eco31I/M.Sen0738I). The underlined nucleotides (including the double underlined nucleotides) may correspond to the guide nucleic acid sequence, for example of dCas9. The double underlined nucleotides may be the 5 bp seed sequence that is critical for specificity of dCas9.

In one embodiment, the methylation-protectable restriction element comprises the sequence

(SEQ ID NO: 125)
NNNNNNNNNNNNNNNNCCAGNNAGAAGGTCTCNNNNN

(dST1Cas9/BsaI/M2.Eco31I/M.Sen0738I). The underlined nucleotides may correspond to the guide nucleic acid sequence, for example of dST1Cas9.

In another embodiment, the methylation-protectable restriction element comprises the sequence

(SEQ ID NO: 126)
CTTCTNNNNNNCCAGNNNNNNGGTCTC.

(dST1Cas9/BsaI/M2.Eco32I/M.Sen0738I). The underlined nucleotides may correspond to the reverse complement sequence of the guide nucleic acid sequence, for example of dST1Cas9.

The skilled person will recognise that the methylation-protectable restriction element may alternatively comprise or consist of the reverse complement sequence of the methylation-protectable restriction element sequences described herein.

Head-to-Head/Opposing Restriction Sites

In one embodiment, the nucleic acid comprising at least one methylation-protectable restriction element further comprises a non-switchable type IIS restriction enzyme recognition sequence. The non-switchable type IIS restriction enzyme recognition sequence may be opposing the type IIS restriction enzyme recognition sequence (i) of the methylation-protectable restriction element. In one embodiment, the non-switchable type IIS restriction enzyme recognition sequence is provided on the opposing side of the cut site of the methylation-protectable restriction element. Such an arrangement may also be termed a “head to head” arrangement, for example where the cut site is flanked by opposing switchable and non-switchable/non-switchable type IIS restriction enzyme recognition sequences. By “opposing” it is intended to mean that the type IIS restriction enzyme recognitions sequences are in a different 5′ to 3′direction relative to each other (e.g. they may be symmetrical) such that their respective cut sites are directed towards each other.

The opposing non-switchable restriction enzyme recognition sequence may also be recognized by a type IIS restriction enzyme. The opposing non-switchable restriction enzyme recognition sequence may not be protected by (or arranged to be protected by) methylation, or at least may not be protected by methylation by the same switch DNA methylase that recognizes the switch DNA methylase recognition sequence of the methylase-switch element. Such an opposing non-switchable restriction enzyme recognition sequence may be provided in a discard sequence (a fragment to be excised and discarded from a vector). Such an opposing non-switchable restriction enzyme recognition sequence may otherwise be referred to as an “inner restriction enzyme recognition sequence”, whereas the type IIS restriction enzyme recognition sequence of the methylation-protectable restriction element may be referred to as an “outer restriction enzyme recognition sequence”.

The type IIS restriction enzyme recognition sequence of the methylation-protectable restriction element may be recognised by the same restriction enzyme species as the opposing non-switchable restriction enzyme recognition sequence. The type IIS restriction enzyme recognition sequence of the methylation-protectable restriction element may comprise the same sequence as the opposing non-switchable restriction enzyme recognition sequence.

In one embodiment, the opposing non-switchable restriction enzyme recognition sequence is not capable of being blocked by methylation by the same switch DNA methylase that recognises the switch DNA methylase recognition sequence of the methylase-switch element. In one embodiment, the switch DNA methylase that recognises the switch DNA methylase recognition sequence of the methylase-switch element may not recognise a sequence within or overlapping with the opposing non-switchable restriction enzyme recognition sequence. For example, the non-switchable opposing restriction enzyme recognition sequence may not overlap with or comprise a sequence that is recognised by the switch DNA methylase that recognises switch DNA methylase recognition sequence of the methylase-switch element.

In one embodiment, the opposing non-switchable restriction enzyme recognition sequence is a BsaI recognition sequence. The type IIS restriction enzyme recognition sequence of the methylation-protectable restriction element and the non-switchable restriction enzyme recognition sequence may be a BsaI recognition sequence (i.e. there are two BsaI sequences in this embodiment).

In an alternative embodiment, the nucleic acid comprising at least one methylation-protectable restriction element comprises an opposing methylation-protectable restriction element. In particular, the type IIS restriction enzyme recognition sequence (i) of one methylation-protectable restriction element may be opposing the type IIS restriction enzyme recognition sequence (i) of the other opposing methylation-protectable restriction element. In one embodiment, the opposing methylation-protectable restriction element is provided on the opposing side of the cut site of the first methylation-protectable restriction element. Such an arrangement may also be termed a “head to head” arrangement, for example where the cut site is flanked by opposing methylation-protectable type IIS restriction enzyme recognition sequences. By “opposing” it is intended to mean that the type IIS restriction enzyme recognitions sequences are in a different 5′ to 3′direction relative to each other (e.g. they may be symmetrical) such that their respective cut sites are directed towards each other. A first methylation-protectable restriction element may be termed an “outside methylation-protectable restriction element”, and the second opposing methylation-protectable restriction element may be termed an “inside methylation-protectable restriction element”, as the inside methylation-protectable restriction element may be in a discard sequence or DNA fragment to be excised from a vector, and the outside methylation-protectable restriction element may be arranged to be retained in the vector backbone.

The type IIS restriction enzyme recognition sequences of the first methylation-protectable restriction element and opposing methylation-protectable restriction element may be the same, such as a BsaI recognition sequence. The type IIS restriction enzyme recognition sequences of the first methylation-protectable restriction element and opposing methylation-protectable restriction element may be arranged to cut at the same site (i.e. leave identical overhangs).

The type IIS restriction enzyme recognition sequences of the first methylation-protectable restriction element and opposing methylation-protectable restriction element may comprise different sequence specific DNA binding protein recognition sequences relative to each other. Advantageously this allows independent control of the methylase-protection by different sequence specific DNA binding proteins. For example, in an embodiment using a nucleic acid guided sequence specific DNA binding protein, such as dCas9, the guide nucleic acid, such as RNA, may be different in sequence.

Maintenance-Type Design of Head-to-Head/Opposing Restriction Sites

In an embodiment wherein the nucleic acid comprises a non-switchable type IIS restriction enzyme recognition sequence opposing the methylation-protectable restriction element (i.e. a head to head arrangement) the opposing non-switchable type IIS restriction enzyme recognition sequence may be arranged to direct the type IIS restriction enzyme to cut the nucleic acid at the same site as the type IIS restriction enzyme recognition sequence of the methylation-protectable restriction element, such that the same overhang/sticky end would be produced at the same position. In an alternative embodiment wherein the nucleic acid comprises a first/outside methylation-protectable restriction element and a second/inside opposing methylation-protectable restriction element (i.e. a head to head arrangement) the opposing type IIS restriction enzyme recognition sequence of the second/inside opposing methylation-protectable restriction element may be arranged to direct the type IIS restriction enzyme to cut the nucleic acid at the same site as the type IIS restriction enzyme recognition sequence of the first/outside methylation-protectable restriction element, such that the same overhang/sticky end would be produced at the same position.

The same overhang/sticky end may be achieved by providing the type IIS restriction enzyme recognition sequence (i.e. of (i)) and the opposing type IIS restriction enzyme recognition sequence at a specific distance apart, depending on the cut site provided by the type IIS restriction enzyme(s) selected. Such an arrangement may be termed herein as a “maintenance-type design element” where the same overhang is produced regardless of which of the type IIS restriction enzyme recognition sequences directs the cut to the nucleic acid.

For example, in an embodiment wherein the type IIS restriction enzyme recognition sequence of the methylation-protectable restriction element and the non-switchable restriction enzyme recognition sequence are BsaI recognition sequences, the opposing BsaI sequences may be distanced apart by 6 base pairs (i.e. 6 bp in the gap between the end of one recognition sequence and the start of the opposing recognition sequence). The skilled person will recognise that such distance depends on the property of the restriction enzyme(s) selected. For example, assuming the enzyme cuts x bases from the recognition sequence and generates y bases of adhesive end/overhang (e.g. for BsaI x=1 and y=4), then the distance (d) for the number of bases between opposing restriction enzyme recognition sequences of the maintenance-type design element may be provided by the following equation: d=(2*x)+y (e.g. d=6 bp for BsaI). The distance (d) for the maintenance-type design element may range from 5 bp (e.g. when using EarI) to 24 bp (e.g. when using BtgZI). In one embodiment, the difference between the opposing restriction enzyme recognition sequences of the maintenance-type design element is d, wherein d=(2*x)+y, and wherein x is the number of base pairs the cut site of a given restriction enzyme is from its recognition sequence, and y is the length of the overhang that would be produced by the restriction enzyme. The same examples and calculations can equally apply to the positioning/distance of opposing type IIS restriction enzyme recognition sequences in embodiments comprising a first/outside methylation-protectable restriction element and a second/inside opposing methylation-protectable restriction element.

In one embodiment, for example involving a maintenance-type design element, the nucleic acid may comprise the sequence of GACNNGGTCTCNNNNNNGAGACC (SEQ ID NO: 11) (BsaI/M2.Eco31I/M.Osp807II and opposing BsaI/M2.Eco31I). In one embodiment involving a maintenance-type design element, the nucleic acid may comprise the sequence of CCAGNNNNNNGGTCTCNNNNNNGAGACC (SEQ ID NO: 12) (BsaI/M2.Eco31I/M.Sen0738I and opposing BsaI/M2.Eco31I). In one embodiment, for example involving a maintenance-type design element, the nucleic acid may comprise or consist of the sequence of TTCANNNNNGGTCTCNNNNNNGAGACC (SEQ ID NO: 13) (M.Csp205I/BsaI/M2.Eco31I and opposing BsaI/M2.Eco31I). In one embodiment, for example involving a maintenance-type design element, the nucleic acid may comprise or consist of the sequence of TTCAGACNNGGTCTCNNNNNNGAGACC (SEQ ID NO: 14) (M.Csp205I/BsaI/M2.Eco31I/M.Osp807II and opposing BsaI/M2.Eco31I). N may mean A, C, T or G. Other sequences may be provided according to the combined/overlapping restriction enzyme and methylase recognition sequences provided herein, wherein following the series of N bases, the sequence further comprises the palindromic/reverse-complement sequence of the restriction enzyme recognition sequence.

Excision-Type Design of Head-to-Head/Opposing Restriction Sites

In another embodiment, it may not be desirable to produce the same overhang from cutting with either type IIS restriction enzyme that recognises the type IIS restriction enzyme recognition sequence of the opposing methylation-protectable restriction element and the non-switchable restriction enzyme recognition sequence, or the opposing type IIS restriction enzyme recognition sequences in embodiments comprising a first/outside methylation-protectable restriction element and a second/inside opposing methylation-protectable restriction element. Therefore, the opposing type IIS restriction enzyme recognition sequences may be positioned closer together (i.e. relative to the maintenance-type design element). Such an embodiment may also be referred to as an “excision-type design element”. For example, the opposing non-switchable type IIS restriction enzyme recognition sequence may be arranged to direct the type IIS restriction enzyme to cut the nucleic acid at a site further towards the type IIS restriction enzyme recognition sequence of the methylation-protectable restriction element such that the cut site does not overlap the cut site of the type IIS restriction enzyme that recognizes the type IIS restriction enzyme recognition sequence of the methylation-protectable restriction element (i.e. such that the position of the subsequent overhangs created by the opposing cut sites do not overlap, and the subsequent overhangs may be different in sequence). In another example, the type IIS restriction enzyme recognition sequence of a second/inside opposing methylation-protectable restriction element may be arranged to direct the type IIS restriction enzyme to cut the nucleic acid at a site further towards the type IIS restriction enzyme recognition sequence of the first/outside methylation-protectable restriction element such that the cut site does not overlap the cut site of the type IIS restriction enzyme that recognizes the type IIS restriction enzyme recognition sequence of the first/outside methylation-protectable restriction element (i.e. such that the position of the subsequent overhangs created by the opposing cut sites do not overlap, and the subsequent overhangs may be different in sequence).

The cut site of the opposing non-switchable restriction enzyme recognition sequence in an excision-type design element may be within the recognition sequence of the type IIS restriction enzyme recognition sequence of the methylation-protectable restriction element, and/or within the nucleotides between the cut site and the recognition sequence of the type IIS restriction enzyme recognition sequence of the methylation-protectable restriction element. In another embodiment, the cut site of the type II restriction enzyme recognition sequence of the second/inside opposing methylation-protectable restriction element may be within the recognition sequence of the type IIS restriction enzyme recognition sequence of the first/outside methylation-protectable restriction element, and/or within the nucleotides between the cut site and the recognition sequence of the type IIS restriction enzyme recognition sequence of the first/outside methylation-protectable restriction element.

In one embodiment, the cleavage point between base pairs at the end of one cut site and the cleavage point between base pairs at the start of the opposing cut site may be in adjacent/neighbouring base pairs (i.e. the cut sites may be immediately adjacent to each other, but do not overlap). This may be achieved by providing the opposing type IIS restriction enzyme recognition sequences at a specific distance apart, depending on the cut site provided by the type IIS restriction enzyme(s) selected. The distance will be close enough to cause cutting in the opposing type IIS restriction enzyme recognition sequence and/or nucleotides between the recognition sequence and cut site, or vice versa (i.e. closer than the opposing type II restriction enzyme recognition sequences in the “maintenance-type design element” for an equivalent/same selected restriction enzyme(s)). In this excision-type design element, the cutting directed by the non-switchable restriction enzyme recognition sequence of the discard fragment will cut into the type IIS restriction enzyme recognition sequence of the methylation-protectable restriction element causing the resulting overhang (herein termed the “pre-assembly overhang”) of the nucleic acid to comprise of some nucleotide sequence of the type IIS restriction enzyme recognition sequence of the methylation-protectable restriction element. In another embodiment of the excision-type design element, the cutting directed by the type IIS restriction enzyme recognition sequence of a second/inside opposing methylation-protectable restriction element in the discard fragment will cut into the type IIS restriction enzyme recognition sequence of the first/outside methylation-protectable restriction element causing the resulting overhang (herein termed the “pre-assembly overhang”) of the nucleic acid to comprise of some nucleotide sequence of the type IIS restriction enzyme recognition sequence of the first/outside methylation-protectable restriction element.

In the excision-type design element, the opposing type IIS restriction enzyme recognition sequences (such as for BsaI) may be distanced apart by 2 base pairs. In another embodiment, the opposing type IIS restriction enzyme recognition sequences (such as for BsaI) may be distanced apart by 1 base pair. In another embodiment, the opposing type IIS restriction enzyme recognition sequences (such as for BsaI) may be distanced apart by 0 base pairs. As above, the skilled person will recognise that such distance depends on the property of the type IIS restriction enzyme(s) selected. For example, assuming the enzyme cuts x bases from the recognition sequence and generates y bases adhesive end (for BsaI x=1 and y=4), the distance (d) for the number of bases between opposing type IIS restriction enzyme recognition sequences of the excision-type design element may be provided by the following equation: d≤2*x (e.g. d≤2 bp for BsaI). The distance (d) for the excision-type design element may range from 2 bp (e.g. when using BsaI) to 20 bp (e.g. when using BtgZI). In one embodiment, the difference between the opposing type IIS restriction enzyme recognition sequences of the excision-type design element is d, wherein d≤2*x, and wherein x is the number of base pairs the cut site of a given restriction enzyme is from its recognition sequence.

In one embodiment, for example involving an excision-type design element, the nucleic acid may comprise the sequence of GACNNGGTCTCNNGAGACC (SEQ ID NO: 15); GACNNGGTCTCNGAGACC (SEQ ID NO: 16); or GACNNGGTCTCGAGACC (SEQ ID NO: 17) (BsaI/M2.Eco31I/M.Osp807II and opposing BsaI). In another embodiment, for example involving an excision-type design element, the nucleic acid may comprise the sequence of CCAGNNNNNNGGTCTCNNGAGACC (SEQ ID NO: 18); CCAGNNNNNNGGTCTCNGAGACC (SEQ ID NO: 19); or CCAGNNNNNNGGTCTCGAGACC (SEQ ID NO: 20); (BsaI/M2.Eco31I/M.Sen0738I and opposing BsaI). In another embodiment, for example involving an excision-type design element, the nucleic acid may comprise the sequence of TTCAGACNNGGTCTCNNGAGACC (SEQ ID NO: 21); TTCAGACNNGGTCTCNGAGACC (SEQ ID NO: 22); or TTCAGACNNGGTCTCGAGACC (SEQ ID NO: 23) (M.Csp205I/BsaI/M2.Eco31I/M.Osp807II and opposing BsaI). In another embodiment, for example involving an excision-type design element, the nucleic acid may comprise the sequence of TTCANNNNNGGTCTCNNGAGACC (SEQ ID NO: 24); TTCANNNNNGGTCTCNGAGACC (SEQ ID NO: 25); or TTCANNNNNGGTCTCGAGACC (SEQ ID NO: 26) (M.Csp205I/BsaI/M2.Eco31I and opposing BsaI). N may mean A, C, T or G. Other sequences may be provided according to the combined/overlapping restriction enzyme and methylase recognition sequences provided herein, wherein following the series of N bases, the sequence further comprises the palindromic/reverse-complement sequence of the restriction enzyme recognition sequence, and wherein the number of N bases between the restriction recognition sequences is reduced by the number overhang bases produced by the selected restriction enzyme. In one embodiment, the distance between the opposing restriction enzyme recognition sequences is d, wherein d≤2*x, and wherein x is the number of base pairs the cut site of a given restriction enzyme is from its recognition sequence.

Excision-Type Design of Head-to-Head/Opposing Restriction Sites with a Partial Type IIS Restriction Enzyme Recognition Sequence

The type IIS restriction enzyme recognition sequence may be a partial type IIS restriction enzyme recognition sequence. In particular, the methylation-protectable restriction element may comprise a partial type IIS restriction enzyme recognition sequence. The partial type IIS restriction enzyme recognition sequence may comprise a type IIS restriction enzyme recognition sequence that is modified to substitute the 3′ nucleotide with an alternative nucleotide (e.g. A, C, T or G), such that the sequence is not recognised or cut by the type IIS restriction enzyme that would normally recognise the full sequence. For example in the case of BsaI, the BsaI recognition sequence of GGTCTC may be modified to GGTCTN, wherein N=A, T or G, such that it is not recognised by BsaI. In another embodiment, the partial type IIS restriction enzyme recognition sequence may comprise a type IIS restriction enzyme recognition sequence that is modified to substitute two, three, four or five 3′ nucleotides with an alternative nucleotide (e.g. A, C, T or G), such that the sequence is not recognised or cut by the type IIS restriction enzyme that would normally recognise the full sequence. For example in the case of BsaI, the BsaI recognition sequence of GGTCTC may be modified to GGTCNN, GGTNNN, or GGNNNN, GNNNNN, wherein N=A, T C, or G, such that it is not recognised by BsaI.

In one embodiment involving an excision-type design element with an opposing type IIS restriction enzyme recognition sequence, the methylation-protectable restriction element may not comprise a type IIS restriction enzyme recognition sequence, for example that is the same as the opposing type IIS restriction enzyme recognition sequence, or a partial sequence thereof. In particular, a destination vector may comprise the elements of the methylation-protectable restriction element, such as the sequence specific DNA binding protein recognition sequence and/or the methylation switch element, but the corresponding type IIS restriction enzyme recognition sequence is not provided. Instead, the discard fragment is released by cutting as by direct by the opposing non-switchable type IIS restriction enzyme recognition sequence, and the DNA insert fragment to be ligated into the destination vector may provide the type IIS restriction enzyme recognition sequence of the methylation-protectable restriction element (i.e. once ligated), thereby effectively restoring a methylation-protectable restriction element according to the invention with a type IIS restriction enzyme recognition sequence.

Advantageously, the provision of a partial type IIS restriction enzyme recognition sequence in the methylation-protectable restriction element of an assembly/destination vector with an opposing non-switchable type IIS restriction enzyme recognition sequence allows the type IIS restriction enzyme to cut the vector as directed by the opposing non-switchable type IIS restriction enzyme recognition sequence, and not by the methylation-protectable restriction element. In the excision-type design, the partial type IIS restriction enzyme recognition sequence can then be functionally restored by ligation of the assembly/destination vector with an insert fragment that has pre-designed complementary overhang and the appropriate base pair sequence to restore the type IIS restriction enzyme recognition sequence of the methylation-protectable restriction element. The purpose of methylation switching in the excision-type design element can be to switch off the reconstituted site in a one-pot reaction.

For example in the case of BsaI, the BsaI recognition sequence of GGTCTC in the methylation-protectable restriction element may be modified to GGTCTN, wherein N=A, T or G, such that it is not recognised by BsaI, and the nucleotide complementary to the underlined base is methylated by methylation-switching. When this is cut by BsaI as directed by an opposing non-switchable type IIS restriction enzyme recognition sequence, the resulting sequence and overhang may be 5′-G-3′/3′-CCAGA-5′, (overhang sequence is underlined, methylated base double underlined). The opposing overhang of the insert fragment may be designed with the sequence 5′-G-3′/3′-GTCTC-5′ (the complementary overhang sequence is underlined), thereby facilitating binding and ligation. The full ligated sequence is 5′-GGTCTC-3′/3′-CCAGAG-5′ (methylated base double underlined), which is a full BsaI recognition sequence blocked by methylation.

The reconstituted full site assembled plasmid once transformed into a non-methylation switching strain will be unmethylated, and therefore can be cut again to generate an ‘excised’ insert fragment with overhang defined by the initial insert sequence, not predetermined by the vector sequence. In particular, the pre-designed elements of the previous insert are kept on the destination vector, and the resulting the overhang sequence of the new insert is the natural/wild-type sequence of the insert, which allows subsequent scarless assembly with other insert fragments. The partial type II restriction enzyme recognition sequence advantageously allows more flexibility in the sequence.

In one embodiment, for example involving an excision-type design element, the nucleic acid may comprise the sequence of GACNNGGTCTDGAGACC (SEQ ID NO: 27) (BsaI/M2.Eco31I/M.Osp807II and opposing BsaI). In another embodiment, for example involving an excision-type design element, the nucleic acid may comprise the sequence of CCAGNNNNNNGGTCTDGAGACC (SEQ ID NO: 28); (BsaI/M2.Eco31I/M.Sen0738I and opposing BsaI). In another embodiment, for example involving an excision-type design element, the nucleic acid may comprise the sequence of TTCAGACNNGGTCTDGAGACC (SEQ ID NO: 29) (M.Csp205I/BsaI/M2.Eco31I/M.Osp807II and opposing BsaI). In another embodiment, for example involving an excision-type design element, the nucleic acid may comprise the sequence of TTCANNNNNGGTCTDGAGACC (SEQ ID NO: 30) (M.Csp205I/BsaI/M2.Eco31I and opposing BsaI). N may mean A, C, T or G. D may mean A, T or G.

Insertional-Type Design of Head-to-Head/Opposing Restriction Sites

In an alternative embodiment to the maintenance-type design and excision-type designs, there is provided an “insertional-type design element”, wherein a sequence-insert is provided in between the methylation-protectable restriction element and the opposing non-switchable type IIS restriction enzyme recognition sequence (i.e. such that the cut site directed by the respective type IIS restriction enzyme recognition sequences are separated by, and flank, the sequence insert). In another embodiment of the insertional-type design element, a sequence-insert is provided in between the first/outside methylation-protectable restriction element and the second/inside opposing methylation-protectable restriction element. The sequence-insert may comprise a functional sequence such that it provides a function. For example, the sequence-insert may comprise a promoter sequence or a terminator sequence. In an embodiment comprising an insertional-type design element the opposing type IIS restriction enzyme recognition sequences may not overlap and may be distanced apart by the sequence-insert. The sequence-insert may be any suitable length. For example, the distance between the opposing type IIS restriction enzyme recognition sequences may be any number of nucleotides as required for the sequence-insert, such as a functional sequence-insert. The distance between the opposing type IIS restriction enzyme recognition sequences in an insertional-type design element may range between 6 bp to 300 kb.

Advantageously, following DNA assembly, the functional sequence-insert of the insertional-type design element will be added into the 5′ or 3′ end of the assembled DNA. This design is convenient for adding common elements to an assembled plasmid by using specialized vectors (for example adding promoter and terminators in a multi-part coding region gene assembly reaction).

Vector and Multiple Methylation-Protectable Restriction Elements in One Nucleic Acid Vector

The nucleic acid may be a vector. Therefore, according to another aspect of the invention, there is provided a vector comprising the nucleic acid of the invention.

The nucleic acid may comprise at least two methylation-protectable restriction elements.

In an embodiment comprising two methylation-protectable restriction elements, each methylation-protectable restriction element may comprise a methylation-switch element. In an embodiment comprising two methylation-protectable restriction elements, the switch DNA methylase recognition sequences of each methylation-switch element may be recognised by the same switch DNA methylase. The switch DNA methylase recognition sequence of the methylation-switch elements may comprise or consist of the same sequence.

In an embodiment comprising two methylation-protectable restriction elements, the type IIS restriction enzyme recognition sequences of each methylation-protectable restriction element may be recognised by the same type IIS restriction enzyme species. Each type IIS restriction enzyme recognition sequence of the methylation-protectable restriction elements may comprise or consist of the same sequence. The two methylation-protectable restriction elements may comprise or consist of the same sequence.

The use of the same type IIS restriction enzyme recognition sequence advantageously provides that a single type IIS restriction enzyme species can be used in a DNA assembly reaction. The use of a single type IIS restriction enzyme species opens the possibility of less complex DNA assembly.

In one embodiment, the methylation-protectable restriction element comprises a sequence according to any one of the overlapping methylation/restriction sites described herein.

In an embodiment comprising two methylation-protectable restriction elements, the nucleic acid sequence between the cut sites of the two methylation-protectable restriction elements may be a discard sequence (i.e. a fragment that will be excised from the nucleic acid during a restriction reaction). For example, the nucleic acid may be an assembly vector that is intended to receive one or more inserts between the cut sites, whereby the existing sequence between the cut sites is to be excised/discarded. In an alternative embodiment, the discard sequence may be previously removed. For example, the nucleic acid may be a previously-cut linearized vector, such as an assembly vector, having a methylation-protectable restriction element at each end.

The discard sequence may comprise a selectable marker, reporter gene, or label. The skilled person will be familiar with typical markers and reporter genes used in DNA assembly and cloning. For example, a selectable marker may comprise a gene encoding an antibiotic resistance, an enzymatic activity, a luciferase, or the like. In one embodiment, the discard sequence encodes lacZ as a reporter. The selection marker may comprise a negative selection marker. In one embodiment, the discard sequence may comprise a suicide gene as a selection marker, such as ccdB. The skilled person will recognise that the discard sequence, which may be replaced by the assembled sequence in a vector, may not contain any functional element at all, as the process of the invention favours generating assembled DNA.

Providing a selectable marker, reporter gene, or label on the discard sequence advantageously allows clones to be selected that have had the discard sequence successfully removed or replaced by a fragment/sequence of interest.

In an embodiment comprising two methylation-protectable restriction elements, the nucleic acid sequence between the cut sites of the two methylation-protectable restriction elements may comprise one or more, preferably two, of the non-switchable type IIS restriction enzyme recognition sequences. For example, in an embodiment comprising two methylation-protectable restriction elements, each may be opposed by an opposing non-switchable type IIS restriction enzyme recognition sequence. In an embodiment comprising a discard sequence, the opposing non-switchable restriction enzyme recognition sequence would be located in the discard sequence. In such an embodiment, the two type IIS restriction enzyme recognition sequences of the methylation-protectable restriction elements may be considered “outside restriction sites” and the opposing non-switchable type IIS restriction enzyme recognition sequences in the discard sequence may be considered the “inside restriction sites”.

In an embodiment comprising two methylation-protectable restriction elements (for example with a discard sequence therebetween), the overhang/sticky end produced by cutting with the type IIS restriction enzyme at the first methylation-protectable restriction element may be different in sequence than the overhang/sticky end produced by cutting with the type IIS restriction enzyme at the second methylation-protectable restriction element. The skilled person will be familiar with providing different overhangs for directional DNA assembly/cloning or controlling which end of the nucleic acid is ligated to an insert/fragment of interest.

In an embodiment wherein the nucleic acid comprises two methylation-protectable restriction elements, both methylation-protectable restriction elements may comprise the maintenance-type design element.

In an embodiment, for example involving two maintenance-type design elements, the nucleic acid may comprise the sequence of GACNNGGTCTCNNNNNNGAGACC(N^{x})GGTCTCNNNNNNGAGACCNNGTC (SEQ ID NO: 31) (BsaI/M2.Eco31I/M.Osp807II and opposing BsaI). In another embodiment involving two maintenance-type design elements, the nucleic acid may comprise the sequence of CCAGNNNNNNGGTCTCNNNNNNGAGACC(N^{x})GGTCTCNNNNNNGAGACCNNN NNNCTGG (SEQ ID NO: 32) (BsaI/M2.Eco31I/M.Sen0738I and opposing BsaI). In another embodiment, for example involving two maintenance-type design elements, the nucleic acid may comprise the sequence of TTCAGACNNGGTCTCNNNNNNGAGACC(N^{x})GGTCTCNNNNNNGAGACCNNGT CTGAA (SEQ ID NO: 33) (M.Csp205I/BsaI/M2.Eco31I/M.Osp807II and opposing BsaI). In another embodiment, for example involving two maintenance-type design elements, the nucleic acid may comprise the sequence of TTCANNNNNGGTCTCNNNNNNGAGACC(N^{x})GGTCTCNNNNNNGAGACCNNNN NTGAA (SEQ ID NO: 34) (M.Csp205I/BsaI/M2.Eco31I and opposing BsaI). N may mean A, C, T or G. (Nₓ) may mean any number of A, C, T or G, or between 0 bp and 300 kbp of A, C, T, or G.

In another embodiment wherein the nucleic acid comprises two methylation-protectable restriction elements, one methylation-protectable restriction element may comprise a maintenance-type design element and the other methylation-protectable restriction element may comprise an excision-type design element.

In one embodiment, for example involving a maintenance-type design element and excision-type design element, the nucleic acid may comprise the sequence of GACNNGGTCTCNNNNNNGAGACC(N^{x})GGTCTCNNGAGACCNNGTC (SEQ ID NO 35); GACNNGGTCTCNNNNNNGAGACC(N^{x})GGTCTCNGAGACCNNGTC (SEQ ID NO: 36); or GACNNGGTCTCNNNNNNGAGACC(N^{x})GGTCTCYAGACCNNGTC (SEQ ID NO: 37) (BsaI/M2.Eco31I/M.Osp807II and opposing BsaI). In another embodiment, for example involving a maintenance-type design element and excision-type design element, the nucleic acid may comprise the sequence of CCAGNNNNNNGGTCTCNNNNNNGAGACC(N^{x})GGTCTCNNGAGACCNNNNNNC TGG (SEQ ID NO: 38); CCAGNNNNNNGGTCTCNNNNNNGAGACC(N^{x})GGTCTCNGAGACCNNNNNNCT GG (SEQ ID NO: 39); or CCAGNNNNNNGGTCTCNNNNNNGAGACC(N^{x})GGTCTCYAGACCNNNNNNCTG G (SEQ ID NO: 40) (BsaI/M2.Eco31I/M.Sen0738I and opposing BsaI). In one embodiment, Y=A, T, C or G. In one embodiment Y=G. In another embodiment, Y=A, T or C.

In another embodiment, for example involving a maintenance-type design element and excision-type design element, the nucleic acid may comprise the sequence of TTCAGACNNGGTCTCNNNNNNGAGACC(N^{x})GGTCTCNNGAGACCNNGTCTGA A (SEQ ID NO: 41); TTCAGACNNGGTCTCNNNNNNGAGACC(N^{x})GGTCTCNGAGACCNNGTCTGAA (SEQ ID NO: 42); or TTCAGACNNGGTCTCNNNNNNGAGACC(N^{x})GGTCTCYAGACCNNGTCTGAA (SEQ ID NO: 43) (M.Csp205I/BsaI/M2.Eco31I/M.Osp807II and opposing BsaI). In another embodiment, for example involving a maintenance-type design element and excision-type design element, the nucleic acid may comprise the sequence of TTCANNNNNGGTCTCNNNNNNGAGACC(N^{x})GGTCTCNNGAGACCNNNNNTGA A (SEQ ID NO: 44); TTCANNNNNGGTCTCNNNNNNGAGACC(N^{x})GGTCTCNGAGACCNNNNNTGAA (SEQ ID NO: 45; or TTCANNNNNGGTCTCNNNNNNGAGACC(N^{x})GGTCTCYAGACCNNNNNTGAA (SEQ ID NO: 46) (M.Csp205I/BsaI/M2.Eco31I and opposing BsaI).

(N^{x}) may mean any number of A, C, T or G, or between 0 bp and 300 kbp of A, C, T, or G. In one embodiment, Y=A, T, C or G. In one embodiment Y=G. In another embodiment, Y=A, T or C.

In another embodiment, for example involving a maintenance-type design element and excision-type design element, the nucleic acid may comprise the sequence of GACNNGGTCTCNNGAGACC(N^{x})GGTCTCNNNNNNGAGACCNNGTC (SEQ ID NO: 47); GACNNGGTCTCNGAGACC(N^{x})GGTCTCNNNNNNGAGACCNNGTC (SEQ ID NO: 48); or GACNNGGTCTYGAGACC(N^{x})GGTCTCNNNNNNGAGACCNNGTC (SEQ ID NO: 49) (BsaI/M2.Eco31I/M.Osp807II and opposing BsaI).

In another embodiment, for example involving a maintenance-type design element and excision-type design element, the nucleic acid may comprise the sequence of CCAGNNNNNNGGTCTCNNGAGACC(N^{x})GGTCTCNNNNNNGAGACCNNNNNNC TGG (SEQ ID NO: 50); CCAGNNNNNNGGTCTCNGAGACC(N^{x})GGTCTCNNNNNNGAGACCNNNNNNCT GG (SEQ ID NO: 51); or CCAGNNNNNNGGTCTYGAGACC(N^{x})GGTCTCNNNNNNGAGACCNNNNNNCTG G (SEQ ID NO: 52) (BsaI/M2.Eco31I/M.Sen0738I and opposing BsaI). In one embodiment, Y=A, T, C or G. In one embodiment Y=C. In another embodiment, Y=A, T or G.

In another embodiment, for example involving a maintenance-type design element and excision-type design element, the nucleic acid may comprise the sequence of TTCAGACNNGGTCTCNNGAGACC(N^{x})GGTCTCNNNNNNGAGACCNNGTCTGA A (SEQ ID NO: 53); TTCAGACNNGGTCTCNGAGACC(N^{x})GGTCTCNNNNNNGAGACCNNGTCTGAA (SEQ ID NO: 54); or TTCAGACNNGGTCTYGAGACC(N^{x})GGTCTCNNNNNNGAGACCNNGTCTGAA (SEQ ID NO: 55) (M.Csp205I/BsaI/M2.Eco31I/M.Osp807II and opposing BsaI). In another embodiment, for example involving a maintenance-type design element and excision-type design element, the nucleic acid may comprise the sequence of TTCANNNNNGGTCTCNNGAGACC(N^{x})GGTCTCNNNNNNGAGACCNNNNNTGA A (SEQ ID NO: 56); TTCANNNNNGGTCTCNGAGACC(N^{x})GGTCTCNNNNNNGAGACCNNNNNTGAA (SEQ ID NO: 57); or TTCANNNNNGGTCTYGAGACC(N^{x})GGTCTCNNNNNNGAGACCNNNNNTGAA (SEQ ID NO: 58) (M.Csp205I/BsaI/M2.Eco31I and opposing BsaI).

N may mean A, C, T or G. (N^{x}) may mean any number of A, C, T or G, or between 0 bp and 300 kbp of A, C, T, or G. In one embodiment, Y=A, T, C or G. In one embodiment Y=C. In another embodiment, Y=A, T or G.

In an embodiment wherein the nucleic acid comprises two methylation-protectable restriction elements, both methylation-protectable restriction elements may comprise the excision-type design element.

In one embodiment, for example involving two excision-type design elements, the nucleic acid may comprise the sequence of GACNNGGTCTCNNGAGACC(N^{x})GGTCTCNNGAGACCNNGTC (SEQ ID NO: 59); GACNNGGTCTCNGAGACC(N^{x})GGTCTCNGAGACCNNGTC (SEQ ID NO: 60); GACNNGGTCTYGAGACC(N^{x})GGTCTCZAGACCNNGTC (SEQ ID NO: 61); GACNNGGTCTCNGAGACC(N^{x})GGTCTCNNGAGACCNNGTC (SEQ ID NO: 62); GACNNGGTCTCNNGAGACC(N^{x})GGTCTCNGAGACCNNGTC (SEQ ID NO: 63); GACNNGGTCTYGAGACC(N^{x})GGTCTCNNGAGACCNNGTC (SEQ ID NO: 64); GACNNGGTCTCNNGAGACC(N^{x})GGTCTCZAGACCNNGTC (SEQ ID NO: 65); GACNNGGTCTCNGAGACC(N^{x})GGTCTCZAGACCNNGTC (SEQ ID NO: 66); or GACNNGGTCTYGAGACC(N^{x})GGTCTCNGAGACCNNGTC (SEQ ID NO: 67) (BsaI/M2.Eco31I/M.Osp807II and opposing BsaI). In one embodiment, Y=A, T, C or G. In one embodiment Y=C. In another embodiment, Y=A, T or G. In one embodiment, Z=A, T, C or G. In one embodiment Z=G. In another embodiment, Z=A, T or C.

In another embodiment, for example involving two excision-type design elements, the nucleic acid may comprise the sequence of CCAGNNNNNNGGTCTCNNGAGACC(N^{x})GGTCTCNNGAGACCNNNNNNCTGG (SEQ ID NO: 68); CCAGNNNNNNGGTCTCNGAGACC(N^{x})GGTCTCNGAGACCNNNNNNCTGG (SEQ ID NO: 69); CCAGNNNNNNGGTCTYGAGACC(N^{x})GGTCTCZAGACCNNNNNNCTGG (SEQ ID NO: 70); CCAGNNNNNNGGTCTCNNGAGACC(N^{x})GGTCTCNGAGACCNNNNNNCTGG (SEQ ID NO: 71); CCAGNNNNNNGGTCTCNGAGACC(N^{x})GGTCTCNNGAGACCNNNNNNCTGG (SEQ ID NO: 72); CCAGNNNNNNGGTCTCNNGAGACC(N^{x})GGTCTCZAGACCNNNNNNCTGG (SEQ ID NO: 73); CCAGNNNNNNGGTCTYGAGACC(N^{x})GGTCTCNNGAGACCNNNNNNCTGG (SEQ ID NO: 74); CCAGNNNNNNGGTCTCNGAGACC(N^{x})GGTCTCZAGACCNNNNNNCTGG (SEQ ID NO: 75); or CCAGNNNNNNGGTCTYGAGACC(N^{x})GGTCTCNGAGACCNNNNNNCTGG (SEQ ID NO: 76) (BsaI/M2.Eco31I/M.Sen0738I and opposing BsaI). In one embodiment, Y=A, T, C or G. In one embodiment Y=C. In another embodiment, Y=A, T or G. In one embodiment, Z=A, T, C or G. In one embodiment Z=G. In another embodiment, Z=A, T or C.

In another embodiment, for example involving two excision-type design elements, the nucleic acid may comprise the sequence of TTCAGACNNGGTCTCNNGAGACC(N^{x})GGTCTCNNGAGACCNNGTCTGAA (SEQ ID NO: 77); TTCAGACNNGGTCTCNGAGACC(N^{x})GGTCTCNGAGACCNNGTCTGAA (SEQ ID NO: 78); TTCAGACNNGGTCTYGAGACC(N^{x})GGTCTCZAGACCNNGTCTGAA (SEQ ID NO: 79); TTCAGACNNGGTCTCNGAGACC(N^{x})GGTCTCNNGAGACCNNGTCTGAA (SEQ ID NO: 80); TTCAGACNNGGTCTCNNGAGACC(N^{x})GGTCTCNGAGACCNNGTCTGAA (SEQ ID NO: 81); TTCAGACNNGGTCTYGAGACC(N^{x})GGTCTCNNGAGACCNNGTCTGAA (SEQ ID NO: 82); TTCAGACNNGGTCTCNNGAGACC(N^{x})GGTCTCZAGACCNNGTCTGAA (SEQ ID NO: 83); TTCAGACNNGGTCTYGAGACC(N^{x})GGTCTCNGAGACCNNGTCTGAA (SEQ ID NO: 84); or TTCAGACNNGGTCTCNGAGACC(N^{x})GGTCTCZAGACCNNGTCTGAA (SEQ ID NO: 85) (M.Csp205I/BsaI/M2.Eco31I/M.Osp807II and opposing BsaI/M2.Eco31I). In one embodiment, Y=A, T, C or G. In one embodiment Y=C. In another embodiment, Y=A, T or G. In one embodiment, Z=A, T, C or G. In one embodiment Z=G. In another embodiment, Z=A, T or C.

In another embodiment, for example involving two excision-type design elements, the nucleic acid may comprise the sequence of TTCANNNNNGGTCTCNNGAGACC(N^{x})GGTCTCNNGAGACCNNNNNTGAA (SEQ ID NO: 86); TTCANNNNNGGTCTCNGAGACC(N^{x})GGTCTCNGAGACCNNNNNTGAA (SEQ ID NO: 87); TTCANNNNNGGTCTYGAGACC(N^{x})GGTCTCZAGACCNNNNNTGAA (SEQ ID NO: 88); TTCANNNNNGGTCTCNNGAGACC(N^{x})GGTCTCNGAGACCNNNNNTGAA (SEQ ID NO: 89); TTCANNNNNGGTCTCNGAGACC(N^{x})GGTCTCNNGAGACCNNNNNTGAA (SEQ ID NO: 90); TTCANNNNNGGTCTCNNGAGACC(N^{x})GGTCTCZAGACCNNNNNTGAA (SEQ ID NO: 91); TTCANNNNNGGTCTYGAGACC(N^{x})GGTCTCNNGAGACCNNNNNTGAA (SEQ ID NO: 92); TTCANNNNNGGTCTCNGAGACC(N^{x})GGTCTCZAGACCNNNNNTGAA (SEQ ID NO: 93); or TTCANNNNNGGTCTYGAGACC(N^{x})GGTCTCNGAGACCNNNNNTGAA (SEQ ID NO: 94) (M.Csp205I/BsaI/M2.Eco31I and opposing BsaI/M2.Eco31I). (Nₓ) may mean any number of A, C, T or G, or between 0 bp and 300 kbp of A, C, T, or G. In one embodiment, Y=A, T, C or G. In one embodiment Y=C. In another embodiment, Y=A, T or G. In one embodiment, Z=A, T, C or G. In one embodiment Z=G. In another embodiment, Z=A, T or C.

In one embodiment, the nucleic acid may comprise a circular nucleic acid, such as a vector, comprising two maintenance-type design elements with a discard sequence therebetween, or a linearized version thereof with the discard sequence cut out. In another embodiment, the nucleic acid may comprise a circular nucleic acid, such as a vector, comprising a maintenance-type design element and an excision-type design element with a discard sequence therebetween, or a linearized version thereof with the discard sequence cut out. In another embodiment, the nucleic acid may comprise a circular nucleic acid, such as a vector, comprising two excision-type design elements with a discard sequence therebetween, or a linearized version thereof with the discard sequence cut out. In another embodiment, the nucleic acid may comprise a circular nucleic acid, such as a vector, comprising an insertional-type design element and an excision-type design element with a discard sequence therebetween, or a linearized version thereof with the discard sequence cut out. In another embodiment, the nucleic acid may comprise a circular nucleic acid, such as a vector, comprising an insertional-type design element and a maintenance-type design element with a discard sequence therebetween, or a linearized version thereof with the discard sequence cut out. In another embodiment, the nucleic acid may comprise a circular nucleic acid, such as a vector, comprising two insertional-type design elements with a discard sequence therebetween, or a linearized version thereof with the discard sequence cut out. In linearized nucleic acid embodiments, the discard sequence may have been cut out by restriction with the restriction enzyme that recognises the opposing non-switchable restriction enzyme sequence present on the discard sequence (i.e. the inner restriction enzyme recognition sequence).

In embodiments comprising two maintenance-type design elements, the restriction enzyme recognition sequences of the two maintenance-type design elements may be the same and/or the DNA methylase recognition sequences of the two maintenance-type design elements may be the same. In embodiments comprising two excision-type design elements, the restriction enzyme recognition sequences of the two excision-type design elements may be the same and/or the DNA methylase recognition sequences of the two excision-type design elements may be the same. In embodiments comprising two insertional-type design elements, the restriction enzyme recognition sequences of the two insertional-type design elements may be the same and/or the DNA methylase recognition sequences of the two insertional-type design elements may be the same. In embodiments comprising a maintenance-type design element and a excision-type design element, the restriction enzyme recognition sequences of the maintenance-type design element and excision-type design element may be the same and/or the DNA methylase recognition sequences of the maintenance-type design element and excision-type design element may be the same. In embodiments comprising a maintenance-type design element and an insertional-type design element, the restriction enzyme recognition sequences of the maintenance-type design element and insertional-type design element may be the same and/or the DNA methylase recognition sequences of the maintenance-type design element and insertional-type design element may be the same. In embodiments comprising an excision-type design element and an insertional-type design element, the restriction enzyme recognition sequences of the excision-type design element and insertional-type design element may be the same and/or the DNA methylase recognition sequences of the excision-type design element and insertional-type design element may be the same. For example, digestion of the nucleic acid to remove a discard sequence or DNA fragment of interest may be carried out by the same restriction enzyme. Additionally the DNA methylation of the nucleic acid to protect all the methylation-protectable restriction enzyme recognition sites in the nucleic acid may be carried out by the same DNA methylase.

The nucleic acid may be isolated nucleic acid. In one embodiment, the nucleic acid may be synthetic (i.e. not found in nature). In one embodiment, the nucleic acid may be isolated or derived from a bacterial strain, such as E. coli, that expresses a DNA methylase that recognises (and methylates) the DNA methylase recognition sequence of the methylation-protectable restriction element. For example, a bacterial strain, such as E. coli, that expresses any one type II DNA methylase identified herein. In one embodiment, the nucleic acid may be isolated or derived from a bacterial strain, such as E. coli, that expresses a switch DNA methylase that recognises (and methylates) the switch DNA methylase recognition sequence of the methylation-protectable restriction element. In one embodiment the nucleic acid may be isolated or derived from a bacterial strain, such as E. coli, that expresses one or more of M.Osp807II; M.Sen0738I co-expressed with S.Sen0738I; and M2.Eco31I. In one embodiment the nucleic acid may be isolated or derived from a bacterial strain, such as E. coli, that expresses M2.Eco31I and does not express a DNA methylase of M.Osp807II and/or M.Sen0738I. In an alternative embodiment the nucleic acid may be isolated or derived from a bacterial strain, such as E. coli, that expresses M.Osp807II and/or M.Sen0738I (co-expressed with S.Sen0738I) and does not express a methylase of M2.Eco31I. The expressed DNA methylase may be functional. The bacterial strain may be genetically modified/engineered to express such DNA methylase.

In one embodiment the nucleic acid may be isolated or derived from a bacterial strain, such as E. coli, that expresses the sequence specific DNA binding protein (and associated guide nucleic acid where necessary). In one embodiment the nucleic acid may be isolated or derived from a bacterial strain, such as E. coli, that expresses the sequence specific DNA binding protein (and associated guide nucleic acid where necessary) and expresses a DNA methylase that recognises (and methylates) the DNA methylase recognition sequence of the methylation-protectable restriction element.

In an embodiment wherein the sequence specific DNA binding protein is a DNA methylase, the nucleic acid may be isolated or derived from a bacterial strain, such as E. coli, that expresses the DNA methylase that recognises the sequence specific DNA binding protein recognition sequence of the methylation-protectable restriction element. In one embodiment the nucleic acid may be isolated or derived from a bacterial strain, such as E. coli, that expresses M.Csp205I. In one embodiment the nucleic acid may be isolated or derived from a bacterial strain, such as E. coli, that expresses M.Csp205I and M2.Eco3II.

The skilled person will understand that in embodiments where M.Csp205I is used, it may be used or co-expressed with S.Csp205I.

In one embodiment the nucleic acid may be isolated or derived from a bacterial strain, such as E. coli, that expresses M2.Eco3II and the sequence-specific DNA-binding protein, such as a nucleic acid binding protein (e.g. dCas9), and a guide nucleic acid where required.

The nucleic acid may be a vector, such as a cloning vector. The skilled person will be familiar with various cloning vectors, which may include, for example, a replication origin, a selectable marker, a reporter gene, expression elements, or combinations thereof. The vector may comprise nucleic acid that comprises a DNA element that supports the replication of DNA that contains it (e.g. a replication origin) and a selection marker (e.g. an antibiotic selection marker). The vector may be a high or low copy number vector. In one embodiment, the vector is a high copy number vector. The skilled person will be familiar with cloning methods and materials/vectors, for example as provided in Green, M. R., Sambrook, J. Molecular Cloning: A Laboratory Manual. 4th. Cold Spring Harbor Laboratory Press, Cold Spring Harbor, N.Y.; 2012, which is herein incorporated by reference.

The nucleic acid described herein may comprise or consist of DNA.

Method of DNA Assembly

According to another aspect of the invention, there is provided a method of assembling DNA comprising:
providing a linearised methylated nucleic acid according to the invention herein, wherein the methylation-protectable restriction element comprises a methylation-switch element that is switched OFF by methylation of the type IIS restriction enzyme recognition sequence with the switch DNA methylase;
providing two or more DNA/insert fragments of interest for assembly with the linearised methylated nucleic acid, wherein a first DNA fragment comprises a complementary overhang for ligation with a first end of the linearised methylated nucleic acid, and a second DNA fragment comprises a complementary overhang for ligation with the other/second end of the linearised methylated nucleic acid; and
(i) the first and second DNA/insert fragments further comprise complementary overhangs for ligation with each other; or
(ii) the first and second DNA/insert fragments further comprise complementary overhangs for ligation with one or more further DNA/insert fragments having complementary overhangs, such that ligating the DNA fragments and the linearised methylated nucleic acid with a DNA ligase would result in a single assembled DNA molecule; and
ligating the DNA fragments and linearised methylated nucleic acid with a ligase to form a single assembled DNA molecule comprising the sequence of the assembled DNA fragments flanked by the restriction enzyme recognition sequences of the methylation-protectable restriction elements.

According to another aspect of the invention, there is provided a method of assembling DNA comprising:
providing a linearised methylated nucleic acid according to the invention herein, wherein the methylation-protectable restriction element comprises a methylation-switch element that is switched OFF by methylation of the type IIS restriction enzyme recognition sequence with the switch DNA methylase;
providing a DNA/insert fragment of interest for assembly with the linearised methylated nucleic acid, wherein the DNA/insert fragment of interest comprises complementary overhangs for ligation with the linearised methylated nucleic acid; and
ligating the DNA fragments and linearised methylated nucleic acid with a ligase to form a single assembled DNA molecule comprising the sequence of the assembled DNA fragments flanked by the restriction enzyme recognition sequences of the methylation-protectable restriction elements.

According to another aspect of the invention, there is provided a method of assembling DNA comprising:
providing a linearised methylated nucleic acid according to the invention herein, wherein the type IIS restriction enzyme recognition sequence of the methylation-protectable restriction element is protected from cutting by methylation with the DNA methylase that recognises the DNA methylase recognition sequence (ii) of the methylation-protectable restriction element;
providing two or more DNA/insert fragments of interest for assembly with the linearised methylated nucleic acid, wherein a first DNA fragment comprises a complementary overhang for ligation with a first end of the linearised methylated nucleic acid, and a second DNA fragment comprises a complementary overhang for ligation with the other/second end of the linearised methylated nucleic acid; and
(i) the first and second DNA/insert fragments further comprise complementary overhangs for ligation with each other; or
(ii) the first and second DNA/insert fragments further comprise complementary overhangs for ligation with one or more further DNA/insert fragments having complementary overhangs, such that ligating the DNA fragments and the linearised methylated nucleic acid with a DNA ligase would result in a single assembled DNA molecule; and
ligating the DNA fragments and linearised methylated nucleic acid with a ligase to form a single assembled DNA molecule comprising the sequence of the assembled DNA fragments flanked by the restriction enzyme recognition sequences of the methylation-protectable restriction elements.

According to another aspect of the invention, there is provided a method of assembling DNA comprising:
providing a linearised methylated nucleic acid according to the invention herein, wherein the type IIS restriction enzyme recognition sequence of the methylation-protectable restriction element is protected from cutting by methylation with the DNA methylase that recognises the DNA methylase recognition sequence (ii) of the methylation-protectable restriction element;
providing a DNA/insert fragment of interest for assembly with the linearised methylated nucleic acid, wherein the DNA/insert fragment of interest comprises complementary overhangs for ligation with the linearised methylated nucleic acid; and
ligating the DNA fragments and linearised methylated nucleic acid with a ligase to form a single assembled DNA molecule comprising the sequence of the assembled DNA fragments flanked by the restriction enzyme recognition sequences of the methylation-protectable restriction elements.

The single assembled DNA molecule may be circular, such as a circular DNA vector.

Advantageously, further cutting of the single assembled DNA molecule by the restriction enzyme that recognises the type IIS restriction enzyme recognition sequences of the methylation-protectable restriction elements is blocked by the methylation of the type IIS restriction enzyme recognition sequences.

The linearised nucleic acid may be provided by providing a nucleic acid according to the invention in the form of a circular destination vector (otherwise referred to as an assembly vector) comprising two methylated methylation-protectable restriction elements and a discard sequence therebetween. Each methylated methylation-protectable restriction element may be opposed by an opposing non-switchable restriction enzyme recognition sequence in the discard sequence. The method may further comprise the step of cutting the circular destination vector with restriction enzymes that recognise the opposing non-switchable restriction enzyme recognition sequences in the discard sequence, thereby leaving a linearised nucleic acid having overhangs defined by the restriction enzymes. The restriction enzymes may be the same species. The restriction enzyme recognition sequences of the methylated methylation-protectable restriction elements may comprise the same sequence. In one embodiment, the restriction enzyme recognition sequences of the methylated methylation-protectable restriction elements and the opposing non-switchable restriction enzyme recognition sequences may comprise the same sequence. The restriction enzyme may be BsaI.

In an embodiment, wherein the type IIS restriction enzyme recognition sequence of the methylation-protectable restriction element is protected from cutting by methylation with the DNA methylase that recognises the DNA methylase recognition sequence (ii) of the methylation-protectable restriction element, the linearised nucleic acid may be provided by providing a nucleic acid according to the invention in the form of a circular destination vector (otherwise referred to as an assembly vector) comprising two pairs of opposing (i.e. head to head) methylation-protectable restriction elements and a discard sequence therebetween, wherein one pair of opposing (i.e. head to head) methylation-protectable restriction elements comprises an outside methylation-protectable restriction element that will remain in the vector after linearization and an opposing inside methylation-protectable restriction element that is in the discard sequence. The second pair of opposing (i.e. head to head) methylation-protectable restriction elements may also comprise an outside methylation-protectable restriction element that will remain in the vector after linearization and an opposing inside methylation-protectable restriction element that is in the discard sequence. In particular, there may be four methylation-protectable restriction elements.

The opposing (i.e. head to head) methylation-protectable restriction elements of a pair may comprise different sequence specific DNA binding recognition sequences. The sequence specific DNA binding recognition sequences of the outside methylation-protectable restriction elements may be the same as each other and/or the sequence specific DNA binding recognition sequences of the inside methylation-protectable restriction elements may be the same as each other.

The outside methylation-protectable restriction elements may be methylated and thereby protected from cutting by the type IIS restriction enzyme that recognises the type IIS recognition sequences of the outside methylation-protectable restriction elements. Additionally or alternatively, the inside methylation-protectable restriction elements may not be methylated and thereby not protected from cutting by the type IIS restriction enzyme that recognises the type IIS recognition sequences of the inside methylation-protectable restriction elements.

The outside methylation-protectable restriction elements may be methylated and thereby protected from cutting by preparing/isolating the vector in a strain that expresses the DNA methylase that recognises the DNA methylase recognition sequence (ii) of the methylation-protectable restriction elements, but the strain does not express a functional sequence specific DNA binding protein that recognises the sequence specific DNA binding protein recognition sequence of the outside methylation-protectable restriction elements. Additionally or alternatively, the inside methylation-protectable restriction elements may not be methylated and thereby not protected from cutting by preparing/isolating the vector in a strain that expresses the DNA methylase that recognises the DNA methylase recognition sequence (ii) of the methylation-protectable restriction elements, and expresses a functional sequence specific DNA binding protein that recognises the sequence specific DNA binding protein recognition sequence of the inside methylation-protectable restriction elements. The same type IIS restriction enzyme (such as BsaI) and/or the same DNA methylase (such as M2.Eco31I) may be used for both inside and outside methylation-protectable restriction elements.

The outside methylation-protectable restriction elements may be methylated and thereby protected from cutting by preparing the vector in vitro with the DNA methylase that recognises the DNA methylase recognition sequence (ii) of the methylation-protectable restriction elements, without a functional sequence specific DNA binding protein that recognises the sequence specific DNA binding protein recognition sequence of the outside methylation-protectable restriction elements. Additionally or alternatively, the inside methylation-protectable restriction elements may not be methylated and thereby not protected from cutting by preparing the vector in vitro with the DNA methylase that recognises the DNA methylase recognition sequence (ii) of the methylation-protectable restriction elements, and a functional sequence specific DNA binding protein that recognises the sequence specific DNA binding protein recognition sequence of the inside methylation-protectable restriction elements. The same type IIS restriction enzyme (such as BsaI) and/or the same DNA methylase (such as M2.Eco31I) may be used for both inside and outside methylation-protectable restriction elements.

The skilled person will recognise that preparing the vector in vitro may comprise reacting the vector/nucleic acid with the appropriate DNA binding protein and/or DNA methylase, for example in a buffer.

The method may comprise the step of cutting the circular destination vector comprising two pairs of opposing (i.e. head to head) methylation-protectable restriction elements with a type IIS restriction enzyme that recognise the type IIS restriction enzyme recognition sequences of the inside methylation-protectable restriction elements that are in the discard sequence, thereby leaving a linearised nucleic acid having overhangs defined by the type IIS restriction enzymes.

The restriction enzyme and methylase (or recognition sequences thereof) used in the method of the invention may be selected in accordance with the restriction enzyme and methylases (or recognition sequences thereof) provided for the nucleic acid aspect of the present invention herein.

The circular destination vector (otherwise referred to as an assembly vector) may be purified/isolated from a bacterial strain that expresses the switch DNA methylase that recognises the switch DNA methylase recognition sequence of the methylation-switch element. In one embodiment, the circular destination vector may be purified/isolated from a bacterial strain that expresses M.Osp8071I or M.Sen0738I that recognises the switch DNA methylase recognition sequence of the methylation-switch element. In another embodiment, the circular or linearised destination vector may be methylated by the switch DNA methylase in vitro, for example by M.Osp8071I or M.Sen0738I.

The circular destination vector (otherwise referred to as an assembly vector) may be prepared in vitro with the switch DNA methylase that recognises the switch DNA methylase recognition sequence of the methylation-switch element. In one embodiment, the circular destination vector may be prepared in vitro with M.Osp8071I or M.Sen0738I that recognises the switch DNA methylase recognition sequence of the methylation-switch element.

The DNA/insert fragments of interest may be methylated. In one embodiment, the DNA fragment(s) of interest may be methylated with the DNA methylase, such as M2.Eco31I, that recognises the DNA methylase recognition sequence (ii) of the methylation-protectable restriction element. Methylation of the DNA fragment(s) of interest by the DNA methylase, such as M2.Eco31I, that recognises the DNA methylase recognition sequence (ii) of the methylation-protectable restriction element may be provided by isolating the DNA fragment(s), preferably in a vector form, from a bacterial strain that expresses the DNA methylase, and optionally the sequence specific binding protein described herein, for example dCas9+guide RNA, Alternatively, methylation of the DNA fragment(s) of interest by the DNA methylase, such as M2.Eco31I, that recognises the DNA methylase recognition sequence (ii) of the methylation-protectable restriction element may be in vitro.

The DNA fragment(s) of interest for assembly with the nucleic acid may be provided in a circular donation vector (otherwise referred to as an insert plasmid), wherein the method comprises the step of cutting the circular donation vector to release the DNA fragment(s) of interest. The cutting of the donor vectors may be with a restriction enzyme that leaves complementary overhangs for ligation with the linearised methylated nucleic acid. The cutting of the donor vector may be with a restriction enzyme that leaves complementary overhangs for ligation with the linearised methylated nucleic acid and with another DNA fragment of interest for insertion. The cutting of the donor vector may be with a restriction enzyme that leaves complementary overhangs for ligation with two other DNA fragments of interest for insertion.

The circular donation vector may comprise two methylation-protectable restriction elements with a DNA fragment of interest therebetween. The circular donation vector may be methylated or at least exposed to methylation by the DNA methylase, such as M2.Eco31I, that recognises the DNA methylase recognition sequence (ii). Therefore, any corresponding type IIS restriction enzyme recognition sequences that may be present internally in the DNA fragment of interest may be methylated, and thereby protected from cutting. The type II restriction enzyme recognition sequences of the circular donation vector may not be methylated by the DNA methylase, such as M2.Eco31I, that recognises the DNA methylase recognition sequence (ii), for example due to protection from methylation by the sequence specific DNA binding protein. The skilled person will recognise that methylation of the circular donation vector and/or the DNA fragment of interest may not be necessary in an embodiment wherein the DNA fragment of interest does not comprise any internal type IIS restriction enzyme recognition sequences that would be recognised and cut by the type IIS restriction enzyme that recognises the type IIS restriction enzyme recognition sequence of the methylation-protectable restriction element.

The method may further comprise the step of cutting the circular donation vector with restriction enzymes that recognise the type IIS restriction enzyme recognition sequence of the methylation-protectable restriction element, thereby releasing a linear DNA fragment of interest having overhangs defined by the restriction enzymes. The restriction enzymes may be the same species. The type IIS restriction enzyme recognition sequences of the methylated methylation-protectable restriction elements may comprise the same sequence. The type IIS restriction enzyme may comprise or consist of BsaI.

Multiple DNA/insert fragments may be provided from a single donor vector, or a donor vector may be provided for each DNA/insert fragment.

The restriction enzyme for cutting the circular donor vector(s) may comprise a type IIS restriction enzyme, such as BsaI. The restriction enzyme for cutting the circular donor vector(s) may comprise the same restriction enzyme, or a restriction enzyme that recognises the same sequence, as the restriction enzyme used to cut the circular destination vector(s). The complementary overhangs of the DNA fragment(s) of interest for ligation with the linearised nucleic acid may be defined by the restriction enzyme.

The circular donor vector(s) may be purified/isolated from a bacterial strain that expresses the sequence-specific DNA-binding protein that recognises the sequence-specific DNA-binding protein recognition sequence of the methylation-protectable restriction element and the DNA methylase that recognises the DNA methylase recognition sequence (ii) of the methylation-protectable restriction element. Alternatively, the circular donor vector(s) may be prepared in vitro with the sequence-specific DNA-binding protein that recognises the sequence-specific DNA-binding protein recognition sequence of the methylation-protectable restriction element and the DNA methylase that recognises the DNA methylase recognition sequence (ii) of the methylation-protectable restriction element.

In one embodiment, the circular destination vector (otherwise referred to as an assembly vector) is purified/isolated from a bacterial strain that expresses the switch DNA methylase (such as M.Sen0728I/S.Sen0728I) that recognises the switch DNA methylase recognition sequence of the methylation-switch element and the circular donor vector(s) may be purified/isolated from a bacterial strain that expresses the sequence-specific DNA-binding protein (such as dCas9 or other) that recognises the sequence-specific DNA-binding protein recognition sequence of the methylation-protectable restriction element, and the DNA methylase (such as M2.Eco1I) that recognises the DNA methylase recognition sequence (ii) of the methylation-protectable restriction element. In an alternative embodiment, the circular destination vector (otherwise referred to as an assembly vector) is prepared in vitro with the switch DNA methylase (such as M.Sen0728I/S.Sen0728I) that recognises the switch DNA methylase recognition sequence of the methylation-switch element and the circular donor vector(s) may be prepared in vitro with the sequence-specific DNA-binding protein (such as dCas9 or other) that recognises the sequence-specific DNA-binding protein recognition sequence of the methylation-protectable restriction element, and the DNA methylase (such as M2.Eco1I) that recognises the DNA methylase recognition sequence (ii) of the methylation-protectable restriction element.

Advantageously, the provision of the DNA methylase that recognises the DNA methylase recognition sequence (ii) of the methylation-protectable restriction element provides that any sequences that are recognised and cut by the type IIS restriction enzyme, such as BsaI, are methylated and thereby protected by such a DNA methylase, which recognises and methylates the same sequence. Therefore, potential internal cut sites are protected from cutting and they do not need to be eliminated or designed out of the desirable insert fragment sequence. The provision of the sequence-specific DNA-binding protein allows cutting of any cut sites that have been provided with an associated recognition sequence for the second sequence-specific DNA-binding protein (i.e. those cut sites of the methylation-protectable restriction element), such that the binding of the sequence-specific DNA-binding protein prevents the DNA-methylase from binding and methylating the cut site, where it remains unprotected. The same restriction enzyme recognition sequence for the methylation-protectable restriction elements, the opposing restriction enzyme recognition sequence on the discard sequence, and the restriction enzyme recognition sequences on the donor vectors provides that a single type of restriction enzyme can be used. This allows for reduced cost/complexity in removing internal type IIS restriction site from DNA parts, because any unwanted restriction sites are protected by methylation. This makes assembly of longer sequence easier and less costly. Furthermore, the invention allows for a so-called “one-pot” reaction, whereby the restriction and ligation can be carried out in one pot in a single step.

Therefore, the method may combine the steps of restricting and ligating. In particular, the circular destination vector, the donor vector(s), the restriction enzyme and the ligase may be provided in the same composition.

For certain embodiments of the methods of the invention, the methylation-protectable restriction element of an assembly vector may not require a sequence-specific DNA binding protein recognition sequence. i.e. such a feature may be optional for the assembly vector. The initial methylation control of the assembly vector may be through the switch DNA methylase recognition sequence of the methylation switch element when preparing for a discard sequence to be removed. For example, “the methylation-protectable restriction element” may comprise either the sequence-specific DNA binding protein recognition sequence or the switch DNA methylase recognition sequence of the methylation switch element, or it may comprise both of these features. However, the assembly vector in the method of the invention may be used in combination with one or more insert plasmids that do comprise the methylation-protectable restriction element comprising a sequence-specific DNA binding protein recognition sequence.

Following assembly of the single assembled DNA molecule, the method may further comprise the step of transforming the single assembled DNA molecule into a bacterial strain that does not express the switch DNA methylase that recognises the switch DNA methylase recognition sequence of the methylation-switch element. Additionally or alternatively, the bacterial strain may express the sequence-specific DNA-binding protein that recognises the recognition sequence of the methylation-protectable restriction element and the DNA methylase, such as M2.Eco31I, that recognises the DNA methylase recognition sequence (ii) of the methylation-protectable restriction element. Alternatively, following assembly of the single assembled DNA molecule, the method may further comprise the step of cloning the single assembled DNA molecule in the absence of the switch DNA methylase that recognises the switch DNA methylase recognition sequence of the methylation-switch element. Additionally or alternatively, the cloning may be in the presence of the sequence-specific DNA-binding protein that recognises the recognition sequence of the methylation-protectable restriction element and the DNA methylase, such as M2.Eco31I, that recognises the DNA methylase recognition sequence (ii) of the methylation-protectable restriction element.

Advantageously, transforming the single assembled DNA molecule into a bacterial strain that does not express the switch DNA methylase that recognises the switch DNA methylase recognition sequence of the methylation-switch element (or cloning in the absence thereof) results in the cloning and production of a nucleic acid sequence, such as a vector, that comprises a non-methylated (i.e. non-blocked) restriction enzyme recognition sequence to allow subsequent restriction reactions.

A further advantage is that if the insert DNA comprises one or more type IIS restriction enzyme recognition sequence that are the same as the type IIS restriction enzyme recognition sequence (i) of the methylation-protectable restriction element, the single assembled DNA molecule can be transformed into a bacterial strain that expresses the DNA methylase, and expresses the sequence-specific DNA-binding protein that recognises the recognition sequence of the methylation-protectable restriction element (or cloning the single assembled DNA molecule in vitro in the presence of such DNA methylase and sequence-specific DNA-binding protein), thereby blocking aberrant cutting within the inserted sequences, but allowing cutting of the methylation-protectable restriction element, with the same type IIS restriction enzyme.

The method may further comprise a multistage assembly process in order to form assembled DNA fragments or vectors of greater sequence length. For example the assembled DNA, which is the product of the method of the invention, may be used further for a higher order assembly. For example, the method may comprise a second round of DNA assembly according to the invention wherein the DNA fragments of interest for assembly with the linearised methylated nucleic acid may comprise assembled DNA from a first/earlier DNA assembly round according to the invention.

Providing multiple rounds of DNA assembly to form larger fragments of DNA advantageously reduces the number of adapter/overhang sequences that are necessary. The success rate of DNA assembly is also increased with fewer DNA fragments to be assembled. Additionally, it can be easier to screen for the successfully assembled DNA with fewer fragments assembled in a single reaction.

In one embodiment, the method of assembling DNA may comprise:
1) providing an assembly vector comprising a nucleic acid according to the invention herein, wherein the assembly vector comprises two head-to-head restriction elements, each comprising:
a) a BsaI/M2.Eco31I recognition sequence,
b) an opposing non-switchable BsaI/M2.Eco31I recognition sequence, which is on the opposite side of the cut site of the BsaI of a).
c) a M.Sen0738I or M.Osp8071I recognition sequence, which partially overlaps with the BsaI/M2.Eco31I recognition sequence of a),
d) optionally a sequence-specific DNA-binding protein recognition sequence, such as a dCas9 or dST1Cas9 recognition sequence, which is arranged to prevent binding and methylation by M2.Eco31I at the BsaI/M2.Eco31I recognition sequence of a), and
and wherein the assembly vector is methylated by M.Sen0738I or M.Osp8071I, for example by isolating the assembly vector from a bacterial strain that expresses M.Sen0738I or M.Osp8071I, and which optionally does not express the sequence-specific DNA-binding protein nor the M2.Eco31I methylase;
2) providing insert plasmids, which comprise two or more DNA fragments of interest for assembly, wherein the insert plasmids comprises two methylation-protectable restriction elements, each comprising:
a) a BsaI/M2.Eco31I recognition sequence, and
b) a sequence-specific DNA-binding protein recognition sequence, such as a dCas9 or dST1Cas9 recognition sequence, which is arranged to prevent binding and methylation by M2.Eco31I at the BsaI/M2.Eco31I recognition sequence of a) and
wherein a sequence-specific DNA-binding protein and M2.Eco31I is provided, for example by preparing the insert plasmid in a strain expressing the sequence-specific DNA-binding protein and M2.Eco31I, such that the BsaI/M2.Eco31I recognition sequence of a) is protected from methylation by the sequence-specific DNA-binding protein preventing methylation by the M2.Eco31I, and
wherein the M2.Eco31I methylates any other BsaI/M2.Eco31I recognition sequence if present in the insert plasmid,
3) cutting non-methylated BsaI sites of the assembly vector and insert plasmids by providing BsaI, optionally in the same reaction, thereby providing linearised assembly vector and releasing the two or more insert fragments from the insert plasmids.
wherein a first insert fragment comprises a complementary overhang for ligation with a first end of the linearised methylated nucleic acid of the assembly vector, and a second insert fragment comprises complementary overhangs for ligation with the other/second end of the linearised methylated nucleic acid of the assembly vector; and
(i) the first and second insert fragments further comprise complementary overhangs for ligation with each other; or
(ii) the first and second insert fragments further comprise complementary overhangs for ligation with one or more further insert fragments having complementary overhangs, such that ligating the DNA fragments and the linearised methylated nucleic acid of the assembly vector with a DNA ligase would result in a single assembled DNA molecule; and
ligating the insert fragments and linearised methylated nucleic acid of the assembly vector with a ligase, optionally in the same reaction as the BsaI restriction, to form a single assembled DNA molecule comprising the sequence of the assembled insert fragments flanked by the BsaI/M2.Eco31I recognition sequences of the methylation-protectable restriction elements of the assembly vector.

In another embodiment, the method of assembling DNA may comprise:
1) providing an assembly vector comprising a nucleic acid according to the invention herein, wherein the assembly vector comprises two methylation-protectable restriction elements, each comprising:
a) a BsaI/M2.Eco31I recognition sequence,
b) an opposing BsaI/M2.Eco31I recognition sequence, which is on the opposite side of the cut site of the BsaI of a).
c) a M.Sen0738I or M.Osp8071I recognition sequence, which partially overlaps with the BsaI/M2.Eco31I recognition sequence of a),
d) optionally a sequence-specific DNA-binding protein recognition sequence, such as a dCas9 or dST1Cas9 recognition sequence, which is arranged to prevent binding and methylation by M2.Eco31I at the BsaI/M2.Eco31I recognition sequence of a), and
and wherein the assembly vector is methylated by M.Sen0738I or M.Osp8071I, for example by isolating the assembly vector from a bacterial strain that expresses M.Sen0738I or M.Osp8071I, and which optionally does not express the sequence-specific DNA-binding protein;
2) providing an insert plasmid, which comprises a DNA/insert fragment of interest for assembly, wherein the insert plasmid comprises two methylation-protectable restriction elements, each comprising:
a) a BsaI/M2.Eco31I recognition sequence, and
b) a sequence-specific DNA-binding protein recognition sequence, such as a dCas9 or dST1Cas9 recognition sequence, which is arranged to prevent binding and methylation by M2.Eco31I at the BsaI/M2.Eco31I recognition sequence of a) and
wherein a sequence-specific DNA-binding protein and M2.Eco31I is provided, for example by preparing the insert plasmid in a strain expressing the sequence-specific DNA-binding protein and M2.Eco31I, such that the BsaI/M2.Eco31I recognition sequence of a) is protected from methylation by the sequence-specific DNA-binding protein preventing methylation by the M2.Eco31I, and
wherein the M2.Eco31I methylates any other BsaI/M2.Eco31I recognition sequence if present in the insert plasmid,
3) cutting non-methylated BsaI sites of the assembly vector and insert plasmid by providing BsaI, optionally in the same reaction, thereby providing linearised assembly vector and releasing the DNA/insert fragment of interest from the insert plasmid, wherein the insert fragment comprises complementary overhangs for ligation with the linearised methylated nucleic acid of the assembly vector; and
ligating the insert fragment and linearised methylated nucleic acid of the assembly vector with a ligase, optionally in the same reaction as the BsaI restriction, to form a single assembled DNA molecule comprising the sequence of the assembled insert fragment flanked by the BsaI/M2.Eco31I recognition sequences of the methylation-protectable restriction elements of the assembly vector.

Alternatively, the assembly vector may be methylated by M.Sen0738I or M.Osp8071I in vitro.

Additionally or alternatively, the sequence-specific DNA-binding protein and M2.Eco31I may be provided by preparing the insert plasmid in vitro with the sequence-specific DNA-binding protein and M2.Eco31I, such that the BsaI/M2.Eco31I recognition sequence of a) is protected from methylation by the sequence-specific DNA-binding protein preventing methylation by the M2.Eco31I.

The methylation steps of the invention may be carried out in vitro, for example by preparing the nucleic acid in the presence of the described methylase(s).

The assembled DNA molecule may be transformed into a bacterial strain that does not express a functional M2.Eco31I. Additionally or alternatively the bacterial strain may not express a functional M.Sen0738I or M.Osp8071I. Additionally or alternatively the bacterial strain may express the sequence-specific DNA-binding protein and/or M2.Eco31I. Alternatively, the assembled DNA molecule may be cloned in the absence of a functional M2.Eco31I. Additionally or alternatively the cloning may not be in the presence of a functional M.Sen0738I or M.Osp8071I. Additionally or alternatively the may be in the presence of the sequence-specific DNA-binding protein and/or M2.Eco31I.

References to the expression or absence of expression of M.Sen0738I herein may also include the respective expression or absence of expression of S.Sen0738I, which the skilled person will understand needs to be co-expressed with M.Sen0738I to form a functional methylase.

The options for final transformation of the assembled DNA molecule may depend on whether the desire is to provide a methylated or non-methylated assembled DNA molecule, where the skilled person would have control over the methylation of any internal BsaI restriction sites and BsaI restriction sites of the methylation-controlled restriction element.

Scarless DNA Assembly Method

According to another aspect of the present invention, there is provided a method of scarless DNA assembly of DNA fragments comprising the steps of:
(A) providing a first intermediate vector, comprising the steps of:
providing a first linearised methylated nucleic acid by providing an assembly vector comprising a nucleic acid according to the invention herein, wherein the assembly vector comprises a maintenance-type design element and an excision-type design element flanking a discard sequence,
wherein the type IIS restriction enzyme recognition sequences (i) of the maintenance-type design element and the excision-type design element are selectively methylated, such that the outside type IIS restriction enzyme recognition sequences (i) of the maintenance-type design element and excision-type design element in the vector backbone are methylated, and the inside type IIS restriction enzyme recognition sequences of the maintenance-type design element and excision-type design element in the discard sequence are not methylated, and
cutting the assembly vector with the type IIS restriction enzyme that recognises the opposing type IIS restriction enzyme recognition sequences of the maintenance-type design element and excision-type design element that are in the discard sequence, and which are not methylated,
further providing a first DNA/insert fragment for assembly with the first linearised methylated nucleic acid, the first DNA/insert fragment having overhang ends that are adapted to ligate to the overhang ends of the first linearised methylated nucleic acid, wherein any DNA methylase recognition sequences in the DNA fragment have been methylated with the DNA methylase that recognises the DNA methylase recognition sequence (i);
ligating the first DNA/insert fragment for assembly and first linearised methylated nucleic acid with a ligase to form a first methylated intermediate vector comprising a first DNA/insert fragment for assembly flanked by methylation-protectable restriction elements;
transforming the first methylated intermediate vector into a bacterial strain that expresses the DNA methylase that recognises the DNA methylase recognition sequence (i) and the sequence-specific DNA binding protein that recognises the sequence-specific DNA binding protein recognition sequence of the methylation-protectable restriction elements in the first methylated intermediate vector, such that any type IIS restriction enzyme recognition sequences in the first DNA/insert fragment are methylated and the type IIS restriction enzyme recognition sequence of the methylation-protectable restriction elements are not protected;
isolating the first intermediate vector;
(B) providing a second intermediate vector, comprising the steps of:
providing a second linearised methylated nucleic acid by providing an assembly vector comprising a nucleic acid according to the invention herein, wherein the assembly vector comprises a maintenance-type design element and an excision-type design element flanking a discard sequence,
wherein the type IIS restriction enzyme recognition sequences (i) of the maintenance-type design element and the excision-type design element are selectively methylated, such that the outside type IIS restriction enzyme recognition sequences (i) of the maintenance-type design element and excision-type design element in the vector backbone are methylated, and the inside type IIS restriction enzyme recognition sequences of the maintenance-type design element and excision-type design element in the discard sequence are not methylated, and
cutting the assembly vector with the type IIS restriction enzyme that recognises the opposing type IIS restriction enzyme recognition sequences of the maintenance-type design element and excision-type design element that are in the discard sequence, and which are not methylated,
further providing a second DNA/insert fragment for assembly with the second linearised methylated nucleic acid, the second DNA/insert fragment having overhang ends that are adapted to ligate to the overhang ends of the second linearised methylated nucleic acid, wherein any DNA methylase recognition sequences in the second DNA/insert fragment have been methylated with the DNA methylase with the DNA methylase that recognises the DNA methylase recognition sequence (i);
ligating the second DNA/insert fragment for assembly and second linearised methylated nucleic acid with a ligase to form a second methylated intermediate vector comprising a second DNA/insert fragment for assembly flanked by methylation-protectable restriction elements;
transforming the second methylated intermediate vector into a bacterial strain that expresses the DNA methylase that recognises the DNA methylase recognition sequence (i), and the sequence-specific DNA binding protein that recognises the sequence-specific DNA binding protein recognition sequence of the methylation-protectable restriction elements in the second methylated intermediate vector, such that any type IIS restriction enzyme recognition sequences in the second DNA/insert fragment are methylated and the type IIS restriction enzyme recognition sequence of the methylation-protectable restriction elements are not protected;
isolating the second intermediate vector;
(C) cutting the first intermediate vector with a type IIS restriction enzyme that recognises the type IIS restriction enzyme recognition sequences of the methylation-protectable restriction elements, thereby forming a first adapted DNA/insert fragment that comprises a maintained-overhang sequence that is determined by the maintenance-type design element and an opposing native-overhang sequence that is determined by the native sequence of the first DNA/insert fragment for assembly;
(D) cutting the second intermediate vector with a type IIS restriction enzyme that recognises the type IIS restriction enzyme recognition sequences of the methylation-protectable restriction elements, thereby forming a second adapted DNA fragment insert that comprises a maintained-overhang sequence that is determined by the maintenance-type design element and an opposing native-overhang sequence that is determined by the native sequence of the second DNA fragment for assembly;
wherein
(i) the first and second adapted DNA/insert fragments are end fragments wherein their native-overhang sequences are complementary, such that they are arranged to ligate together; or
(ii) one or more middle DNA/insert fragments for assembly are provided wherein the first and second adapted DNA/insert fragments are respective end fragments in the assembly, and the one or more middle DNA fragments are arranged to be ligated between the first and second adapted DNA/insert fragments via complementary native-overhang sequences;
further comprising the step of ligating together, with a ligase, the first and second adapted DNA/insert fragments, or the first and second adapted DNA/insert fragments and one or more middle DNA/insert fragments, to form an assembled DNA fragment having maintained-overhangs at each end.

The excision-type design elements of the assembly vectors may comprise a partial type IIS restriction enzyme recognition sequence as described herein.

The one or more middle DNA/insert fragments may comprise native-overhang sequences at both ends that are determined by their native sequence. In an embodiment comprising a single middle DNA/insert fragment for assembly, the middle DNA/insert fragment may comprise a first native-overhang sequence that is complementary to the native-overhang of the first adapted DNA/insert fragment and a second native-overhang sequence that is complementary to the native-overhang of the second adapted DNA/insert fragment.

In an embodiment comprising a two or more middle DNA/insert fragments for assembly, the middle DNA/insert fragments may each comprise native-overhang sequences that are complementary to the native-overhang of a neighbouring middle DNA/insert fragment, such that they can be ligated together in a pre-determined order. The first and last middle DNA/insert fragments in a sequence will be arranged to ligate to the respective first adapted DNA/insert fragment and second adapted DNA/insert fragment via complementary native-overhang sequences.

In one embodiment, a middle DNA/insert fragment for assembly may be provided by
providing a further intermediate vector, comprising the steps of:
providing a further linearised methylated nucleic acid by providing an assembly vector comprising a nucleic acid according to the invention herein, wherein the assembly vector comprises a pair of excision-type design elements flanking a discard sequence,
wherein the type IIS restriction enzyme recognition sequences (i) of the excision-type design elements are selectively methylated, such that the outside type IIS restriction enzyme recognition sequences (i) of the excision-type design elements in the vector backbone are methylated, and the inside type IIS restriction enzyme recognition sequences of the excision-type design elements in the discard sequence are not methylated, and
cutting the assembly vector with the type IIS restriction enzyme that recognises the opposing type IIS restriction enzyme recognition sequences of the excision-type design elements that are in the discard sequence, and which are not methylated,
further providing a middle DNA/insert fragment for assembly with the further linearised methylated nucleic acid, the middle DNA/insert fragment having overhang ends that are adapted to ligate to the overhang ends of the further linearised methylated nucleic acid, wherein any DNA methylase recognition sequences in the second DNA/insert fragment have been methylated with a DNA methylase that recognises the DNA methylase recognition sequence (ii) of the methylation-protectable restriction element;
ligating the middle DNA/insert fragment for assembly and further linearised methylated nucleic acid with a ligase to form a further methylated intermediate vector comprising a middle DNA/insert fragment for assembly flanked by methylation-protectable restriction elements;
transforming the further methylated intermediate vector into a bacterial strain that expresses the DNA methylase that recognises the DNA methylase recognition sequence (ii) of the methylation-protectable restriction element, and expresses the sequence-specific DNA binding protein that recognises the sequence-specific DNA binding protein recognition sequences of the methylation-protectable restriction element, such that any type IIS restriction enzyme recognition sequences in the second DNA/insert fragment are methylated and the type IIS restriction enzyme recognition sequence of the methylation-protectable restriction elements are not protected;
isolating the further intermediate vector;
cutting the further intermediate vector with a type IIS restriction enzyme that recognises the type IIS restriction enzyme recognition sequences of the methylation-protectable restriction elements, thereby forming a middle adapted DNA/insert fragment that comprises opposing native-overhang sequences that are determined by the native sequences of the middle DNA/insert fragment for assembly.

The excision-type design elements of the assembly vectors may comprise a partial type IIS restriction enzyme recognition sequence as described herein.

In one embodiment different restriction enzymes and/or DNA methylases may be used for different methylation-protectable restriction elements. Preferably the same restriction enzymes and/or DNA methylases may be used for different methylation-protectable restriction elements.

The DNA/insert fragments for assembly may comprise double stranded DNA comprising the overhangs/stick ends at each end. A DNA/insert fragment for assembly may be prepared by PCR from a template, by gene synthesis, or by annealing of two oligonucleotides. Such processes, e.g. PCR or gene synthesis, may generate blunt ended double stranded DNA. For assembly starting with PCR product, an appropriate adaptor sequence containing the restriction site may be included in the PCR primer to trim the DNA to generate the overhangs for cloning into the intermediate vectors. For embodiments comprising the use of annealed oligonucleotides there are more choices. The sequence design could be the same as for PCR/gene synthesis with restriction sites at both ends for trimming with a restriction enzyme to generate the overhangs. Alternatively, two annealed oligos may form the overhangs themselves.

In one embodiment, the DNA/insert fragments for assembly, such as the first DNA/insert fragment for assembly, the second DNA/insert fragment for assembly, and the one or more adapted middle DNA/insert fragments for assembly, may be provided by PCR from a template sequence (i.e. the DNA fragments for assembly may be PCR products). The template sequence may provide the full sequence that is desired to be cloned as the assembled DNA/insert fragment. The DNA/insert fragments for assembly may be replicated, for example by PCR, from different sections of the template, and generated neighbouring PCR products may partially overlap in sequence (i.e. the sequence near the end of one PCR product may comprise the same sequence near the start of the next PCR product for assembly). The overlap may be partial, but sufficient in length to provide a complementary sticky end. In one embodiment, the overlap sequence within one PCR product (DNA/insert fragment for assembly) and the overlap in the next neighbouring PCR product (DNA/insert fragment for assembly) may be at least 2 base pairs in length. In another embodiment, the overlap sequence within one PCR product (DNA/insert fragment for assembly) and the overlap in the next neighbouring PCR product (DNA/insert fragment for assembly) may be at least 3 base pairs in length. In another embodiment, the overlap sequence within one PCR product (DNA/insert fragment for assembly) and the overlap in the next neighbouring PCR product (DNA/insert fragment for assembly) may be 3-6 base pairs in length. In another embodiment, the overlap sequence within one PCR product (DNA/insert fragment for assembly) and the overlap in the next neighbouring PCR product (DNA/insert fragment for assembly) may be 3-5 base pairs in length. In another embodiment, the overlap sequence within one PCR product (DNA/insert fragment for assembly) and the overlap in the next neighbouring PCR product (DNA/insert fragment for assembly) may be 4-5 base pairs in length. In another embodiment, the overlap sequence within one PCR product (DNA/insert fragment for assembly) and the overlap in the next neighbouring PCR product (DNA/insert fragment for assembly) may be 3-4 base pairs in length. In another embodiment, the overlap sequence within one PCR product (DNA/insert fragment for assembly) and the overlap in the next neighbouring PCR product (DNA/insert fragment for assembly) may be 4 base pairs in length.

PCR primers may be arranged to amplify the partially overlapping sections of template DNA in order to form the DNA/insert fragments for assembly that would comprise complementary overhangs. The PCR reaction may add adaptor sequences containing the restriction recognition sequence (i.e. the universal adapter sequence, for example the restriction enzyme recognition sequence may comprise the same sequence as the restriction enzyme recognition sequence of the maintained composite element and/or truncating composite element) plus the sequence for the overhang that would be complementary to the overhang of the neighbouring/consecutive fragment for assembly. The overhang comprising the sequence that overlaps, for example by 4 bp, between consecutive/neighbouring fragments may be formed following digestion with the restriction enzyme (i.e. the overlap among PCR product is referring to the sequence, for example of 4 bp, that inside the whole adaptor sequence comprising the restriction enzyme recognition sequence).

DNA/insert fragments for assembly may be methylated in vivo or in vitro with the DNA methylase that recognises the DNA methylase recognition sequence (ii) of the methylation-protectable restriction element. In one embodiment any DNA methylase recognition sequences of DNA/insert fragments for assembly may be methylated in vivo or in vitro with the DNA methylase that recognises the DNA methylase recognition sequence (ii) of the methylation-protectable restriction element, such as M2.Eco31I.

The PCR primers may further provide the adapter sequences for the DNA/insert fragments, which form the complementary overhang for ligation with the linearised methylated nucleic acids (such as the first linearised methylated nucleic acid, second linearised methylated nucleic acid or further linearised methylated nucleic acid) and formation of the intermediate vectors. The PCR primers may further provide a type IIS restriction enzyme recognition sequence that is arranged to direct the restriction enzyme to cut the PCR product to form the adapter sequences. The restriction enzyme recognition sequence provided by the PCR primers may be a type IIS restriction enzyme recognition sequence. The type IIS restriction enzyme recognition sequence may comprise the same sequence as the type IIS restriction enzyme recognition sequence of the maintenance-type design element and/or excision-type design element.

In one embodiment, the adapted middle DNA/insert fragment comprises a region of overlapping native sequence that is the same sequence as a native overhang sequence provided in a consecutive/neighbouring DNA/insert fragment intended for ligation. For example, where neighbouring/consecutive DNA/insert fragments for assembly with each other are encoded from a template, they may each comprise an overlapping/same sequence region from the template, which would form the complementary native overhangs between the two neighbouring/consecutive sequences in a subsequent ligation. In one embodiment, the adapted middle DNA/insert fragment comprises two regions of overlapping native sequence, that are arranged to form the native-overhangs of the middle DNA/insert fragment once cut by the recognising restriction enzyme.

The method may further comprise the step of providing a linearised destination vector for insertion of the assembled DNA fragments. The linearised destination vector may comprise respective overhang sequences that are complementary to the maintained overhangs of the assembled DNA fragment. The overhang sequences of the linearised destination vector may comprise a first overhang that is complementary to the maintained-overhang sequence of the first adapted DNA/insert fragment and a second overhang that is complementary to the second the maintained-overhang sequence of the second adapted DNA/insert fragment.

The linearised destination vector may be provided by cutting a circular destination vector with the restriction enzyme(s), such as BsaI, that is arranged to provide the same complementary overhangs as the assembled DNA fragments. For example, the linearised destination vector may be provided by cutting a circular destination vector with the restriction enzyme(s) that recognise the restriction enzyme recognition sequences of the maintenance-type design element and/or excision-type design element. The linearised destination vector may be a cloning vector.

The type IIS restriction enzyme recognition sequences (i) of the maintenance-type design element and/or the excision-type design element(s) may be selectively methylated by methylation with the switch DNA methylase, or by the DNA methylase that recognises the DNA methylase recognition sequence (ii) of the methylation-protectable restriction element.

The methylation with the switch DNA methylase may comprise methylating the nucleic acid in a strain that expresses the switch DNA methylase, or methylating in vitro with the switch DNA methylase. The methylation with the switch DNA methylase may comprise isolating the nucleic acid from a strain that expresses the switch DNA methylase.

The selective methylation with the DNA methylase that recognises the DNA methylase recognition sequence (ii) of the methylation-protectable restriction element may comprise methylating the nucleic acid in a strain that expresses the DNA methylase that recognises the DNA methylase recognition sequence (ii) of the methylation-protectable restriction element and the sequence-specific DNA binding protein, or methylating in vitro with the DNA methylase that recognises the DNA methylase recognition sequence (ii) of the methylation-protectable restriction element in the presence of the sequence-specific DNA binding protein. The selective methylation with the DNA methylase that recognises the DNA methylase recognition sequence (ii) of the methylation-protectable restriction element may comprise isolating the nucleic acid from a strain that expresses the DNA methylase that recognises the DNA methylase recognition sequence (ii) of the methylation-protectable restriction element and the sequence-specific DNA binding protein.

In an embodiment wherein the maintenance-type design element and/or the excision-type design element(s) comprise a first/outside methylation-protectable restriction element and an opposing second/inside methylation-protectable restriction element, only the type IIS restriction enzyme recognition sequence of the first/outside methylation-protectable restriction element may be methylated, and thereby protected. The type IIS restriction enzyme recognition sequence of the first/outside methylation-protectable restriction element may be methylated and the type IIS restriction enzyme recognition sequence of the second/inside methylation-protectable restriction element may not be methylated (i.e. protected) by isolating the nucleic acid from (or methylating the nucleic acid with) a strain that expresses the DNA methylase that recognises the DNA methylase recognition sequence (ii) of the first/outside methylation-protectable restriction element, wherein the strain does not express the sequence-specific DNA binding protein and/or any necessary guide nucleic acid that recognises the sequence-specific DNA binding protein recognition sequence of the first/outside methylation-protectable restriction element. Additionally or alternatively, the strain may expresses the DNA methylase that recognises the DNA methylase recognition sequence (ii) of the second/inside methylation-protectable restriction element, and expresses the sequence-specific DNA binding protein and/or any necessary guide nucleic acid that recognises the sequence-specific DNA binding protein recognition sequence of the second/inside methylation-protectable restriction element. The DNA methylase and the DNA methylase recognition sequence (ii) may be the same for both first/outside and second/inside methylation-protectable restriction elements. Alternatively, the methylation may be carried out in vitro with the DNA methylase in the presence of the relevant sequence specific DNA binding protein.

In one embodiment, the maintenance-type design element comprises the same sequence in all intermediate vectors that comprise the maintenance-type design element. In one embodiment, the excision-type design element comprises the same sequence in all intermediate vectors. In one embodiment, the maintenance-type design element comprises the same restriction enzyme recognition sequence as the excision-type design element. In one embodiment, the restriction enzyme recognition sequences of the maintenance-type design elements and/or the excision-type design elements of the intermediate vectors are the same. In particular the same restriction enzyme species may be used for restriction of the intermediate vectors, and optionally the destination vector.

The vectors used in the scarless assembly method of the invention may be high copy number vectors. For example the first and/or second linearised methylated nucleic acid may comprise a linearised methylated high copy number vector.

In one embodiment, the DNA assembly method of the invention is carried out in a single composition (i.e. so called “one-pot” process). For example, the DNA assembly process (including digestion with restriction enzyme and ligation) may be carried out in a single composition containing the restriction enzyme and DNA ligase using circular insert plasmids or linearized insert fragments and circular intermediate and/or destination vectors, or linearized versions thereof.

In one embodiment the methylation of the nucleic acid is carried out in a bacterial strain that is modified to expresses the methylase that recognises the methylase recognition sequence. In one embodiment the methylation of the nucleic acid is carried out in vitro.

For certain embodiments of the methods of the invention involving maintenance-type design elements and/or the excision-type design elements, the methylation-protectable restriction element of an assembly vector comprising a maintenance-type design element and/or the excision-type design element may not require a sequence-specific DNA binding protein recognition sequence. i.e. such a feature may be optional for the assembly vector. The initial methylation control of the assembly vector may be through the switch DNA methylase of the methylation-switch element when preparing for a discard sequence to be removed. For example, “the methylation-protectable restriction element” may comprise either the sequence-specific DNA binding protein recognition sequence or the switch DNA methylase recognition sequence of the methylation-switch element, or it may comprise both of these features. However, the assembly vector, in the methods of the invention requiring maintenance-type design elements and/or the excision-type design elements, can be used in combination with one or more insert/donor plasmids that do comprise the methylation-protectable restriction element comprising a sequence-specific DNA binding protein recognition sequence.

In embodiments not requiring sequence-specific DNA binding protein recognition sequences, the step of transforming the first methylated vector, second methylated vector and/or further methylated intermediate vector into a bacterial strain, maybe be in a strain that does not expresses the DNA methylase that recognises the DNA methylase recognition sequence (ii) of the methylation-protectable restriction element. For example, if the DNA fragment for assembly does not comprise internal restriction sites for the type IIS restriction enzyme that recognises the type IIS restriction enzyme recognition sequence (i) of the methylation-protectable restriction element. Such a strain may or may not expresses the sequence-specific DNA binding protein that recognises the sequence-specific DNA binding protein recognition sequences of the methylation-protectable restriction element.

In one embodiment, the nucleic acid comprises the sequence

(SEQ ID NO: 95)
NNNNNNNNNNNNNNNNGACN

(last 5 bp are the seed sequence that determines the specificity).

In one embodiment, for example for a VL (left insert vector), the nucleic acid comprises the sequence:

(SEQ ID NO: 96)
NNNNNNNNNNNNNNNNGACNNGGTCTCNCTCCNGAGACC(N^{x})GGTCTCN
AGACCNNGTCNNNNNNNNNNNNNNNN

In one embodiment, for example for a VM (middle insert vector), the nucleic acid comprises the sequence:

(SEO ID NO: 97)
NNNNNNNNNNNNNNNNGACNNGGTCTCCNGAGACC(N^{x})GGTCTCNAGAC
CNNGTCNNNNNNNNNNNNNNNN

In one embodiment, for example for a VR (right insert vector), the nucleic acid comprises the sequence:

(SEO ID NO: 98)
NNNNNNNNNNNNNNNNGACNNGGTCTCCNGAGACC(N^{x})GGTCTCNAGAC
NGAGACCNNGTCNNNNNNNNNNNNNNNN

In one embodiment, the nucleic acid comprises the sequence

| (SEQ ID NO: 99) | |
|---|---|
| | NNNNNNNNNNNCCAGNNNNN |

(last 5 bp are the seed sequence that determines the specificity).

In one embodiment, for example for a VL (left insert vector), the nucleic acid comprises the sequence:

(SEQ ID NO: 100)
NNNNNNNNNNNCCAGNNNNNNGGTCTCNCTCCNGAGACC(N^{X})GGTCTCN
AGACCNNNNNNCTGGNNNNNNNNNNN

In one embodiment, for example for a ML (middle insert vector), the nucleic acid comprises the sequence:

(SEQ ID NO: 101)
NNNNNNNNNNNCCAGNNNNNNGGTCTCCNGAGACC(N^{X})GGTCTCNAGAC
CNNNNNNCTGGNNNNNNNNNNN

In one embodiment, for example for a RL (right insert vector), the nucleic acid comprises the sequence:

(SEQ ID NO: 102)
NNNNNNNNNNNCCAGNNNNNNGGTCTCCNGAGACC(N^{X})GGTCTCNAGAC
NGAGACCNNNNNNCTGGNNNNNNNNNNN

(N^{x}) may mean any number of A, C, T or G, or between 0 bp and 300 kbp of A, C, T, or G.

The skilled person will recognise that other types of nucleic acid design can be made using sequences in Table 2.

Any methylation steps of the nucleic acid described herein in a bacterial strain, may alternatively be carried out in vitro, for example by exposing the nucleic acid directly to the desired methylase, with or without the presence of the sequence specific DNA binding protein (and any associated guide nucleic acid where necessary). In embodiments wherein methylation is carried out in vitro in the presence of the sequence specific DNA binding protein (and any associated guide nucleic acid where necessary), the nucleic acid may be incubated with the sequence specific DNA binding protein (and any associated guide nucleic acid where necessary) before the methylase.

In embodiments wherein methylation is carried out in vitro with or without the presence of the sequence specific DNA binding protein, the methylase (and the sequence specific DNA binding protein if present) may be inactivated, for example by heat. Additionally or alternatively, the nucleic acid may be isolated from such proteins.

Other Aspects

According to another aspect of the invention, there is provided the use of a sequence-specific DNA binding protein, such as dCas9, for controlling the methylation and/or restriction of the methylation-protectable restriction element according to the invention herein.

According to another aspect of the invention, there is provided the use of a sequence-specific DNA binding protein for steric hindrance of methylation and/or restriction of a first type IIS restriction enzyme recognition sequence in a nucleic acid, wherein the sterically blocking is effected by binding to or near the first type IIS restriction enzyme recognition sequence in the nucleic acid, wherein the nucleic acid comprises a second type IIS restriction enzyme recognition sequence that is the same sequence as the first type IIS restriction enzyme recognition sequence and wherein the second type IIS restriction enzyme recognition sequence is not arranged to be bound by or sterically hindered by the sequence-specific DNA binding protein.

The second type IIS restriction enzyme recognition sequence may be opposing (e.g. head to head) the first type IIS restriction enzyme recognition sequence. The second type IIS restriction enzyme recognition sequence may be arranged to cut at the same site as the first type IIS restriction enzyme recognition sequence. Alternatively, the second type IIS restriction enzyme recognition sequence may be arranged to the nucleic acid with the first type IIS restriction enzyme recognition sequence, or within the bases between the first type IIS restriction enzyme recognition sequence and its cut site.

The use may be for preventing the methylation of the methylation-protectable restriction element according to the invention herein. The use may be in combination with the DNA methylase described herein, for example M2.Eco31I. Additionally or alternatively, the use may be in combination with the type IIS restriction enzyme described herein, such as BsaI.

The sequence-specific DNA binding protein may be used to sterically prevent the binding of the DNA methylase that recognises the DNA methylase recognition sequence (ii) of the methylation-protectable restriction element.

According to another aspect of the invention, there is provided the use of a nucleic acid comprising opposing (i.e. head to head) BsaI/M2.Eco31I recognition sequences in combination with a sequence-specific DNA binding protein.

Guide-nucleic acid may also be used, where required.

According to another aspect of the invention, there is provided method of methylation protecting BsaI recognition sequences in a vector comprising the methylation-protectable restriction element according to the invention, wherein the vector comprises at least one BsaI recognition sequence that is not part of the methylation-protectable restriction element according to the invention
wherein the methylation comprises methylating the vector with M2.Eco31I in the presence of the sequence-specific DNA binding protein which recognises and binds to the sequence-specific DNA binding protein recognition sequence of the methylation-protectable restriction element according to the invention.

According to another aspect of the invention, there is provided a modified bacterial strain that is modified to express a switch DNA methylase of the methylation-switch element described herein.

According to another aspect of the invention, there is provided a modified bacterial strain that is modified to express the DNA methylase that recognises the DNA methylase recognition sequence (ii) of the methylation-protectable restriction element, such as M2.Eco31I, and the sequence-specific DNA binding protein described herein, such as dCas9 or dST1Cas9 (optionally with the guide nucleic acid).

The modified bacterial strain that is modified to express a DNA methylase may be an E. coli strain. The modification may comprise insertion of the methylase gene in the arsB locus of E. coli genome. The modified bacterial strain that is modified to express a DNA methylase may be formed by assembly of a vector with a methylase expression cassette followed by an antibiotic selection cassette, such as a zeocin antibiotic selection cassette, and flanked by homologous sequence, such as the homologous sequence from the arsb gene of E. coli, followed by PCR using this as a template to generate linear DNA containing the above element, and recombineering with this fragment followed by selection with the antibiotic, such as zeocin, to generate bacteria strains with the methylase expression cassette and selection marker stably inserted into the genome, such as the arsB locus of E. coli genome.

According to another aspect of the invention, there is provided the use of a modified bacterial strain that is modified to express a DNA methylase that recognises the DNA methylase recognition sequence (ii) of the methylation-protectable restriction element, for manufacturing a nucleic acid molecule according to the invention.

The modified bacterial strain may further be modified to express the sequence-specific DNA-binding protein described herein, for example dCas9 (optionally with the guide nucleic acid).

The DNA methylase and sequence-specific DNA-binding protein may be promoted from a different promoter. The sequence-specific DNA-binding protein may be promoted from a stronger promoter (such as J23119) relative to a weaker promoter (such as J23112) for the DNA methylase.

The modified bacterial strain may be E. coli.

According to another aspect of the invention, there is provided a method of producing a nucleic acid in the form of a vector according to the invention, the method comprising transforming a nucleic acid in the form of a vector according to the invention into a bacterial strain that is capable of replicating the vector, and wherein the bacterial strain is modified to express:
a) a DNA methylase that recognises the DNA methylase recognition sequence (ii) of the methylation-protectable restriction element, and
b) a sequence-specific DNA-binding protein described herein, for example dCas9 (optionally with the guide nucleic acid); and
optionally growing the bacteria, such that the nucleic acid is replicated and/or isolating the nucleic acid from the bacteria.

The nucleic acid may be methylated during production in the bacterial strain.

According to another aspect of the inventions, there is provided a composition comprising the nucleic acid according to the invention, or combinations of the vectors according to the invention.

The composition may further comprise one or more of:

a) a DNA methylase that recognises the DNA methylase recognition sequence (ii) of the methylation-protectable restriction element;

b) a sequence-specific DNA-binding protein described herein; and

c) a switch DNA methylase described herein.

The composition may further comprise a DNA ligase. The composition may comprise buffer.

According to another aspect of the inventions, there is provided a kit comprising one or more nucleic acids and/or vectors according to the invention herein.

The kit may further comprise one or more of:

a) a DNA methylase that recognises the DNA methylase recognition sequence (ii) of the methylation-protectable restriction element;

b) a sequence-specific DNA-binding protein described herein; and

c) a switch DNA methylase described herein.

The kit may further comprise a restriction enzyme and/or a ligase, such as T4 DNA ligase. Additionally or alternatively, the kit may comprise a modified bacterial strain that is modified to express one or more DNA methylases described herein and/or a sequence-specific DNA binding protein described herein (optionally with any guide nucleic acid as necessary).

Additionally or alternatively, the kit may comprise one or more buffer solutions. Additionally or alternatively, the kit may comprise antibiotics for clone selection.

One or more, or all, of the nucleic acid, buffer, bacterial strain, restriction enzyme, ligase, and methylase may be provided in a container, such as an Eppendorf tube.

According to another aspect of the invention, there is provided a host cell comprising nucleic acid according to the invention.

The host cell may further comprise nucleic acid for the expression of one or more of:

a) a DNA methylase that recognises the DNA methylase recognition sequence (ii) of the methylation-protectable restriction element;

b) a sequence-specific DNA-binding protein described herein; and

c) a switch DNA methylase described herein.

According to another aspect of the invention, there is provided the use of the nucleic acid according to the invention for assembling DNA fragments of interest, optionally wherein the use is in vitro.

The use of the nucleic acid may be with one or more of:

a) a DNA methylase that recognises the DNA methylase recognition sequence (ii) of the methylation-protectable restriction element;

b) a sequence-specific DNA-binding protein described herein; and

c) a switch DNA methylase described herein.

According to another aspect of the invention, there is provided the use of a sequence-specific DNA binding protein, such as nucleic acid-guided DNA binding protein, to protect a type IIS restriction enzyme recognition site from methylation by a DNA methylase that is capable of recognition and methylation of the type IIS restriction enzyme recognition site.

The protection of the type IIS restriction enzyme recognition site from methylation may be by steric hindrance. The protection of the type IIS restriction enzyme recognition site from methylation by the sequence-specific DNA binding protein may be by binding the sequence-specific DNA binding protein upstream of the type IIS restriction enzyme recognition at a distance sufficient to cause steric hindrance of the DNA methylase.

Reference to the term “scarless” or “scarless assembly” herein is intended to refer to a sequence of DNA that has been assembled from multiple sequences, wherein the joint between the sequences does not comprise artefacts, such as unwanted base pairs, of the restriction and ligation of the DNA assembly process. For example, scarless assembly of DNA does not introduce additional sequence into the assembled DNA fragment. In some literatures it is also called seamless DNA assembly. In one example, an adapter sequence designed to provide complementary sticky ends to facilitate the joining of DNA fragments may be considered a “scar” between the joined sequences.

Reference to the term “adapter” herein is intended to refer to a sequence designed to provide complementary sticky ends to facilitate the joining of DNA fragments.

Reference to the term “overlap” or “overlapping” in the context of overlapping recognition sequences is intended to refer to at least part of the recognition sequence of one enzyme (for example any nucleotide other than N in a recognition sequence) being in a position of the nucleotide sequence that specifies the recognition sequence of another enzyme. (i.e. overlapping sequences share at least some common nucleotides).

Reference to the term “native” in the context of an overhang/sticky end sequence is intended to refer to the natural, non-adapted, sequence of the DNA fragment of interest to be inserted or joined to the nucleic acid. For example, the sequence will not be designed or amended relative to the sequence of the template DNA of interest. This is in contrast with overhangs determined by the vector sequence to facilitate DNA assembly, which might be introduced as a scar into the assembled sequence.

Reference to a “single assembled DNA molecule” is intended to refer to a single type of molecule, and is not intended to refer to the number of copies of the same molecule. In particular, in any given reaction or composition, there may be a plurality of copies of the single assembled DNA molecule.

Reference to “high copy number” plasmids/vectors may refer to those with a copy number of 100 or more copies per cell, including but not limiting to plasmids with replication of origins such as 1. mutated pMB1 replication origin from plasmid pUC plasmids (Vieira and Messing 1982. Gene 19:259-268; Vieira and Messing 1987. Methods Enzymol. 153:3-11; Messing 1983. Methods Enzymol. 101:20-78; Lin-Chao et al. 1992. Mol. Microbiol. 6:3385-3393, each of which are incorporated by reference herein); and 2. ColE1-derived replication origin from pBluescript plasmids (https://www.chem-agilent.com/pdf/strata/212205.pdf—pBluescript II Phagemid Vectors, Instruction Manual, Catalog #212205, #212206. #212207 and #212208, Revision A.01, which is incorporated by reference herein). The replication origin carried by pbluescript is from pUC with a single nucleotide mutation. Particular guides on low and high copy plasmid/vector regulation can be found in Molecular Cloning A Laboratory Manual 3rd edition (2001), Joseph Sambrook and David W Russell, volume 1 page 1.4 Table 1-1 and volume 1 page 4.2 Table 4-1.

Reference to “low copy number” plasmids/vectors may refer to those with a copy number of less than 100 copies per cell, including but not limiting to plasmids with replication of origin such as 1. p15A replication origin from pACYC plasmids (Chang and Cohen 1978. J. Bacteriol. 134:1141-1156, which is incorporated by reference herein); 2. pSC101 replication origin from pSC101 plasmids (Stoker et al. 1982. Gene 18:335-341, which is incorporated by reference herein); 3. F replication origin from F plasmids (Kim et al. 1996. Genomics 34:213-218; Asakawa et al. 1997. Gene 191: 69-79, each of which are incorporated by reference herein); 4. pMB1 replication origin from pBR322 plasmids (Bolivar et al. 1977. Gene 2:95-113, which is incorporated by reference herein); and 5. ColE1 replication origin from ColE1 plasmid (Kahn et al. 1979. Methods Enzymol. 68:268-280, which is incorporated by reference herein)

Where reference is made to a variant polypeptide/protein or nucleotide sequence, the skilled person will understand that one or more amino acid residue or nucleotide substitutions, deletions or additions, may be tolerated, optionally two substitutions may be tolerated in the sequence, such that it maintains its function. The skilled person will appreciate that 1, 2, 3, 4, 5 or more amino acid residues or nucleotides may be substituted, added or removed without affecting function References to sequence identity may be determined by BLAST sequence alignment (www.ncbi.nlm.nih.gov/BLAST/) using standard/default parameters. For example, the sequence may have at least 99% identity and still function according to the invention. In other embodiments, the sequence may have at least 98% identity and still function according to the invention. In another embodiment, the sequence may have at least 95% identity and still function according to the invention. In another embodiment, the sequence may have at least 90%, 85%, or 80% identity and still function according to the invention. In one embodiment, the variation and sequence identity may be according the full length sequence. In other embodiments, the variation may be limited to non-conserved sequences and/or sequences outside of active sites, such as binding domains. Therefore, an active site or binding site of a protein may be 100% identical, whereas the flanking sequences may comprise the stated variations in identity. Such variants may be termed “conserved active site variants”.

Amino acid substitutions may be conservative substitutions. For example, a modified residue may comprise substantially similar properties as the wild-type substituted residue. For example, a substituted residue may comprise substantially similar or equal charge or hydrophobicity as the wild-type substituted residue. For example, a substituted residue may comprise substantially similar molecular weight or steric bulk as the wild-type substituted residue. With reference to “variant” nucleic acid sequences, the skilled person will appreciate that 1, 2, 3, 4, 5 or more codons may be substituted, added or removed without affecting function. For example, conservative substitutions may be considered.

The skilled person will understand that optional features of one embodiment or aspect of the invention may be applicable, where appropriate, to other embodiments or aspects of the invention.

Embodiments of the invention will now be described in more detail, by way of example only, with reference to the accompanying drawings.

FIG. 1. The methylation switching approach in the original MetClo system

A. A standard type IIS restriction site such as BsaI site (boxed) can be combined with a ‘switch’ methylase (M.Osp807II) recognition sequence (highlighted in grey, with the methylated base in bold) that partially overlaps with the restriction site to create a combined type IIS restriction site. The combined site is switchable by the ‘switch’ methylase in that methylation of the site by the M.Osp807II methylase blocks the restriction of the site by BsaI. The methylation can be removed by producing the plasmid in a normal E. coli strain that lacks the M.Osp807II switch methylase activity. An overlapping site that can be switched on or off by the M.Osp807II switch methylase is referred to as an M.Osp807II methylase-switchable site.

B. In contrast, a typical standard BsaI site does not have an overlap with an M.Osp807II switch methylase recognition site and so will not be methylated by the M.Osp807II switch methylase. Such sites can be restricted by BsaI and this cleavage is unaffected by the presence of absence of the of M.Osp807II switch methylase. The switching process can be represented using the symbols to the right, where the open triangles depict an unmethylated BsaI site, and the black triangles depict a methylated BsaI sites.

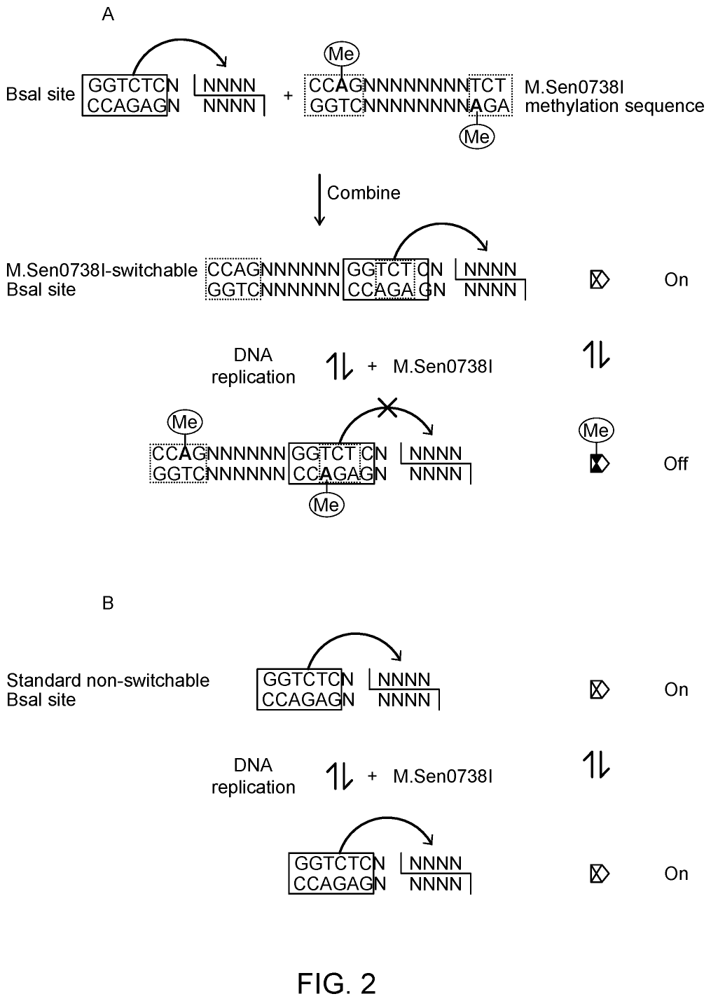

FIG. 2. Methylation switching using M.Sen0738I

A. A standard type IIS restriction site such as BsaI site (boxed) can be combined with a ‘switch’ methylase (M.Sen0738I) recognition sequence (highlighted in grey, with the methylated base in bold) that partially overlaps with the restriction site to create a ‘switchable’ type IIS restriction site. The combined site is switchable because methylation of the site by the M.Sen0738I switch methylase blocks the restriction of the site by BsaI. The methylation can be removed by producing the plasmid in a normal E. coli strain that does not express the M.Sen0738I switch methylase. An overlapping site that can be switched on or off in this way by the M.Sen0738I switch methylase is referred to as an M.Sen0738I methylase-switchable site.

B. In contrast, a typical standard BsaI site does not have an overlap with an M.Sen0738I switch methylase recognition site and so will not be methylated by the M.Sen0738I switch methylase. Such sites can be restricted by BsaI and this cleavage is unaffected by the presence of absence of the of M.Sen0738I switch methylase. The switching process can be represented using the symbols to the right, where the open triangles depict an unmethylated BsaI site, and the black triangles depict a methylated BsaI sites.

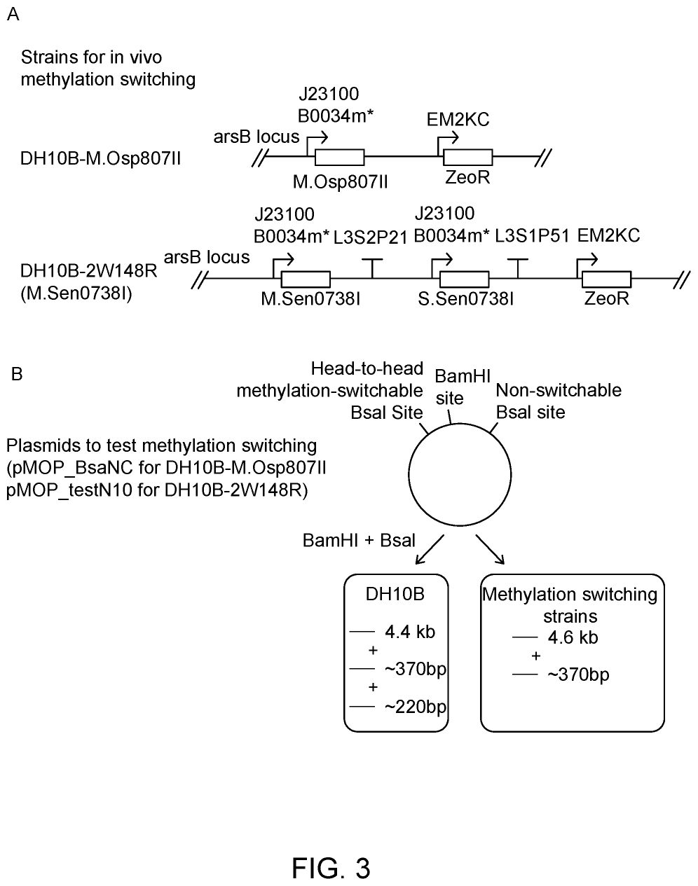

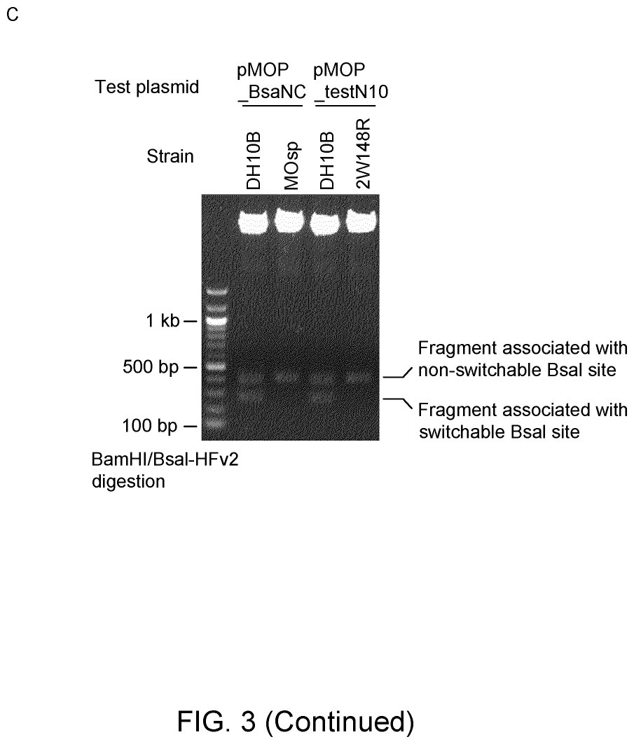

FIG. 3. Methylation switching in vivo using M.Osp807II and M.Sen0738I

A. Diagram depicting the strains for methylation switching in vivo. The DH10B-M.Osp807II expresses M.Osp807II from the arsB locus under the J23100 promoter, and a zeocin selection marker under EM2KC promoter. The strain DH10B-2W148R for M.Sen0738I-based methylation switching expresses M.Sen0738I and S.Sen0738I under J23100 promoter in tandem along with a zeocin selection marker under EM2KC promoter.

B. Experimental designs to test methylation switching in vivo. The test plasmids (pMOP_BsaNC for DH10B-M.Osp807II, and pMOP_testN10 for DH10B-2W148R) contain a head-to-head methylation-switchable BsaI site ˜220 bp away from an internal BamHI site, and a non-switchable BsaI site ˜370 bp away from the BamHI site. Restriction digestion by BamHI and BsaI of test plasmid prepared from a normal E. coli strain that does not express the switch methylase would result in cutting at both BsaI sites and the internal BamHI site resulting in release of both the ˜220 bp and ˜370 bp fragments from the vector backbone. Restriction digestion of test plasmid prepared from a strain expressing the switch methylase would not release the ˜220 bp fragment due to blocking of the methylation-switchable BsaI restriction sites by in vivo methylation. The head-to-head arrangement of overlapping methylation/restriction site allows the same assay to be used to detect any residual single strand nicking activity of the restriction enzyme towards the methylated restriction site.

C. Agarose gel electrophoresis analysis of the test plasmids prepared in E. coli strains DH10B-M.Osp807II (MOsp) or DH10B-2W148R (2W148R) that express the switch methylases M.Osp807II and M.Sen0738I respectively and digested with BamHI and BsaI-HFv2. The results show that in vivo methylation by each of the methylases successfully blocked the switchable BsaI restriction site.

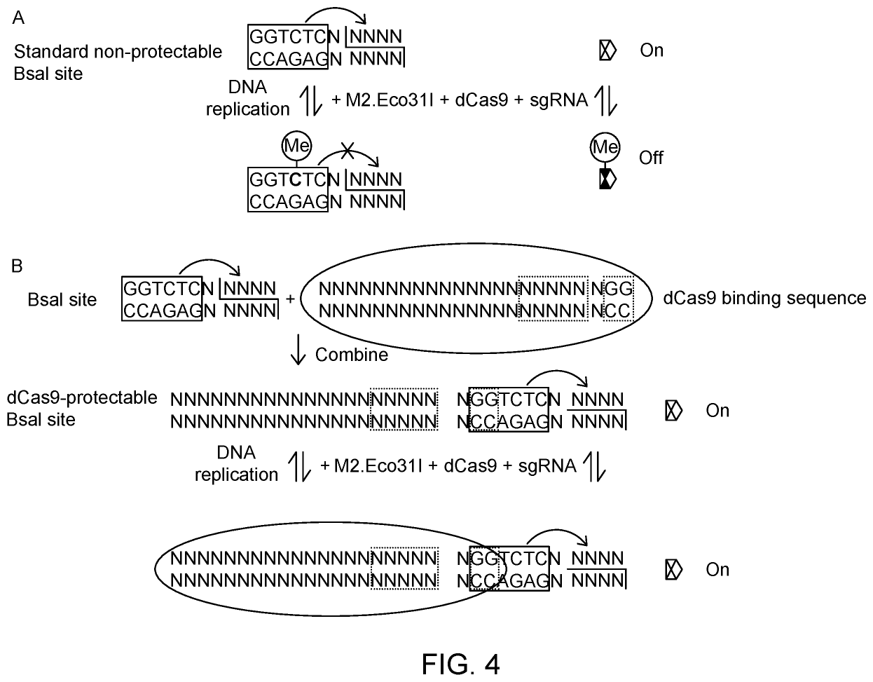

FIG. 4. The methylation protection approach

A. A standard type IIS restriction enzyme site such as BsaI (boxed) can be methylated by the M2.Eco31I ‘protection’ methylase at the 4th base of the top strand (in bold) when the plasmid is prepared in an E. coli strain that expresses the M2.Eco31I methylase, dCas9 and a synthetic guide RNA (sgRNA) targeting a specific sequence (the standard BsaI site must not overlap with the sgRNA-guided dCas9 binding sequence).

B. Combining a BsaI site (boxed) with the sgRNA-guided dCas9 binding site (the seed sequence and PAM sequence critical for dCas9 specificity are highlighted in grey) creates a BsaI site that overlaps with the dCas9-binding site. Preparation of the plasmid in an E. coli strain that expresses the M2.Eco31I protection methylase, dCas9 and the sgRNA that recognizes this combined site results in dCas9 binding to this site, which blocks the site from methylation by the M2.Eco31I protection methylase. Such site is referred to as a dCas9-protectable site. The methylation protection process can be represented by the symbols to the right; open triangles depicts an unmethylated BsaI site, and black triangles depict a methylated BsaI site.

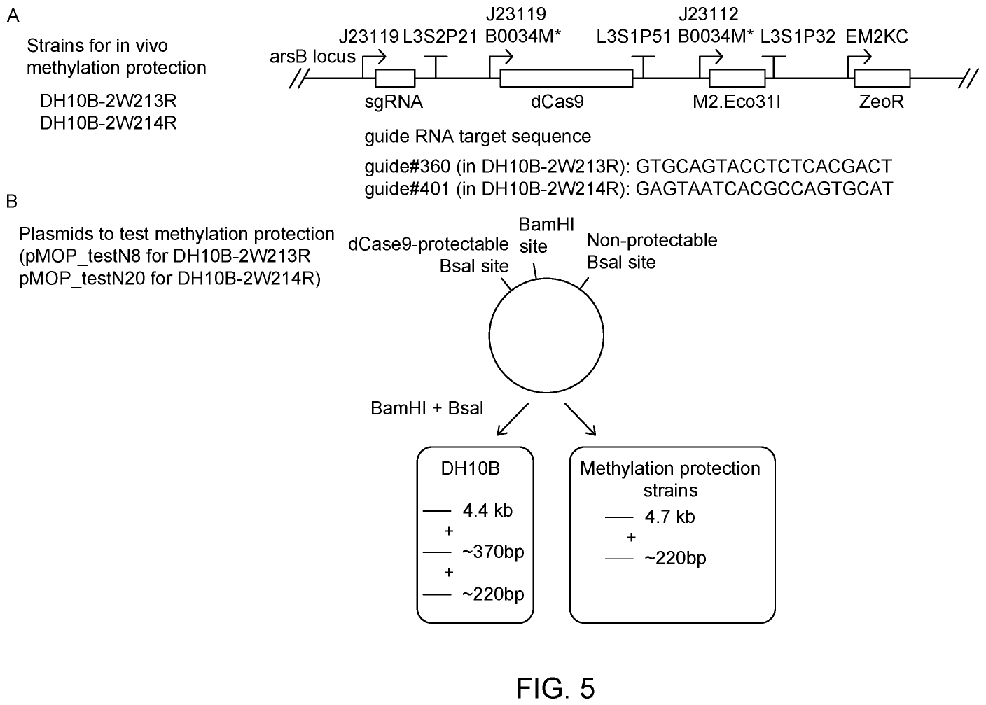

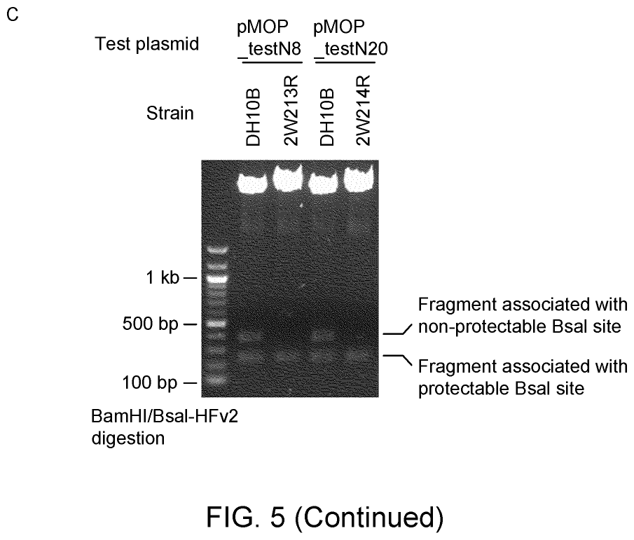

FIG. 5. Methylation protection using dCas9

A. Diagram depicting the E. coli strains for methylation protection. The strains express a synthetic guide RNA (guide #360 for strain DH10B-2W213R or guide #401 for strain DH10B-2W214R, driven by a J23119 promoter and with L3S2P21 terminator), dCas9 (driven by a J23119 promoter with a B0034m* ribosomal binding sequence (RBS) and L3S1P51 terminator), the M2.Eco31I methylase (driven by a J23112 promoter with a B0034m* RBS and L3S1P32 terminator) and a zeocin-resistance gene (ZeoR, driven by an EM2KC promoter) from the arsB locus of the E. coli chromosome.

B. Diagram of the plasmids used to test the methylation protection approach. The test plasmids (pMOP_testN8 for DH10B-2W213R and pMOP_testN20 for DH10B-2W214R) carry a dCas9-protectable BsaI site for the corresponding guide RNA sequence, a BamHI site, and a normal (non-switchable) BsaI site. If this test plasmid is prepared in a normal strain, such as DH10B, then both BsaI sites should be cut by BsaI. Therefore, digestion with BamHI and BsaI should generate two small fragments of ˜370 bp and ˜220 bp. If this test plasmid is prepared in the methylation protection strain that expresses the protection methylase, dCas9 and the corresponding guide RNA, then the normal BsaI site will be methylated and so will not be cut, but the methylation-protectable BsaI site will bound by the dCas9 and so will not be methylated. When the plasmid is exposed to BamHI and BsaI, of the two BsaI sites, only the unmethylated methylation-protectable BsaI site can be cut and this results in only one small fragment of ˜220 bp.

C. Gel electrophoresis of the test plasmids prepared from the DH10B or methylation protection strains following BamHI and BsaI-HFv2 digestion. The digested samples demonstrate the expected pattern as predicted in B, confirming successful protection from methylation of the dCas9-protectable BsaI site by the guided dCas9 in the DH10B-2W213R and DH10B-2W214R strains.

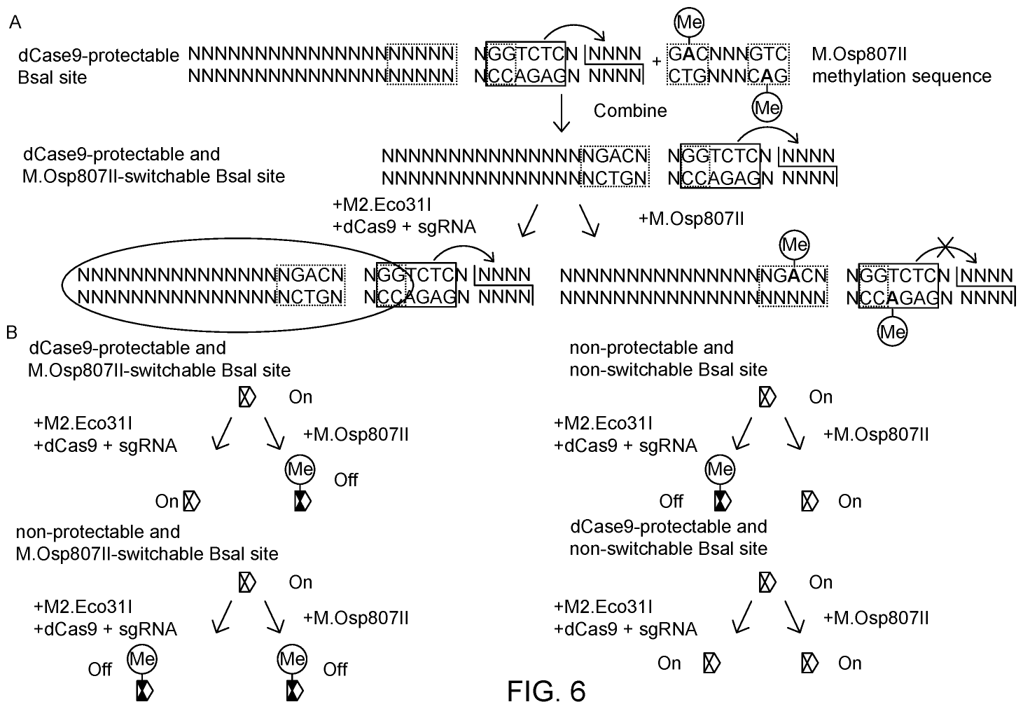

FIG. 6. Combined BsaI sites invoking both methylation-protection and methylation-switching. (compatible with M.Osp807II-based methylation switching)

A. Combining a dCas9-protectable BsaI site (the nucleotides required for dCas9 binding specificity are highlighted in grey and the BsaI site is boxed) and an M.Osp807II protection methylase site (highlighted in grey with the methylated bases in bold) generates a combined BsaI site that is both dCas9-protectable and M.Osp807II-switchable.

B. Systems deploying both methylation-protection and methylation-switching may contain four different types of BsaI sites. BsaI sites that are dCas9-protectable can be cut if the plasmid was produced in a strain that coexpresses the M2.Eco31I protection methylase, dCas9 and the appropriate sgRNA. However, the other standard BsaI sites in a plasmid produced in this strain are methylated by the M2.Eco31I protection methylase. BsaI sites that are methylation-switchable are not cut by BsaI if the plasmid has been produced in a strain that expresses the M.Osp807II switch methylase as these sites are then methylated and so protected from digestion by BsaI. Any non-switchable BsaI sites will not be methylated and can be cut by the enzyme if the plasmid has been produced in such a strain that expresses the M.Osp807II switch methylase.

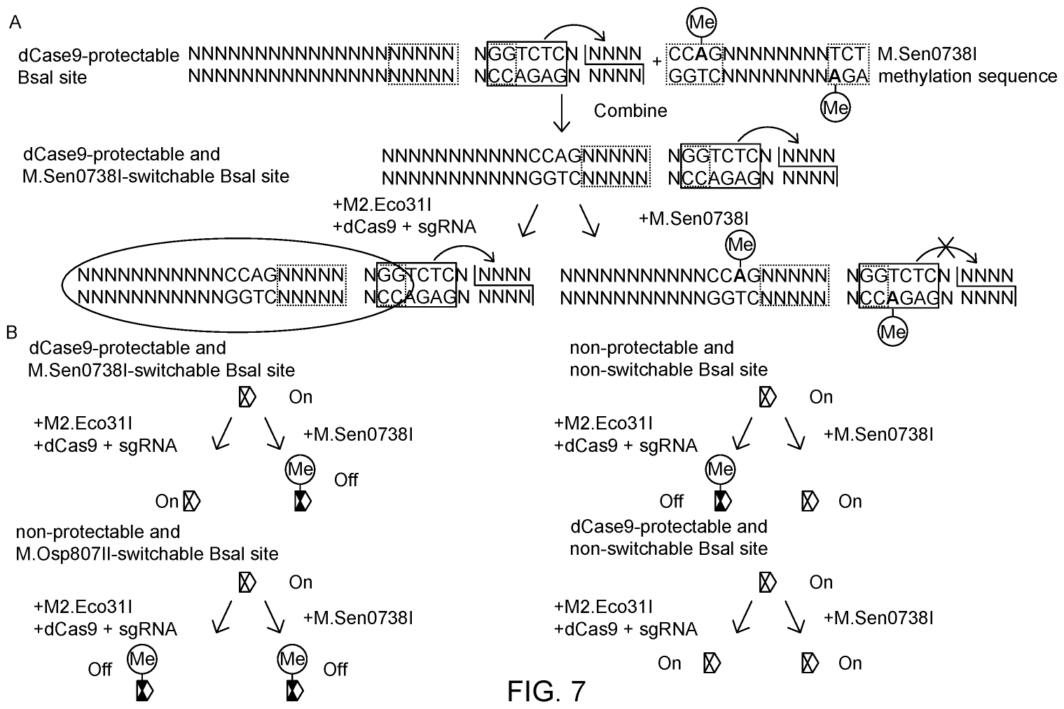

FIG. 7. Combined BsaI sites invoking both methylation-protection and methylation-switching. (compatible with M.Sen0738I-based methylation switching)

A. Combining a dCas9-protectable BsaI site (the nucleotides required for dCas9 binding specificity are highlighted in grey and the BsaI site is boxed) and an M.Sen0738I protection methylase site (highlighted in grey with the methylated bases in bold) generates a combined BsaI site that is both dCas9-protectable and M.Sen0738I-switchable.

B. Systems deploying both methylation-protection and methylation-switching may contain four different types of BsaI sites. BsaI sites that are dCas9-protectable can be cut if the plasmid was produced in a strain that coexpresses the M2.Eco31I protection methylase, dCas9 and the appropriate sgRNA. However, the other standard BsaI sites in a plasmid produced in this strain are methylated by the M2.Eco31I protection methylase. BsaI sites that are methylation-switchable are not cut by BsaI if the plasmid has been produced in a strain that expresses the M.Sen0738I switch methylase as these sites are then methylated and so protected from digestion by BsaI. Any non-switchable BsaI sites will not be methylated and can be cut by the enzyme if the plasmid has been produced in such a strain that expresses the M.Sen0738I switch methylase.

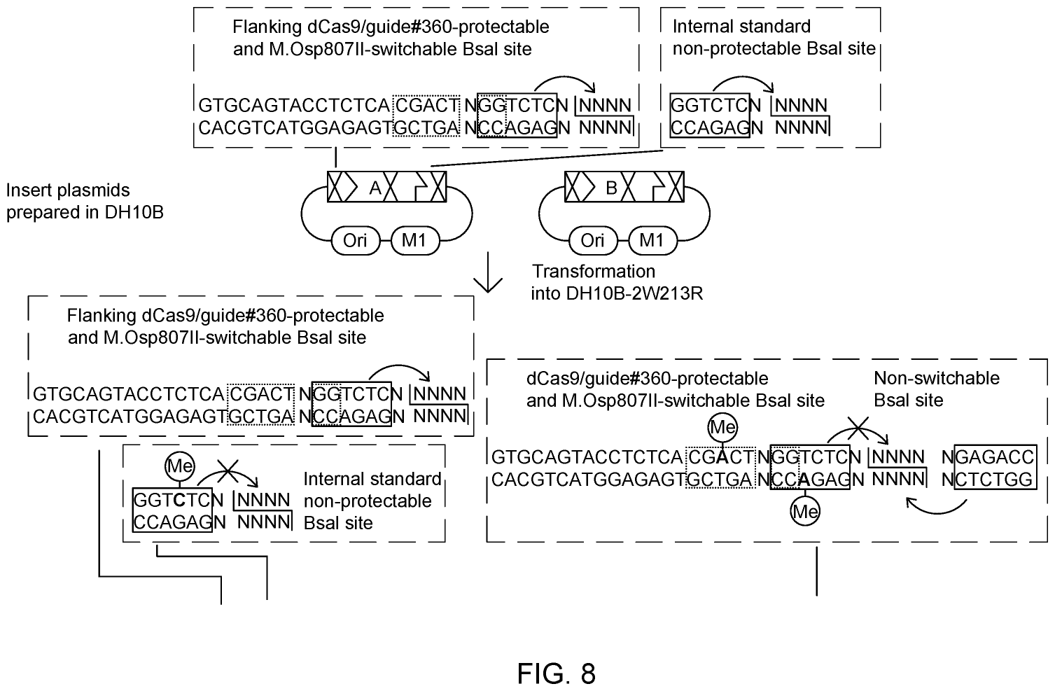

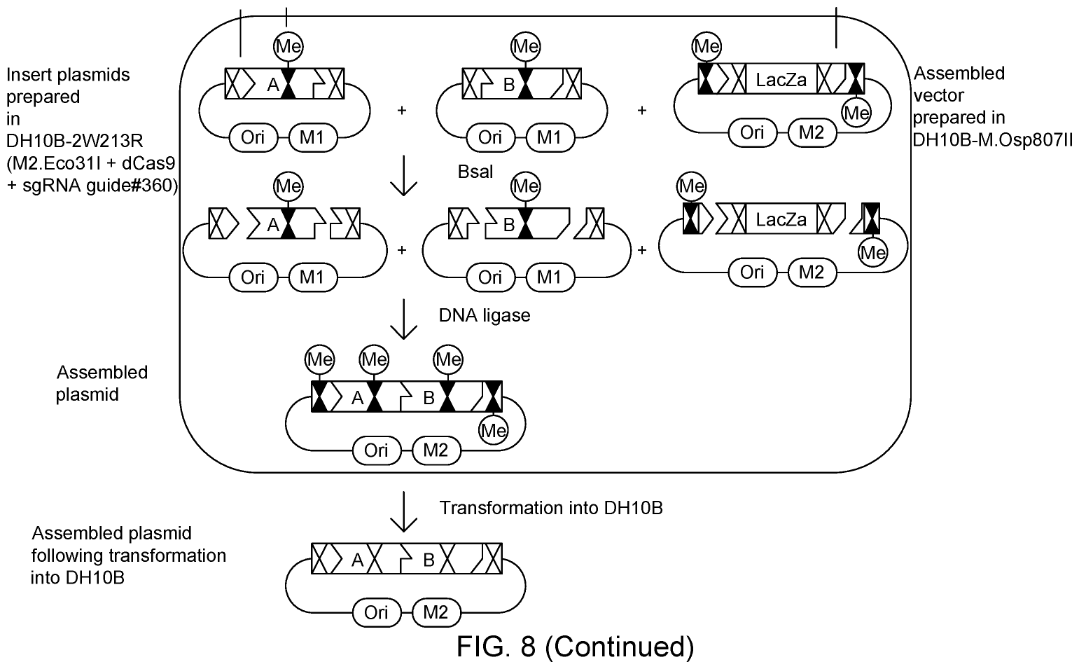

FIG. 8. Universal Assembly system based on both methylation-switching and methylation-protection (M.Osp807II-based methylation switching and dCas9/guide #360-based methylation protection as an example)

The diagram depicts the design of the implemented Universal Assembly system using methylation-protection and methylation-switching. The donor plasmids contain inserts flanked by dCas9/guide #360-protectable and M.Osp807II-switchable BsaI sites (the BsaI site is boxed and the nucleotides critical for dCas9 binding specificity are highlighted in grey) that would generate compatible adhesive ends following BsaI restriction. The internal standard BsaI sites in the insert do not overlap with guided-dCas9 binding sequence and therefore are not protected from methylation by the M2.Eco31I protection methylase. Transformation of the insert plasmids into an E. coli strain (DH10B-2W213R) that expresses the M2.Eco31I protection methylase, dCas9 and a sgRNA guide #360 targeting the dCas9-protectable BsaI site results in selective methylation of internal BsaI sites within the insert (the methylated base is shown in bold). The assembly vector contains a negative selection marker LacZalpha flanked by head-to-head BsaI sites that generate the same adhesive end as that produced by BsaI-based excision of the insert from the donor plasmid. The outer pair of BsaI sites are dCas9/guide #360-protectable and M.Osp807II methylation-switchable, whereas the inner pair of BsaI sites are nonswitchable. Preparation of the assembly recipient vector in an E. coli strain (DH10B-M.Osp807II) expressing the M.Osp807II switch methylase results in specific methylation of the outer pair of BsaI sites. The methylated insert donor plasmids and assembly recipient vector plasmids can be cut with BsaI. Ligation of the cut fragments in a one-pot reaction that contains BsaI favours the generation of correctly assembled DNA comprising the inserts ligated together with the assembly vector backbone, in which all the BsaI sites are methylated. Transformation into a normal E. coli (DH10B) that lacks dCas9-protection and methylation-switching activity removes methylation of all the BsaI sites in the assembled plasmid.

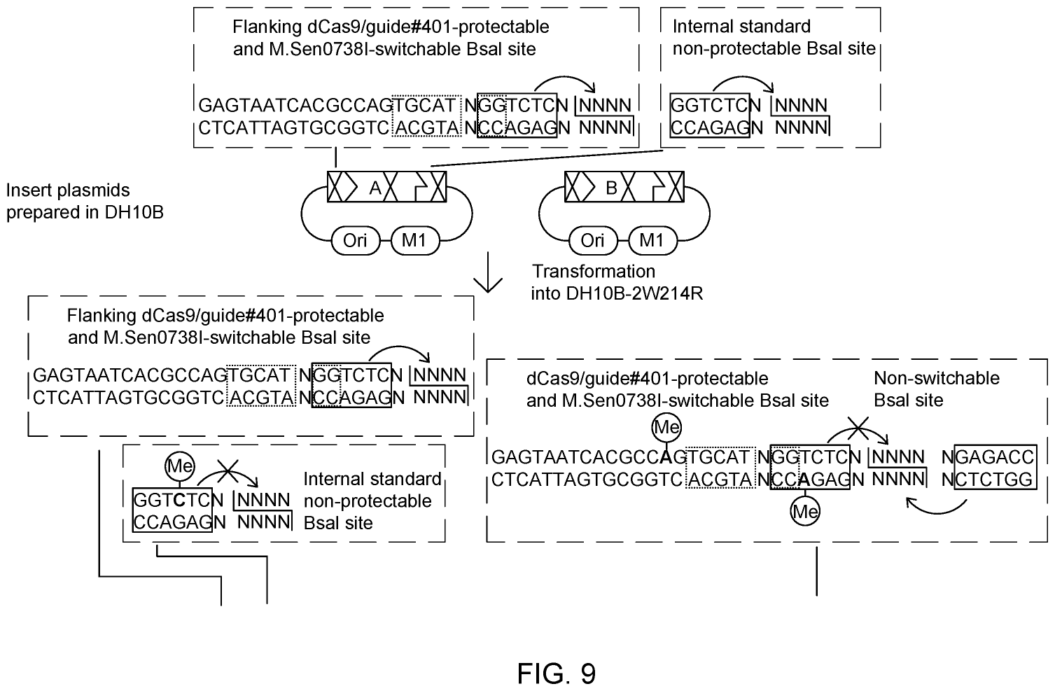

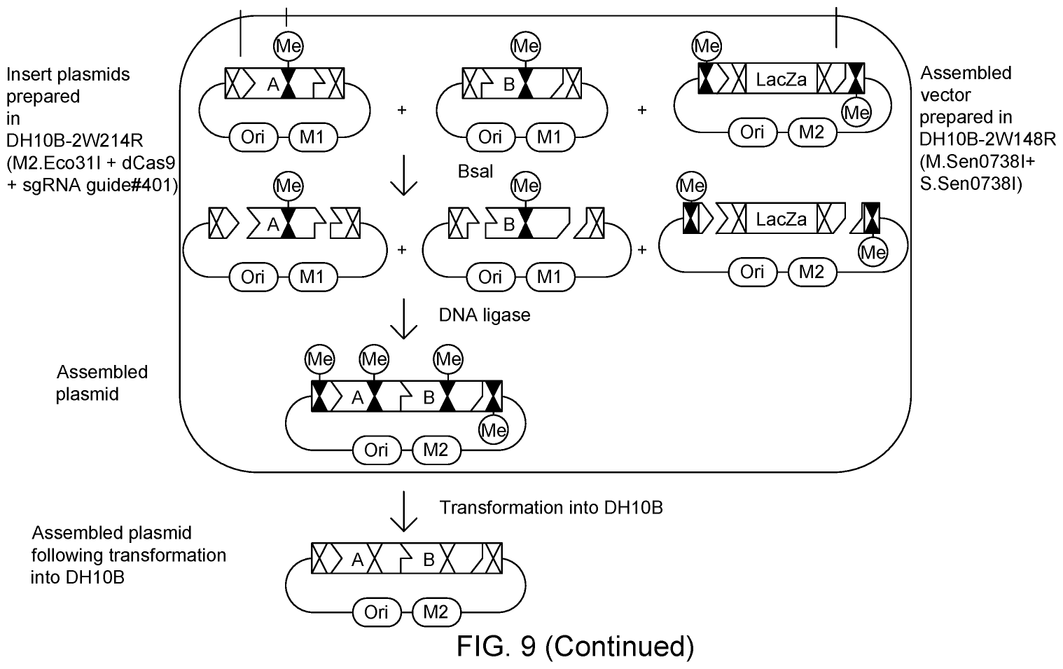

FIG. 9. Universal Assembly system based on both methylation-switching and methylation-protection (M.Sen0738I-based methylation switching and dCas9/guide #360-based methylation protection as an example)

The diagram depicts the design of the implemented Universal Assembly system using methylation-protection and methylation-switching. The donor plasmids contain inserts flanked by dCas9/guide #401-protectable and M.Sen0738I-switchable BsaI sites (the BsaI site is boxed and the nucleotides critical for dCas9 binding specificity are highlighted in grey) that would generate compatible adhesive ends following BsaI restriction. The internal standard BsaI sites in the insert do not overlap with guided-dCas9 binding sequence and therefore are not protected from methylation by the M2.Eco31I protection methylase. Transformation of the insert plasmids into an E. coli strain (DH10B-2W214R) that expresses the M2.Eco31I protection methylase, dCas9 and a sgRNA guide #401 targeting the dCas9-protectable BsaI site results in selective methylation of internal BsaI sites within the insert (the methylated base is shown in bold). The assembly vector contains a negative selection marker LacZalpha flanked by head-to-head BsaI sites that generate the same adhesive end as that produced by BsaI-based excision of the insert from the donor plasmid. The outer pair of BsaI sites are dCas9/guide #401-protectable and M.Sen0738I methylation-switchable, whereas the inner pair of BsaI sites are nonswitchable. Preparation of the assembly recipient vector in an E. coli strain (DH10B-2W148R) expressing the M.Sen0738I switch methylase results in specific methylation of the outer pair of BsaI sites. The methylated insert donor plasmids and assembly recipient vector plasmids can be cut with BsaI. Ligation of the cut fragments in a one-pot reaction that contains BsaI favours the generation of correctly assembled DNA comprising the inserts ligated together with the assembly vector backbone, in which all the BsaI sites are methylated. Transformation into a normal E. coli (DH10B) that lacks dCas9-protection and methylation-switching activity removes methylation of all the BsaI sites in the assembled plasmid.

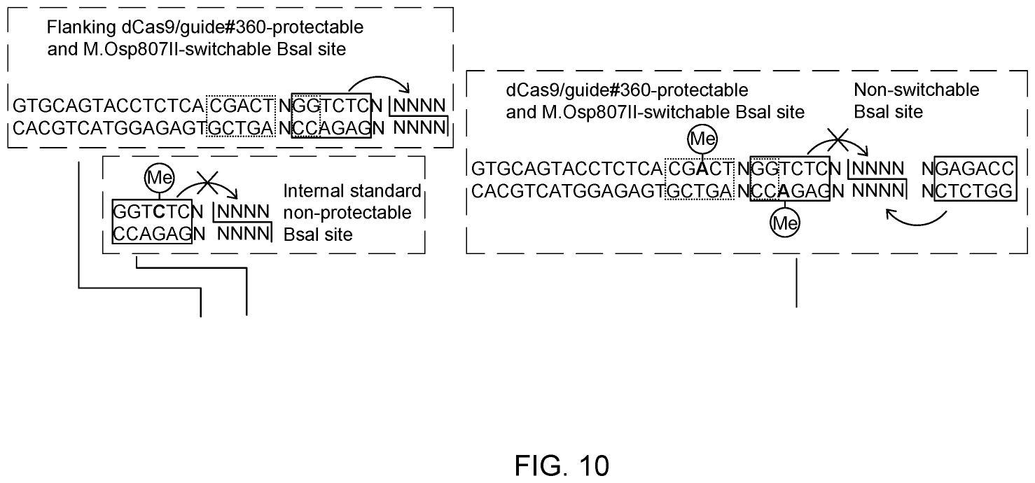

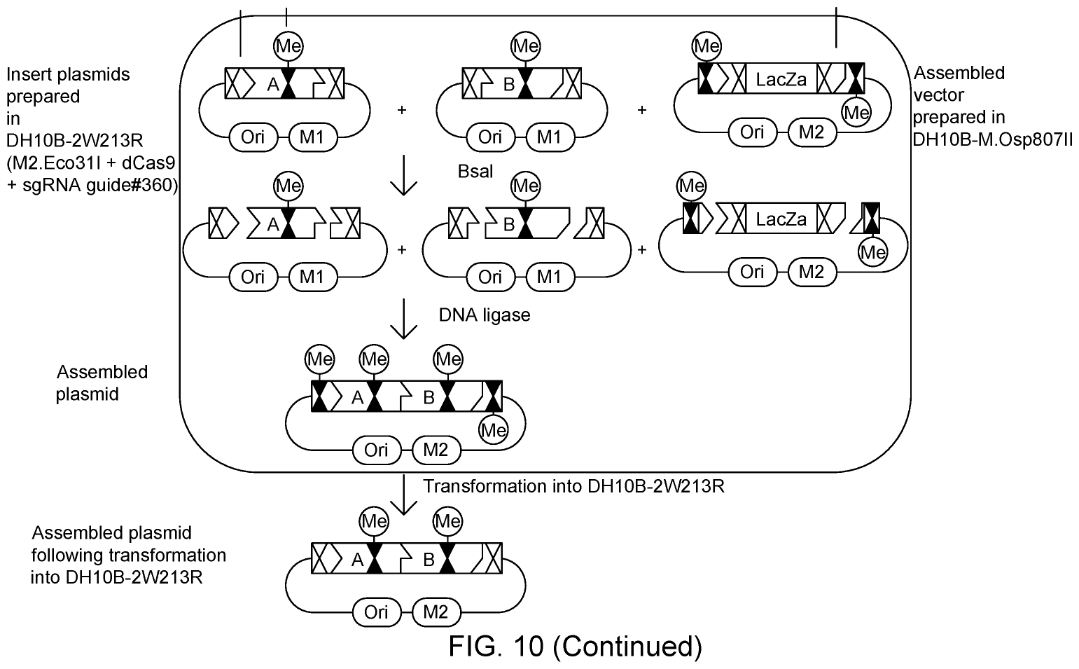

FIG. 10. Universal Assembly system based on both methylation-switching and methylation-protection (M.Osp807II-based methylation switching and dCas9/guide #360-based methylation protection as an example, direct transformation into DH10B-2W213R).

The diagram depicts the basic design of Universal Assembly with the assembled DNA directly transformed into an E. coli strain that deploys methylation-protection. The insert donor plasmids contain inserts flanked by dCas9/guide #360 methylation-protectable and M.Osp807II methylase-switchable BsaI sites (the BsaI site is boxed, the nucleotides critical for dCas9 specificity are highlighted in grey) that would generate mutually compatible adhesive ends following BsaI restriction. The internal standard BsaI sites in the insert do not overlap with the sequence that is the target of the guide RNA for the dCas9 and therefore are not bound by dCas9 and so are methylated by the M2.Eco31I protection methylase. Preparation of the insert plasmids in an E. coli strain (DH10B-2W213R) that expresses the M2.Eco31I protection methylase, dCas9 and an sgRNA guide #360 targeting the dCas9/guide #360 methylation-protectable BsaI results in specific methylation of standard internal BsaI sites within the insert (the methylated base is shown in bold). The assembly vector contains a negative selection marker LacZalpha flanked by head-to-head BsaI sites that generate the same adhesive end. The outer pair of BsaI sites are dCas9/guide #360-protectable and M.Osp807II methylase-switchable, whereas the inner pair are non-switchable. Preparation of the assembly vector in an E. coli strain expressing M.Osp807II (DH10B-M.Osp807II) results in specific methylation of the outer pair of BsaI sites. The methylated insert plasmids and assembly vector can be cut with BsaI. Ligation of the cut fragments in a one-pot reaction that contains BsaI favours the generation of correctly assembled DNA comprising the inserts ligated together with the assembly vector backbone, in which all the BsaI sites are methylated. Transformation into in the E. coli strain (DH10B-2W213R) that expresses the M2.Eco31I protection methylase, dCas9 and an sgRNA guide #360 targeting the dCas9/guide #360-protectable BsaI site, results in a plasmid that has methylation at all the BsaI sites in the insert, but no methylation at the dCas9/guide #360-protectable insert-flanking BsaI sites. The assembled DNA carries a similar methylation pattern on its BsaI sites to the original insert plasmids and so, therefore can be used directly for the next round of Universal Assembly.

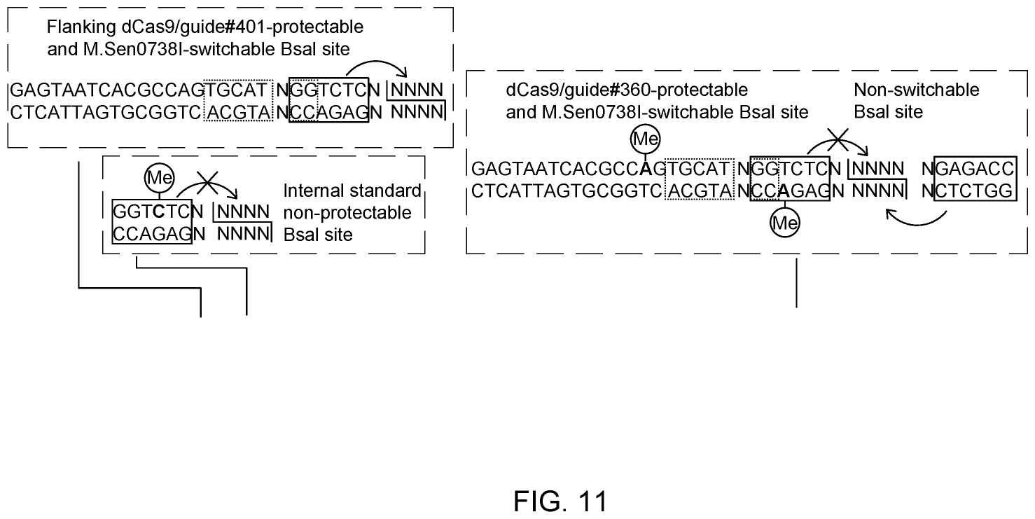

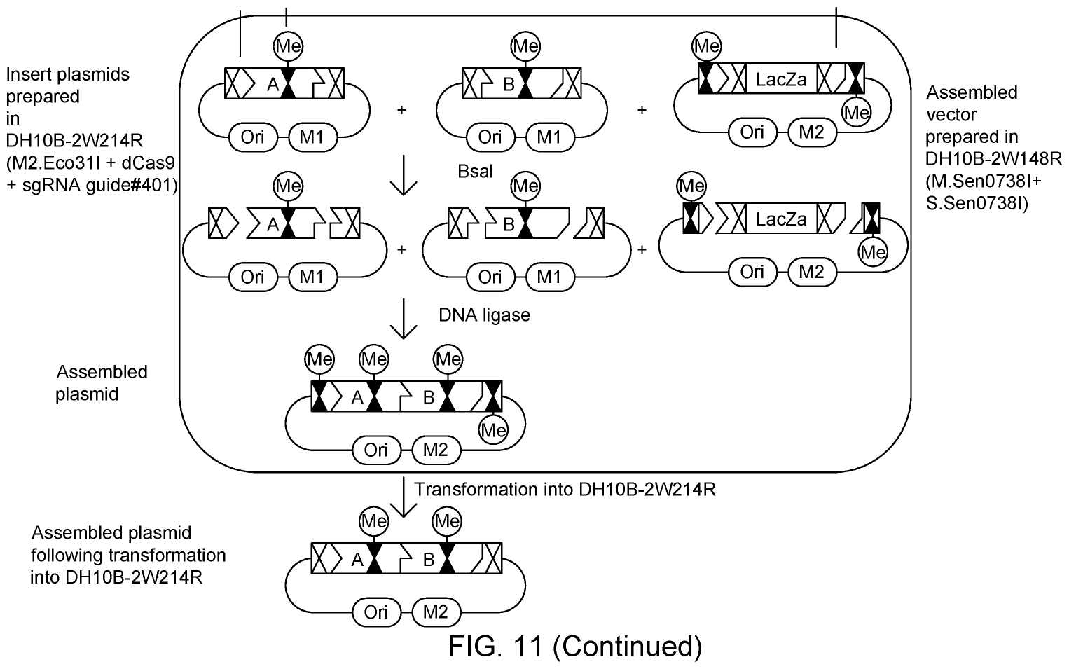

FIG. 11. Universal Assembly system based on both methylation-switching and methylation-protection (M.Sen0738I-based methylation switching and dCas9/guide #401-based methylation protection as an example, direct transformation into DH10B-2W214R).

The diagram depicts the basic design of Universal Assembly with the assembled DNA directly transformed into an E. coli strain that deploys the methylation-protection principle. The insert donor plasmids contain inserts flanked by dCas9/guide #401-protectable and M.Sen0738I methylase-switchable BsaI sites (the BsaI site is boxed, the nucleotides critical for dCas9 specificity are highlighted in grey) that would generate mutually compatible adhesive ends following BsaI restriction. The internal standard BsaI sites in the insert do not overlap with the sequence that is the target of the guide RNA for the dCas9 and therefore are not bound by dCas9 and so are methylated by the M2.Eco31I protection methylase. Preparation of the insert plasmids in an E. coli strain (DH10B-2W214R) that expresses the M2.Eco31I protection methylase, dCas9 and an sgRNA guide #401 targeting the dCas9/guide #401-protectable BsaI results in specific methylation of standard internal BsaI sites within the insert (the methylated base is shown in bold). The assembly vector contains a negative selection marker LacZalpha flanked by head-to-head BsaI sites that generate the same adhesive end. The outer pair of BsaI sites are dCas9/guide #401-protectable and M.Sen0738I methylase-switchable, whereas the inner pair are non-switchable. Preparation of the assembly vector in an E. coli strain expressing M.Sen0738I (DH10B-2W148R) results in specific methylation of the outer pair of BsaI sites. The methylated insert plasmids and assembly vector can be cut with BsaI. Ligation of the cut fragments in a one-pot reaction that contains BsaI favours the generation of correctly assembled DNA comprising the inserts ligated together with the assembly vector backbone, in which all the BsaI sites are methylated. Transformation into in the E. coli strain (DH10B-2W214R) that expresses the M2.Eco31I protection methylase, dCas9 and an sgRNA guide #401 targeting the dCas9/guide #401-protectable BsaI site, results in a plasmid that has methylation at all the BsaI sites in the insert, but no methylation at the dCas9/guide #360-protectable insert-flanking BsaI sites. The assembled DNA carries a similar methylation pattern on its BsaI sites to the original insert plasmids and so, therefore can be used directly for the next round of Universal Assembly.

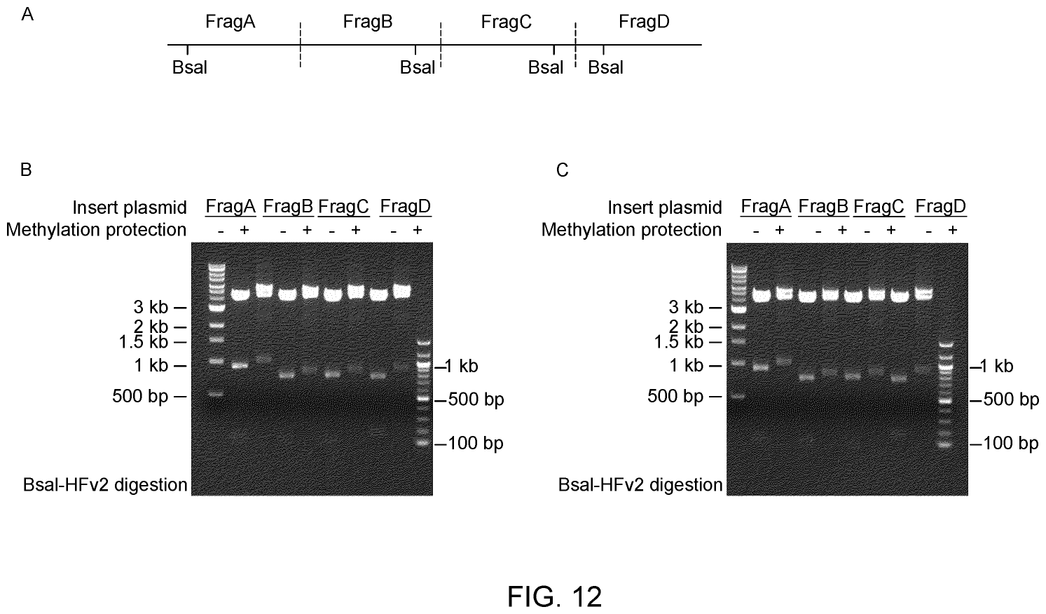

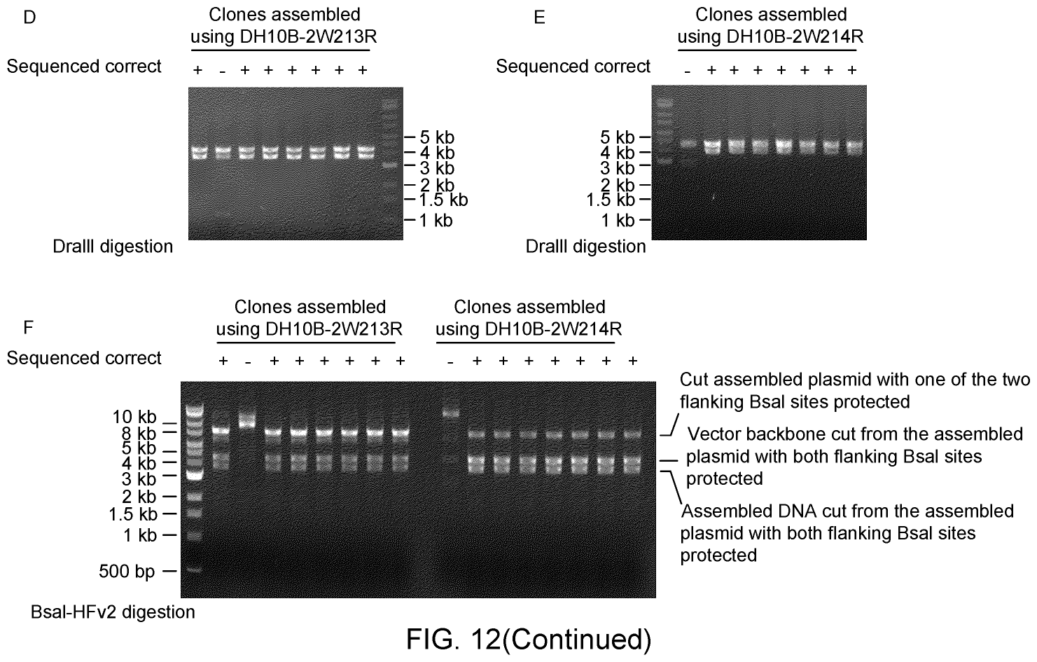

FIG. 12. Practical DNA assembly using Universal Assembly

A. Diagram of the DNA fragment to assemble. Four fragments of human DNA (FragA, FragB, FragC and FragD), each containing an internal BsaI site were assembled together to produce a 3.6 kb fragment as the final product.

B. Gel electrophoresis of BsaI digestion of insert plasmids with pMOK360 backbone prepared in the normal E. coli strain, DH10B, (−) or the E. coli strain DH10B-2W213R (+) that expresses dCas9, a guide RNA guide #360 and the M2.Eco31I protection methylase following BsaI-HFv2 digestion. dCas9-protection prevents methylation of the insert-flanking dCas9-protectable BsaI sites, but the internal BsaI sites are methylated and so cannot be cut by BsaI, resulting in larger complete insert fragments that are suitable for assembly. For insert plasmids prepared in DH10B-2W213R, BsaI-HFv2 digestion of insert plasmids with both flanking BsaI sites fully protected from methylation generates a ˜4.3 kb vector backbone, whereas BsaI-HFv2 digestion of insert plasmids with one of the two flanking BsaI sites protected from methylation generates a single ˜5.3 kb fragment.

C. Gel electrophoresis of BsaI digestion of insert plasmids with pMOK401 backbone prepared in the normal E. coli strain, DH10B, (−) or the E. coli strain DH10B-2W214R (+) that expresses dCas9, a guide RNA guide #401 and the M2.Eco31I protection methylase following BsaI-HFv2 digestion. The sequence-specific methylation-protection prevents methylation of the insert-flanking dCas9-protectable BsaI sites, but the internal BsaI sites are methylated and so cannot be cut by BsaI, resulting in larger complete insert fragments that are suitable for assembly. For insert plasmids prepared in DH10B-2W214R, BsaI-HFv2 digestion of insert plasmids with both flanking BsaI sites fully protected from methylation generates a ˜4.3 kb vector backbone, whereas BsaI-HFv2 digestion of insert plasmids with one of the two flanking BsaI sites protected from methylation generates a single ˜5.3 kb fragment.

D. Gel electrophoresis of DNA clones assembled into DH10B-2W213R cells by Universal Assembly from insert plasmids prepared in DH10B-2W213R following digestion by DraIII, which releases the assembled fragment through DraIII sites in the vector backbones, but does not cut inside the assembled 3.6 kb fragment as the assembled 3.6 kb DNA lacks DraIII sites. 7 out 8 of the assembled 8 clones were verified by DNA sequencing (+).

E. Gel electrophoresis of DNA clones assembled into DH10B-2W214R cells by Universal Assembly from insert plasmids prepared in DH10B-2W214R following digestion by DraIII, which releases the assembled fragment through DraIII sites in the vector backbones, but does not cut inside the assembled 3.6 kb fragment as the assembled 3.6 kb DNA lacks DraIII sites. 7 out 8 of the assembled 8 clones were verified by DNA sequencing (+).

F. Gel electrophoresis of DNA clones assembled into DH10B-2W213R or DH10B-2W214R by Universal Assembly following digestion by BsaI-HFv2. For correctly assembled DNA with internal BsaI sites completely blocked by M2.Eco31I methylation and both flanking BsaI sites fully protected from methylation, BsaI-HFv2 digestion generates a 4.4 kb vector backbone and a 3.6 kb insert DNA, whereas for assembled DNA with one of the two flanking BsaI sites protected from methylation, BsaI-HFv2 digestion generates a single 8.0 kb fragment.

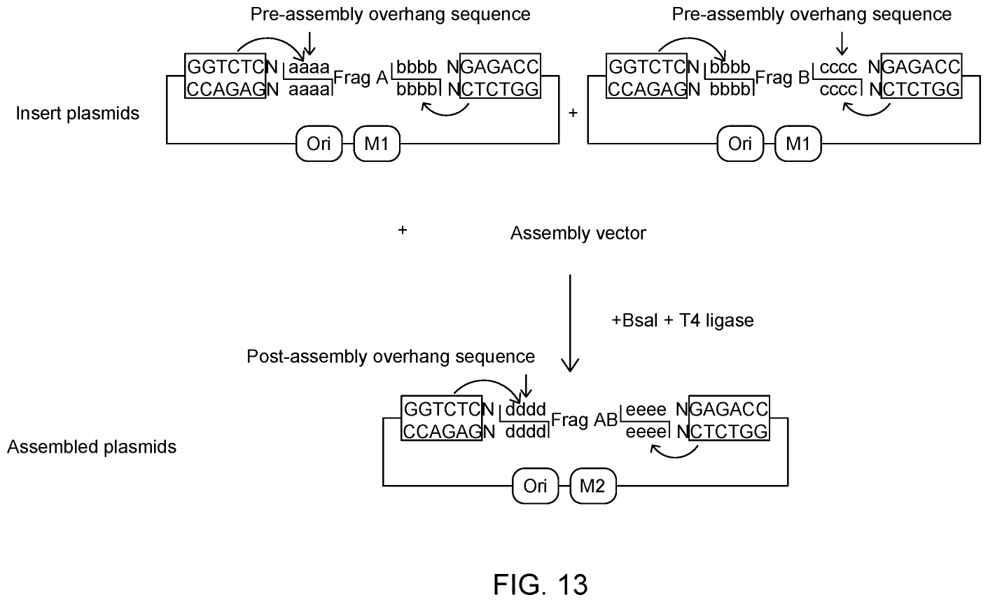

FIG. 13. Basic concepts of hierarchical assembly vector design

In a hierarchical assembly process using a single type IIS restriction enzyme, the insert fragments are flanked by type IIS restriction sites (boxed) in insert plasmids. One pot assembly with assembly vector generates assembled fragment flanked by the same restriction site in the assembled plasmids. The overhang sequence flanking the insert fragment and involved in ligation with the assembly vector backbone is referred to as “pre-assembly overhang” (‘aaaa’ and ‘cccc’), and the overhang sequence flanking the assembled fragment after DNA assembly is referred to as “post-assembly overhang” (‘dddd’ and ‘eeee’). The pre-assembly overhang and post-assembly overhang may or may not be the same depending on the vector design.

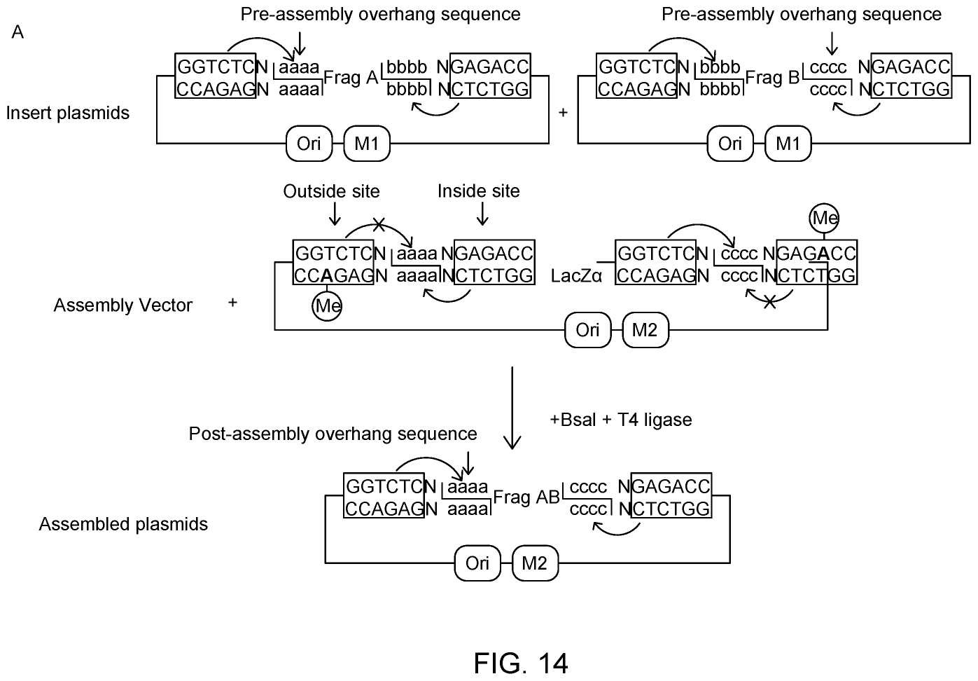

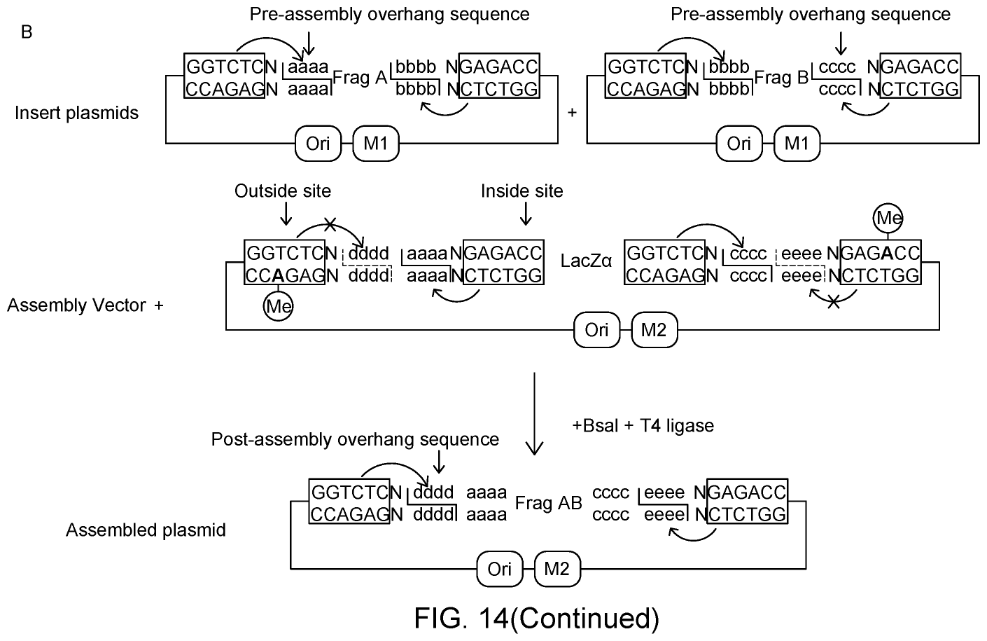

FIG. 14. Typical designs of hierarchical assembly vector using head-to-head restriction sites

Head-to-head arrangement of type IIS restriction sites can be used for design of assembly vector for hierarchical DNA assembly using a single type IIS restriction site. In the assembly vector, the negative selection marker LacZalpha is flanked by head-to-head type IIS restriction sites (BsaI sites as an example). The inside sites (boxed in solid line) close to LacZalpha are functional and once cut with BsaI generates adhesive ends compatible with the insert fragments (‘aaaa’ and ‘cccc’) for ligation. The outside sites (boxed in dotted line) close to the assembly vector backbone are blocked by DNA methylation by the switch methylase (methylated base in bold). Different designs of assembly vector can generate assembled fragments with post-assembly overhang sequence identical to (A), or completely different from (B) the pre-assembly overhang sequence.

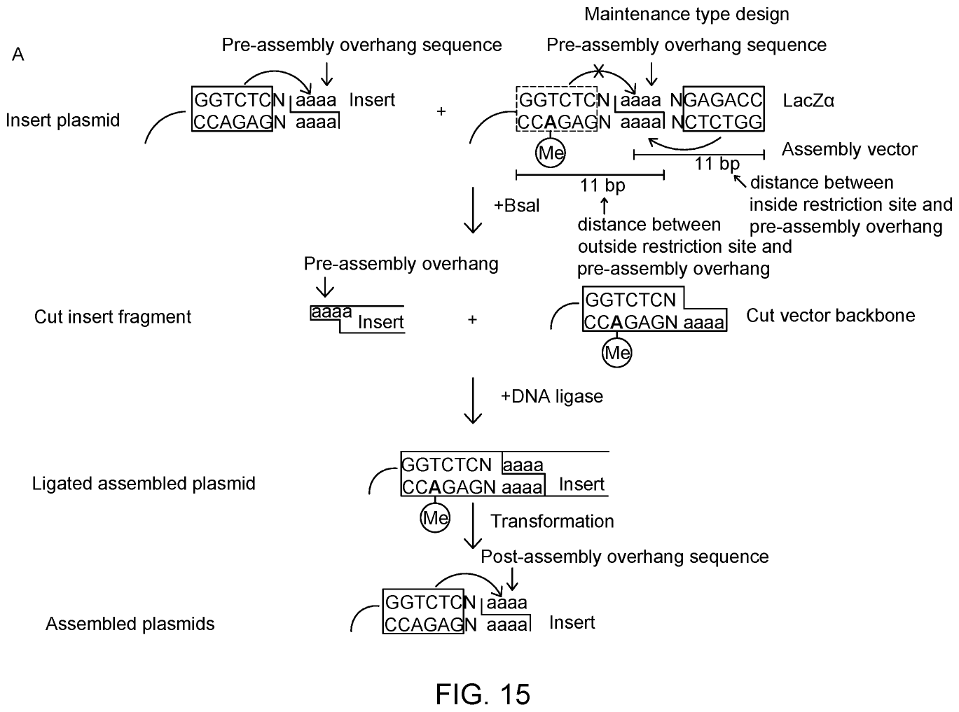

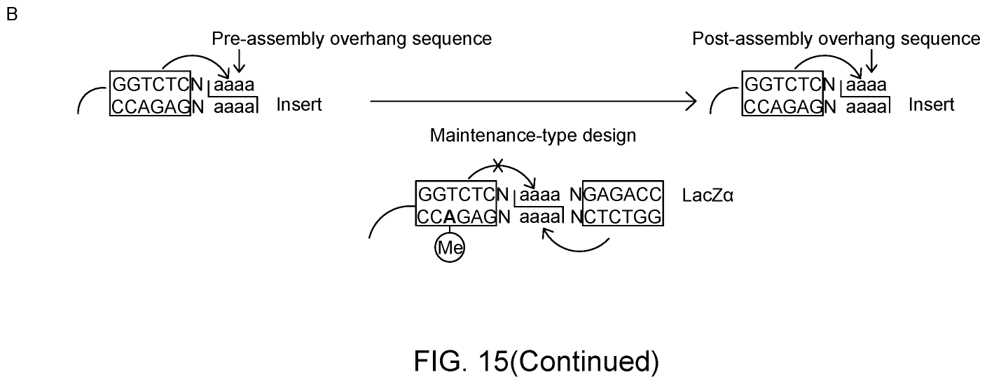

FIG. 15. Maintenance type design

A. DNA assembly process using vectors with maintenance type design. The maintenance type design contains a functional inside restriction site (boxed in solid line) and a methylated outside restriction site (boxed in dotted line, methylated base in bold) blocked by methylation switching using M.Osp807II or M.Sen0738I in a head-to-head arrangement. The distance between the outside restriction site and the pre-assembly overhang sequence ‘aaaa’ (11 bp) is the same as the distance between the inside restriction site and the pre-assembly overhang sequence (11 bp). Restriction with BsaI generates vector backbone containing the methylated restriction site, which can be ligated with the cut insert fragment flanked by pre-assembly overhang sequence (‘aaaa’). Following transformation into an E. coli strain that lacks methylation switching activity, methylation in the outside restriction site is lost and the assembled fragment can be cut with BsaI and generates assembled fragment flanked by post-assembly overhang sequence identical to the pre-assembly overhang sequence (‘aaaa’).

B. Abbreviation of the assembly process.

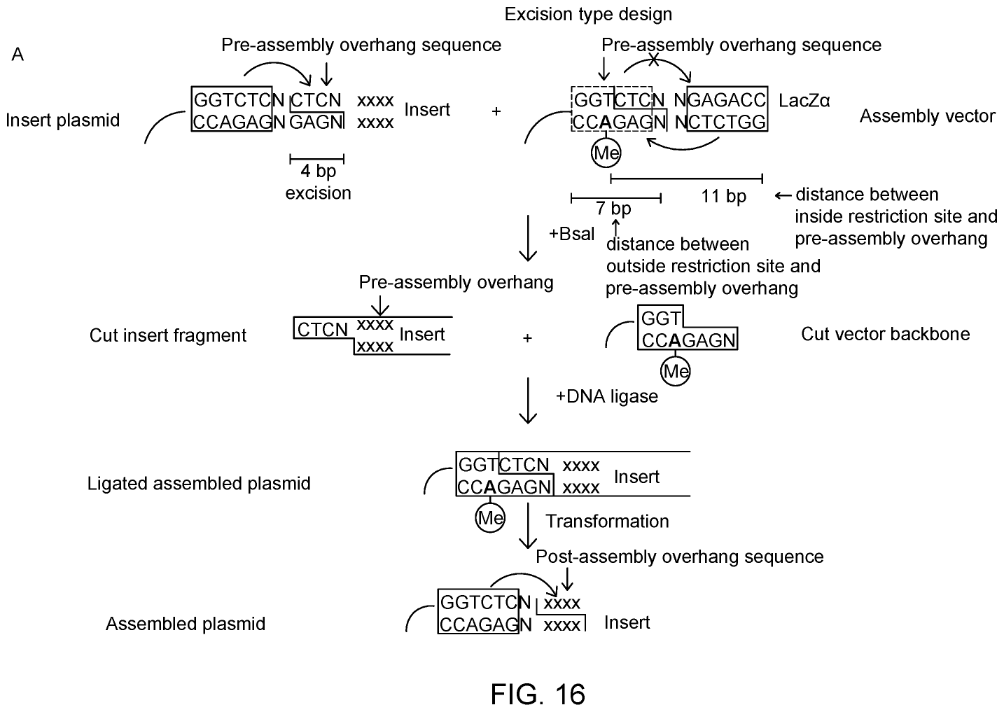

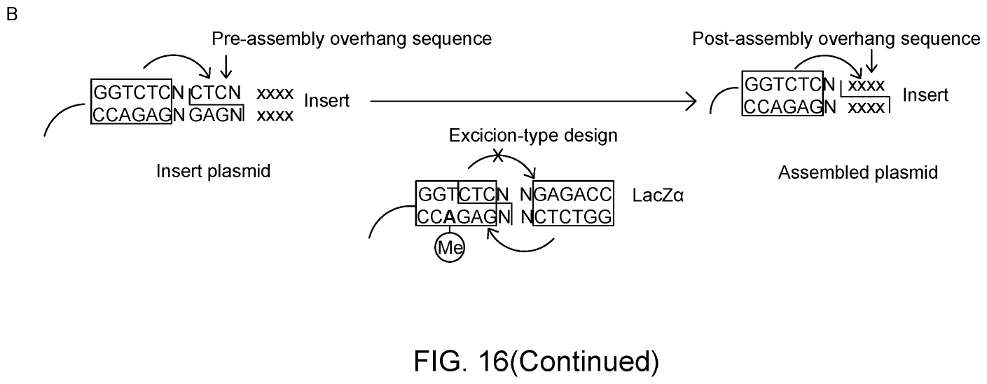

FIG. 16. Excision-type design of BsaI site with 4 bp excision

A. DNA assembly process using vectors with an excision-type design. The excision-type design contains a functional inside restriction site (boxed in solid line) and a methylated outside restriction site (boxed in dotted line, methylated base in bold) blocked by methylation switching using M.Osp807II or M.Sen0738I in a head-to-head arrangement. The distance between the outside restriction site and the pre-assembly overhang sequence ‘CTCN’ (7 bp) is 4 bp less than the distance between the inside restriction site and the pre-assembly overhang sequence (11 bp). Restriction with BsaI generates vector backbone containing a partial methylated restriction site, which can be ligated with the cut insert fragment flanked by pre-assembly overhang sequence (‘CTCN’) to reconstitute a functional outside restriction site. Following transformation into an E. coli strain that lacks methylation switching activity, methylation in the outside restriction site is lost and the assembled fragment can be cut with BsaI and generates assembled fragment flanked by post-assembly overhang sequence (‘xxxx’). Compared with the insert fragment, the assembled fragment has had 4 bp excised from the end of the sequence.

B. Abbreviation of the assembly process.

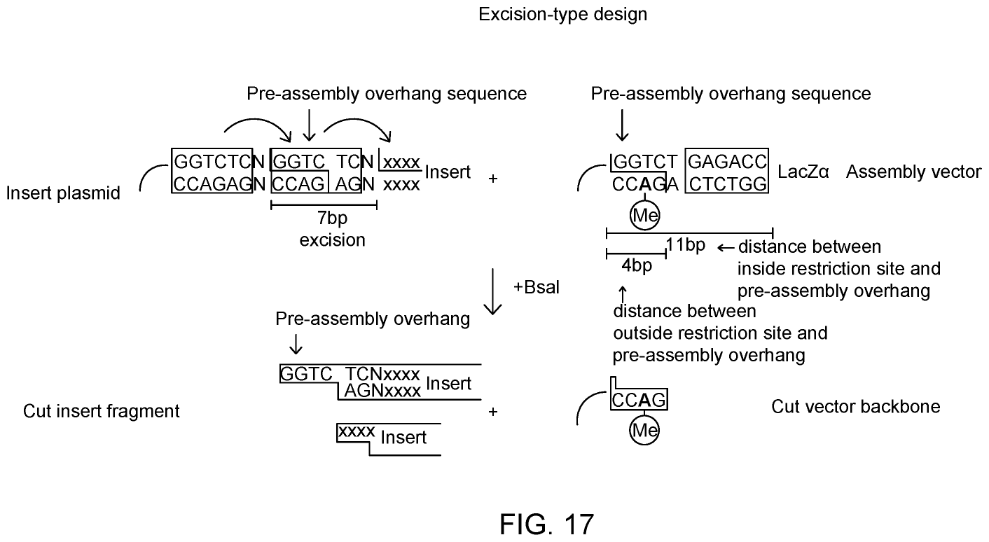

FIG. 17. Problem with 7 bp excision-type design of methylation-switching BsaI system

With a design of assembly vector that potentially leads to excision of 7 bp at the end of the insert DNA during the assembly process, the insert fragment must contain a functional BsaI site inside the BsaI site flanking the insert fragment, in order to reconstitute the outside BsaI site in the assembly vector backbone. Because the insert plasmid contains two functional BsaI sites, digestion with BsaI generates a mixture of two insert fragments with different flanking overhang sequences, which will interfere with the assembly process.

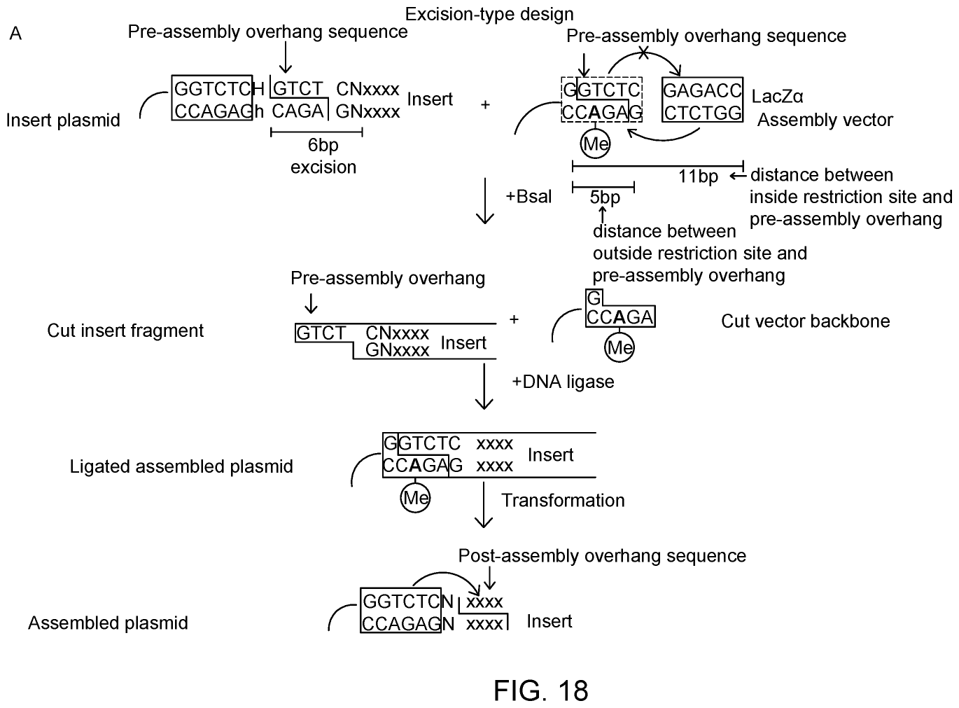

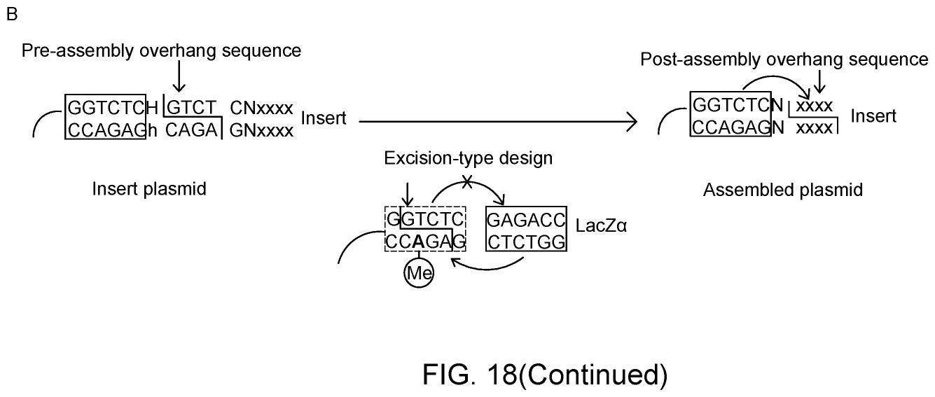

FIG. 18. Excision-type design of BsaI site with 6 bp excision

A. DNA assembly process using vectors with excision-type design. The excision-type design contains a functional inside restriction site (boxed in solid line) and a methylated outside restriction site (boxed in dotted line, methylated base in bold) blocked by methylation switching using M.Osp807II or M.Sen0738I in a head-to-head arrangement. The distance between the outside restriction site and the pre-assembly overhang sequence ‘GTCT’ (5 bp) is 6 bp less than the distance between the inside restriction site and the pre-assembly overhang sequence (11 bp). Restriction with BsaI generates vector backbone containing a partial methylated restriction site, which can be ligated with the cut insert fragment flanked by pre-assembly overhang sequence (‘GTCT’) to reconstitute a functional outside restriction site. Following transformation into an E. coli strain that lacks methylation switching activity, methylation in the outside restriction site is lost and the assembled fragment can be cut with BsaI and generates assembled fragment flanked by post-assembly overhang sequence (‘xxxx’). Compared with the insert fragment, the assembled fragment has 6 bp excised from the end of the sequence. ‘N’ represents A, T, C, or G. ‘H’ represents A, T or C, and ‘h’ represents the nucleotide complementary to ‘H’.

B. Abbreviation of the assembly process.

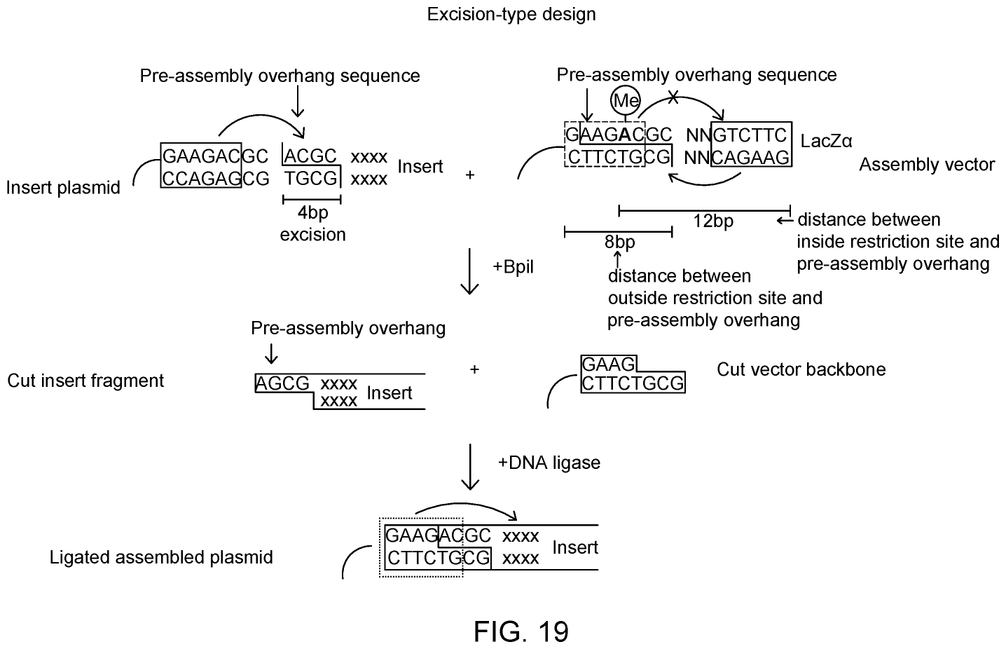

FIG. 19. Problem with excision-type design of M2.Nme-based methylation switching of BpiI system

The 4 bp excision-type design of BpiI assembly system based on M2.NmeMC58II methylation switching is unfeasible because digestion of the assembly vector generates a partial outside BpiI site that lacks DNA methylation due to the position of base methylated by switch methylase M2.NmeMC58II. Ligation of the vector backbone with the insert fragment generates assembled plasmid with functional BpiI site (highlighted in grey), which will be repeatedly cut during the assembly process.

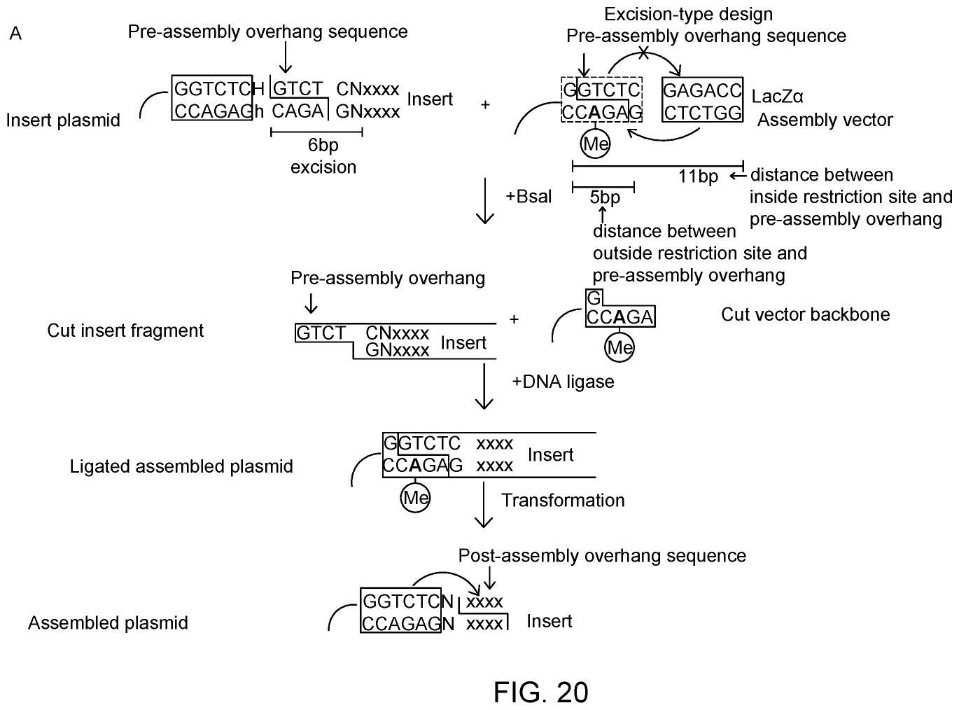

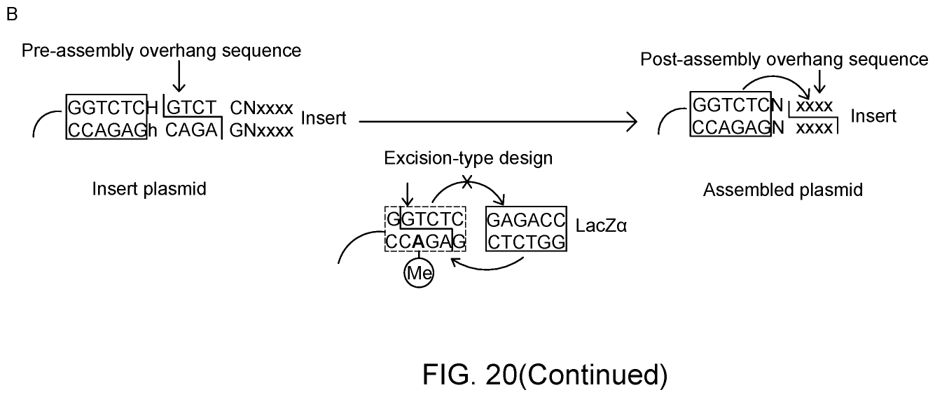

FIG. 20. Excision-type design of BsaI site with 6 bp excision using partial BsaI site as outside site

A. DNA assembly process using vectors with excision-type design. The excision-type design contains a functional inside restriction site (boxed in solid line) and a methylated partial restriction site (boxed in dotted line, methylated base in bold) methylated by M.Osp807II or M.Sen0738I in a head-to-head arrangement. The distance between the outside restriction site and the pre-assembly overhang sequence ‘GTCT’ (5 bp) is 6 bp less than the distance between the inside restriction site and the pre-assembly overhang sequence (11 bp). Restriction with BsaI generates vector backbone containing a methylated partial restriction site, which can be ligated with the cut insert fragment flanked by pre-assembly overhang sequence (‘GTCT’) to reconstitute a functional outside restriction site. Following transformation into an E. coli strain that lacks methylation switching activity, methylation in the outside restriction site is lost and the assembled fragment can be cut with BsaI and generates assembled fragment flanked by post-assembly overhang sequence (‘xxxx’). Compared with the insert fragment, the assembled fragment has 6 bp excised from the end of the sequence. ‘N’ represents A, T, C, or G. ‘H’ represents A, T or C, and ‘h’ represents the nucleotide complementary to ‘H’.

B. Abbreviation of the assembly process.

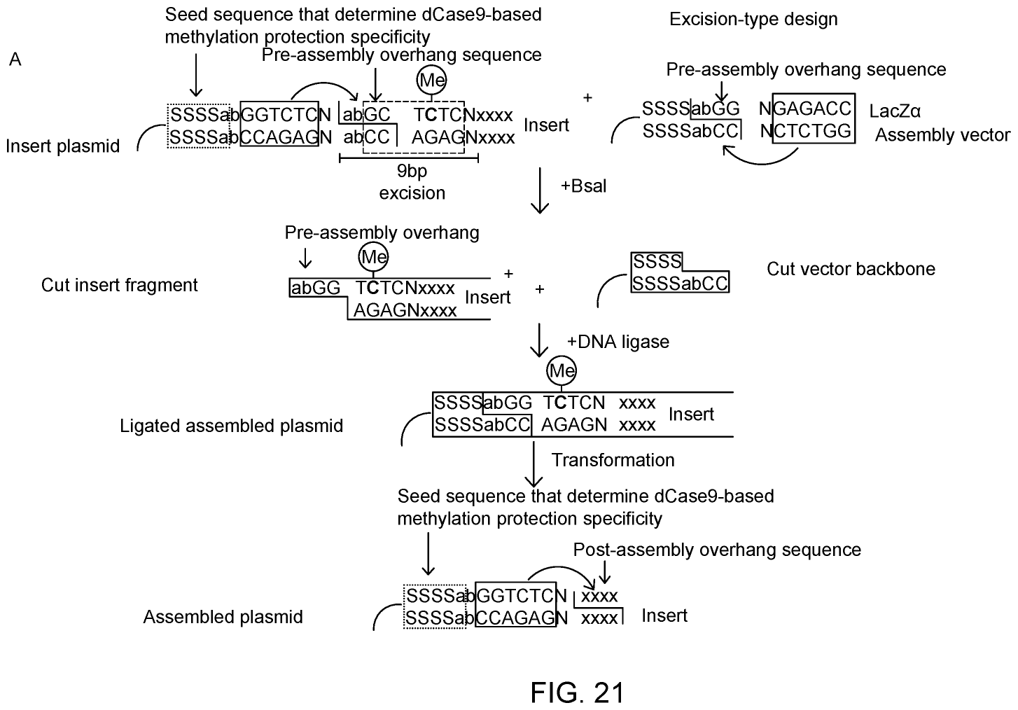

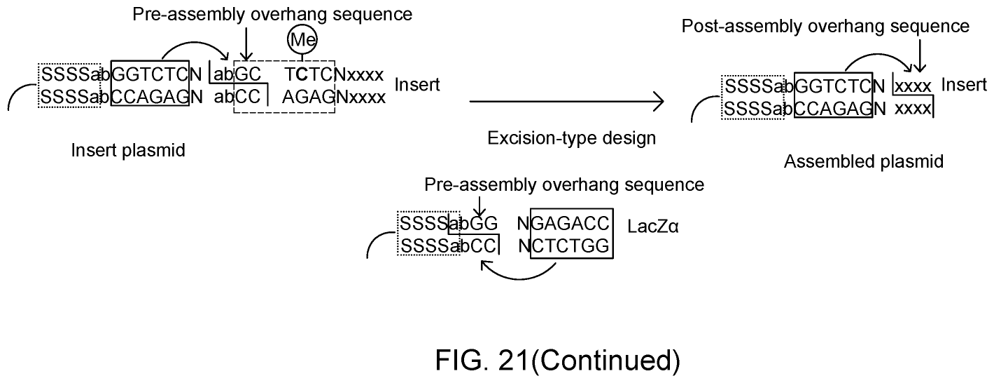

FIG. 21. Excision-type design with methylation protection mechanism

A. DNA assembly process using vectors with excision-type design based on methylation protection mechanism. The excision-type assembly vector design contains a functional inside restriction site (boxed in solid line) but lacks outside restriction site. The insert DNA contains 9 bp sequence to be excised (‘abGGTCTCN’) which itself provides an intact BsaI site (boxed in dotted line) to be used as the reconstituted outside BsaI site in the assembled plasmid. This site is blocked in the insert plasmid by methylation due to the methylation protection mechanism (boxed in dotted line, methylated base in bold), whereas the flanking BsaI site to release the insert fragment is functional due to methylation protection (boxed in solid line). The 5 bp seed sequence that defines the specificity of the methylation protection mechanism (‘SSSSa’) is highlighted in grey. Restriction with BsaI generates vector backbone containing overhang sequence compatible with the pre-assembly overhang sequence (‘abGG’) of the insert fragment for ligation. Following transformation into strains that deploys methylation protection mechanism, the reconstituted BsaI site in the assembled plasmid lost methylation due to methylation protection mechanism. As a result, compared with the insert fragment, the assembled fragment has 9 bp excised from the end of the sequence. The assembly vector sequence was designed so that following DNA assembly, the reconstituted outside restriction site is protected from methylation protection mechanism using dCas9-based methylation protection with the same specificity as the insert plasmid (dCas9 seed sequence for methylation protection ‘SSSSa’ remains the same).

B. Abbreviation of the assembly process.

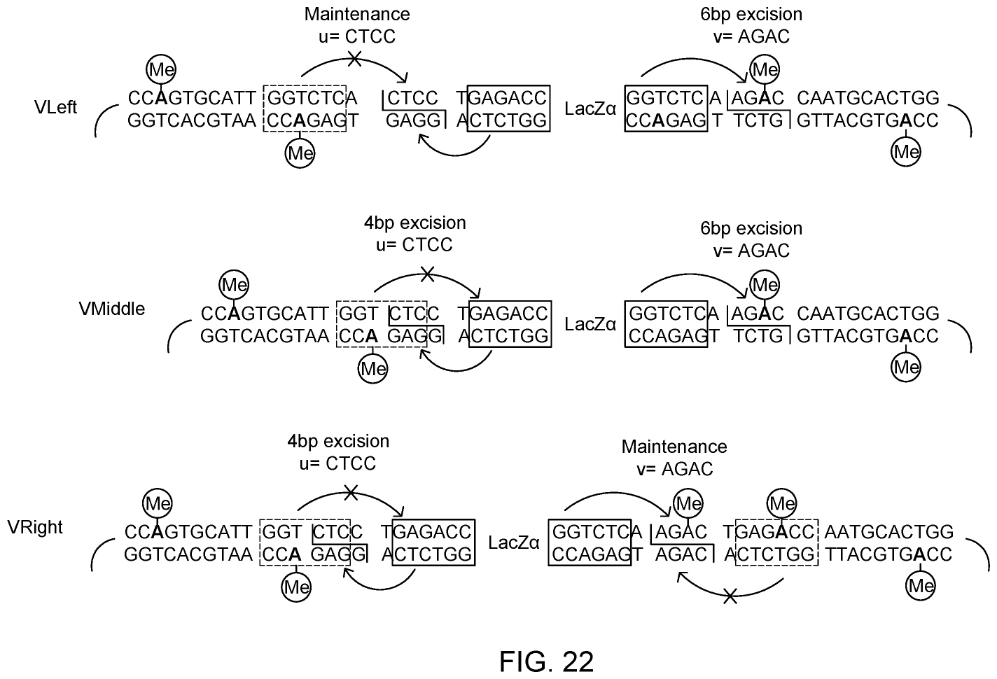

FIG. 22. Three types of scarless assembly vectors

Diagram for the three types of scarless assembly vectors. Bases methylated by switch methylase M.Sen0738I are in bold, blocked BsaI sites boxed in dotted line and functional BsaI sites boxed in solid line. Vector VLeft carries maintenance type design at the left arm with overhang sequence ‘CTCC’ (‘u’), and 6 bp excision-type design at the right arm with overhang sequence ‘AGAC’ (‘v’). Vector VMiddle carries 4 bp excision-type design at the left arm with overhang sequence ‘CTCC’ (‘u’), and 6 bp excision-type design at the right arm with overhang sequence ‘AGAC’ (‘v’). Vector VRight carries 4 bp excision-type design at the left arm with overhang sequence ‘CTCC’ (‘u’), and maintenance type design at the right arm with overhang sequence ‘AGAC’ (‘v’).

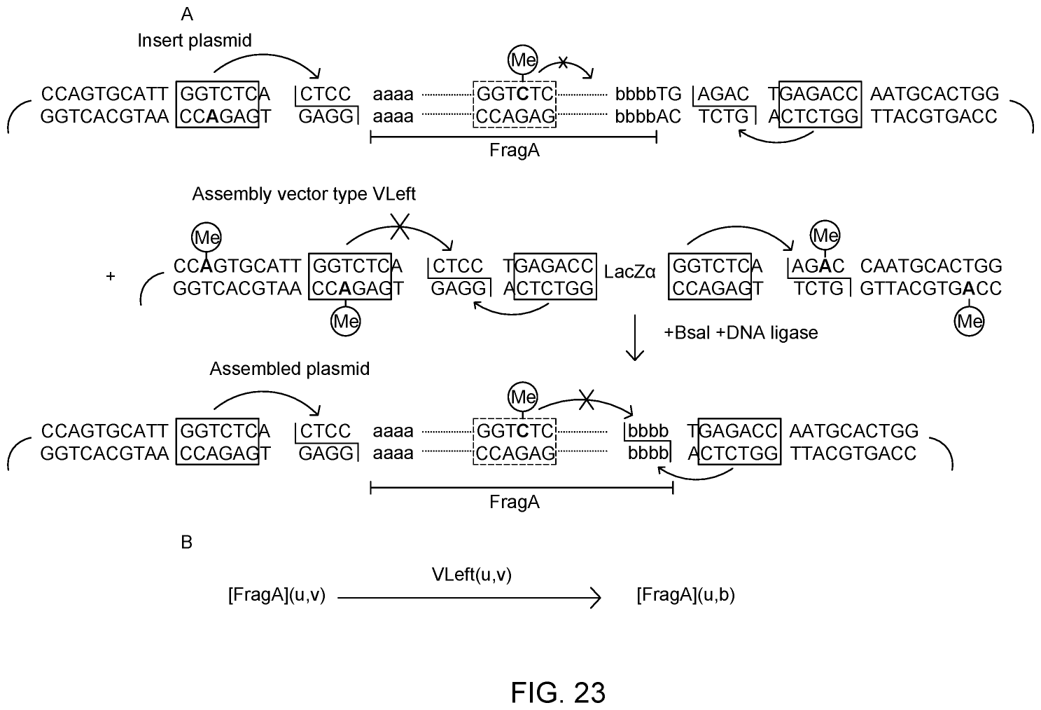

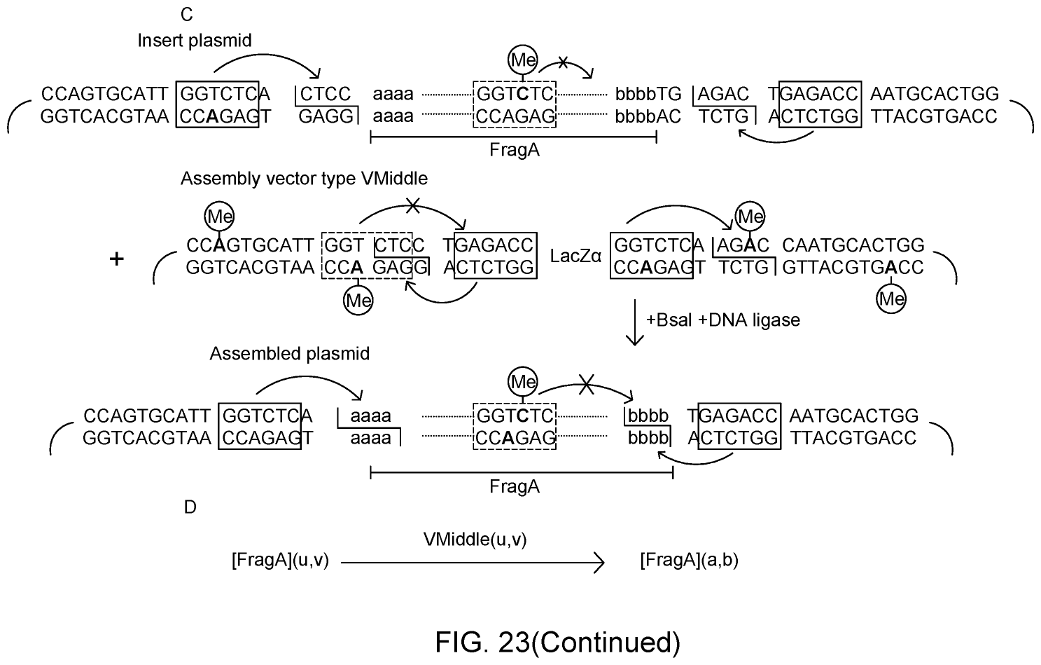

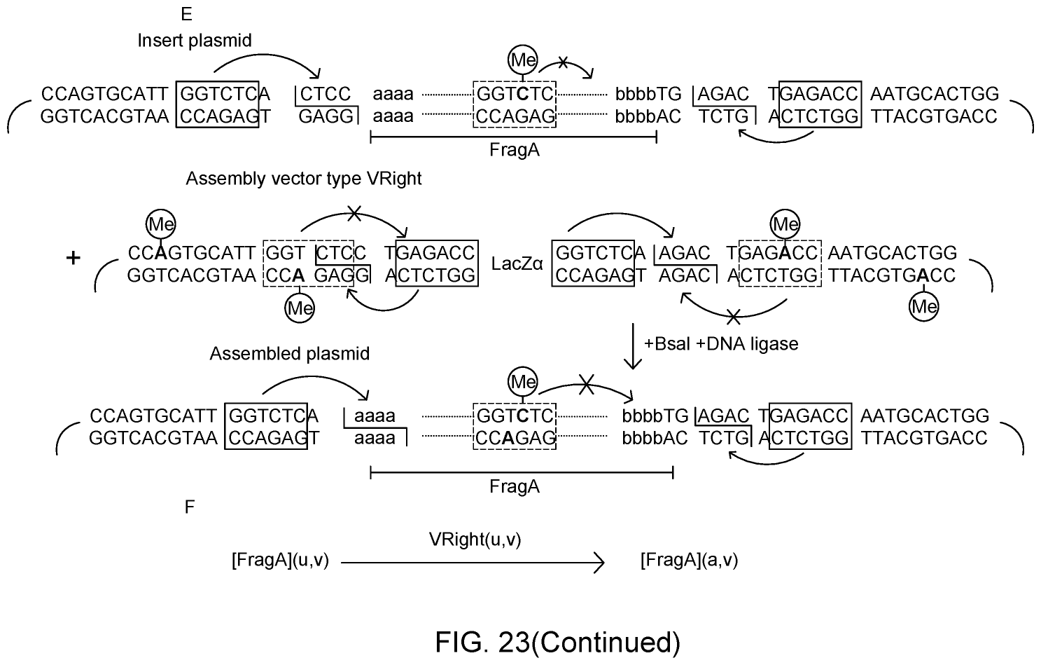

FIG. 23. Directional insert DNA excision using the three types of scarless assembly vectors

A. Cloning of a single insert DNA fragment FragA with compatible adhesive ends (‘CTCC’ and ‘AGAC’) into scarless assembly vector VLeft. Internal BsaI sites in the middle of the insert fragments are methylated by methylation protection (boxed in dotted line, methylated base in bold). Cloning into vector VLeft excises 6 bp adaptor sequence (‘TGAGAC’) at the right end of the sequence, generating insert fragment flanked by post-assembly overhang sequence ‘CTCC’ at left end and ‘bbbb’ at right end.

B. Symbolic representation of the assembly reaction. [FragA](u,v) represents FragA flanked by overhang sequence ‘u’ (‘CTTC’) and ‘v’ (‘AGAC’) in the insert plasmid. Assembling into VLeft generates assembled plasmid [FragABC](u,d) with assembled fragment flanked by overhang sequences ‘u’ (‘CTCC’) and ‘bbbb’.

C and E. Cloning of FragA into assembly vector VMiddle (C) and VRight (E).

D and F. Symbolic representation of the cloning process using vector VMiddle (D) and VRight (F).

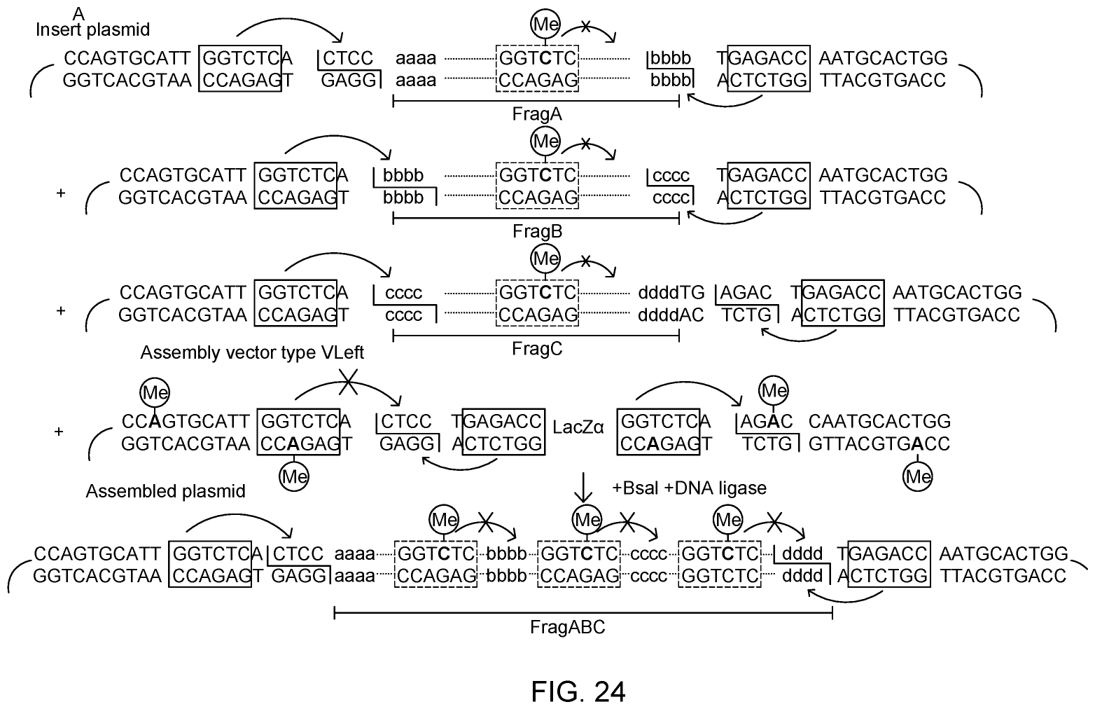

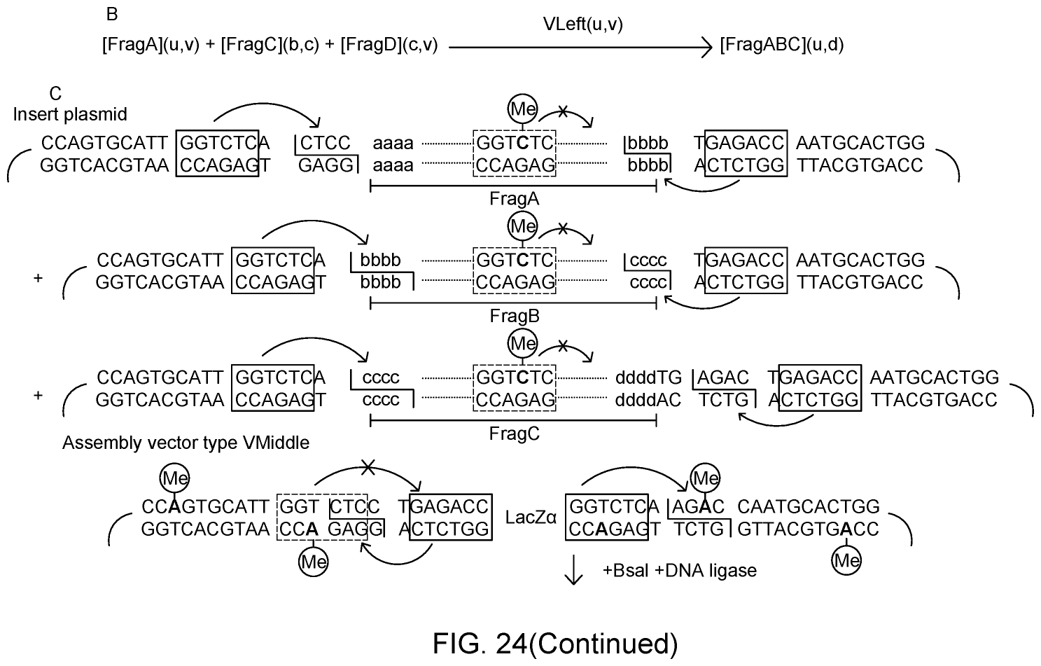

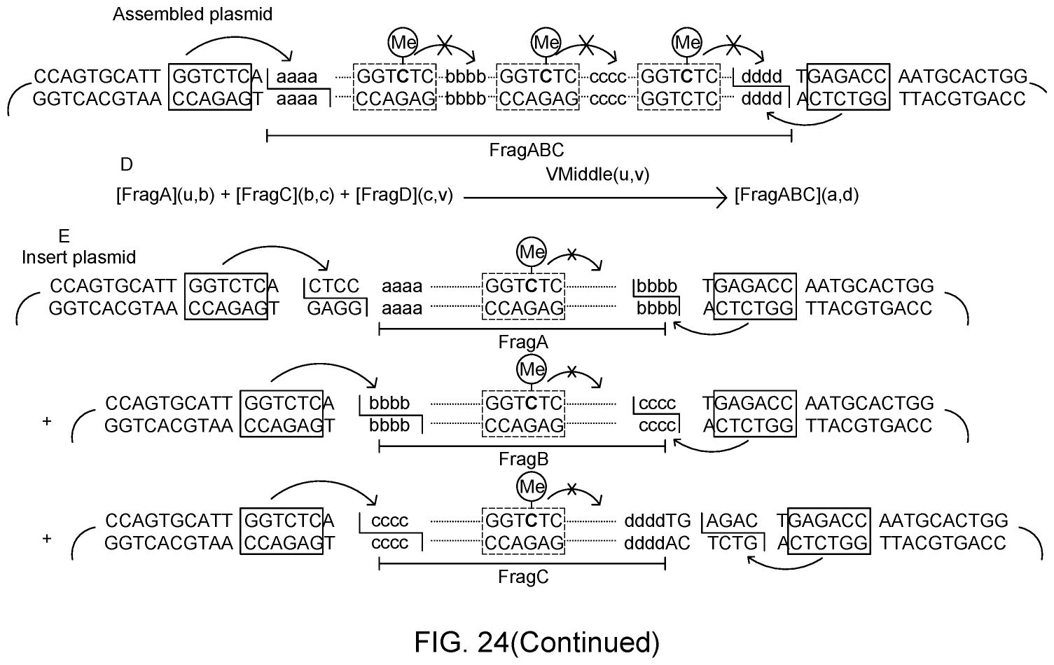

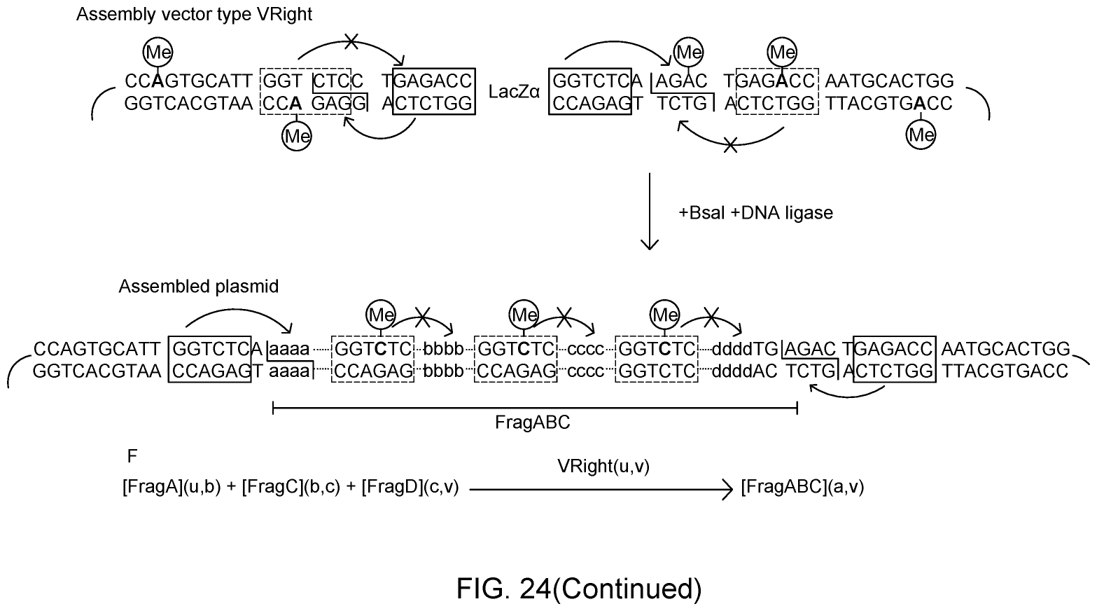

FIG. 24. DNA assembly and excision using the three types of scarless assembly vectors

A. Assembly of three fragments (FragA, FragB and FragC) with compatible adhesive ends (‘CTCC’, ‘bbbb, ‘cccc’, ‘AGAC’) into scarless assembly vector VLeft. Internal BsaI sites in the middle of the insert fragments are methylated by methylation protection (boxed in dotted line, methylated base in bold). Assembly into vector VLeft excises 6 bp at the right end of the sequence, generating assembled fragment flanked by post-assembly overhang sequence ‘CTCC’ at left end and ‘dddd’ at right end.

B. Symbolic representation of the assembly reaction. [FragA](u,a) represents Fragment A flanked by overhang sequence ‘u’ (‘CTCC’) and ‘aaaa’ in the insert plasmid. Assembling into VLeft generates assembled plasmid [FragABC](u,d) with assembled fragment flanked by overhang sequences ‘u’ (‘CTCC’) and ‘dddd’.

C and E. Assembly of three fragments into assembly vector VMiddle (C) and VRight (E).

D and F. Symbolic representation of the assembly process using vector VMiddle (D) and VRight (F).

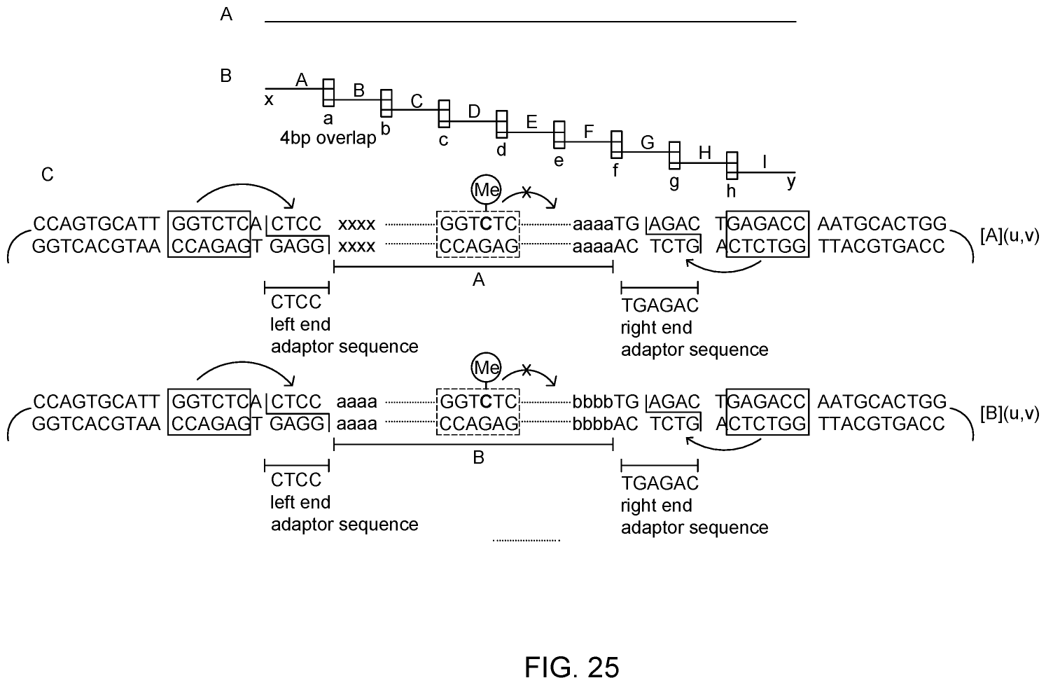

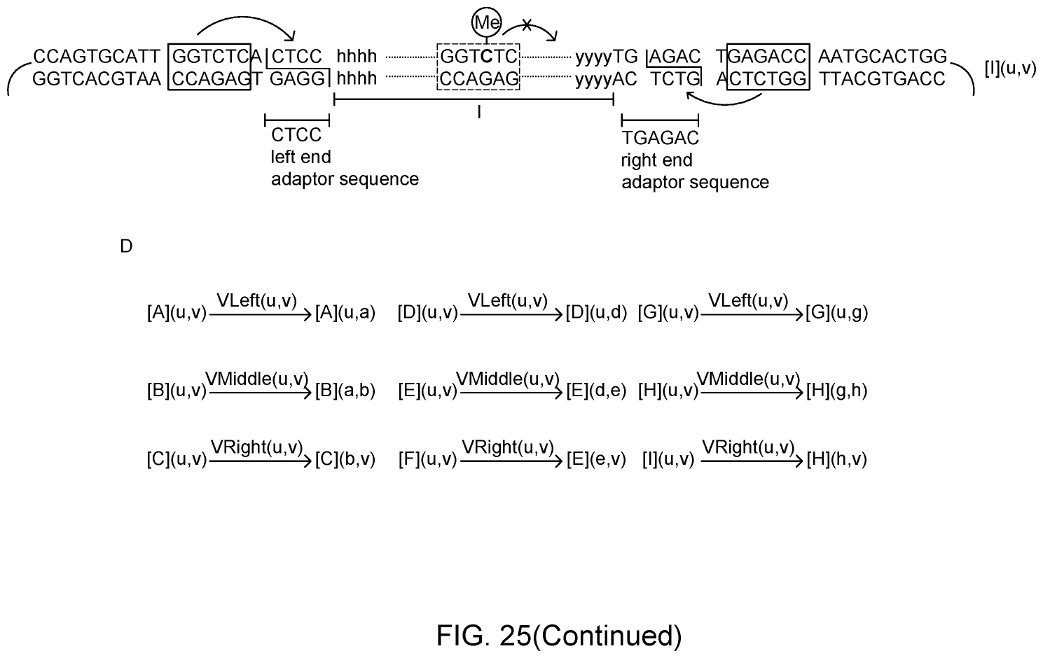

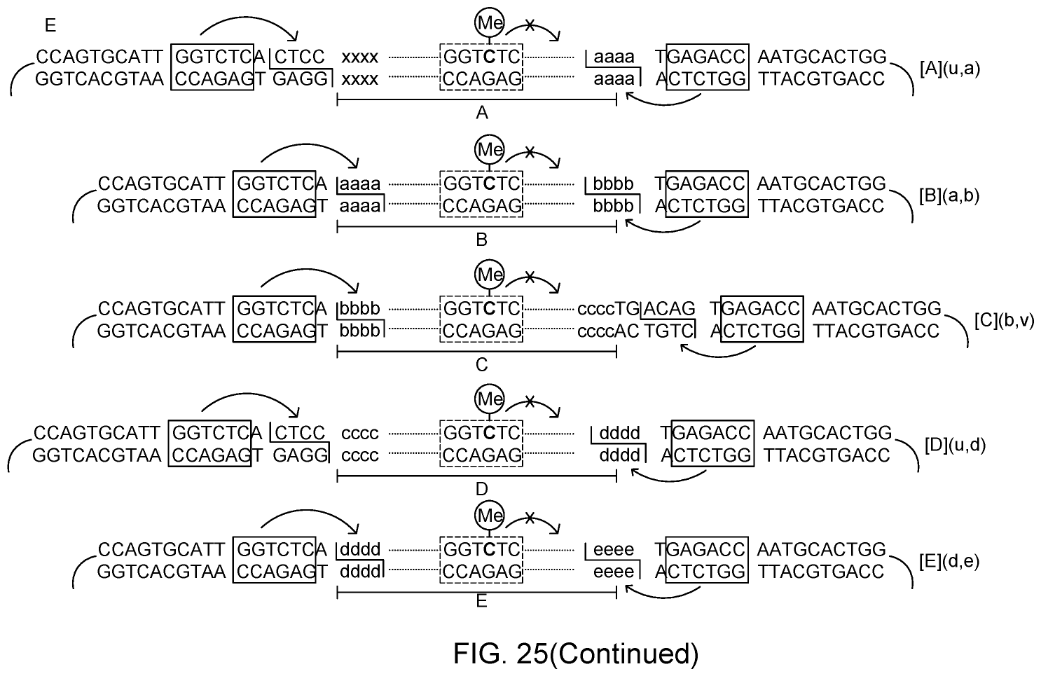

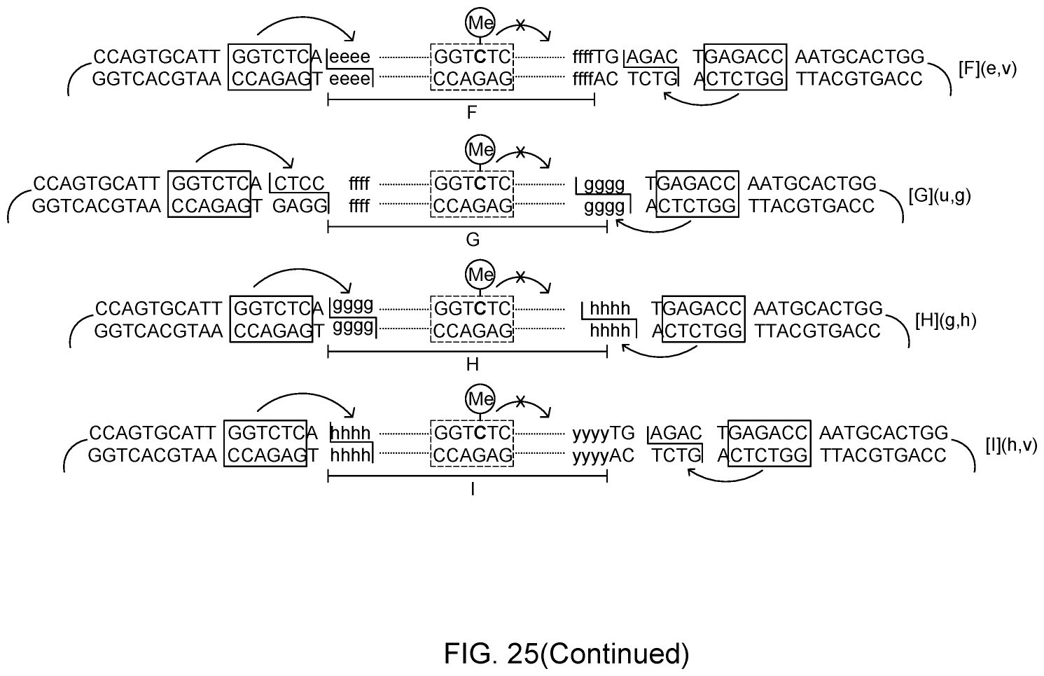

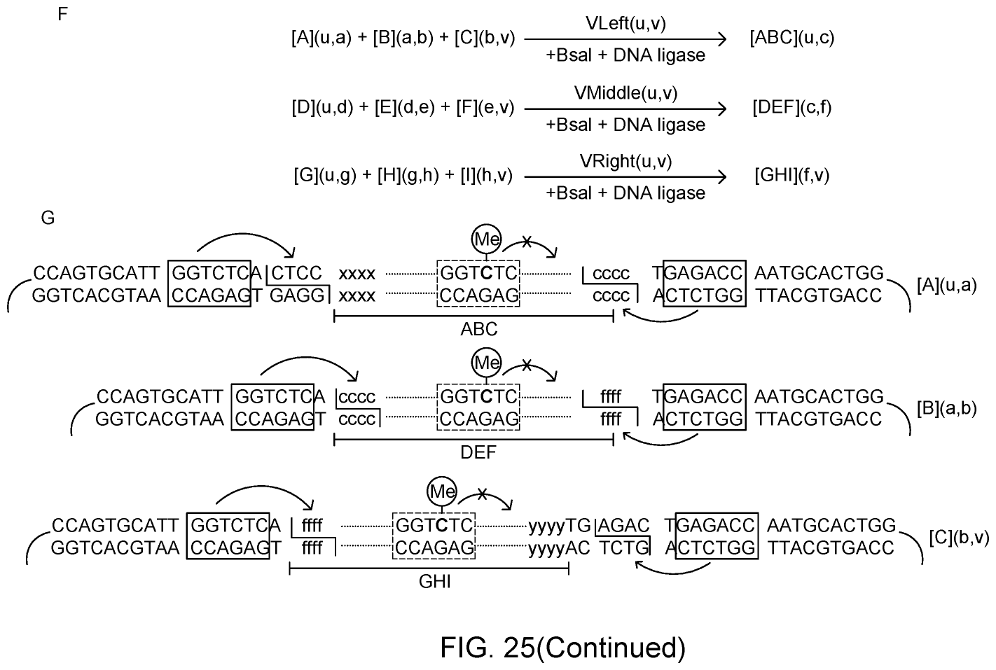

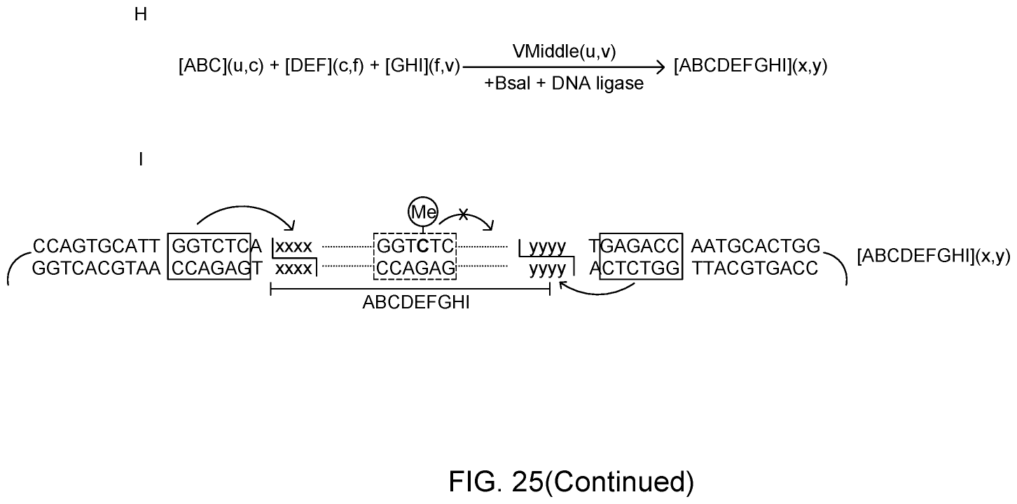

FIG. 25. Hierarchical scarless assembly scheme

A and B. The starting fragment is first of all digitally broken into 9 fragments with 4 bp overlapping sequence.

C. For each starting fragment, the 4 bp adaptor sequence ‘CTCC’ and 6 bp adaptor sequence ‘TGAGAC’ is added to the left and right end of the sequence respectively, and cloned into plasmids to generate insert plasmids with insert DNA flanked by overhang sequences ‘u’ (‘CTCC’) and ‘v’ (‘AGAC’). The insert plasmids are prepared in methylation protection strain, so that internal BsaI sites inside the insert are methylated (boxed in dotted line, methylated base in bold), whereas the flanking BsaI sites are protected from methylation (boxed in solid line).

D and E. Each insert fragment is then cloned as single fragment into appropriate scarless assembly vectors depending on the position of the fragment in the next round of assembly.

F and G. In the first round of multi-fragment assembly, the fragments were assembled three per group into appropriate scarless assembly vectors depending on the position of the assembled fragment in the next round of assembly to generate assembled fragment [ABC], [DEF] and [GHI].

H and I. These three intermediate fragments can then be assembled into vector VMiddle to generate the fully assembled fragment [ABCDEFGHI] corresponding to the starting fragment with flanking overhang sequence ‘xxxx’ and ‘yyyy’ defined by the input sequence.

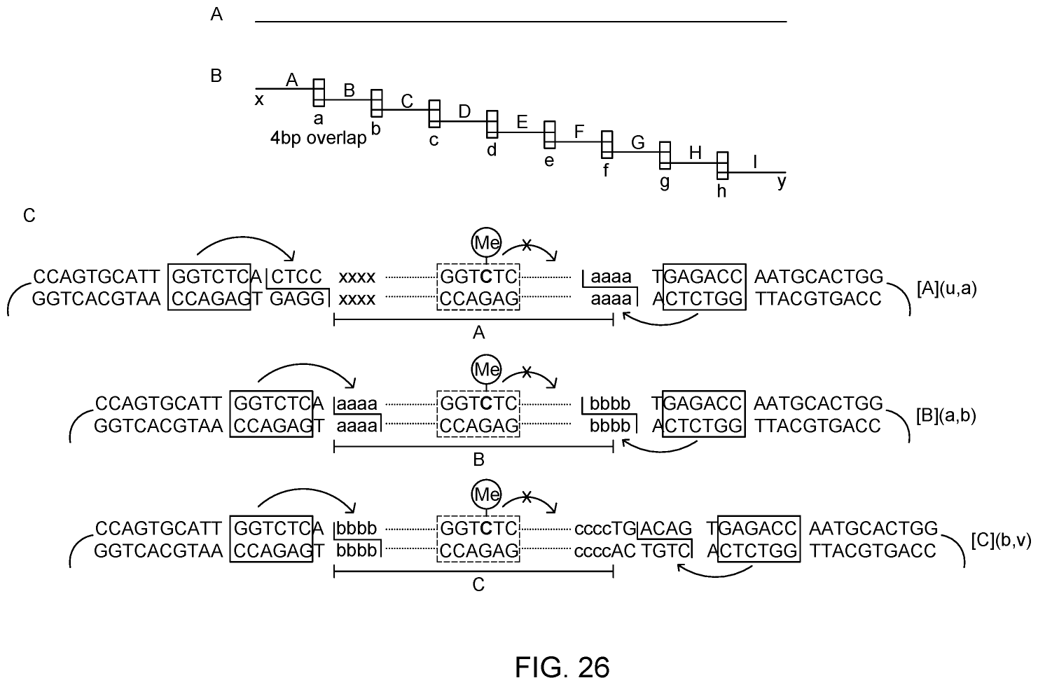

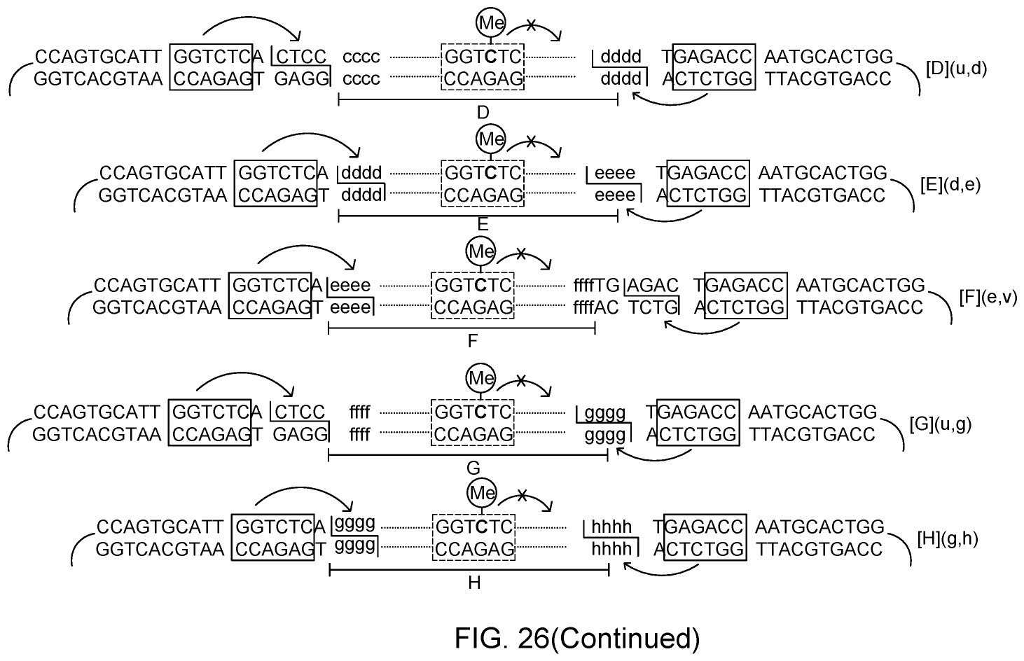

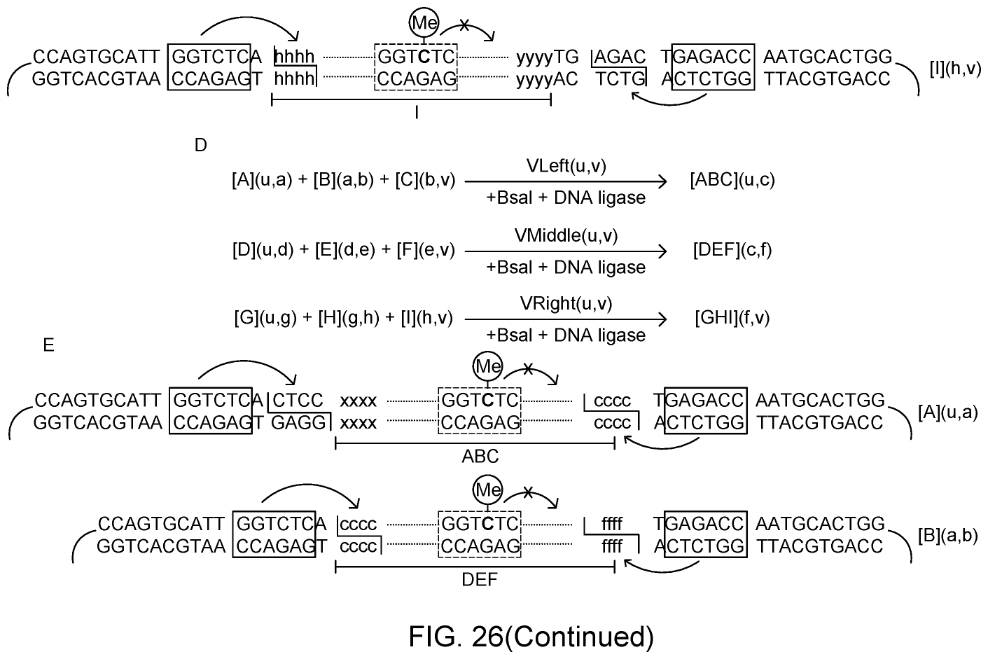

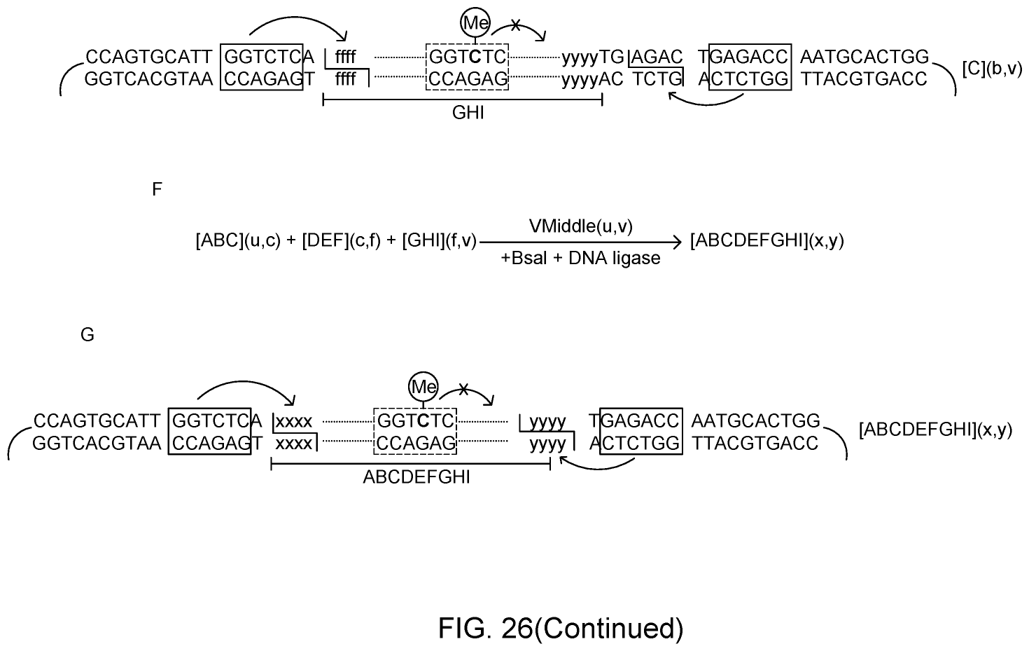

FIG. 26. Hierarchical scarless assembly scheme with simplified preparative cloning process

A and B. The starting fragment is first of all digitally broken into 9 fragments with 4 bp overlapping sequence.

C. Each fragment is cloned into insert plasmid using methylation protection strain, with adaptor sequence added depending on the position of the fragment in the first round of assembly. The left most fragments (Fragments A, D and G) has 4 bp adaptor sequence ‘CTCC’ added to the left end of the fragment. The rightmost fragments (Fragments C, F and I) has 6 bp adaptor sequence ‘TGAGAC’ added to the right end of the fragment. The middle fragments (Fragments B, E and H) have no adaptor sequences added at either end.

D and E. In the first round assembly, the fragments were assembled three per group into appropriate scarless assembly vectors depending on the position of the assembled fragment in the next round of assembly to generate assembled fragment [ABC], [DEF] and [GHI].

F and G. These three intermediate fragments can then be assembled into vector VMiddle to generate the fully assembled fragment [ABCDEFGHI] corresponding to the starting fragment with flanking overhang sequence ‘xxxx’ and ‘yyyy’ defined by the input sequence.

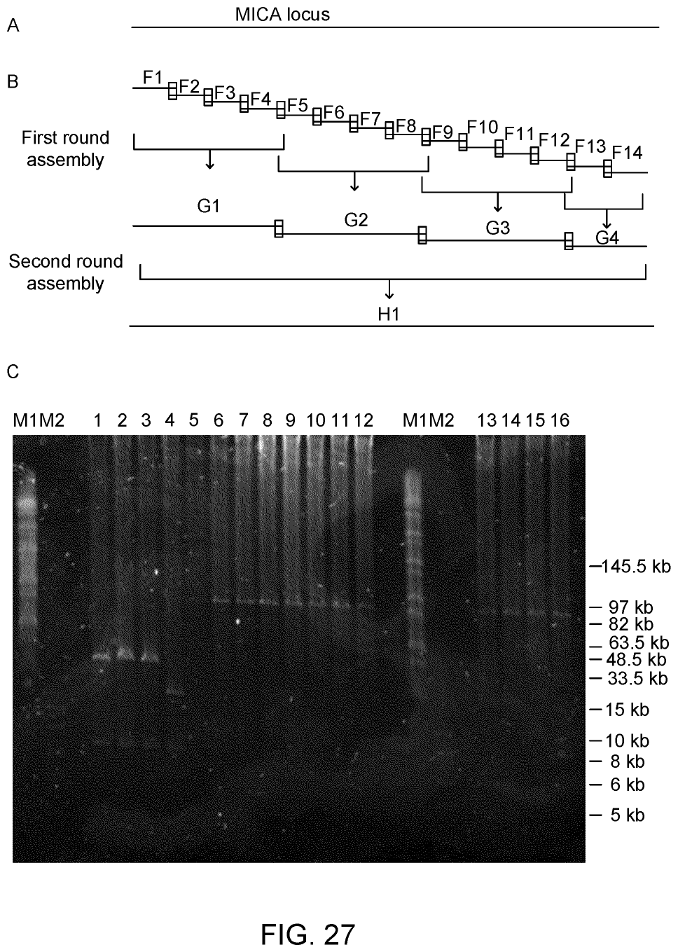

FIG. 27. Practical scarless assembly using Universal Assembly

A and B. A ˜90 kb sequence corresponding to the MICA locus was first digitally broken into 14 fragments with 4 bp overlapping sequence (F1-F14). The fragments were cloned by PCR as insert plasmids to be used for first round DNA assembly, and assembled 2-4 fragments per group using scarless Universal Assembly into intermediate fragments G1-G4. The four intermediate fragments were further assembled into a single ˜90 kb fragment H1.

C. Pulsed field electrophoresis of assembled plasmids carrying intermediate fragments G1-G4 (lane 1-4), or fully assembled fragments H1 (lane 5-12) following restriction using NotI, along with MidRange I PFG Marker (NEB, M1) or 1 kb DNA ladder (NEB, M2). 12 independent clones of fully assembled plasmids were screened and the assembled clones are 100% correct (12 out of 12 correct).

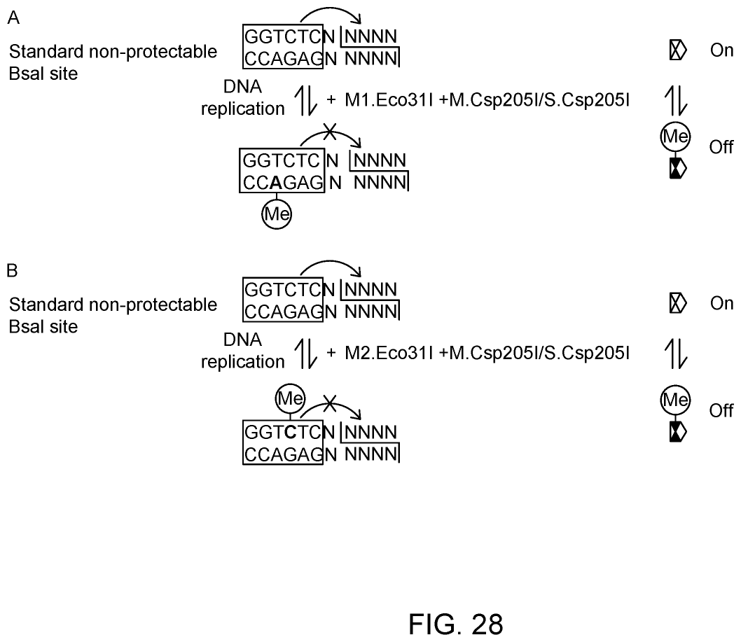

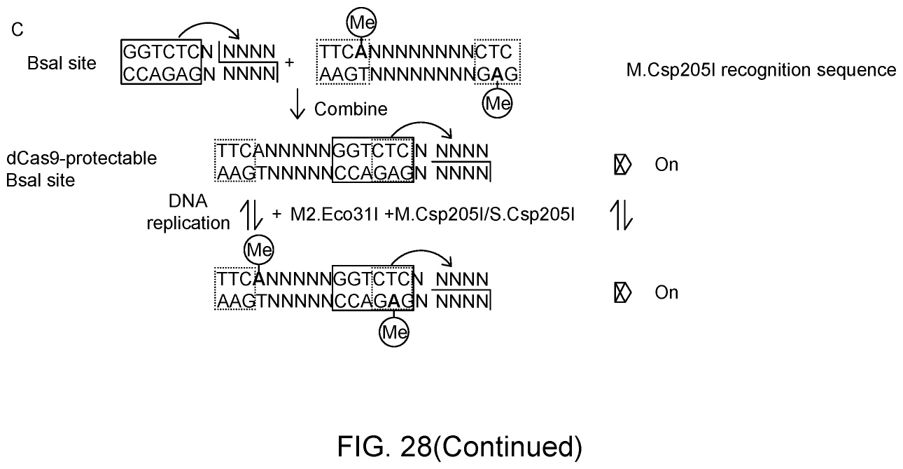

FIG. 28. Methylation protection using DNA methylase as the specific DNA binding protein

A. A standard type IIS restriction enzyme site such as BsaI (boxed) can be methylated by the M1.Eco31I ‘protection’ methylase at the 3rd base of the bottom strand (in bold) when the plasmid is prepared in an E. coli strain that expresses the M1.Eco31I methylase, M.Csp205I and S.Csp205I (the standard BsaI site must not overlap with M.Csp205I recognition sequence).

B. A standard type IIS restriction enzyme site such as BsaI (boxed) can be methylated by the M2.Eco31I ‘protection’ methylase at the 4th base of the top strand (in bold) when the plasmid is prepared in an E. coli strain that expresses the M2.Eco31I methylase, M.Csp205I and S.Csp205I (the standard BsaI site must not overlap with M.Csp205I recognition sequence).

C. Combining a BsaI site (boxed) with the M.Csp205I recognition sequence (highlighted in grey, methylated base in bold) creates a BsaI site that overlaps with the M.Csp205I recognition sequence. Preparation of the plasmid in an E. coli strain that expresses the M1.Eco31I or M2.Eco31I methylase, M.Csp205I and S.Csp205I that recognizes this combined site results in M.Csp205I binding to the site, which may block the site from methylation by the M1.Eco31I or M2.Eco31I protection methylase. Such site is referred to as a M.Csp205I-protectable site. M.Csp205I methylates the 5^{th} base at bottom strand of the BsaI site within the M.Csp205I-protectable site. This methylation itself does not affect BsaI activity towards the BsaI site. The methylation protection process can be represented by the symbols to the right; open triangles depicts an unmethylated BsaI site, and black triangles depict a methylated BsaI site.

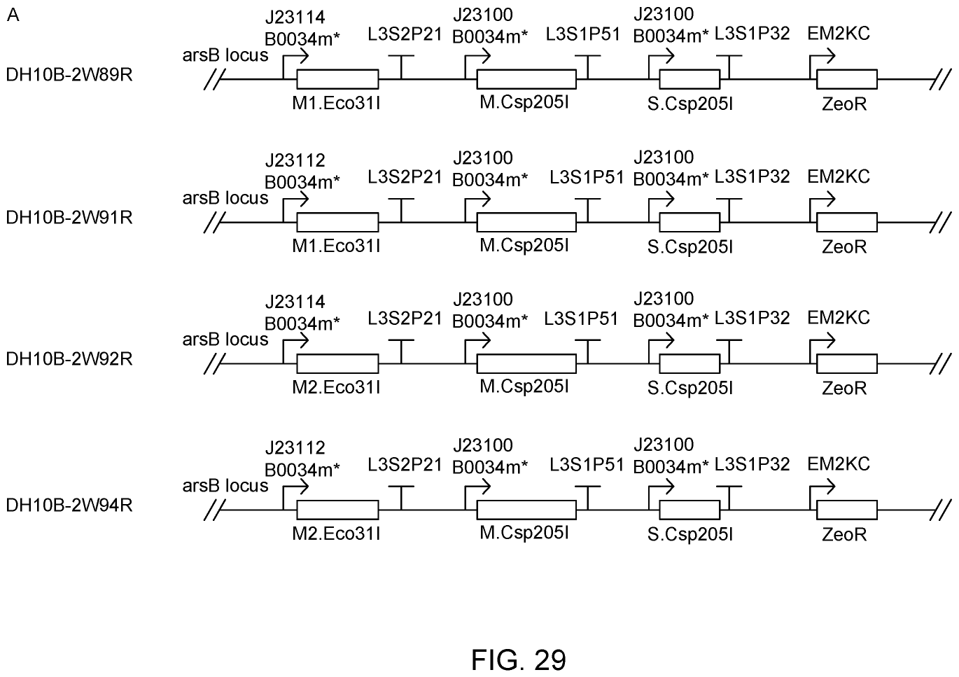

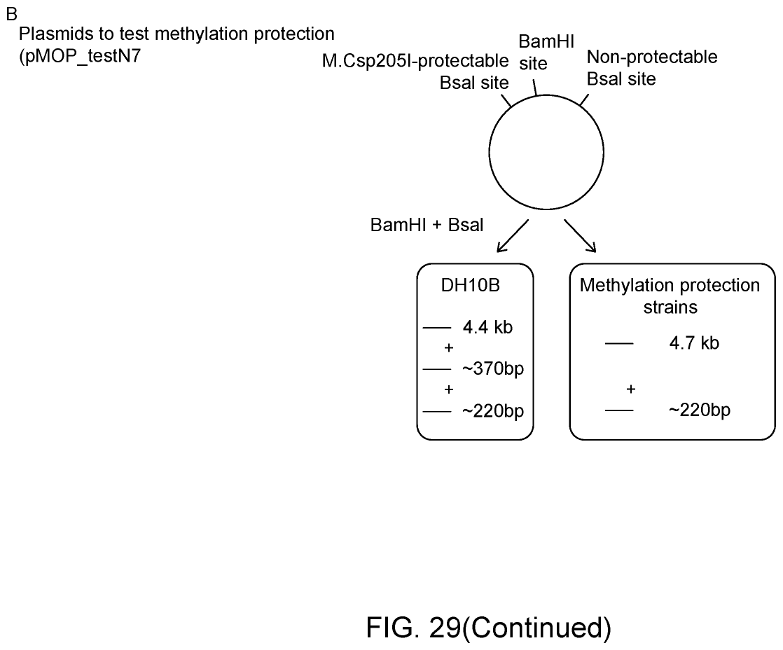

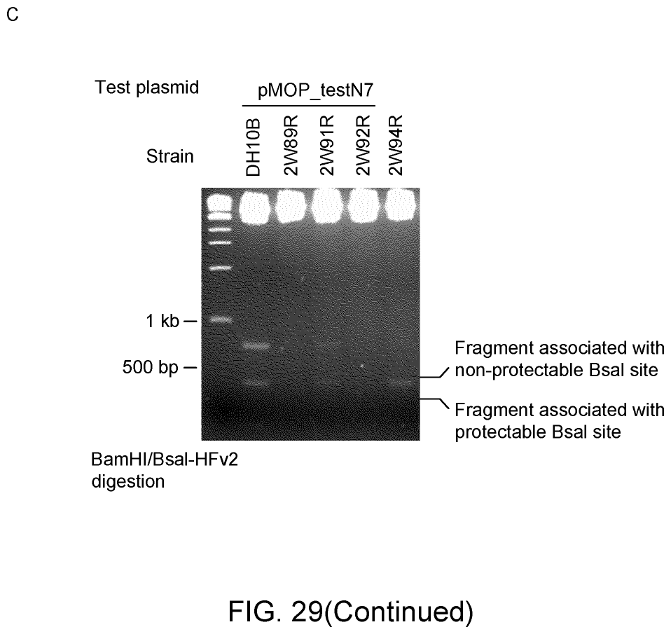

FIG. 29. In vivo methylation protection using M.Csp205I

A. Diagram depicting the E. coli strains tested for methylation protection using M.Csp205I. The strains express either M1.Eco31I or M2.Eco31I with either J23114 or J23112 promoters, B0034m* ribosomal binding sequence (RBS) and L3S2P21 terminator, M.Csp205I and S.Csp205I (both driven by a J23100 promoter with a B0034m* RBS, and with L3S1P51 terminator and L3S1P32 terminator respectively), and a zeocin-resistance gene (ZeoR, driven by an EM2KC promoter) from the arsB locus of the E. coli chromosome.

B. Diagram of the plasmids used to test the methylation protection approach. The test plasmid pMOP_testN7 carries a M.Csp205I-protectable BsaI site, a BamHI site, and a normal (non-switchable) BsaI site. If this test plasmid is prepared in a normal strain, such as DH10B, then both BsaI sites should be cut by BsaI. Therefore, digestion with BamHI and BsaI should generate two small fragments of ˜370 bp and ˜220 bp. If this test plasmid is prepared in the methylation protection strain that expresses the protection methylase, and the second methylase, then the normal BsaI site will be methylated and so will not be cut, but the methylation-protectable BsaI site will bound by the second methylase. With successful methylation protection, when the plasmid is exposed to BamHI and BsaI, of the two BsaI sites, only the unmethylated methylation-protectable BsaI site can be cut and this results in only one small fragment of ˜220 bp. C. Gel electrophoresis of the test plasmids prepared from the DH10B or methylation protection strains following BamHI and BsaI-HFv2 digestion. The digested samples demonstrate the expected pattern as predicted in B only in the strain that expresses M2.Eco31I under J23112 promoter (2W94R), confirming successful protection from methylation of the dCas9-protectable BsaI site by M.Csp205I in this strain.

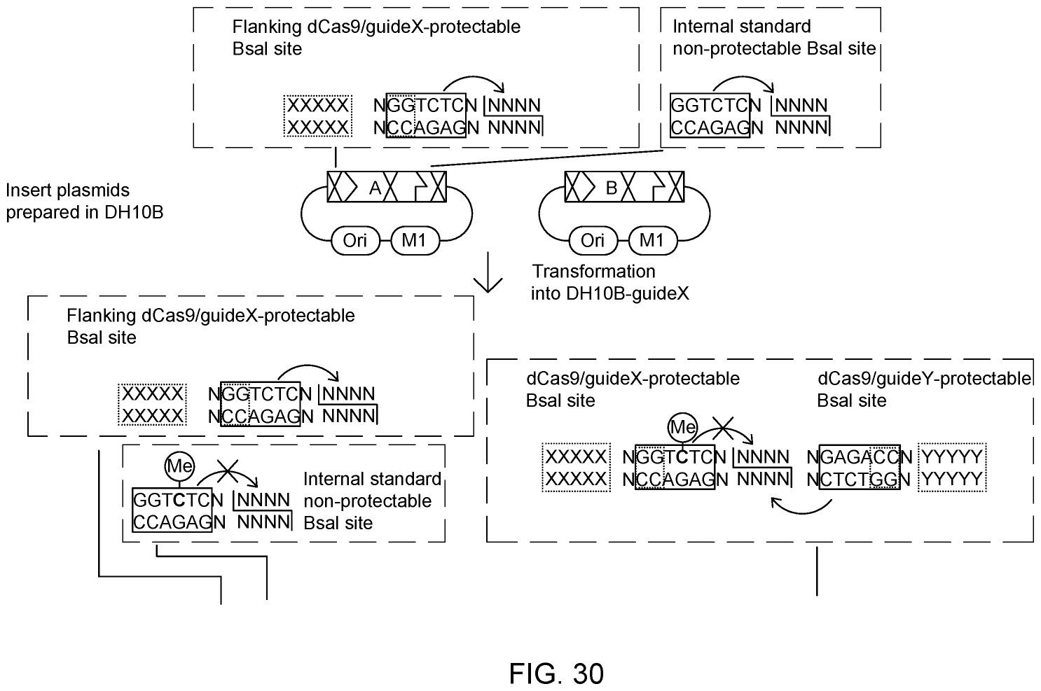

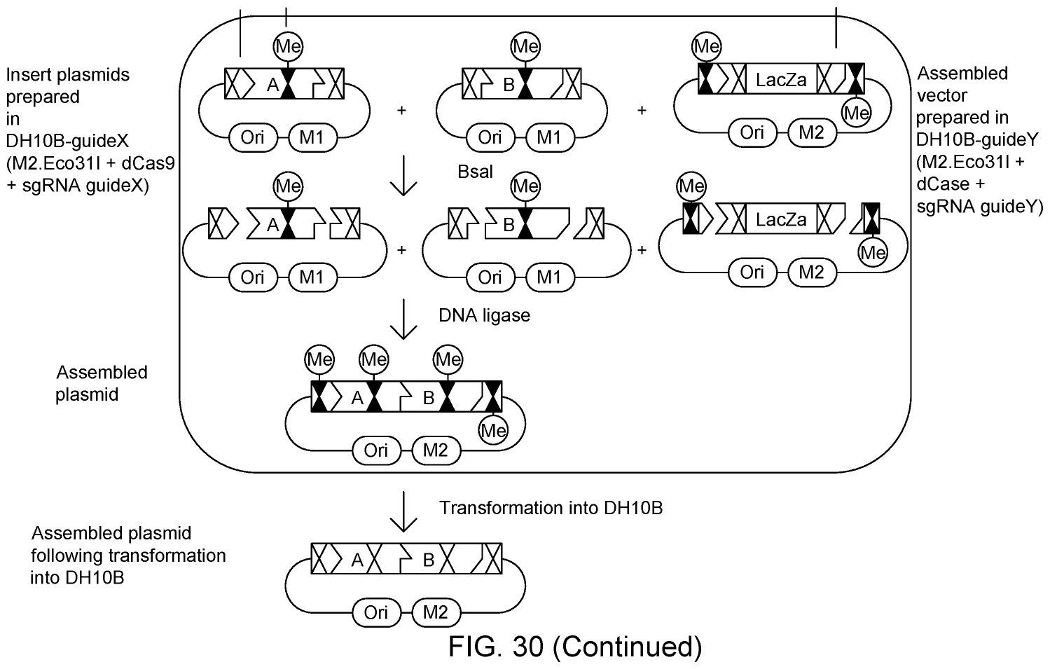

FIG. 30. Universal Assembly system based on methylation-protection approach alone

The diagram depicts the design of the Universal Assembly system using methylation-protection approach alone. The donor plasmids contain inserts flanked by dCas9/guideX-protectable BsaI sites (the BsaI site is boxed and the nucleotides critical for dCas9 binding specificity guided by guide RNA guide X are highlighted in grey) that would generate compatible adhesive ends following BsaI restriction. The internal standard BsaI sites in the insert do not overlap with guideX-guided-dCas9 binding sequence and therefore are not protected from methylation by the M2.Eco31I protection methylase. Transformation of the insert plasmids into an E. coli strain (DH10B-guideX) that expresses the M2.Eco31I protection methylase, dCas9 and a sgRNA guideX targeting the seed sequence XXXXX of dCas9-protectable BsaI site results in selective methylation of internal BsaI sites within the insert (the methylated base is shown in bold). The assembly vector contains a negative selection marker LacZalpha flanked by head-to-head BsaI sites that generate the same adhesive end as that produced by BsaI-based excision of the insert from the donor plasmid. The outer pair of BsaI sites are dCas9/guideX-protectable, whereas the inner pair of BsaI sites are dCas9/guideY-protectable (seed sequence XXXXX is critical for selective binding of dCas9 in the presence of guide RNA guideX to the outer pair BsaI sites, and seed sequence YYYYY is critical for selective binding of dCas9 in the presence of guideY to the inner pair BsaI sites). Preparation of the assembly recipient vector in an E. coli strain (DH10B-guideY) expressing the M2.Eco31I protection methylase, dCas9 and a sgRNA guideY targeting the seed sequence YYYYY results in specific methylation of the outer pair of BsaI sites. The inner pair of BsaI sites are protected from M2.Eco31I by the guideY-guided dCas9. The methylated insert donor plasmids and assembly recipient vector plasmids can be cut with BsaI. Ligation of the cut fragments in a one-pot reaction that contains BsaI favours the generation of correctly assembled DNA comprising the inserts ligated together with the assembly vector backbone, in which all the BsaI sites are methylated. Transformation into a normal E. coli (DH10B) that lacks dCas9-protection and methylation-switching activity removes methylation of all the BsaI sites in the assembled plasmid. Seed sequences XXXXX and YYYYY represents two different 5 bp sequence.

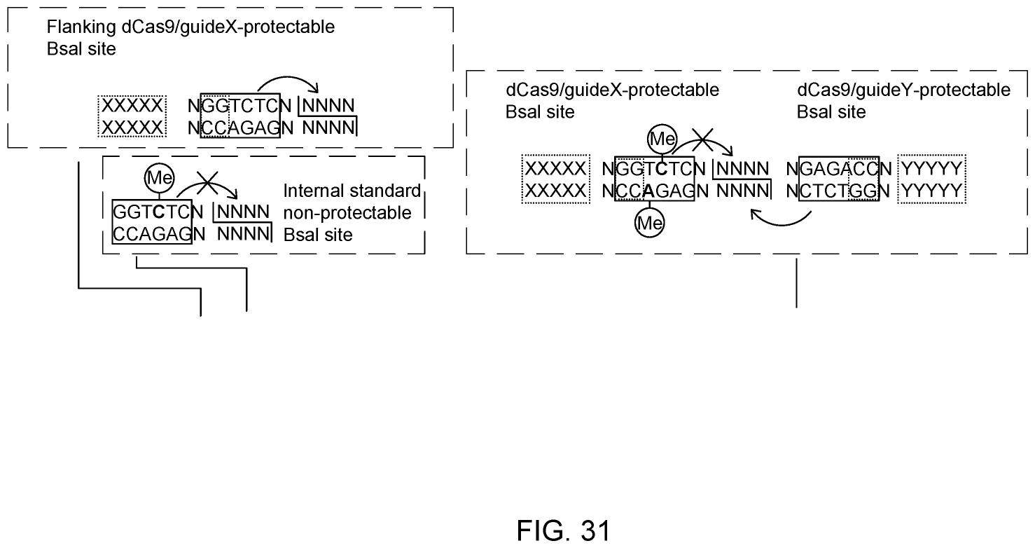

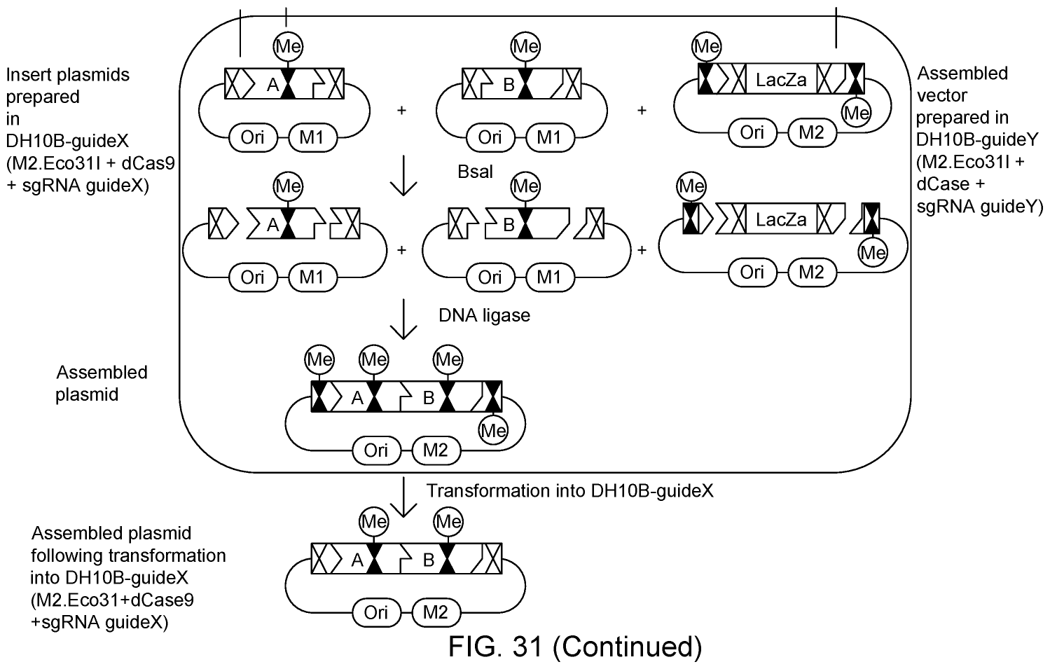

FIG. 31. Universal Assembly system based on methylation protection approach alone

The diagram depicts the basic design of Universal Assembly with the assembled DNA directly transformed into an E. coli strain that deploys methylation-protection approach alone.

The donor plasmids contain inserts flanked by dCas9/guideX-protectable BsaI sites (the BsaI site is boxed and the nucleotides critical for dCas9 binding specificity guided by guide RNA guide X are highlighted in grey) that would generate compatible adhesive ends following BsaI restriction. The internal standard BsaI sites in the insert do not overlap with guideX-guided-dCas9 binding sequence and therefore are not protected from methylation by the M2.Eco31I protection methylase. Preparation of the insert plasmids into an E. coli strain (DH10B-guideX) that expresses the M2.Eco31I protection methylase, dCas9 and a sgRNA guideX targeting the seed sequence XXXXX of dCas9-protectable BsaI site results in selective methylation of internal BsaI sites within the insert (the methylated base is shown in bold). The assembly vector contains a negative selection marker LacZalpha flanked by head-to-head BsaI sites that generate the same adhesive end as that produced by BsaI-based excision of the insert from the donor plasmid. The outer pair of BsaI sites are dCas9/guideX-protectable, whereas the inner pair of BsaI sites are dCas9/guideY-protectable (seed sequence XXXXX is critical for selective binding of dCas9 in the presence of guide RNA guideX to the outer pair BsaI sites, and seed sequence YYYYY is critical for selective binding of dCas9 in the presence of guideY to the inner pair BsaI sites). Preparation of the assembly recipient vector in an E. coli strain (DH10B-guideY) expressing the M2.Eco31I protection methylase, dCas9 and a sgRNA guideY targeting the seed sequence YYYYY results in specific methylation of the outer pair of BsaI sites. The inner pair of BsaI sites are protected from M2.Eco31I by the guideY-guided dCas9. The methylated insert donor plasmids and assembly recipient vector plasmids can be cut with BsaI. Ligation of the cut fragments in a one-pot reaction that contains BsaI favours the generation of correctly assembled DNA comprising the inserts ligated together with the assembly vector backbone, in which all the BsaI sites are methylated. Transformation into in the E. coli strain (DH10B-guideX) that expresses the M2.Eco31I protection methylase, dCas9 and an sgRNA guideX targeting the dCas9/guideX-protectable BsaI site, results in a plasmid that has methylation at all the BsaI sites in the insert, but no methylation at the dCas9/guideX-protectable insert-flanking BsaI sites. The assembled DNA carries a similar methylation pattern on its BsaI sites to the original insert plasmids and so, therefore can be used directly for the next round of Universal Assembly with the same system. Seed sequences XXXXX and YYYYY represents two different 5 bp sequence.

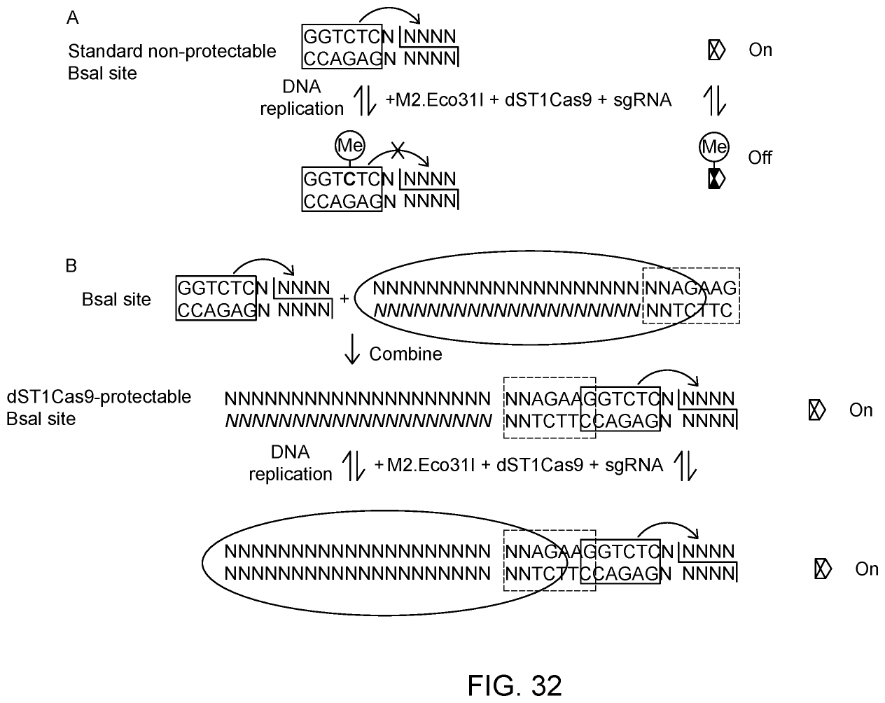

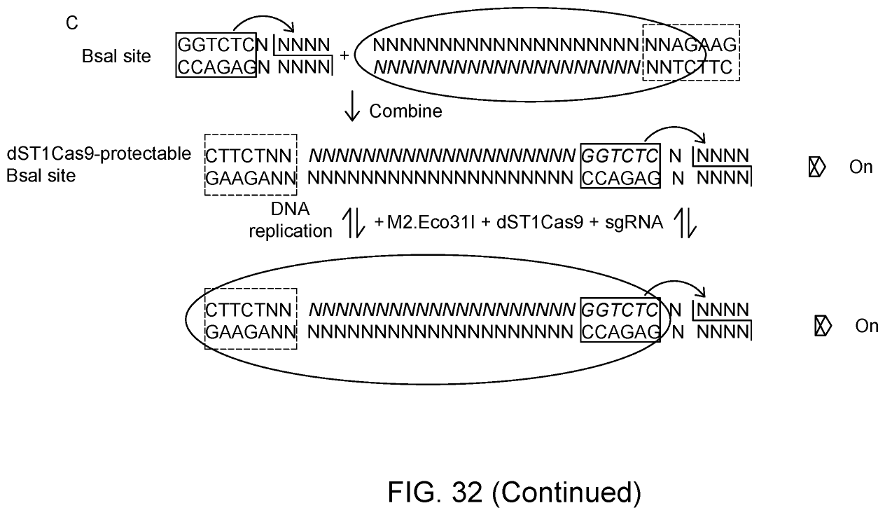

FIG. 32. Methylation protection using dST1Cas9

A. A standard type IIS restriction enzyme site such as BsaI (boxed in solid line) can be methylated by the M2.Eco31I ‘protection’ methylase at the 4th base of the top strand (in bold) when the plasmid is prepared in an E. coli strain that expresses the M2.Eco31I methylase, dST1Cas9 and a chimeric single guide RNA (sgRNA) targeting a specific sequence (the standard BsaI site must not overlap with the sgRNA-guided dST1Cas9 binding sequence).

B. Combining a BsaI site (boxed in solid line) with the sgRNA-guided dST1Cas9 binding site (the 7 bp PAM motif is boxed in dotted line, and the nucleotides that form Watson-Crick base pairing with the sgRNA are shown in italic) creates a BsaI site that overlaps with the dST1Cas9-binding site, in which the sgRNA targets the bottom strand of the combined BsaI site, and the BsaI site overlaps with the PAM motif of the dST1Cas9 binding site. Preparation of the plasmid in an E. coli strain that expresses the M2.Eco31I protection methylase, dST1Cas9 and the sgRNA that recognizes this combined site results in dST1Cas9 binding to this site, which blocks the site from methylation by the M2.Eco31I protection methylase. Such site is referred to as a dST1Cas9-protectable site. The methylation protection process can be represented by the symbols to the right; open triangles depicts an unmethylated BsaI site, and black triangles depict a methylated BsaI site.

C. dST1Cas9-protectable BsaI sites can also be formed by combining a BsaI site (boxed in solid line) with a sgRNA-guided dST1Cas9 binding site (the 7 bp PAM motif is boxed in dotted line, and the nucleotides that form Watson-Crick base pairing with the sgRNA are shown in italic), whereas the sgRNA targets the top strand of the combined BsaI site, and the BsaI site lies within the sgRNA targeted sequence. Preparation of the plasmid in an E. coli strain that expresses the M2.Eco31I protection methylase, dST1Cas9 and the sgRNA that recognizes this combined site results in dST1Cas9 binding to this site, which blocks the site from methylation by the M2.Eco31I protection methylase.

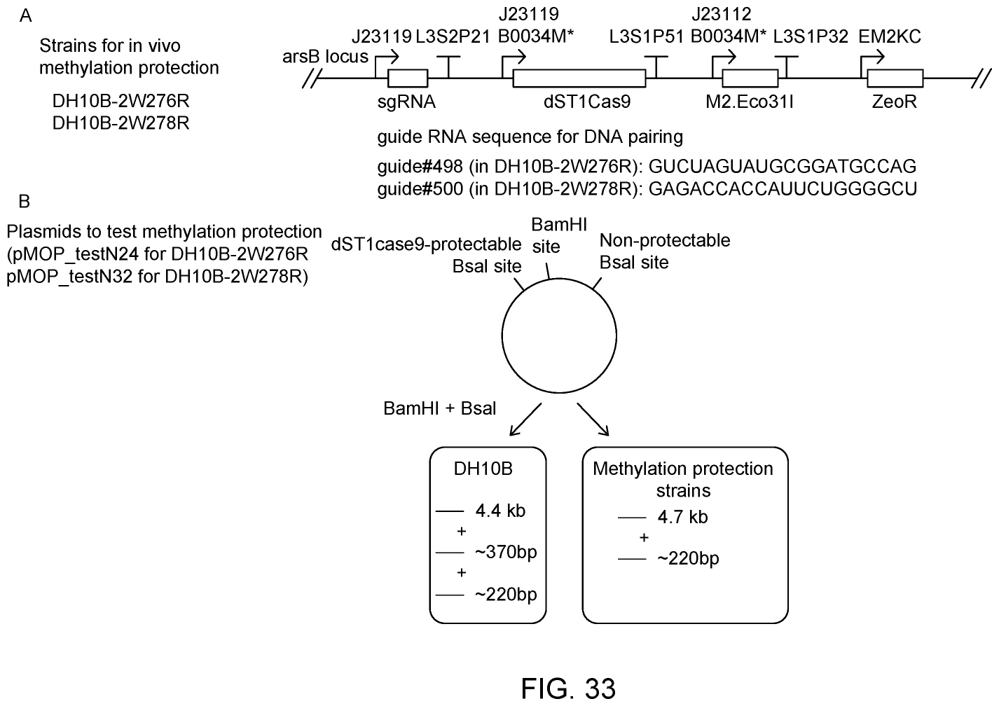

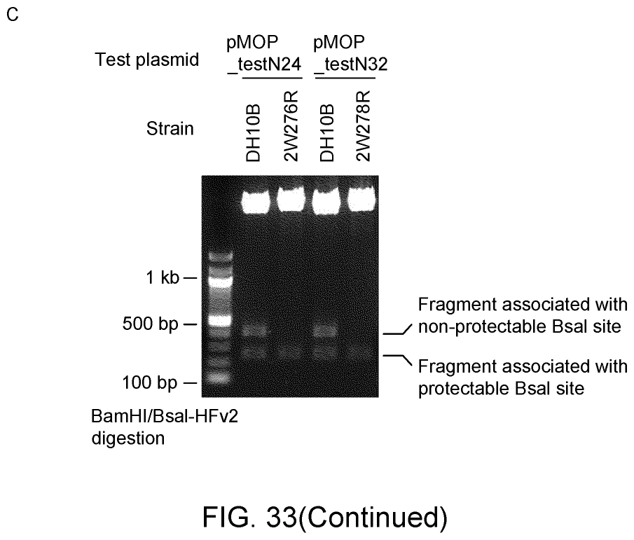

FIG. 33. Methylation protection using dST1Cas9

A. Diagram depicting the E. coli strains for methylation protection. The strains express a sgRNA (guide #498 for strain DH10B-2W276R or guide #500 for strain DH10B-2W278R, driven by a J23119 promoter and with L3S2P21 terminator), dST1Cas9 (driven by a J23119 promoter with a B0034m* ribosomal binding sequence (RBS) and L3S1P51 terminator), the M2.Eco31I methylase (driven by a J23112 promoter with a B0034m* RBS and L3S1P32 terminator) and a zeocin-resistance gene (ZeoR, driven by an EM2KC promoter) from the arsB locus of the E. coli chromosome.

B. Diagram of the plasmids used to test the methylation protection approach. The test plasmids (pMOP_testN24 for DH10B-2W276R and pMOP_testN32 for DH10B-2W278R) carry a dST1Cas9-protectable BsaI site (GTCTAGTATGCGGATGCCAGTGAGAAGGTCTC (SEQ ID NO: 112) in pMOP_testN24, CTTCTCAAGCCCCAGAATGGTGGTCTC (SEQ ID NO: 113) in pMOP_testN32, with the BsaI recognition sequence underlined) for the corresponding guide RNA sequence, a BamHI site, and a non-protectable BsaI site. If this test plasmid is prepared in a normal strain, such as DH10B, then both BsaI sites should be cut by BsaI. Therefore, digestion with BamHI and BsaI should generate two small fragments of ˜370 bp and ˜220 bp. If this test plasmid is prepared in the methylation protection strain that expresses the protection methylase, dST1Cas9 and the corresponding guide RNA, then the normal BsaI site will be methylated and so will not be cut, but the methylation-protectable BsaI site will bound by the dST1Cas9 and so will not be methylated. When the plasmid is exposed to BamHI and BsaI, of the two BsaI sites, only the unmethylated methylation-protectable BsaI site can be cut and this results in only one small fragment of ˜220 bp.

C. Gel electrophoresis of the test plasmids prepared from the DH10B or methylation protection strains following BamHI and BsaI-HFv2 digestion. The digested samples demonstrate the expected pattern as predicted in B, confirming successful protection from methylation of the dST1Cas9-protectable BsaI site by the guided dST1Cas9 in the DH10B-2W276R and DH10B-2W278R strains.

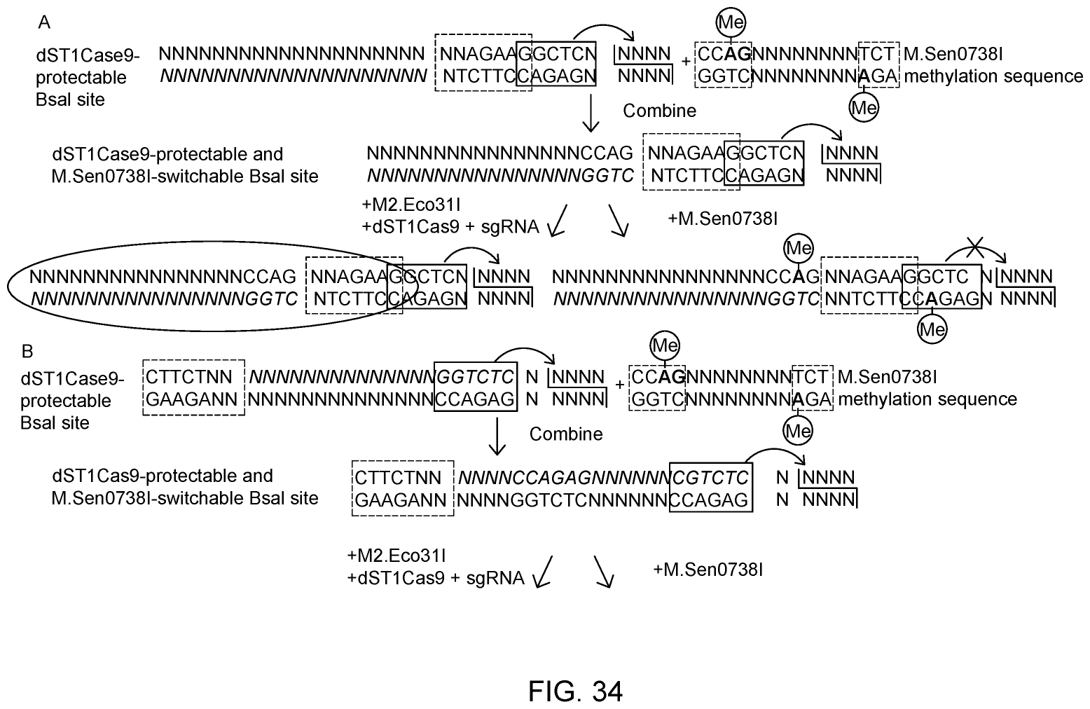

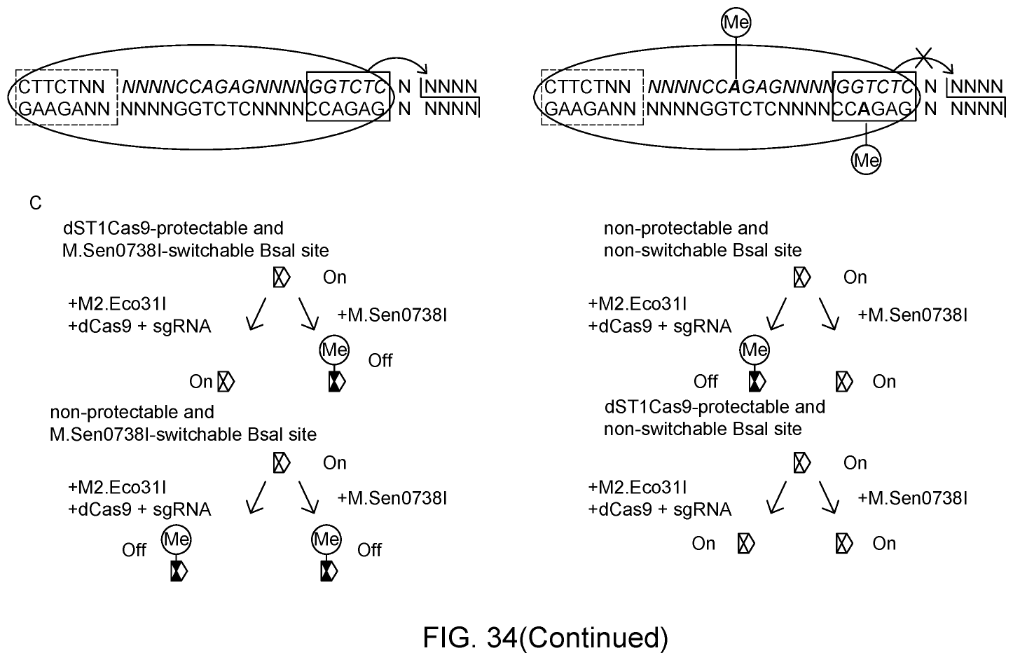

FIG. 34. Combined BsaI sites invoking both dST1Cas9-based methylation-protection and M.Sen0738I-based methylation-switching.

A. Combining a dST1Cas9-protectable BsaI site (the 7 bp PAM motif boxed in dotted line, the nucleotides that form Watson-Crick base pairing with the sgRNA in italic, and the BsaI site boxed in solid line) in which the guide RNA targets the bottom strand of the combined BsaI site, and an M.Sen0738I switch methylase recognition sequence (methylase recognition sequence boxed in dotted line, and the methylated bases in bold) generates a combined BsaI site that is both dST1Cas9-protectable and M.Sen0738I-switchable.

B. A dST1Cas9-protectable and M.Sen0738I-switchable BsaI site can also be generated by combining a dST1Cas9-protectable BsaI site (the 7 bp PAM motif boxed in dotted line, the nucleotides that form Watson-Crick base pairing with the sgRNA in italic, and the BsaI site boxed in solid line) in which the guide RNA targets the top strand of the combined BsaI site, and an M.Sen0738I switch methylase recognition sequence (methylase recognition sequence boxed in dotted line, and the methylated bases in bold).

C. Systems deploying both methylation-protection and methylation-switching may contain four different types of BsaI sites. BsaI sites that are dST1Cas9-protectable can be cut if the plasmid was produced in a strain that coexpresses the M2.Eco31I protection methylase, dST1Cas9 and the appropriate sgRNA. However, the other standard BsaI sites in a plasmid produced in this strain are methylated by the M2.Eco31I protection methylase. BsaI sites that are methylation-switchable are not cut by BsaI if the plasmid has been produced in a strain that expresses the M.Sen0738I switch methylase as these sites are then methylated and so protected from digestion by BsaI. Any non-switchable BsaI sites will not be methylated and can be cut by the enzyme if the plasmid has been produced in such a strain that expresses the M.Sen0738I switch methylase.

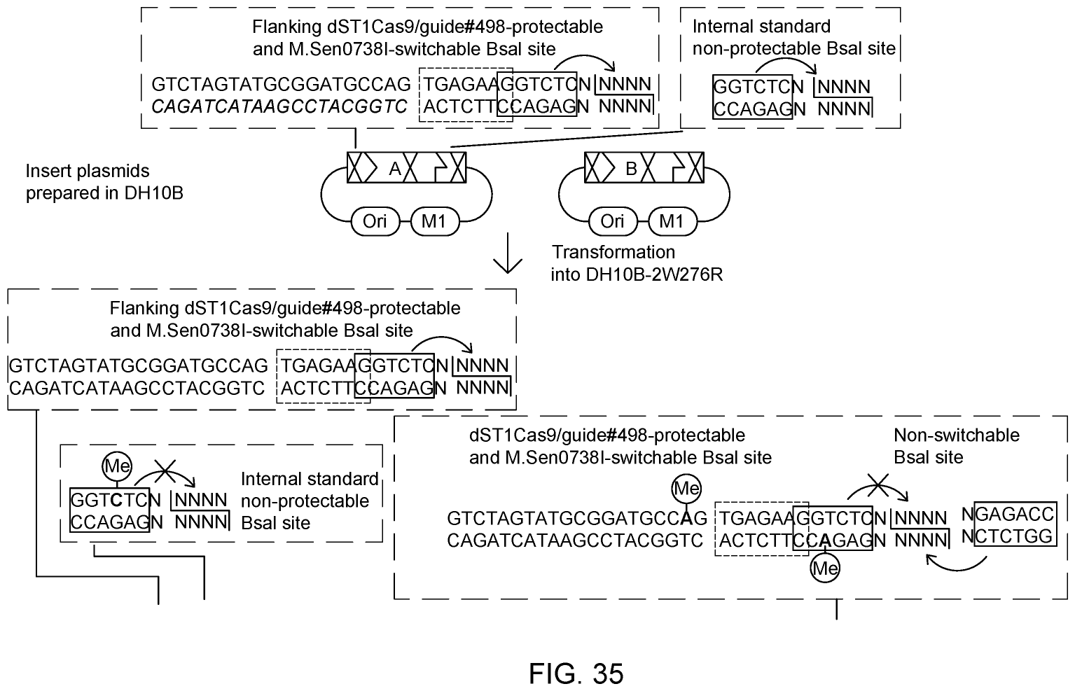

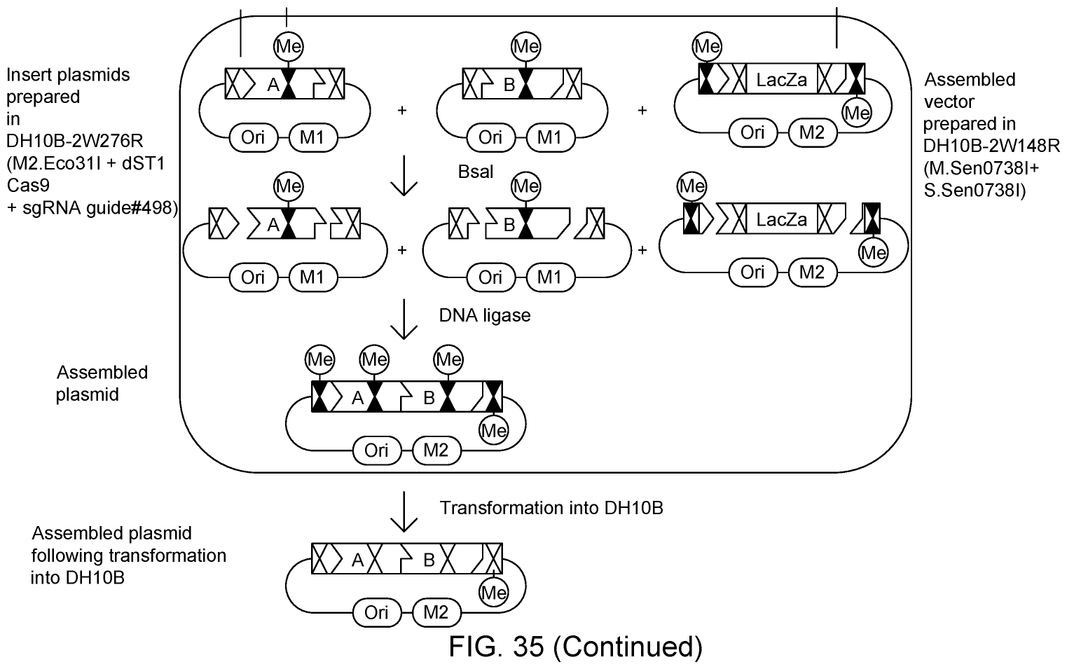

FIG. 35. Universal Assembly system based on both methylation-switching and methylation-protection (M.Sen0738I-based methylation switching and dST1Cas9/guide #498-based methylation protection as an example)

The diagram depicts the design of the implemented Universal Assembly system using methylation-protection and methylation-switching. The donor plasmids contain inserts flanked by dST1Cas9/guide #498-protectable and M.Sen0738I-switchable BsaI sites (the 7 bp PAM motif boxed in dotted line, the nucleotides that form Watson-Crick base pairing with the sgRNA in italic, and the BsaI site boxed in solid line) that would generate compatible adhesive ends following BsaI restriction. The internal standard BsaI sites in the insert do not overlap with guided-dST1Cas9 binding sequence and therefore are not protected from methylation by the M2.Eco31I protection methylase. Transformation of the insert plasmids into an E. coli strain (DH10B-2W276R) that expresses the M2.Eco31I protection methylase, dST1Cas9 and a sgRNA guide #498 targeting the dST1Cas9-protectable BsaI site results in selective methylation of internal BsaI sites within the insert (the methylated base is shown in bold). The assembly vector contains a negative selection marker LacZalpha flanked by head-to-head BsaI sites that generate the same adhesive end as that produced by BsaI-based excision of the insert from the donor plasmid. The outer pair of BsaI sites are dST1Cas9/guide #498-protectable and M.Sen0738I methylation-switchable, whereas the inner pair of BsaI sites are nonswitchable. Preparation of the assembly recipient vector in an E. coli strain (DH10B-2W148R) expressing the M.Sen0738I switch methylase results in specific methylation of the outer pair of BsaI sites. The methylated insert donor plasmids and assembly recipient vector plasmids can be cut with BsaI. Ligation of the cut fragments in a one-pot reaction that contains BsaI favours the generation of correctly assembled DNA comprising the inserts ligated together with the assembly vector backbone, in which all the BsaI sites are methylated. Transformation into a normal E. coli (DH10B) that lacks dST1Cas9-protection and methylation-switching activity removes methylation of all the BsaI sites in the assembled plasmid.

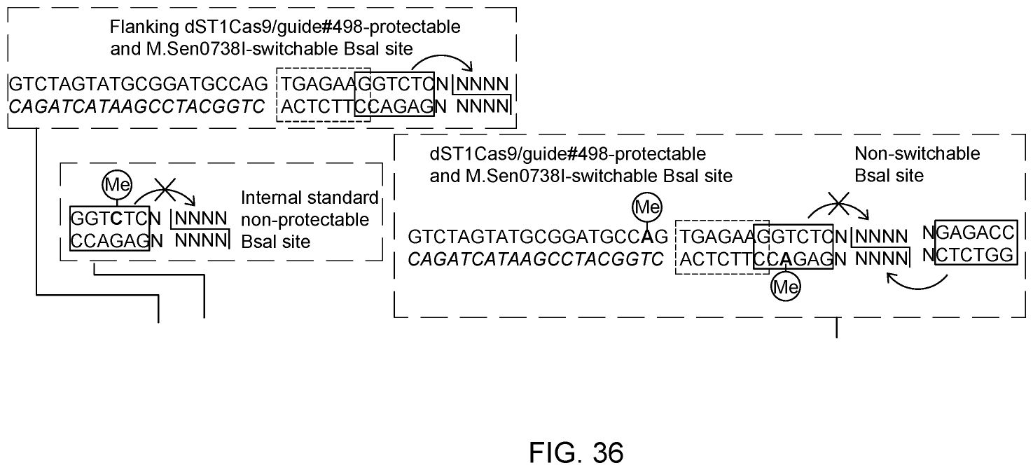

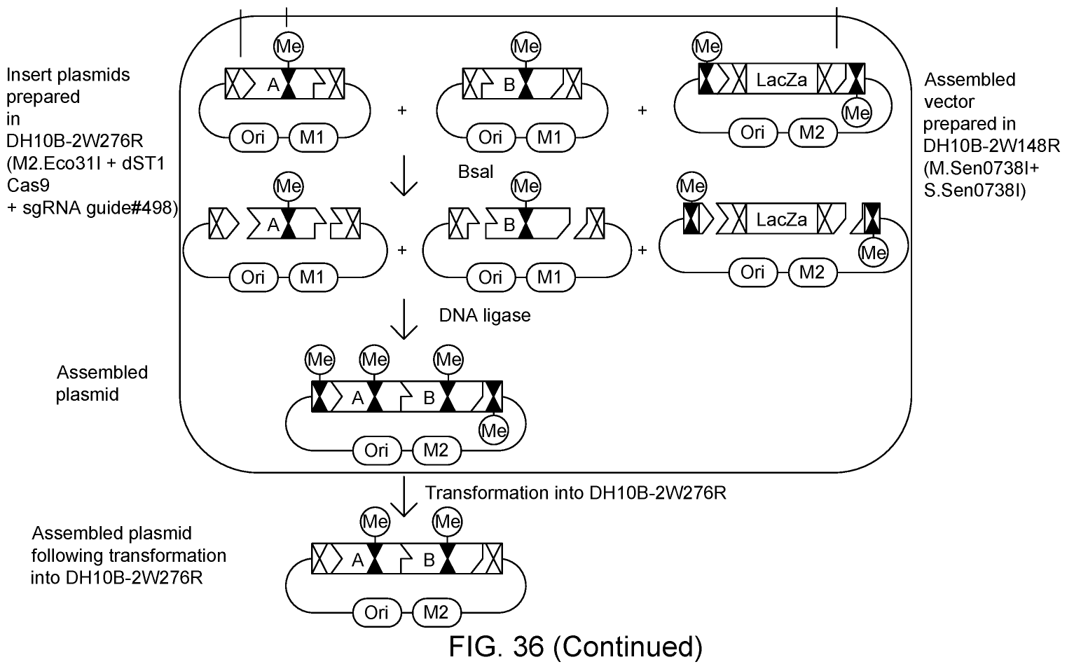

FIG. 36. Universal Assembly system based on both methylation-switching and methylation-protection (M.Sen0738I-based methylation switching and dST1Cas9/guide #498-based methylation protection as an example, direct transformation into DH10B-2W276R).

The diagram depicts the basic design of Universal Assembly with the assembled DNA directly transformed into an E. coli strain that deploys the methylation-protection principle. The insert donor plasmids contain inserts flanked by dST1Cas9/guide #498-protectable and M.Sen0738I methylase-switchable BsaI sites (the 7 bp PAM motif boxed in dotted line, the nucleotides that form Watson-Crick base pairing with the sgRNA in italic, and the BsaI site boxed in solid line) that would generate mutually compatible adhesive ends following BsaI restriction. The internal standard BsaI sites in the insert do not overlap with the sequence that is the target of the guide RNA for the dST1Cas9 and therefore are not bound by dST1Cas9 and so are methylated by the M2.Eco31I protection methylase. Preparation of the insert plasmids in an E. coli strain (DH10B-2W276R) that expresses the M2.Eco31I protection methylase, dST1Cas9 and an sgRNA guide #498 targeting the dST1Cas9/guide #498-protectable BsaI results in specific methylation of standard internal BsaI sites within the insert (the methylated base is shown in bold). The assembly vector contains a negative selection marker LacZalpha flanked by head-to-head BsaI sites that generate the same adhesive end. The outer pair of BsaI sites are dST1Cas9/guide #498-protectable and M.Sen0738I methylase-switchable, whereas the inner pair are non-switchable. Preparation of the assembly vector in an E. coli strain expressing M.Sen0738I (DH10B-2W148R) results in specific methylation of the outer pair of BsaI sites. The methylated insert plasmids and assembly vector can be cut with BsaI. Ligation of the cut fragments in a one-pot reaction that contains BsaI favours the generation of correctly assembled DNA comprising the inserts ligated together with the assembly vector backbone, in which all the BsaI sites are methylated. Transformation into in the E. coli strain (DH10B-2W276R) that expresses the M2.Eco31I protection methylase, dST1Cas9 and an sgRNA guide #498 targeting the dST1Cas9/guide #498-protectable BsaI site, results in a plasmid that has methylation at all the BsaI sites in the insert, but no methylation at the dST1Cas9/guide #498-protectable insert-flanking BsaI sites. The assembled DNA carries a similar methylation pattern on its BsaI sites to the original insert plasmids and so, therefore can be used directly for the next round of Universal Assembly.

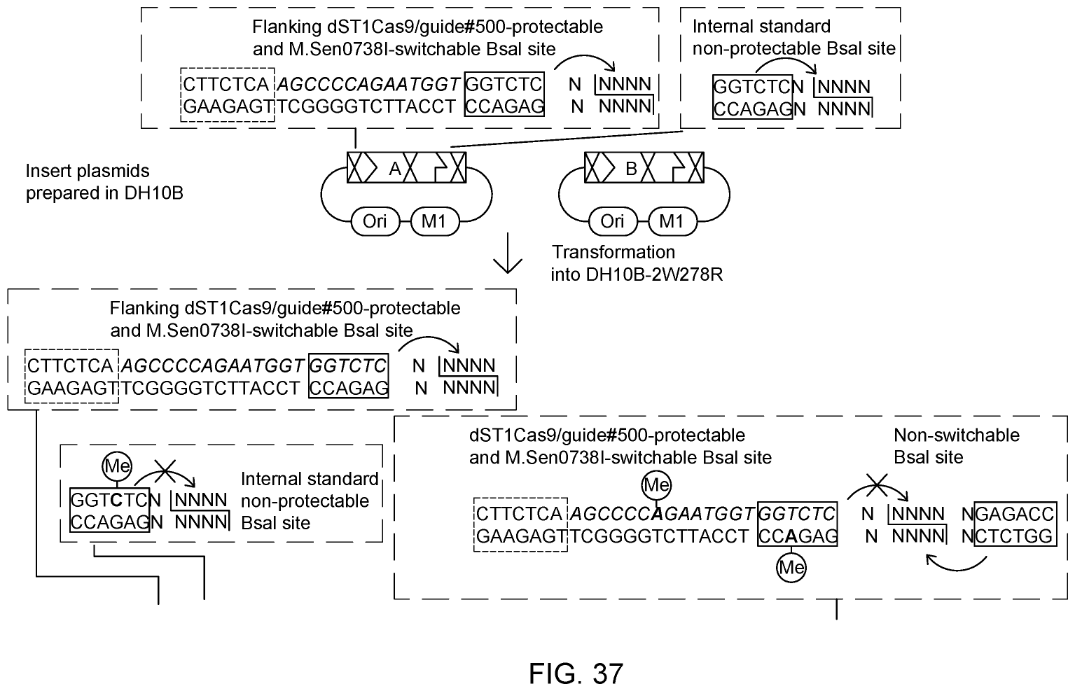

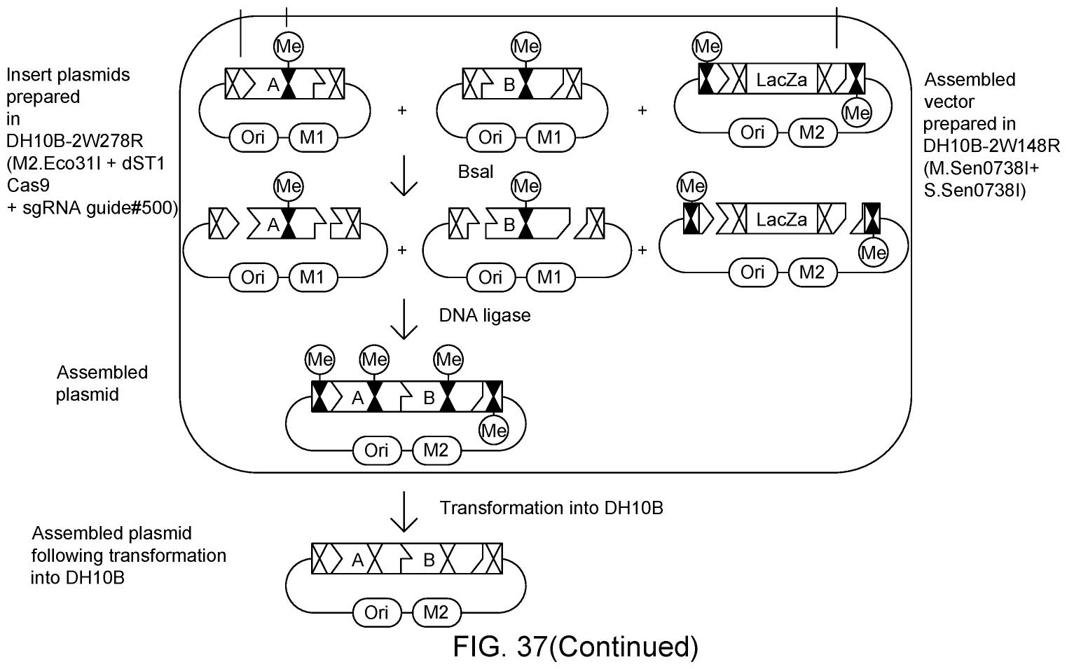

FIG. 37. Universal Assembly system based on both methylation-switching and methylation-protection (M.Sen0738I-based methylation switching and dST1Cas9/guide #500-based methylation protection as an example)

The diagram depicts the design of the implemented Universal Assembly system using methylation-protection and methylation-switching. The donor plasmids contain inserts flanked by dST1Cas9/guide #500-protectable and M.Sen0738I-switchable BsaI sites (the 7 bp PAM motif boxed in dotted line, the nucleotides that form Watson-Crick base pairing with the sgRNA in italic, and the BsaI site boxed in solid line) that would generate compatible adhesive ends following BsaI restriction. The internal standard BsaI sites in the insert do not overlap with guided-dST1Cas9 binding sequence and therefore are not protected from methylation by the M2.Eco31I protection methylase. Transformation of the insert plasmids into an E. coli strain (DH10B-2W278R) that expresses the M2.Eco31I protection methylase, dST1Cas9 and a sgRNA guide #500 targeting the dST1Cas9-protectable BsaI site results in selective methylation of internal BsaI sites within the insert (the methylated base is shown in bold). The assembly vector contains a negative selection marker LacZalpha flanked by head-to-head BsaI sites that generate the same adhesive end as that produced by BsaI-based excision of the insert from the donor plasmid. The outer pair of BsaI sites are dST1Cas9/guide #500-protectable and M.Sen0738I methylation-switchable, whereas the inner pair of BsaI sites are nonswitchable. Preparation of the assembly recipient vector in an E. coli strain (DH10B-2W148R) expressing the M.Sen0738I switch methylase results in specific methylation of the outer pair of BsaI sites. The methylated insert donor plasmids and assembly recipient vector plasmids can be cut with BsaI. Ligation of the cut fragments in a one-pot reaction that contains BsaI favours the generation of correctly assembled DNA comprising the inserts ligated together with the assembly vector backbone, in which all the BsaI sites are methylated. Transformation into a normal E. coli (DH10B) that lacks dST1Cas9-protection and methylation-switching activity removes methylation of all the BsaI sites in the assembled plasmid.

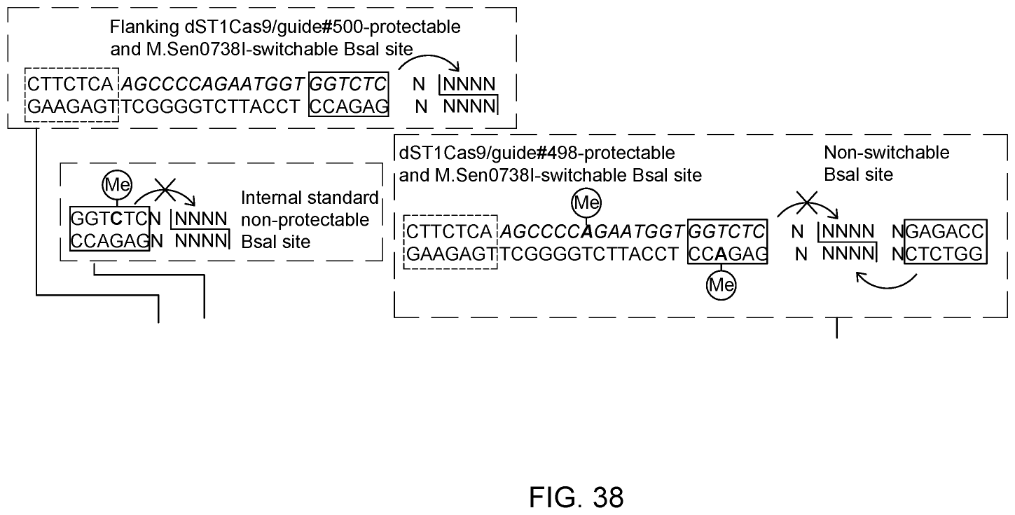

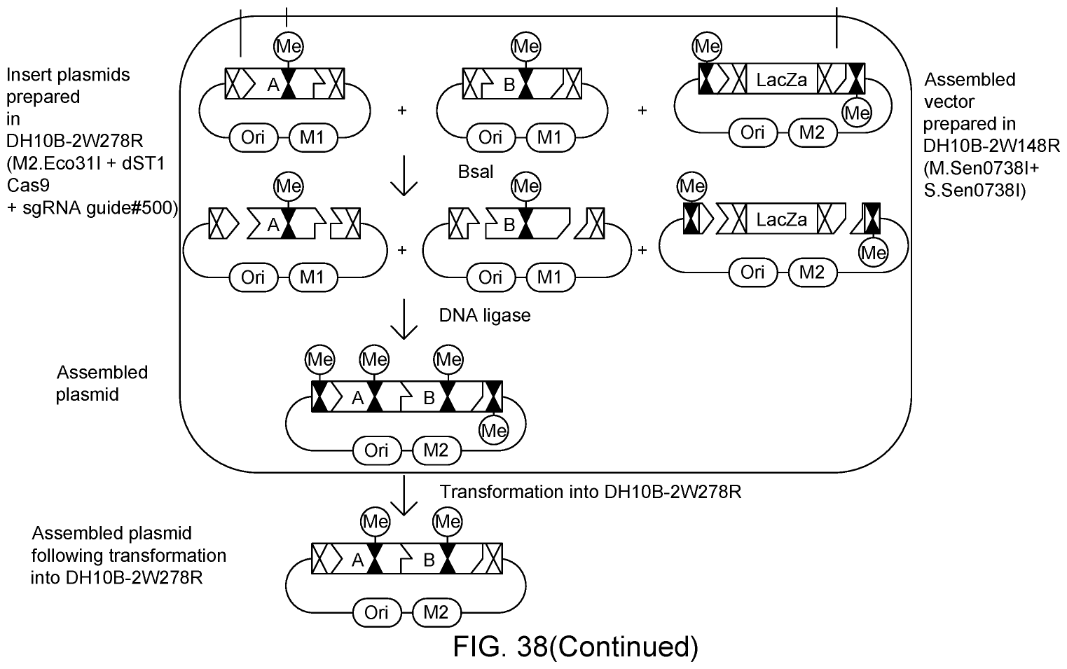

FIG. 38. Universal Assembly system based on both methylation-switching and methylation-protection (M.Sen0738I-based methylation switching and dST1Cas9/guide #500-based methylation protection as an example, direct transformation into DH10B-2W278R).

The diagram depicts the basic design of Universal Assembly with the assembled DNA directly transformed into an E. coli strain that deploys the methylation-protection principle. The insert donor plasmids contain inserts flanked by dST1Cas9/guide #500-protectable and M.Sen0738I methylase-switchable BsaI sites (the 7 bp PAM motif boxed in dotted line, the nucleotides that form Watson-Crick base pairing with the sgRNA in italic, and the BsaI site boxed in solid line) that would generate mutually compatible adhesive ends following BsaI restriction. The internal standard BsaI sites in the insert do not overlap with the sequence that is the target of the guide RNA for the dST1Cas9 and therefore are not bound by dST1Cas9 and so are methylated by the M2.Eco31I protection methylase. Preparation of the insert plasmids in an E. coli strain (DH10B-2W278R) that expresses the M2.Eco31I protection methylase, dST1Cas9 and an sgRNA guide #500 targeting the dST1Cas9/guide #500-protectable BsaI results in specific methylation of standard internal BsaI sites within the insert (the methylated base is shown in bold). The assembly vector contains a negative selection marker LacZalpha flanked by head-to-head BsaI sites that generate the same adhesive end. The outer pair of BsaI sites are dST1Cas9/guide #500-protectable and M.Sen0738I methylase-switchable, whereas the inner pair are non-switchable. Preparation of the assembly vector in an E. coli strain expressing M.Sen0738I (DH10B-2W148R) results in specific methylation of the outer pair of BsaI sites. The methylated insert plasmids and assembly vector can be cut with BsaI. Ligation of the cut fragments in a one-pot reaction that contains BsaI favours the generation of correctly assembled DNA comprising the inserts ligated together with the assembly vector backbone, in which all the BsaI sites are methylated. Transformation into in the E. coli strain (DH10B-2W278R) that expresses the M2.Eco31I protection methylase, dST1Cas9 and an sgRNA guide #500 targeting the dST1Cas9/guide #500-protectable BsaI site, results in a plasmid that has methylation at all the BsaI sites in the insert, but no methylation at the dST1Cas9/guide #500-protectable insert-flanking BsaI sites. The assembled DNA carries a similar methylation pattern on its BsaI sites to the original insert plasmids and so, therefore can be used directly for the next round of Universal Assembly.

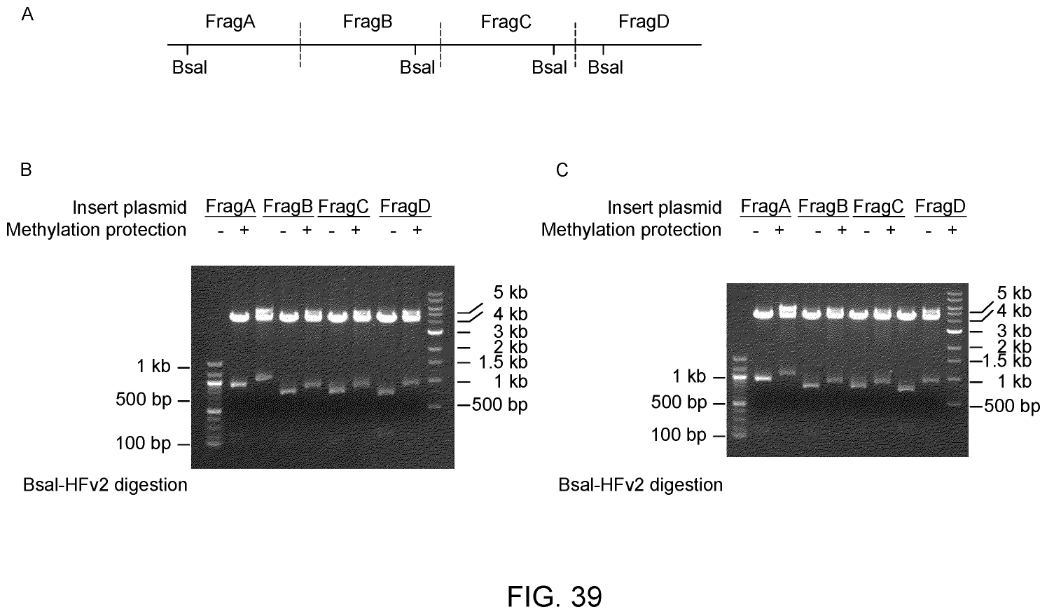

FIG. 39. Practical DNA assembly using Universal Assembly with dST1Cas9-based methylation protection

A. Diagram of the DNA fragment to assemble. Four fragments of human DNA from the MICA locus (FragA, FragB, FragC and FragD), each containing an internal BsaI site were assembled together to produce a 3.6 kb fragment (Genbank KF724576.1:23-3633) as the final product. The coordinates of the DNA fragment to assemble are FragA (KF724576.1:23-1048), FragB (KF724576.1:1045-1894), FragC (KF724576.1:1891-2744), FragD (KF724576.1:2741-3633).

B. Gel electrophoresis of BsaI digestion of insert plasmids with pMOK498 backbone (pMOK498_F5A, pMOK498_F5B, pMOK498_F5C, pMOK498_F5D) prepared in the normal E. coli strain, DH10B, (−) or the E. coli strain DH10B-2W276R (+) that expresses dST1Cas9, a guide RNA guide #498 and the M2.Eco31I protection methylase following BsaI-HFv2 digestion. dST1Cas9-based methylation protection prevents methylation of the insert-flanking dST1Cas9-protectable BsaI sites, but the internal BsaI sites are methylated and so cannot be cut by BsaI, resulting in larger complete insert fragments that are suitable for assembly. For insert plasmids prepared in DH10B-2W276R, BsaI-HFv2 digestion of insert plasmids with both flanking BsaI sites fully protected from methylation generates a ˜4.3 kb vector backbone, whereas BsaI-HFv2 digestion of insert plasmids with one of the two flanking BsaI sites protected from methylation generates a single ˜5.3 kb fragment.

C. Gel electrophoresis of BsaI digestion of insert plasmids with pMOK500 backbone (pMOK500_F6A, pMOK500_F6B, pMOK500_F6C, pMOK500_F6D) prepared in the normal E. coli strain, DH10B, (−) or the E. coli strain DH10B-2W278R (+) that expresses dST1Cas9, a guide RNA guide #500 and the M2.Eco31I protection methylase following BsaI-HFv2 digestion. dST1Cas9-based methylation-protection prevents methylation of the insert-flanking dST1Cas9-protectable BsaI sites, but the internal BsaI sites are methylated and so cannot be cut by BsaI, resulting in larger complete insert fragments that are suitable for assembly. For insert plasmids prepared in DH10B-2W278R, BsaI-HFv2 digestion of insert plasmids with both flanking BsaI sites fully protected from methylation generates a ˜4.3 kb vector backbone, whereas BsaI-HFv2 digestion of insert plasmids with one of the two flanking BsaI sites protected from methylation generates a single ˜5.3 kb fragment.

D. Gel electrophoresis of DNA clones assembled into DH10B-2W276R or DH10B-2W278R cells by Universal Assembly from insert plasmids prepared in DH10B-2W276R or DH10B-2W278R following digestion by DraIII, which releases the assembled fragment through DraIII sites in the vector backbones, but does not cut inside the assembled 3.6 kb fragment as the assembled 3.6 kb DNA lacks DraIII sites. 6 out of 8 of clones assembled in DH10B-2W276R, and 8 out of 8 clones assembled in DH10B-2W278R were verified by DraIII digestion and by DNA sequencing.

E. Gel electrophoresis of DNA clones assembled into DH10B-2W276R or DH10B-2W278R by Universal Assembly following digestion by BsaI-HFv2. For correctly assembled DNA with internal BsaI sites completely blocked by M2.Eco31I methylation and both flanking BsaI sites fully protected from methylation, BsaI-HFv2 digestion generates a 4.4 kb vector backbone and a 3.6 kb insert DNA, whereas for assembled DNA with one of the two flanking BsaI sites protected from methylation, BsaI-HFv2 digestion generates a single 8.0 kb fragment.

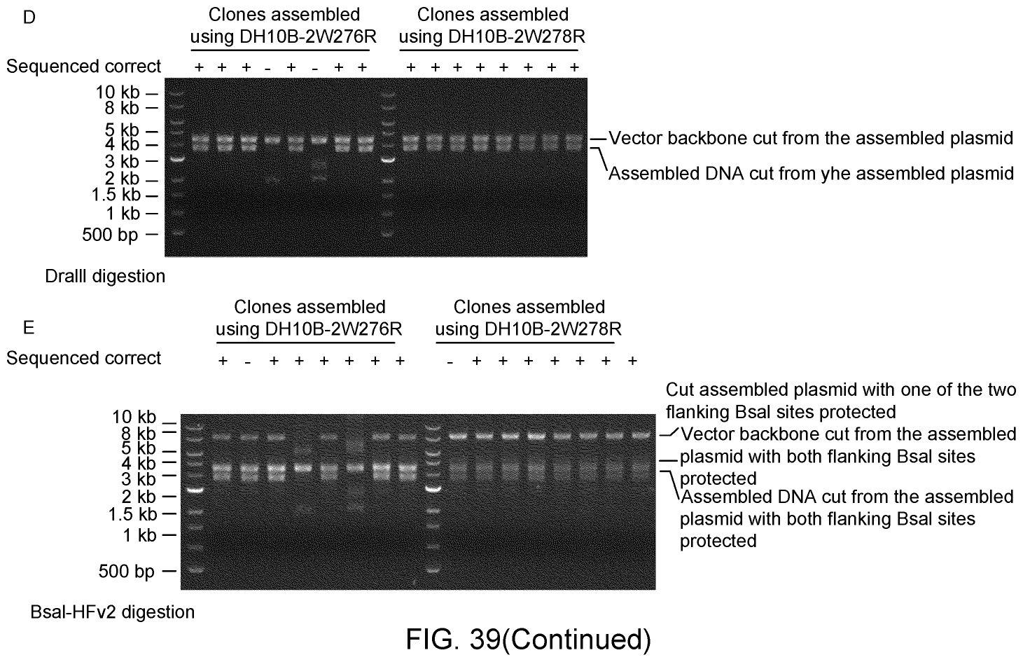

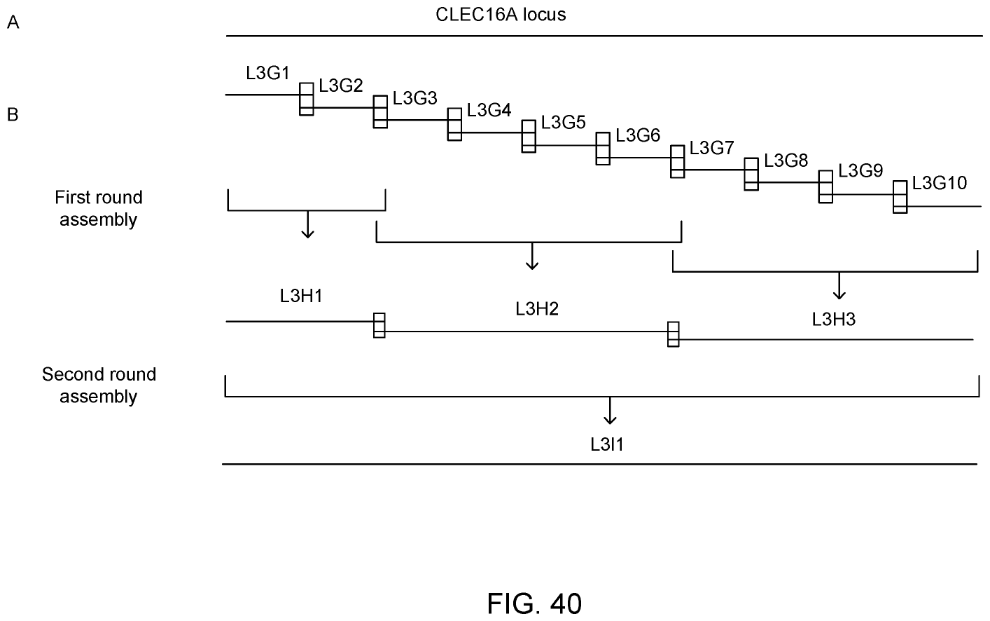

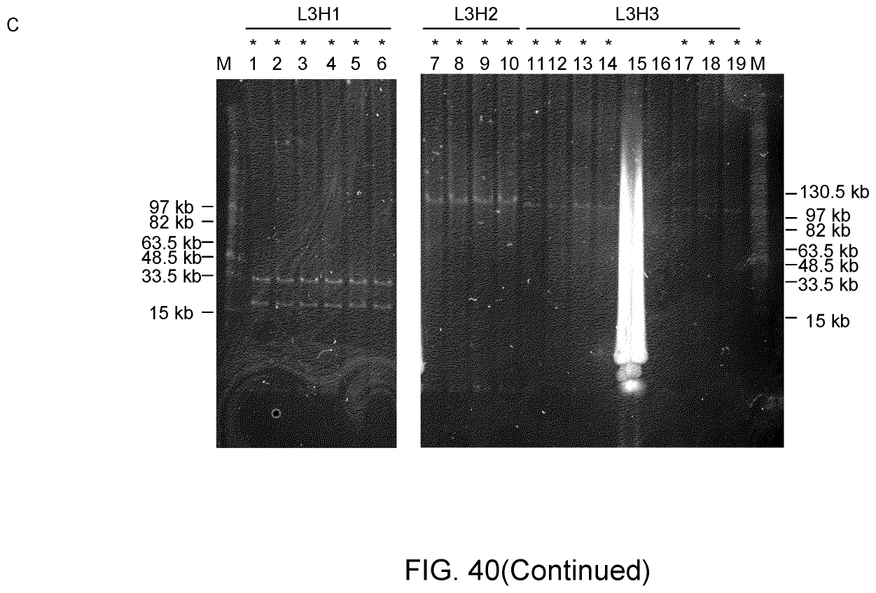

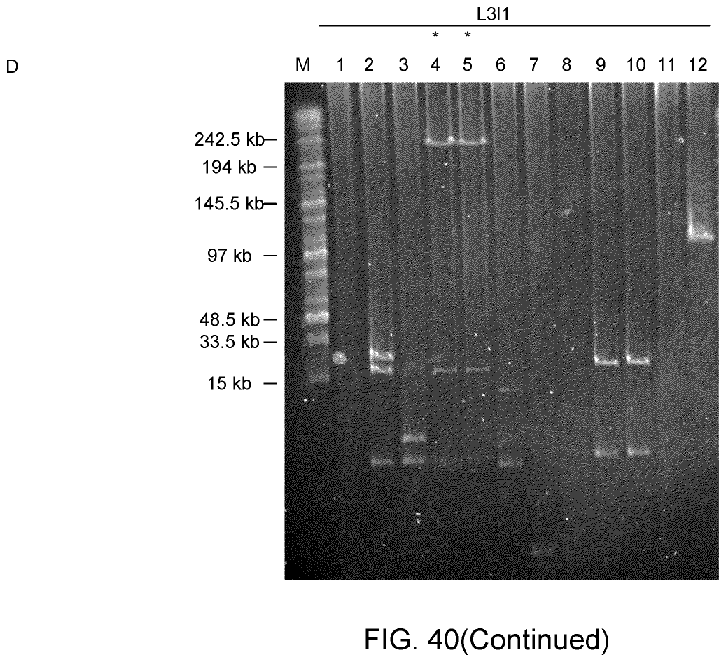

FIG. 40. Practical scarless assembly of a ˜260 kb DNA using Universal Assembly

A and B. A ˜260 kb sequence corresponding to the CLEC16A locus was first digitally broken into 10 fragments with 4 bp overlapping sequence (L3G1-L3G10). The 10 starting fragments cloned in insert plasmids with flanking dCas9-guide #401 methylation protectable and M.Sen0738I methylation switchable BsaI sites carry suitable overhang sequences depending on position of the assembled fragment will take up in the next round of assembly. The fragments were assembled 2-4 fragments per group using scarless Universal Assembly into intermediate fragments L3H1, L3H2 and L3H3. The three intermediate fragments were further assembled into a single ˜260 kb fragment L3I1.

C. Pulsed field electrophoresis of assembled plasmids carrying intermediate fragments L3H1 (lane 1-6), L3H2 (lane 7-10) and L3H3 (lane 11-19), following restriction using NotI, along with MidRange I PFG Marker (NEB, ‘M’). Clones with correct restriction pattern are marked with an asterisk. The assembly vector backbone contains flanking NotI sites. Restriction of the assembled clones with NotI therefore generates the ˜6.3 kb assembly vector backbone and the insert fragment (˜51 kb for L3H1, ˜105 kb for L3H2 and ˜105 kb for L3H3). Fragment L3H1 contains an internal NotI site therefore restriction of L3H1 generates two fragments (˜33 kb and ˜19 kb) as predicted.

D. Pulsed field electrophoresis of assembled plasmids carrying fully assembled fragment L3I1 (lane 1-12), following restriction using NotI, along with MidRange I PFG Marker (NEB, ‘M’). Clones with correct restriction pattern are marked with an asterisk. The assembly vector backbone contains flanking NotI sites, and the fully assembled L3I1 (˜261 kb) contains an internal NotI site. Restriction of the assembled clones with NotI generates the ˜6.3 kb assembly vector backbone, and two fragments (˜242 kb and ˜19 kb) as predicted.

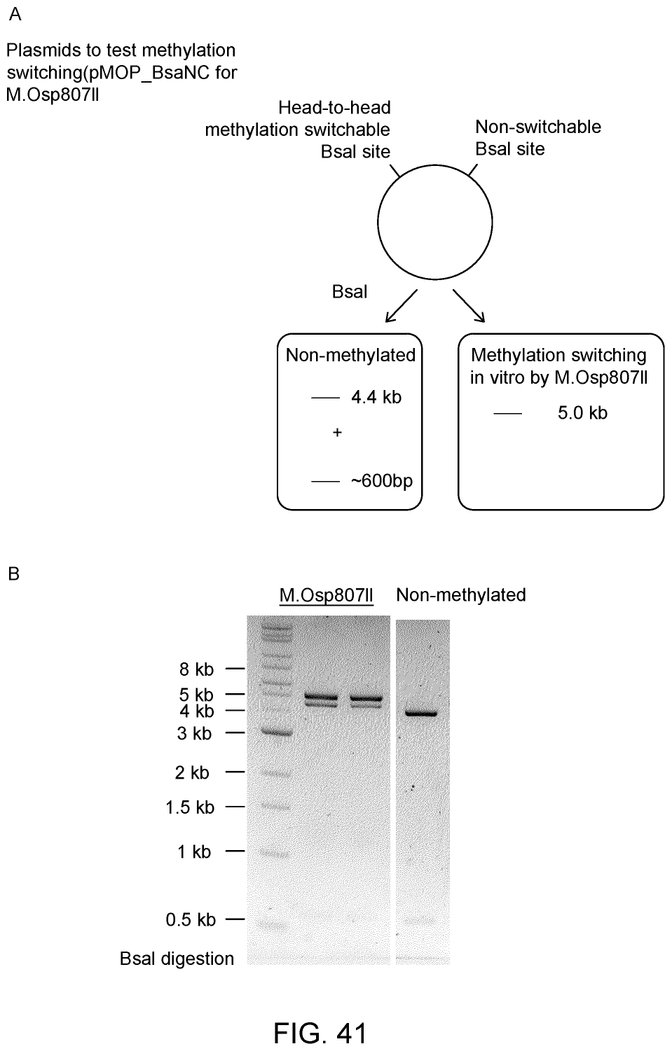

FIG. 41. Testing of methylation switching activity of recombinant M.Osp807II

A. Experimental designs to test methylation switching activity of M.Osp807II in vitro. The test plasmid (pMOP_BsaNC) contain a head-to-head M.Osp807II-methylation-switchable BsaI site (GACTTGGTCTCATGCCTGAGACCAAGTC) ˜600 bp away from a non-switchable BsaI site. Restriction digestion by BsaI of test plasmid would result in cutting at both BsaI sites resulting in generation of a 4.4 kb and a 600 bp fragment. Restriction digestion of test plasmid that has been methylated by M.Osp807II in vitro generate a single 5 kb fragment due to blocking of the methylation-switchable BsaI restriction sites by in vitro methylation. The head-to-head arrangement of overlapping methylation/restriction site allows the same assay to be used to detect any residual single strand nicking activity of the restriction enzyme towards the methylated restriction site.

B. Agarose gel electrophoresis analysis of the test plasmid (prepared in DH10B) methylated by M.Osp807II in vitro followed by digested with BsaI-HFv2. The results show that in vitro methylation by M.Osp807II successfully blocked the switchable BsaI restriction site.

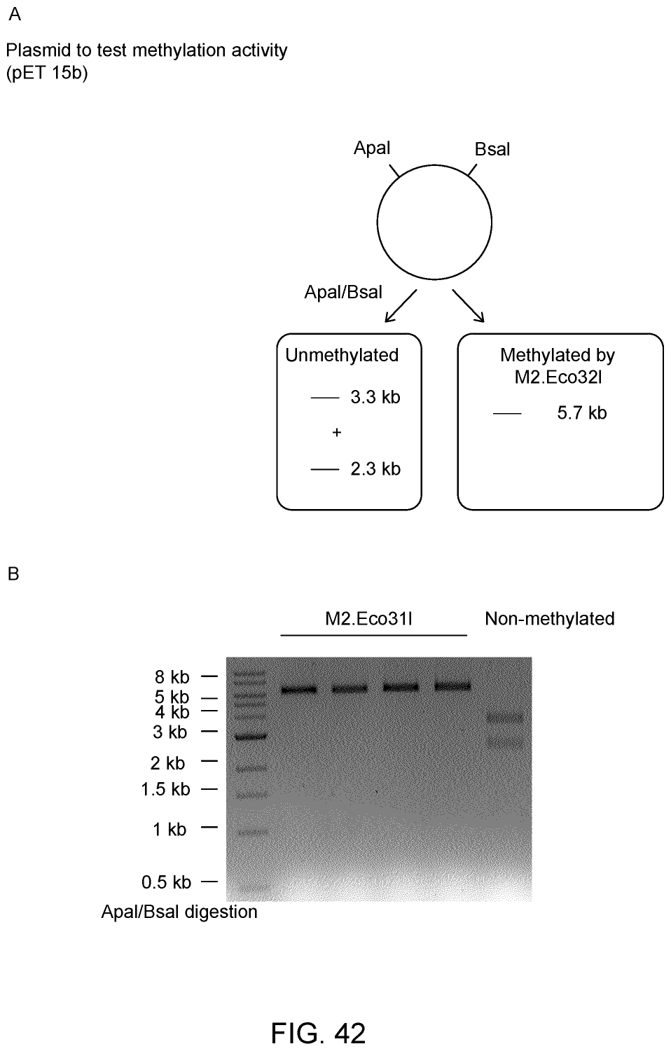

FIG. 42. Testing of methylation activity of recombinant M2.Eco31I

A. Experimental designs to test methylation activity of M2.Eco31I in vitro. The test plasmid (pET-15b) contain an ApaI site and a BsaI site. Restriction digestion by ApaI/BsaI would generate a 3.3 kb and a 2.3 kb fragment. Restriction digestion of test plasmid that has been methylated by M2.Eco31I in vitro would generate a single 5.6 kb fragment due to blocking of the BsaI site by in vitro methylation.

B. Agarose gel electrophoresis analysis of the test plasmid (prepared in DH10B) methylated by M2.Eco31I in vitro followed by digested with BsaI-HFv2. The results show that in vitro methylation by M2.Eco31I successfully blocked BsaI restriction site.

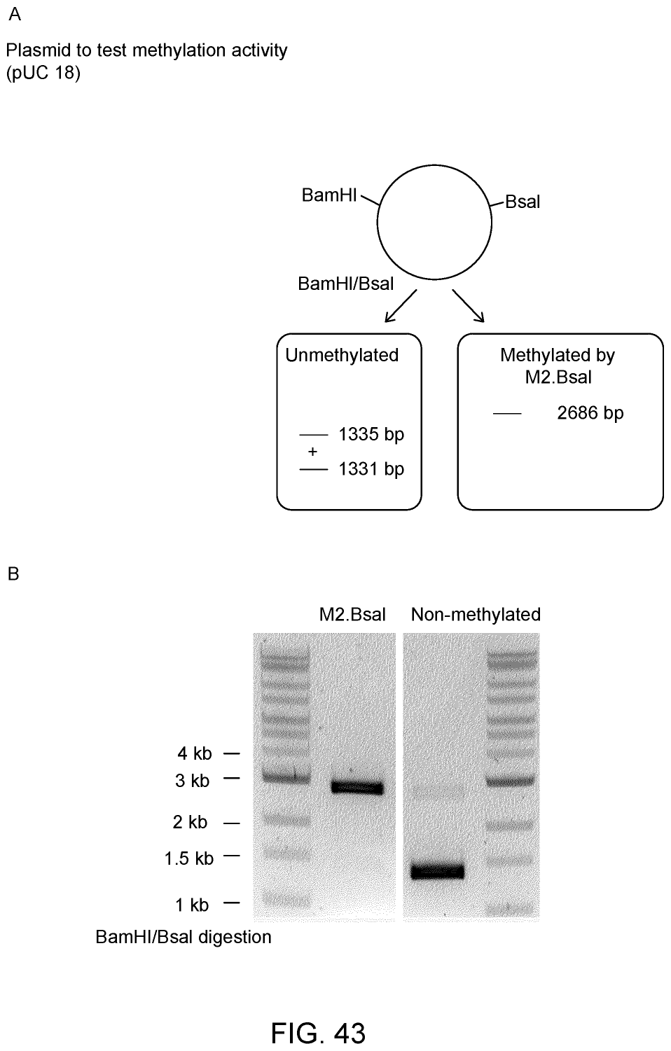

FIG. 43. Testing of methylation activity of recombinant M2.BsaI

A. Experimental designs to test methylation activity of M2.BsaI in vitro. The test plasmid (pUC18) contain a BamHI site and a BsaI site. Restriction digestion by BamHI/BsaI would generate a 1355 bp and a 1331 bp fragment. Restriction digestion of test plasmid that has been methylated by M2.BsaI in vitro would generate a single 2686 bp fragment due to blocking of the BsaI site by in vitro methylation. B. Agarose gel electrophoresis analysis of the test plasmid (prepared in DH10B) methylated by M2.BsaI in vitro followed by digested with BsaI-HFv2. The results show that in vitro methylation by M2.BsaI successfully blocked BsaI restriction site.

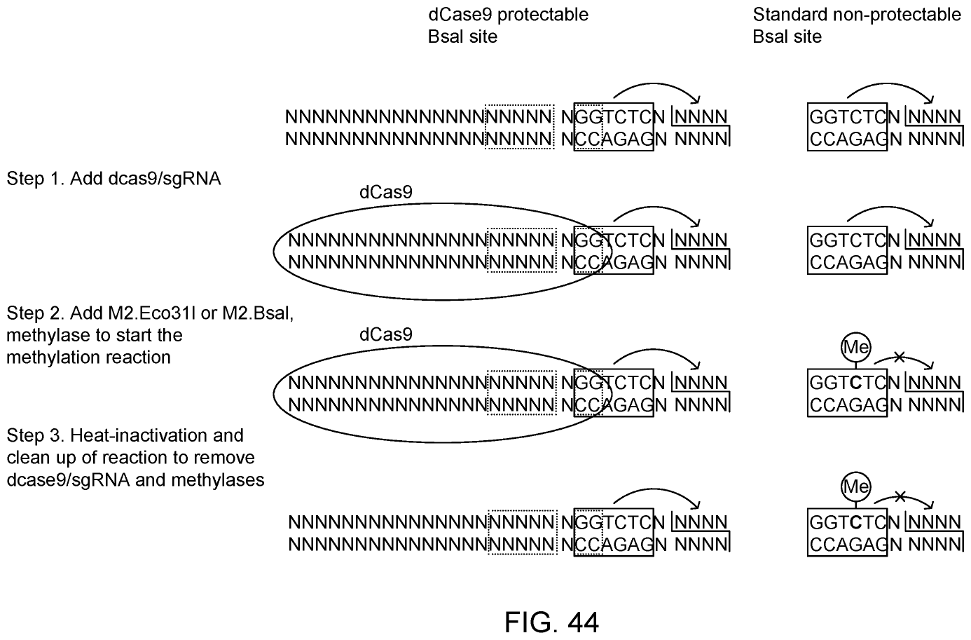

FIG. 44. In vitro methylation protection process

The in vitro methylation protection consists of 3 steps. In step 1 DNA molecules were incubated with dCas9/sgRNA complex which selectively binds to the dCas9-protectable BsaI sites but not non-protectable standard BsaI sites. In step 2 methylases such as M2.Eco31I or M2.BsaI that blocks BsaI sites were added initiate the methylation reaction in vitro. This selectively methylates non-protectable standard BsaI sites, not the dCas9-protectable BsaI sites which were protected from methylation by the stably bound dCas9. In step 3 the sample was subject to heat-inactivation and cleaned up to remove the dCas9 and methylases from the reaction, generating purified DNA with selective methylation of non-protectable standard BsaI sites only.

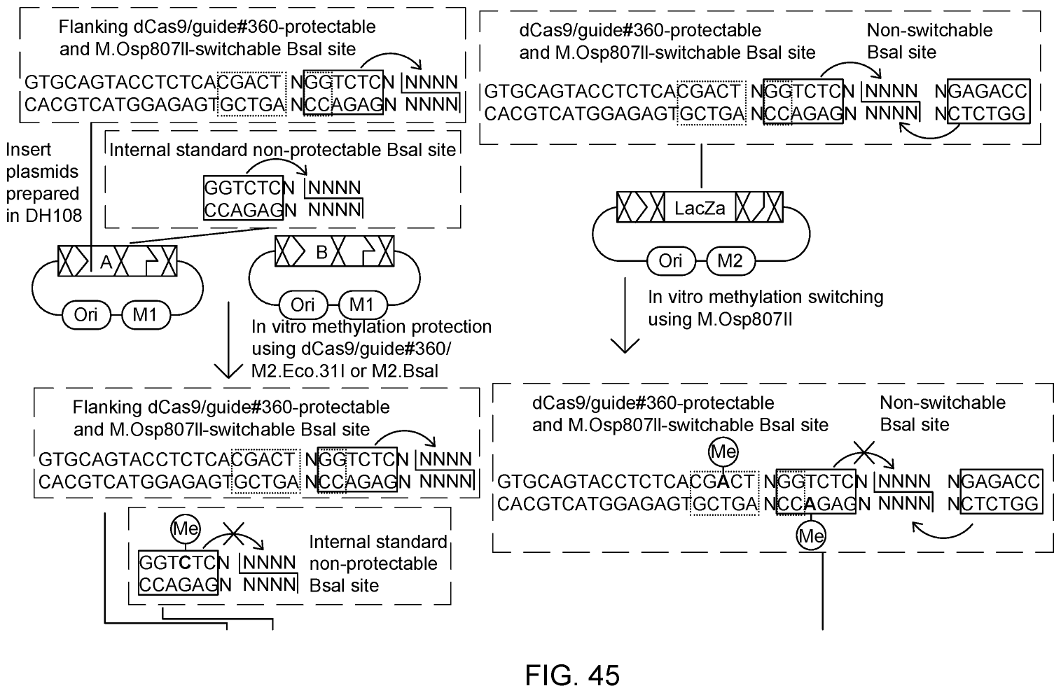

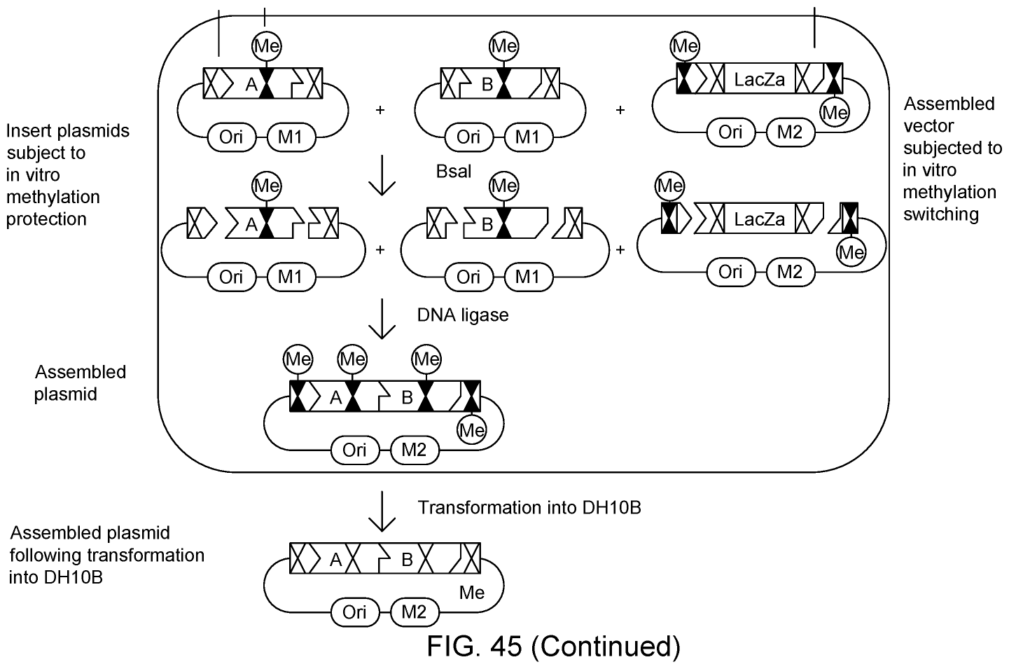

FIG. 45. In vitro Universal Assembly based on in vitro methylation switching and in vitro methylation protection

The diagram depicts the design of the implemented Universal Assembly system using in vitro methylation-protection and in vitro methylation-switching. The donor plasmids contain inserts flanked by dCas9/guide #360-protectable and M.Osp807II-switchable BsaI sites (the BsaI site is boxed and the nucleotides critical for dCas9 binding specificity are highlighted in grey) that would generate compatible adhesive ends following BsaI restriction. The internal standard BsaI sites in the insert do not overlap with guided-dCas9 binding sequence and therefore are not protected from methylation by the M2.Eco31I protection methylase. In vitro methylation protection of the donor plasmids results in selective methylation of internal BsaI sites within the insert (the methylated base is shown in bold) but not the flanking dCas9-protectable BsaI sites. The assembly vector contains a negative selection marker LacZalpha flanked by head-to-head BsaI sites that generate the same adhesive end as that produced by BsaI-based excision of the insert from the donor plasmid. The outer pair of BsaI sites are dCas9/guide #360-protectable and M.Osp807II methylation-switchable, whereas the inner pair of BsaI sites are nonswitchable. In vitro methylation switching of the assembly vector results in specific methylation of the outer pair of BsaI sites only. The methylated insert donor plasmids and assembly recipient vector plasmids can be cut with BsaI. Ligation of the cut fragments in a one-pot reaction that contains BsaI favours the generation of correctly assembled DNA comprising the inserts ligated together with the assembly vector backbone, in which all the BsaI sites are methylated. Transformation into a normal E. coli (DH10B) that lacks dCas9-protection and methylation-switching activity removes methylation of all the BsaI sites in the assembled plasmid.

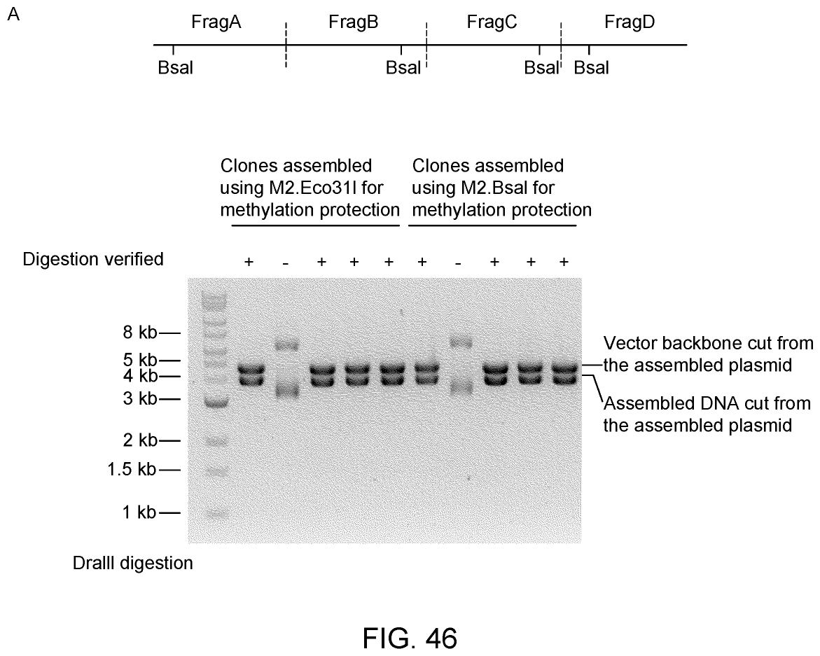

FIG. 46. Practical DNA assembly using in vitro Universal Assembly with in vitro methylation switching using M.Osp807II and in vitro methylation protection using dCas9/guide #360 with either M2.Eco31I or M2.BsaI

A. Diagram of the DNA fragment to assemble. Four fragments of human DNA from the MICA locus (FragA, FragB, FragC and FragD), each containing an internal BsaI site were assembled together to produce a 3.6 kb fragment (Genbank KF724576.1:23-3633) as the final product. The coordinates of the DNA fragment to assemble are FragA (KF724576.1:23-1048), FragB (KF724576.1:1045-1894), FragC (KF724576.1:1891-2744), FragD (KF724576.1:2741-3633).

B. Gel electrophoresis of DNA clones assembled by in vitro Universal Assembly following digestion by DraIII, which releases the assembled fragment through DraIII sites in the vector backbones, but does not cut inside the assembled 3.6 kb fragment as the assembled 3.6 kb DNA lacks DraIII sites. 4 out 5 clones screened were correctly assembled for using either M2.Eco31I or M2.BsaI-based system.

### EXAMPLE 1—UNIVERSAL ASSEMBLY: SEQUENCE-INDEPENDENT DNA ASSEMBLY USING A TYPE IIS RESTRICTION ENZYME, WITH DCAS9 AS THE SPECIFIC DNA BINDING PROTEIN FOR METHYLATION PROTECTION OF THE METHYLATION-PROTECTABLE RESTRICTION ELEMENT, AND M.OSP807II OR M.SEN0738I AS THE FURTHER METHYLASE FOR METHYLATION SWITCHING

Summary

Efficient DNA assembly is of great value in biological research and biotechnology. Type IIS restriction enzyme-based assembly systems allow assembly of multiple DNA fragments in a one-pot reaction, but suffer from the limitation that the DNA fragments to assemble need to be free of restriction sites for the type IIS restriction enzyme used for the assembly. Here we developed a new system named Universal Assembly that overcomes this problem. The Universal Assembly system is built on the methylation protection approach, whereby a DNA methylase is used to methylate sites for the given type IIS restriction enzyme in the DNA, but a DNA binding protein is used to selectively protect type IIS restriction sites that overlap with a DNA binding protein recognition sequence from methylation. We have developed practical Universal Assembly systems for BsaI-based one-pot assembly using dCas9-based methylation protection. The Universal Assembly system effectively eliminates the need to remove internal restriction sites from DNA to be assembled. The versatile system has potential to become a standard for modular DNA assembly, and has wide applications given the ability of the system to assemble DNA with no sequence constraints.

Introduction

Here we describe a new method for type IIS restriction enzyme-based DNA assembly that overcomes the problem of sequence constraint and so eliminates the requirement to remove internal type IIS restriction sites from DNA parts to assemble. The method, termed Universal Assembly, is based on the methylation protection approach, whereby a DNA methylase is used to methylate and so block any internal restriction sites for the type IIS restriction enzyme in any DNA fragment to be assembled. In parallel, a programmable DNA binding protein, such as a deactivated CRISPR Cas9, is used to bind to and so protect from methylation, the particular type IIS restriction sites that are positioned on the flanks of the DNA fragment and are used for restriction digestion to release the insert DNA fragment during the assembly process. The use of several Universal Assembly systems with different guide RNA sequences effectively eliminates the need to remove internal forbidden sequences and so allows DNA assembly without sequence constraint. We have designed and constructed a practical Universal Assembly system using the type IIS enzyme BsaI and used it to assemble multiple fragments of DNA each containing an internal BsaI site.

Material and Methods

Reagents

Enzymes for molecular biology are from NEB unless otherwise stated. A high-fidelity version of BsaI restriction enzyme named BsaI-HFv2 is used in place of BsaI for all the experiments.

Plasmid Construction

Plasmids for testing specific strains and for proof-of-principle DNA assembly were constructed using standard restriction enzyme/ligation-based cloning techniques. DNA fragments for cloning were generated by gene synthesis (Integrated DNA Technologies or Invitrogen), or by PCR with Q5 polymerase (NEB). Plasmids for functional testing, and the assembly vector for proof-of-principle DNA assembly were based on the ampicillin resistant MoClo vector pICH47732. Insert plasmids for proof-of-principle assembly were based on the kanamycin-resistant vector backbone described previously for MetClo proof-of-principle assembly (Lin D et al. Nucleic Acids Res. 2018 Nov. 2; 46(19):e113. doi: 10.1093/nar/gky596. PMID 29986052, which is herein incorporated by reference).

Low copy number plasmid pMOBKZ-2W148 was constructed for coexpression of M.Sen0738I (under a J23100 promoter, a modified RBS based on B0034m named B0034m* and an L3S2P21 terminator) and S.Sen0738I (under a J23100 promoter, a B0034m* RBS and an L3S1P51 terminator), and plasmid pMOBKZ-2W213 or pMOBKZ-2W214 was constructed for coexpression of guide RNA guide #360 or guide #401 (under a J23119 promoter and an L3S2P21 terminator), dCas9 (under a J23119 promoter, a B0034m* RBS and an L3S1P51 terminator), and M2.Eco31I (under a J23112 promoter, a B0034m* RBS and an L3S1P32 terminator). Briefly, for the construction of these plasmids, the transcription units were each assembled from individual cloned DNA parts using BsaI-based MetClo with the MetClo vector set described previously (Lin D et al. Nucleic Acids Res. 2018 Nov. 2; 46(19):e113. doi: 10.1093/nar/gky596. PMID 29986052). The transcription units were then assembled using BsaI into a low copy number kanamycin-resistant vector with an F replication origin, insert-flanking arsB homologous sequences and a zeocin-selection marker. In each round of assembly, insert DNA fragments containing M2.Eco31I were generated by PCR to remove M2.Eco31I methylation in the DNA. The dCas9 transcription unit for DNA assembly was generated by PCR using a flanking primer containing the J23119 promoter to avoid toxicity of the dCas9 transcription unit in the p15A-based low copy plasmid.

Generation of E. coli Strain

The E. coli strain that constitutively expresses M.Osp807II (DH10B-M.Osp807II) has been described previously (Lin D et al. Nucleic Acids Res. 2018 Nov. 2; 46(19):e113. doi: 10.1093/nar/gky596. PMID 29986052). The E. coli strain that constitutively expresses M.Sen0738I and S.Sen0738I (DH10B-2W148R) was constructed by recombineering using a linear DNA fragment amplified by PCR from pMOBKZ-2W148 using primers TTCTGTTACCCATCCAATTGTTC and CAGGCGCTTACCCGCTTCAT. The E. coli strains that deploy dCas9-based methylation protection mechanisms (DH10B-2W213R and DH10B-2W214R) were generated by lambda-red recombineering into DH10B cells using a linear DNA fragment amplified by PCR from pMOBKZ-2W213 and pMOBKZ-2W214 respectively using primers TTCTGTTACCCATCCAATTGTTC and CAGGCGCTTACCCGCTTCAT.

Modular Assembly

For assembly of a ˜3.6 kb DNA element from 4 fragments, the standard assembly reaction was 30 fmol of assembly vector, 60 fmol of each insert plasmid, 15 U T4 DNA ligase HC (Thermo Fisher) and 10 U BsaI-HFv2 (NEB) in 20 ul 1×T4 ligase buffer (NEB). The reaction condition was: 3TC 15 min, followed by 45 cycles of 3TC 2 min plus 16° C. 5 min, then 3TC 20 min, and 80° C. 5 min. Assembly reactions were transformed into specific chemically competent cells, and plated on LB plates with AIX selection (ampicillin 100 μg/ml, IPTG 100 μM, X-gal 50 μg/ml) at 3TC overnight. White colonies were expanded and screened by restriction digestion using DraIII or BsaI and by DNA sequencing.

For modular assembly of plasmid pMOP360_F3W using M.Osp807II and a dCas9-guide #360-based system, insert plasmids pMOK360_F3A, pMOK360_F3B, pMOK360_F3C and pMOK360_F3D were prepared in DH10B-2W213R from overnight culture in LB medium supplemented with 1% glucose and 30 μg/ml kanamycin. Assembly vector pMOP360_F3V was prepared in DH10B-M.Osp807II from overnight culture in LB medium with 100 μg/ml ampicillin, and the assembly reaction was transformed into DH10B-2W213R. Assembled clones were cultured in LB medium supplemented with 1% glucose and 100 μg/ml ampicillin.

For modular assembly of plasmid pMOP401_F4W using M.Sen0738I and a dCas9-guide #401-based system, insert plasmids pMOK401_F4A, pMOK401_F4B, pMOK401_F4C and pMOK401_F4D were prepared in DH10B-2W214R from overnight culture in LB medium supplemented with 1% glucose and 30 μg/ml kanamycin. Assembly vector pMOP401_F4V was prepared in DH10B-2W148R from overnight culture in LB medium with 100 μg/ml ampicillin, and the assembly reaction was transformed into DH10B-2W214R. Assembled clones were cultured in LB medium supplemented with 1% glucose and 100 μg/ml ampicillin.

For scarless assembly of 90 kb DNA resulting in plasmid pMOBC401_L1H1 using M.Sen0738I and a dCas9-guide #401-based system, the DNA was assembled in two stages from 14 fragments. In stage one, the insert plasmids carrying the initial ˜7 kb fragments were prepared in DH10B-2W214R, and the recipient assembly vector in DH10B-2W148R. 30 fmol of each insert plasmid and 15 fmol of the recipient assembly vector were used in a 20 ul assembly reaction with BsaI-HFv2 and T4 ligase. 20 U BsaI-HFv2 was then added to the assembly reaction for incubation at 37° C. for 45 min followed by heat inactivation at 80° C. for 5 min. The assembly reaction was dialysed for 1 h with water, and transformed into NEB10b competent cells by electroporation. Plasmid DNA from positive clones were transformed into DH10B-2W214R, and used as insert plasmids for the stage two assembly. In stage two, the assembly reaction set up and transformation procedure was the same as stage one. Clones were screened by NotI digestion followed by pulsed-field electrophoresis.

Results

Design of Universal Assembly: Requirement for Selective Methylation Protection of Insert-Flanking Type IIS Sites

For the original MetClo approach that we described previously, we developed a method for switching the insert flanking type IIS restriction enzyme recognition sites on and off using methylation of these sites by a specific methylase (a ‘switch’ methylase) whose own recognition site sequence overlaps that of the restriction enzyme (FIG. 1 and FIG. 2). The methylation blocks the type IIS restriction enzyme activity towards type IIS restriction sites that overlap with the methylation sequence. In practice, other different methylases such as M.Osp807II and M.Sen0738I can also be used as switch methylases with appropriate design of switchable BsaI sites (FIGS. 1, 2 and 3).

The approach that we have developed and termed Universal Assembly uses a DNA methylase to methylate, and so block from restriction digestion, all the internal sites for the type IIS restriction enzyme used for the assembly. The insert-flanking restriction sites used for the assembly are protected from this methylation so that they can remain active when required during the assembly. To protect these flanking restriction sites from methylation, a DNA-binding protein such as an appropriately guided dCas9 is used to bind to and so block the flanking restriction sites from the action of the methylase. (FIG. 4). This protection from methylation must only happen at the insert-flanking type IIS restriction enzyme sites. In this way, all the internal type IIS sites are blocked by DNA methylation, whereas the flanking protected-type IIS restriction sites are free for the release of intact insert DNA fragment. The specificity of this protection is determined by the sequence that overlaps between the restriction site and the DNA-binding protein recognition sequence.

Site-Specific Protection from Methylation by Sequence-Programmable dCas9

To develop Universal Assembly using BsaI-based DNA assembly we chose the methylase M2.Eco31I from E. coli because it methylates the 4^{th} position of the BsaI site (GGTCTC) and methylation at this position is known to block BsaI activity (Storch M et al. ACS Synth Biol. 2015 Jul. 17; 4(7):781-7. doi: 10.1021/sb500356d PMID 25746445). We then chose dCas9 as the DNA-binding protein because its DNA-binding specificity can be programmed by the sequence of the guide RNA used—dCas9 binding specificity is determined by the NGG PAM motif and an adjacent >=5 bp seed sequence (O'Geen H et al. Nucleic Acids Res. 2015 Mar. 31; 43(6):3389-404. doi: 10.1093/nar/gkv137 PMID 25712100).

To build this system, we first constructed a new E. coli strain (DH10B-2W213R) which coexpresses a CRISPR guide RNA guide #360, dCas9 and the M2.Eco31I methylase (FIG. 5A). We used the strong J23119 for both the guide RNA and dCas9 expression, and the weak J23112 promoter for the M2.Eco31I methylase expression. We constructed a test plasmid (pMOP_testN8) that contains a BsaI site that overlaps with the guide RNA sequence, and a standard BsaI site (FIG. 5B) and prepared this plasmid in the new strain (DH10B-2W213R). The test plasmid prepared in this strain was cut by BsaI at the BsaI site that overlaps with the guide RNA sequence, but not at the standard BsaI site (FIG. 5C). This demonstrates that when a plasmid is prepared in this strain, the dCas9 is able to selectively protect the site to which the guide RNA directs its DNA-binding activity from methylation by the M2.Eco31I methylase.

A similar system with a different guide RNA sequence (guide #401 comprising strain DH10B-2W214R and compatible test plasmid pMOP_testN20) also shows efficient sequence-specific methylation protection (FIGS. 5A, B and C), confirming that the principle of methylation protection is universal for the dCas9-M2.Eco31I system.

Design of Universal Assembly: One-Pot DNA Assembly Using the Methylation Protection

The methylation protection approach can be combined with the methylation switching approach used in the original MetClo method in a one-pot DNA assembly system, which effectively eliminates the need to remove internal type IIS restriction sites in the DNA fragments to be assembled. This can be done by constructing an insert-flanking type IIS restriction site that is both dCas9-protectable and methylation-switchable (FIG. 6 and FIG. 7). This site can be blocked through the methylation-switching mechanism, or be protected from methylation by the methylase that inactivates sites for the type IIS restriction enzyme that lie within the insert sequence through the dCas9-protection mechanism.

A one-pot assembly system can then be designed, with the donor plasmids containing inserts flanked by this methylation-switchable and dCas9-protectable type IIS restriction sites, and the assembly vector plasmid containing a negative selection marker flanked by head-to-head type IIS restriction sites, with the outer pair of the sites closer to the vector backbone being the methylation-switchable and dCas9-protectable type IIS restriction sites, and the inner pair being the non-switchable sites.

A one-pot assembly reaction can then be undertaken using donor plasmid(s) prepared in the strain that expresses the insert-blocking methylase, dCas9 and an appropriate guide RNA, and vector plasmid prepared in a strain that expresses the switch methylase. In this reaction, the type IIS enzyme does not cut any internal sites for the type IIS enzyme that lie within the insert in the donor plasmid as these sites have been methylated and so blocked by the insert-blocking methylase. In contrast, the type IIS enzyme does cut the insert-flanking type IIS sites in the donor plasmid because these sites were protected from methylation by the dCas9 which was bound to them. Thus, the type IIS restriction enzyme releases the insert from the donor plasmid, but does not cut the insert at any type IIS restriction sites within the insert itself. The recipient vector plasmid contains a negative selection marker which will be replaced with the assembled inserts in the correctly assembled plasmid. At each end of the marker there are two head-to-head sites type IIS sites, all for the same enzyme. The outer two of these four sites are the methylation-switchable and dCas9-protectable sites, which have been methylated by the switch methylase and so are not cut by the type IIS enzyme. However, the inner type IIS sites are not methylation-switchable therefore will be cut, and this releases the selectable marker to be replaced by the inserts. In the fully assembled plasmid, the assembled insert is now flanked by the two outer methylation-switchable type IIS restriction enzyme sites which cannot be cut by the type IIS enzyme because they are methylated. Thus the assembly is completed. The newly formed plasmid can then be transformed into a normal strain that does not express the switch methylase nor the dCas9-protection mechanism, which will remove all the specific methylation of the type IIS restriction sites for the given enzyme in the plasmid. The assembled plasmid can then be used for next stage assembly starting with transformation into the strain that expresses the insert-blocking methylation, dCas9 and appropriate guide RNA, because the assembled insert is flanked by the same methylation-switchable and dCas9-protectable type IIS restriction sites as the donor plasmids before the assembly (FIG. 8 and FIG. 9). Alternatively, the assembly reaction mixture can be transformed directly into the strain that expresses the insert-blocking methylase, dCas9 and appropriate guide RNA, which yields assembled plasmids with the internal type IIS restriction sites blocked and the flanking sites protected by dCas9 (FIG. 10 and FIG. 11). This assembled plasmid can be used directly as donor plasmid for next stage one-pot DNA assembly (FIG. 10 and FIG. 11).

It should be noted that the above systems described that utilize flanking BsaI sites that are both methylation-switchable and dCas9-protectable are designed for sequence-independent hierarchical DNA assembly whereas the assembled DNA can be used as insert plasmid for next stage of DNA assembly. If only one-round of DNA assembly is required such that the assembled DNA will not be used for next round DNA assembly, then the design requirements can be relaxed that in the donor plasmids the flanking BsaI sites must still be dCas9-protectable but not necessarily methylation-switchable, and in the assembly vector the outside pair of BsaI sites must be methylation-switchable but not necessarily dCas9-protectable.

DNA Assembly Using Universal Assembly

To demonstrate the utility of Universal Assembly for DNA assembly, we built a proof-of-principle Universal Assembly system using appropriately guided dCas9 binding to protect the insert-flanking BsaI type IIS restriction enzyme site from methylation by the M2.Eco31I methylase. This used in vivo methylation in the strain DH10B-2W213R. The guide RNA sequence (guide #360) was designed to protect a methylation-switchable insert-flanking BsaI site that can be switched on and off using the M.Osp807II methylase (FIG. 5A, FIG. 6, Table 1). This system was used to assemble a ˜3.6 kb fragment from four DNA fragments, each of which contained a single internal BsaI site (FIG. 12A, Table 1). The internal BsaI sites do not overlap with the guide RNA sequence used to protect the dCas9-protectable insert-flanking BsaI sites, and so are not protected from methylation when the plasmid is prepared in the DH10B-2W213R strain which expresses the guide RNA (guide #360) for the insert-flanking sites, dCas9 and the M2.Eco31I1 methylase (FIG. 5 and FIG. 10). The resulting methylation that occurs at any BsaI sites within the insert subsequently protects them from digestion by BsaI during the assembly process. However, the insert-flanking BsaI sites have not been methylated because of the bound dCas9 and so are cut during the assembly process (FIG. 6B). For insert plasmids prepared in DH10B-2W213R, the internal BsaI site is 100% methylated, and the percentage of insert plasmids with both flanking BsaI sites protected by dCas9 from M2.Eco31I methylation is around 50% (FIG. 12B). When the insert plasmids prepared in DH10B-2W213R were assembled into assembly vector prepared in a strain that expresses the M.Osp807II (DH10B-M.Osp807II), and the assembly reaction transformed into DH10B-2W213R, 50% of the colonies are white and >80% (7 out of 8) of the white colonies screened were correctly assembled as verified by restriction digest and DNA sequencing (FIG. 12D). The correctly assembled DNA was still flanked by the same guide-RNA (guide #360)-protectable BsaI sites. Test digestion shows that the internal BsaI sites in the assembled DNA prepared in DH10B-2W213R strain that deploys guide #360-based dCas9 methylation protection were completely blocked by M2.Eco31I methylation, whereas the flanking BsaI sites were protected from M2.Eco31I methylation. The percentage of the assembled plasmids with both flanking BsaI sites protected from M2.Eco31I methylation is over 50%. This prepares the assembled DNA for use as donor plasmid in the next stage one-pot assembly (FIG. 12F).

Similar results were obtained using a Universal Assembly system with a different guide RNA sequence (guide #401) and different switch methylase (M.Sen0738I). In this system, the guide RNA sequence (guide #401) is designed to be compatible with M.Sen0738I-based methylation switching (FIG. 5A and FIG. 7, Table 1). The M.Sen0738I switch methylase was chosen from a set of potentially suitable switch methylases for methylation-switching of BsaI sites, because unlike the M.Osp807II-compatible Universal Assembly system in which the 5 bp seed sequence of guide RNA is constrained by the M.Osp807II methylation motif (NGACN) (FIG. 6A), the seed sequence of guide RNA for methylation compatible with M.Sen0738I can be freely chosen (NNNNN) (FIG. 7A). This increases the design space of guide RNA seed sequence compatible with the same switch methylase considerably (FIG. 7). With the RNA guide #401-compatible insert plasmids prepared in strain DH10B-2W214R that deploys guide #401-based methylation protection (FIG. 12C), and an assembly vector prepared in strain DH10B-2W148R for M.Sen0738I-based methylation switching, a 3.7 kb fragment can be assembled from 4×˜1 kb fragments with high efficiency (7/8 correct) (FIG. 12E). As the assembled DNA is flanked by the same guide #401-protectable BsaI sequence, when prepared in DH10B-2W214R, the internal BsaI sites of the assembled DNA were completely methylated, whereas the flanking BsaI sites in the plasmid were protected from methylation (FIG. 12F), therefore DNA assembled using the guide #401 based assembly system can be used as donor plasmid in next stage of one-pot Universal Assembly.

These data together demonstrate that the Universal Assembly system can efficiently assemble DNA with internal type IIS restriction sites using different guide RNA sequences that are compatible with a suitable switch methylase. The choice of guide RNA sequence therefore can be tailored to the DNA sequence to assemble, which enables the system to assemble DNA with no sequence constraints.

Hierarchical Scarless DNA Assembly Using a Fixed Set of Sequence-Independent Assembly Vectors

The Universal Assembly system enables hierarchical scarless DNA assembly with type IIS restriction enzymes using a fixed set of assembly vectors. Here we describe the scheme.

For hierarchical DNA assembly systems that use a single type IIS restriction enzyme for different stages of DNA assembly, the insert DNA fragments are cut to have compatible adhesive ends (‘overhangs’) with the assembly vector, are assembled into the assembly vector using the type IIS restriction enzyme, and the assembled DNA can then be released with a pair of flanking type IIS restriction sites for the same enzyme. We term the overhang sequence used to insert DNA into the vector plasmid backbone during the assembly process as the “pre-assembly overhang”, and the overhang sequence at the end of the assembled DNA when released from the vector backbone in the next stage DNA assembly as “post-assembly overhang” (FIG. 13). For clarity, the sequence of the top strand or ‘forward’ strand is used to define the overhang sequence, but it will be represented by the reverse complement of this on the opposite strand.

A simple design for a recipient assembly vector to implement scarless hierarchical assembly could have a negative selection marker flanked by head-to-head type IIS restriction sites, to enable the assembled DNA to be released as an insert for next stage DNA assembly. The inner pair of type IIS restriction sites closer to the interposing negative selection maker is used to cut the assembly vector open and so allow ligation of insert DNA into the vector. These inner sites are within the fragment containing the negative selection marker that is removed from the assembly vector. The outer pair of type IIS restriction sites closer to the vector backbone are left in place and after the assembly they will be flanking the assembled insert DNA. These outer sites can then be used to release the assembled DNA which can then be used as an insert for next stage of a hierarchical DNA assembly process. In this design, the pre-assembly overhang sequence depends on the inner type IIS restriction site closer to the negative selection marker, and the post-assembly overhang sequence depends on the outer type IIS restriction site closer to the vector backbone (FIGS. 14A and 14B).

The pre-assembly overhang and post-assembly overhang may or may not be the same depending on the distance between the head-to-head type IIS restriction sites. When the two restriction sites in the head-to-head arrangement are equidistant from the pre-assembly overhang, they will cut at the same position and so generate the same adhesive end; the pre-assembly overhang sequence and post-assembly overhang sequence are then the same. We term this arrangement of inner and outer sites as a ‘maintenance-type’ head-to-head arrangement (FIG. 15). In a maintenance-type design, the choice of pre-assembly overhang sequence is totally flexible.

If the head-to-head sites are arranged such that the distance between the outer site and the pre-assembly overhang is less than the distance between the inner site and the pre-assembly overhang then one or more bases will be excised from the end of the insert DNA following the DNA assembly process. If the distance between the outer site and the pre-assembly overhang is reduced enough the post-assembly overhang sequence can be from within the insert DNA sequence itself and totally independent of the pre-assembly overhang. This will arise when the number of excised bases is greater than the size of the pre-assembly overhang. We term this arrangement of inner and outer sites as an ‘excision-type’ head-to-head arrangement (FIG. 16). For example, BsaI leaves a 4 bp overhang, so for a BsaI-based MetClo recipient vector, the excision-type design requires the distance between the outer site and the pre-assembly overhang to be at least 4 bp less than the distance between the inner site and the pre-assembly overhang (FIG. 16).

With an excision-type arrangement the inner restriction site cuts open the recognition sequence of the outer restriction site when the negative selection marker is cut out during the assembly process. This outer restriction site is then reconstructed when the overhang of the insert DNA is ligated to the overhang of the assembly vector (FIG. 16). There are various implications of the design, which in part depend on whether the insert DNA preparation involves methylation protection.

If methylation protection is not used for insert DNA preparation, then for a hierarchical assembly system using only a single type IIS restriction enzyme, the insert DNA must not contain any sites for this type IIS restriction enzyme. This is because if the insert contains one or more sites for this enzyme, then the enzyme will generate multiple DNA fragments with different adhesive ends, which may interfere with the assembly process. This places a limit on the number of bases that can be excised in the excision-type design. For a BsaI-based assembly systems, the upper limit for excision is 6 bp (FIG. 17, FIG. 18).

Furthermore, the initial outer restriction site in the vector backbone and the reconstructed outer restriction site following assembly must be protected by methylation-switching. The feasibility of the excision-type design thus depends on the location and strand of the methylated base that is generated by the switch methylase.

This is because the methylated base must remain in the assembly vector backbone following digestion of the assembly vector by the type IIS restriction enzyme acting from the inner site. For example, both the M.Osp807II and M.Sen0738I switch methylases allows 4-6 basepairs excision-type design in a BsaI-based system (FIG. 16 and FIG. 18), whereas the M2.NmeMC58II switch methylase in a BpiI-based system does not allow excision-type design at all because BpiI cuts such that the methylated base is retained with the negative selection marker rather than in the assembly vector backbone (FIG. 19).

The outer restriction site does not need to be a full restriction site and a partial site can be reconstituted to a full site by the insert DNA, which increases the freedom of sequence design for the excision-type head-to-head restriction sites (FIG. 20). A partial site that deploys methylation-switching still needs to carry the switch methylase recognition motif for the excision-type design to be feasible. Both M.Osp807II and M.Sen0738I-based methylation switching in a BsaI-based system allows 4-6 basepair excision-type design using a partial outside restriction site (FIG. 20). Table 2 lists the allowable pre-assembly overhang sequences for all the excision-type designs that can be used for M.Osp807II and M.Sen0738I-based methylation switching in a BsaI-based assembly system.

If methylation protection is used for the insert DNA preparation, then all the internal type IIS restriction sites in the insert are methylated and so inactivated. This allows for excision of more base pairs at the end of the insert sequence. During assembly process, the outer restriction site is provided by an internal restriction site inside the insert DNA sequence, which is methylated by the methylation protection mechanism during insert DNA preparation (FIG. 21).

With the maintenance-type and excision-type design of head-to-head restriction sites, it is possible to design a scarless hierarchical DNA assembly scheme that requires only a fixed set of assembly vectors for assembly of any DNA. This set of assembly vectors consists of three special vector types. all carrying an negative selection marker flanked by head-to-head type IIS restriction sites with the same pre-assembly overhangs ‘u’ and ‘v’ on the ‘left’ and ‘right’ end respectively (FIG. 22). The head-to-head restriction sites flanking the negative selection marker are of the maintenance-type or excision-type design depending on the vector type. In vector type VLeft, the negative selection marker is flanked on the ‘left’ by a maintenance-type head-to-head arrangement and on the ‘right’ by an excision-type head-to-head arrangement. The ‘right’ excision-type arrangement leads to excision of 6 base pairs. In vector type VRight, the negative selection marker is flanked on the ‘left’ by an excision-type head-to-head arrangement and on the ‘right’ by a maintenance-type head-to-head arrangement. The ‘left’ excision-type arrangement leads to excision of 4 base pairs. In vector type VMiddle, the negative selection marker is flanked on both sides by an excision-type head-to-head arrangement. These two excision-type arrangements differ, such that the ‘left’ excision-type arrangement leads to excision of 4 base pairs, but the ‘right’ excision-type arrangement leads to excision of 6 base pairs. In these three vectors, the pre-assembly overhang sequences ‘u’ and ‘v’ and their reverse-complement sequences are designed to be different to minimize the probability of self-ligation of the assembly vector backbone, and to ensure the directionality of assembly relative to the vector backbone sequence, thus the directionality of the excision process. For scarless BsaI-based assembly using the M.Sen0738I switch methylase, we chose ‘CTCC’ as the 4 bp overhang sequence ‘u’ (the physical overhang associated with the vector backbone is the reverse complement GGAG), and ‘AGAC’ as the 6 bp overhang sequence ‘v’ (FIG. 22).

The design of these three types of assembly vectors allows any given insert DNA which has the appropriate overhangs ‘u’ (‘CTCC’) and ‘v’ (‘AGAC’) to be cloned into any one of the three vectors, but with different outcome depending on the type of vector (FIG. 23). For an insert DNA plasmid with insert DNA carrying overhang ‘u’ and ‘v’ at the left and right hand of the fragment, cloning into vector VLeft excises the 6 bp adaptor sequence ‘TGAGAC’ at the right end of the fragment only (FIGS. 23A and B). Cloning into vector VMiddle excises both the 4 bp adaptor sequence ‘CTCC’ at the left end of the fragment and the 6 bp adaptor sequence ‘TGAGAC’ at the right end of the fragment (FIGS. 23C and D). Cloning into vector VRight excises the 4 bp adaptor sequence ‘CTCC’ at the left end of the fragment only (FIGS. 23E and F).

The multi-fragment assembly process and adaptor excision process can be combined in a single reaction. FIG. 24 illustrates an example of the outcomes from assembling the same set of three inserts in each of the three vector types. The leftmost insert DNA fragment starts with overhang sequence ‘u’ (‘CTCC’) and the last insert DNA fragment ends with overhang sequence ‘v’ (‘AGAC’). Assembly into vector VLeft generates an assembled fragment with excision of 6 bp adaptor sequence (‘TGAGAC’) at the right end of the assembled sequence (FIGS. 24A and B). Assembly into vector VMiddle generates an assembled fragment with excision of 4 bp adaptor sequence (‘CTCC’) at the left end, and 6 bp adaptor sequence (‘TGAGAC’) at the right end of the assembled sequence (FIGS. 24C and D). Assembly into vector VRight generates assembled fragment with excision of 4 bp adaptor sequence (‘CTCC’) at the left end of the assembled sequence (FIGS. 24E and F).

With these three type of assembly vectors and the Universal Assembly system that deploys methylation protection and methylation switching, any DNA can be assembled scarlessly with no sequence constraints. FIG. 25 illustrates an example of DNA assembly of 9 fragments in two rounds with 3 fragments per group in each round using the above vector set design (‘CTCC’ as ‘u’ and ‘AGAC’ as ‘v’) with a BsaI-based Universal Assembly system. Vector DNA is prepared in a strain expressing the M.Sen0738I switch methylase and insert DNA in a strain expressing the guide #401-based methylation protection system. The antibiotic selection marker of the assembly vector differs between consecutive rounds of DNA assembly. The DNA sequence to be assembled (FIG. 25A) is divided into 9 fragments with 4 bp overlapping sequences that define the overhang sequences for ordered DNA assembly (FIG. 25B). These 8 ‘internal’ overhang sequences must differ from the pre-assembly overhang sequences ‘CTCC’ and ‘AGAC’ (and their reverse complements). In addition, each internal overhang sequence used in each round of the assembly must be different from each other (and their reverse-complement sequences). The starting fragments can then be synthesized by PCR or gene-synthesis with appropriate adaptor sequences added, and cloned into suitable vectors that encode methylation-protection/methylation-switching motifs. A sequence-independent cloning method can be used for this purpose such as blunt-end cloning. The adaptor sequences added are the adaptor sequences that will be excised when cloned into vectors with excision-type designs. For the current design, the 4 bp adaptor sequence ‘CTCC’ and 6 bp adaptor sequence ‘TGAGAC’ are added to the left and right end of each starting fragment respectively, generating insert plasmids carrying the insert fragments with pre-assembly overhang sequence ‘u’ (‘CTCC’) and ‘v’ (‘AGAC’) (FIG. 25C). The insert plasmids were then cloned as single fragment into a suitable type of vectors depending on the position the cloned fragment would take up in the next round assembly (FIGS. 25D and E). In this case, fragment ‘A’, ‘D’ and ‘G’ are cloned as single fragment into vector ‘VLeft’ because they are the left fragment in the next round assembly; fragments ‘B’, ‘E’ and ‘H’ are cloned into vector ‘VMiddle’ because they are the middle fragment in the next round; fragments ‘C’, ‘F’ and ‘I’ are cloned into vector ‘VRight’ because they are the right fragment in the next round. The cloned fragments are then assembled three fragments per group into a suitable vector which was chosen on the basis of the position that the assembled fragment would take up in the next round assembly (FIGS. 25F and G); that is, whether it would be the left, middle or right fragment in the next round. In this case, fragments ‘A’, ‘B’, ‘C’ are assembled as fragment ‘ABC’ into vector ‘VLeft’ because fragment ‘ABC’ is the left fragment in the next round assembly. Fragments ‘D’, ‘E’, ‘F’ are assembled into vector ‘VMiddle’ as ‘DEF’ is the middle fragment in the next round. Fragments ‘G’, ‘H’, ‘I’ are assembled into vector ‘VRight’ as ‘GHI’ is the right fragment in the next round. In the last round, the three intermediate fragments ‘ABC’, ‘DEF’ and ‘GHI’ are assembled into a single fragment in vector ‘VMiddle’, generating fragment ‘ABCDEFGHI’ as required (FIGS. 25H and I).

In practice, the cloning of linear double strand DNA into a vector and cloning of this insert into a suitable vector type can be combined into a single step (FIG. 26C), simplifying the scarless assembly scheme (FIG. 26).

In the scarless assembly scheme, the design of the assembly vector is independent of the DNA sequence to be assembled. Therefore, the scheme allows using a fixed set of assembly vectors to assemble any DNA with no sequence constraints. A small set of six assembly vectors representing the three different types and two antibiotic selection markers (e.g. pMOBK401_VL, pMOBK401_VM, pMOBK401_VR, pMOBC401_VL, pMOBC401_VM, pMOBC401_VR representing VLeft, VMiddle and VRight with kanamycin or chloramphenicol selection markers) is sufficient for hierarchical scarless assembly with arbitrary rounds of assemblies.

Hierarchical DNA Assembly Using the Scarless Scheme

To demonstrate the feasibility of this scarless assembly scheme, we assembled a ˜90 kb DNA fragment from the human MICA locus from 14 fragments in two stages (FIGS. 27A and B, Table 3). 14 starting fragments with suitable overhang sequences were generated by PCR and cloned by blunt-end ligation to generate insert plasmids that carry the starting fragments with suitable overhang sequences added depending on the position that the resulting fragment will take up in the next round of assembly. In round one of the assembly, groups of 2-4 fragments were assembled into suitable assembly vectors depending on the position that the assembled fragment would take up in the next round of assembly. In round two, the four intermediate insert plasmids prepared in DH10B-2W214R were assembled in a single reaction into the final product. The success rate of each round of assembly exceeds 80% (FIG. 27C and Table 3). This demonstrates that the scarless assembly scheme combined with Universal assembly can be used for efficient scarless assembly of an arbitrary DNA sequence.

Scarless Assembly Vector Examples

Vector with M.Osp807II Methylation Switching, 4 bp Excision on the Left, 6 bp on the Right.

M.Osp807II used for methylation switching for preparation of assembly vector. Insert prepared by methylation protection using M2.Eco31I and dCas9 with guide RNA sequence

| (SEQ ID NO: 95) | |
|---|---|
| | NNNNNNNNNNNNNNNNGACN |

(last 5 bp are the seed sequence that determines the specificity).

VL (vector left insert)
(SEQ ID NO: 96)
NNNNNNNNNNNNNNNNGACNNGGTCTCNCTCCNGAGACC(N^{X})GGTCTCN
AGACCNNGTCNNNNNNNNNNNNNNNN
VM (vector middle insert)
(SEQ ID NO: 97)
NNNNNNNNNNNNNNNNGACNNGGTCTCCNGAGACC(N^{X})GGTCTCNAGAC
CNNGTCNNNNNNNNNNNNNNNN
VR (vector right insert)
(SEQ ID NO: 98)
NNNNNNNNNNNNNNNNGACNNGGTCTCCNGAGACC(N^{X})GGTCTCNAGAC
NGAGACCNNGTCNNNNNNNNNNNNNNNN

Vector with M.Sen0738I Methylation Switching, 4 bp Excision on the Left, 6 bp on the Right

M.Sen0738I used for methylation switching for preparation of assembly vector. Insert prepared by methylation protection using M2.Eco31I and dCas9 with guide RNA sequence

| (SEQ ID NO: 99) | |
|---|---|
| | NNNNNNNNNNNCCAGNNNNN |

(last 5 bp are the seed sequence that determines the specificity).

VL (vector left insert)
(SEQ ID NO: 100)
NNNNNNNNNNNCCAGNNNNNNGGTCTCNCTCCNGAGACC(N^{X})GGTCTCN
AGACCNNNNNNCTGGNNNNNNNNNNN
VM (vector middle insert)
(SEQ ID NO: 101)
NNNNNNNNNNNCCAGNNNNNNGGTCTCCNGAGACC(N^{X})GGTCTCNAGAC
CNNNNNNCTGGNNNNNNNNNNN
VR (vector right insert)
(SEQ ID NO: 102)
NNNNNNNNNNNCCAGNNNNNNGGTCTCCNGAGACC(N^{X})GGTCTCNAGAC
NGAGACCNNNNNNCTGGNNNNNNNNNNN

(N^{x}) may mean any number of A, C, T or G, or between 0 bp and 300 kbp of A, C, T, or G.

Discussions

Here we described a system for DNA assembly using type IIS restriction enzymes with no sequence constraints.

The design of the system is based on the methylation protection principle, whereas a sequence-specific DNA binding protein can protect specific type IIS restriction site that overlaps with the binding sequence from being methylated by a methylase that would otherwise methylate all the type IIS restriction sites for the given enzyme. This can be achieved by coexpressing the DNA binding protein and the methylase in the strain used for DNA propagation (in vivo), or carrying out the methylation reaction in the presence of the DNA binding protein (in vitro). In theory a number of different types of sequence specific DNA binding proteins can be used. This includes two types: one that has no enzymatic activities toward the DNA substrate, such as CRISPR based dCas9, TALEN, Zinc finger protein etc. (pure DNA binding protein). The other may have enzymatic activity toward the DNA substrate (for example sequence specific DNA methylase), however as long as the modification (for example methylation) does not affect restriction enzyme activity of the type IIS restriction enzyme towards the type IIS restriction site then it can be used (See Example 2, FIGS. 28 and 29).

With the methylation protection principle, double strand DNA can be generated where a linear insert fragment is flanked by “protected” unmethylated type IIS restriction sites, but all the internal type IIS restriction sites are methylated—the insert can therefore be cut out by the type IIS restriction enzyme intact from the vector backbone in the double stranded DNA when the DNA is prepared through the methylation protection mechanism. In this way we can release intact linear DNA without sequence constraints, with any adhesive ends (4 bp for BsaI with no sequence constraints), from a closed (circular) DNA molecule. This linear DNA can then be used for ligation in cloning. The linear DNA can also be used for other DNA assembly methods such as Gibson assembly or homologous recombination in yeast or B. subtilis etc.

This approach can be applied to one-pot DNA assembly using type IIS restriction enzyme. For a single stage DNA assembly, the design is very flexible. For example, all the fragments for DNA assembly including the vector backbone can be prepared using the methylation protection approach, as long as the assembled plasmid sequence does not contain methylation-protectable type IIS restriction sites protectable in the strain used to prepare the insert fragments.

For an idempotent hierarchical assembly system, whereas both the insert fragments and the assembled fragments at all the stages contain the same flanking protectable type IIS restriction sites, two different designs are feasible. One design combines the methylation protection principle with the methylation switching principle, whereas a combined methylation-protectable and switchable flanking type IIS restriction sites are used as outer pair of the head-to-head type IIS restriction sites in the assembly vector, and non-switchable type IIS restriction sites for the same enzyme as the inner pair. In this design the assembly vector is prepared in strains that deploy methylation switching to selectively block the outer pair type IIS restriction sites in the assembly vector backbone, the insert plasmids are prepared in, and assembled plasmid transformed into strains that deploys methylation protection to selectively block internal type IIS restriction sites within the insert while leaving flanking type IIS restriction sites active (FIG. 10 and FIG. 11).

Another idempotent design (Example 3) uses the methylation protection principle alone for both vector and insert DNA preparation. Here the assembly vector contains head-to-head type IIS restriction sites with the outer and inner pair of type IIS restriction sites protected with two different methylation-protection specificity. The insert plasmids are prepared in and the assembled plasmid transformed into strains that deploy methylation protection for one specificity to block internal type IIS restriction sites while leaving the flanking methylation-protectable restriction sites open. The assembly vector is prepared in strains that deploy methylation protection with a different specificity to selectively protect the inner pair of type IIS restriction sites from methylation (FIG. 30 and FIG. 31). Compared with the previous design, this design has the advantage that the assembly vector backbone sequence can also contain internal type IIS restriction sites. The potential drawbacks are potential high background uncut assembly vector in the transformation reaction due to inefficient methylation protection of inner type IIS restriction sites within the assembly vector. This may be eliminated by using suicide genes such as ccdB as negative selection marker.

The idempotent hierarchical assembly system that deploys both methylation protection and methylation switching can be further adapted into an assembly system for scarless DNA assembly of DNA without sequence constraints using a limited set assembly vectors. This unique design is based on the ‘maintenance type’ and ‘excision type’ head-to-head type IIS restriction sites for assembly vector design, which enables assembly of the same set of insert fragments into different types of assembly vectors, at the same time excising fixed number of nucleotides at specific ends of the assembled DNA depending on the choice of assembly vector (FIG. 24). With this design, hierarchical scarless assembly can be carried out using only a fixed set of assembly vectors for any DNA sequence with no sequence constraints (FIG. 25 and FIG. 26). The utility of such system is demonstrated by hierarchical assembly of a large 90 kb DNA.

### EXAMPLE 2—DNA METHYLASE AS THE SPECIFIC DNA-BINDING PROTEIN FOR METHYLATION PROTECTION OF THE METHYLATION-PROTECTABLE RESTRICTION ELEMENT

Alternatively, a second DNA methylase can be used as the specific DNA-binding protein for methylation protection of type IIS restriction sites within methylation-protectable restriction sites, providing that DNA methylation by the second methylase itself does not interfere with the restriction activity of the type IIS restriction enzyme (FIG. 28).

Methods

Reagents

Enzymes for molecular biology are from NEB unless otherwise stated. A high-fidelity version of BsaI restriction enzyme named BsaI-HFv2 is used in place of BsaI for all the experiments.

Plasmid Construction

The plasmid for testing methylation protection of M.Csp205I (pMOP_testN7) was constructed using standard restriction enzyme/ligation-based cloning techniques. DNA fragments for cloning were generated by PCR with Q5 polymerase (NEB). The vector backbone was based on the ampicillin resistant MoClo vector pICH47732.

Low copy number plasmid pMOBKZ-2W89, pMOBKZ-2W91, pMOBKZ-2W92 and pMOBKZ-2W94 was constructed for coexpression of M1.Eco31I or M2.Eco31I (under a J23112 or J23114 promoter, a modified RBS based on B0034m named B0034m* and an L3S2P21 terminator), M.Csp205I (under a J23100 promoter, a B0034m* RBS and an L3S1P51 terminator), and S.Csp205I (under a J23100 promoter, a B0034m* RBS and an L3S1P32 terminator). Briefly, for the construction of these plasmids, the transcription units were each assembled from individual cloned DNA parts using BsaI-based MetClo with the MetClo vector set described previously (Lin D et al. Nucleic Acids Res. 2018 Nov. 2; 46(19):e113. doi: 10.1093/nar/gky596 PMID 29986052). The transcription units were then assembled using BsaI into a low copy number kanamycin-resistant vector with an F replication origin, insert-flanking arsB homologous sequences and a zeocin-selection marker. In each round of assembly, insert DNA fragments containing M1.Eco31I or M2.Eco31I were generated by PCR to remove M1.Eco31I or M2.Eco31I methylation in the DNA.

Generation of E. coli Strain

The E. coli strain that constitutively expresses M1.Eco31I or M2.Eco31I together with M.Csp205I and S.Csp205I (DH10B-2W89R, DH10B-2W91R, DH10B-2W92R and DH10B-2W94R) was constructed by recombineering using a linear DNA fragment amplified by PCR from the corresponding plasmids (pMOBKZ-2W89, pMOBKZ-2W91, pMOBKZ-2W92 and pMOBKZ-2W94 respectively) using primers TTCTGTTACCCATCCAATTGTTC (SEQ ID NO: 103) and CAGGCGCTTACCCGCTTCAT (SEQ ID NO: 104).

Results

To develop practical methylation protection system using a second DNA methylase, we designed systems using type I methylase M.Csp205I (with methylase subunit M.Csp205I and specificity subunit S.Csp205I) as the second DNA methylase for methylation protection of BsaI restriction sites from methylation by M1.Eco31I or M2.Eco31I. Methylation of BsaI sites by M1.Eco31I or M2.Eco31I blocks BsaI activity, whereas methylation of the 5^{th} base adenine at the bottom strand of an overlapping BsaI site by M.Csp205I does not itself affect restriction of the BsaI site by BsaI. The aim is to set up a system to test whether M.Csp205I can be used for in vivo methylation protection in combination with either M1.Eco31I or M2.Eco31I. We first constructed new E. coli strains that coexpress M.Csp205I, S.Csp205I and M1.Eco31I (DH10B-2W89R and DH10B-2W91R) or M2.Eco31I methylases (DH10B-2W92R and DH10B-2W94R). We used the strong J23100 promoter for both M.Csp205I and S.Csp205I expression, and tested weaker promoter J23114 or J23112 for M1.Eco31I or M2.Eco31I expression (FIG. 29A). We then constructed a test plasmid (pMOP_testN7) that contains a BsaI site that overlaps with M.Csp205I recognition sequence, and a standard BsaI site (FIG. 29B) and prepared this plasmid in the new strains. Only in the strain that express M2.Eco31I under the weak J23112 promoter (DH10B-2W94R), the test plasmid prepared was cut by BsaI at the BsaI site that overlaps with the guide RNA sequence, but not at the standard BsaI site (FIG. 29C). This demonstrates that M2.Eco31I, not M1.Eco31I under the control of suitable promoter in combination M.Csp205I can be used for methylation protection of BsaI sites.

### EXAMPLE 3 DESIGN OF DNA ASSEMBLY PROCESS BASED ON METHYLATION PROTECTION APPROACH ALONE

Alternatively, a one-pot DNA assembly system can be designed based on the methylation protection approach alone (FIG. 4), without using the methylation switching approach (FIGS. 1 and 2). The system requires a specific design of assembly vector, in which the negative selection marker is flanked by two pairs of type IIS restriction sites in head-to-head arrangement, all of which are methylation protectable. The outer pair of type IIS restriction sites closer to the vector backbone are methylation-protectable by dCas9-based methylation protection approach guided by guide RNA guideX, with 5 bp seed sequence specificity (‘XXXXX’), and the inside pair of BsaI sites closer to the negative selection marker LacZalpha are methylation-protectable by dCas9 through a different guide RNA guideY with a different specificity (‘YYYYY’) (FIG. 30). Preparation of the vector in strains that expression dCas9-methylation protection mechanism with guide RNA guideY will result in selective methylation of the outer pair of type IIS restriction sites, whereas preparation in strains with guideX will result in selective methylation of the inner pair of type IIS restriction sites.

With this unique assembly vector design, a one-pot assembly reaction can then be undertaken using donor plasmid(s) prepared in the strain that expresses the insert-blocking methylase, dCas9 and an appropriate guide RNA guideX, and vector plasmid prepared in the strain that expresses the same methylase, dCas9 and a different guide RNA guideY. The donor plasmid contains inserts flanked by methylation-protectable type IIS restriction sites protectable by dCas9 guided by guide RNA guideX. Preparation of the donor plasmid in methylation protection strain that coexpresses the insert-blocking methylase (M2.Eco31I), dCas9 and guide RNA guideX blocks the internal type IIS restriction sites that are not bound by dCas9 within the insert, whereas the flanking type IIS restriction sites are bound by dCas9/guideX therefore protected from methylation by M2.Eco31I. This enables the release of the insert fragment intact from the donor plasmid by the type IIS restriction enzyme for DNA assembly. The assembly vector on the other hand are prepared in methylation protection strain that expresses a different guide RNA guideY, resulting in selective methylation of outer, but not inner pair of type IIS restriction sites from methylation by M2.Eco31I. This enables the release of the negative selection marker from the vector backbone using the inner pair type IIS restriction sites to be replaced by the assembled insert fragments. Ligation of the vector backbone with the insert fragments generates fully assembled plasmid, in which the assembled insert is now flanked by the two outer type IIS restriction enzyme sites which cannot be cut by the type IIS enzyme because they are methylated. Thus the assembly is completed. The newly formed plasmid can then be transformed into a normal strain that does not express the dCas9-protection mechanism, which will remove all the specific methylation of the type IIS restriction sites for the given enzyme in the plasmid. The assembled plasmid can then be used for next stage assembly starting with transformation into the strain that expresses the insert-blocking methylation, dCas9 and guide RNA guideX, because the assembled insert is flanked by the dCas9-protectable type IIS restriction sites with the same seed sequence specificity as the donor plasmids before the assembly (FIG. 30). Alternatively, the assembly reaction mixture can be transformed directly into the strain that expresses the insert-blocking methylase (M2.Eco31I), dCas9 and guide RNA guideX, which yields assembled plasmids with the internal type IIS restriction sites blocked and the flanking sites protected by dCas9/guideX (FIG. 31). This assembled plasmid can be used directly as donor plasmid for next stage one-pot DNA assembly in the same system.

### EXAMPLE 4—UNIVERSAL ASSEMBLY BASED ON DST1CAS9

Additionally, instead of dCas9 from the CRISPR-Cas9 system of Streptococcus pyogenes, other RNA-guided DNA binding proteins can be used as the specific DNA-binding protein for methylation protection of type IIS restriction sites within methylation-protectable restriction sites. Here we demonstrated that RNA-guided dST1Cas9 from the Streptococcus thermophiles CRISPR1-Cas9 system can be used for methylation protection of BsaI sites from methylation by M2.Eco31I in vivo using E. coli strains coexpressing dST1Cas9, guide RNA and M2.Eco31I methylase. Universal assembly systems based on dST1Cas9-based methylation protection and M.Sen0738I-based methylation switching were then designed and used for practical one-pot BsaI-based assembly of DNA fragments that contain internal BsaI sites.

Methods

Reagents

Enzymes for molecular biology are from NEB unless otherwise stated. A high-fidelity version of BsaI restriction enzyme named BsaI-HFv2 is used in place of BsaI for all the experiments.

Plasmids Construction

Plasmids for testing specific strains and for proof-of-principle DNA assembly were constructed using standard restriction enzyme/ligation-based cloning techniques. DNA fragments for cloning were generated by gene synthesis (Integrated DNA Technologies or Invitrogen), or by PCR with Q5 polymerase (NEB). Plasmids for functional testing, and the assembly vector for proof-of-principle DNA assembly were based on the ampicillin resistant MoClo vector pICH47732 (Addgene plasmid #48000; http://n2t.net/addgene:48000; RRID:Addgene_48000—Weber et al PLoS One. 2011 Feb. 18; 6(2):e16765. doi: 10.1371/journal.pone.0016765, which is herein incorporated by reference). Insert plasmids for proof-of-principle assembly were based on the kanamycin-resistant vector backbone described previously for MetClo proof-of-principle assembly (Lin D et al. Nucleic Acids Res. 2018 Nov. 2; 46(19):e113. doi: 10.1093/nar/gky596. PMID 29986052, which is herein incorporated by reference).

Plasmid pMOBKZ-2W276 or pMOBKZ-2W278 was constructed for coexpression of guide RNA guide #498 or guide #500 (under a J23119 promoter and an L3S2P21 terminator), dST1Cas9 (under a J23119 promoter, a B0034m* RBS and an L3S1P51 terminator), and M2.Eco31I (under a J23112 promoter, a B0034m* RBS and an L3S1P32 terminator). These plasmids were constructed in two stages. In stage one, an intermediate plasmid pMOBKZ-2W274 was constructed, which carries a LacZalpha selection cassette flanked by LguI restriction sites in place of the 20 bp guide RNA sequence in pMOBKZ-2W276 or pMOBKZ-2W278. Briefly, for the construction of pMOBKZ-2W274, the transcription units were each assembled from individual cloned DNA parts using BsaI-based MetClo with the MetClo vector set described previously (Lin D et al. Nucleic Acids Res. 2018 Nov. 2; 46(19):e113. doi: 10.1093/nar/gky596. PMID 29986052). The transcription units were then assembled using BsaI into a low copy number kanamycin-resistant vector with an F replication origin, insert-flanking arsB homologous sequences and a zeocin-selection marker. In each round of assembly, insert DNA fragments containing M2.Eco31I were generated by PCR to remove M2.Eco31I methylation in the DNA. The dST1Cas9 transcription unit for DNA assembly was generated by PCR using a flanking primer containing the J23119 promoter to avoid toxicity of the dST1Cas9 transcription unit in the p15A-based low copy plasmid. In the final round assembly of pMOBKZ-2W274, light blue colonies were screened and verified by sequencing.

In stage two, annealed oligo nucleotides designed based on the guide RNA sequences were cloned into LguI-digested pMOBKZ-2W274 to generate pMOBKZ-2W276 and pMOBKZ-2W278.

Strains

The E. coli strain that constitutively expresses M.Sen0738I and S.Sen0738I (DH10B-2W148R) was described in Example 1. The E. coli strains that deploy dST1Cas9-based methylation protection mechanisms (DH10B-2W276R and DH10B-2W278R) were generated by lambda-red recombineering into DH10B cells using a linear DNA fragment amplified by PCR from pMOBKZ-2W276 and pMOBKZ-2W278 respectively using primers TTCTGTTACCCATCCAATTGTTC (SEQ ID NO: 114) and CAGGCGCTTACCCGCTTCAT (SEQ ID NO: 115). Linear sequences of the PCR product used for recombineering to generate DH10B-2W276R and DH10B-2W278R are listed as 2W276R and 2W278R respectively.

DNA Assembly

The standard assembly reaction was 30 fmol of assembly vector, 60 fmol of each insert plasmid, 15 U T4 DNA ligase HC (Thermo Fisher) and 10 U BsaI-HFv2 (NEB) in 20 ul 1×T4 ligase buffer (NEB). The reaction condition was: 37° C. 15 min, followed by 45 cycles of 37° C. 2 min plus 16° C. 5 min, then 37° C. 20 min, and 80° C. 5 min. Assembly reactions were transformed into specific chemically competent cells, and plated on LB plates with AIX selection (ampicillin 100 μg/ml, IPTG 100 μM, X-gal 50 μg/ml) at 37° C. overnight. White colonies were expanded and screened by restriction digestion using DraIII or BsaI and by DNA sequencing.

For modular assembly of plasmid pMOP498_F5W using M.Sen0738I and a dST1Cas9-guide #498-based system, insert plasmids pMOK498_F5A, pMOK498_F5B, pMOK498_F5C and pMOK498_F5D were prepared in DH10B-2W276R from overnight culture in LB medium supplemented with 1% glucose and 30 μg/ml kanamycin. Assembly vector pMOP498_F5V was prepared in DH10B-2W148R from overnight culture in LB medium with 100 μg/ml ampicillin, and the assembly reaction was transformed into DH10B-2W276R. Assembled clones were cultured in LB medium supplemented with 1% glucose and 100 μg/ml ampicillin.

For modular assembly of plasmid pMOP500_F6W using M.Sen0738I and a dST1Cas9-guide #500-based system, insert plasmids pMOK500_F6A, pMOK500_F6B, pMOK500_F6C and pMOK500_F6D were prepared in DH10B-2W278R from overnight culture in LB medium supplemented with 1% glucose and 30 μg/ml kanamycin. Assembly vector pMOP500_F6V was prepared in DH10B-2W148R from overnight culture in LB medium with 100 μg/ml ampicillin, and the assembly reaction was transformed into DH10B-2W278R. Assembled clones were cultured in LB medium supplemented with 1% glucose and 100 μg/ml ampicillin.

Results

To develop practical Universal assembly systems based on RNA-guided DNA binding proteins other than dCas9, we designed systems using RNA-guided DNA binding protein dST1Cas9 as the specific DNA binding protein for methylation protection of BsaI restriction sites from methylation by M2.Eco31I. RNA guided DNA binding protein dST1Cas9 (also known as dCas9_{Sth1}) is a variant of Cas9 from the CRISPR1 locus of Streptococcus thermophiles, carrying point mutations (D9A, H599A) that inactivate the nuclease activity (Rock J M et al. Nat Microbiol. 2017 Feb. 6; 2:16274. doi: 10.1038/nmicrobiol.2016.274. PMID 28165460, which is herein incorporated by reference). dST1Cas9 can recognise a 27 bp target sequence composed of a 20 bp sequence specified by the chimeric single guide RNA (sgRNA) and a 7 bp PAM motif (NNAGAAG, whereas N can be any of A, T, C or G) (Rock J M et al. Nat Microbiol. 2017 Feb. 6; 2:16274. doi: 10.1038/nmicrobiol.2016.274. PMID 28165460).

Methylation-protectable BsaI site can be designed by combining the 27 bp dST1Cas9 recognition sequence with a BsaI recognition sequence so that the two sequences overlap (FIG. 32). Two different designs were explored. In the first design (FIG. 32B), the last base of the 7 bp PAM motif in the dST1Cas9 recognition sequence overlaps with the first base pair of the BsaI restriction site, to form a methylation protectable BsaI site

| (SEQ ID NO: 107) | |
|---|---|
| | (NNNNNNNNNNNNNNNNNNNN NNAGAA [symbol] GTCTC, |

with the 20 bp sequence that forms Watson-Crick base pairing with the guide RNA sequence underlined, 7 bp PAM motif in italic, and BsaI restriction site in bold). In this design the guide RNA pairs with the bottom strand of the methylation protectable BsaI site when the BsaI site lies at the right end of the methylation protectable BsaI site. In the second design (FIG. 32C), the first 6 bp of the 20 bp sequence specified by the guide RNA in the 27 bp dST1Cas9 recognition sequence contains the BsaI restriction site ‘GAGACC’ to form a 27 bp methylation protectable BsaI site

| (SEQ ID NO: 108) | |
|---|---|
| | (GAGACCNNNNNNNNNNNNNNNNAGAAG, |

with the 20 bp sequence that forms Watson-Crick base pairing with the guide RNA sequence underlined, 7 bp PAM motif in italic, and BsaI restriction site in bold, reverse complement as CTTCTNNNNNNNNNNNNNNNNGGTCTC (SEQ ID NO: 109), so that the guide RNA pairs with the top strand of the methylation protectable BsaI site when the BsaI site lies at the right end of the methylation protectable BsaI site.

To test the above designs of methylation-protectable BsaI sites for dST1Cas9 based methylation protection of BsaI sites from methylation by M2.Eco31I, we constructed E. coli strains that stably coexpress dST1Cas9, guide RNA and M2.Eco31I methylase, with both dST1Cas9 and guide RNA expression driven by the strong J23119 promoter, and M2.Eco31I driven by the weak J23112 promoter (FIG. 33A). We also constructed test plasmids that contain a BsaI site that overlaps with the dST1Cas9 recognition sequence corresponding to the guide RNA sequence (methylation-protectable BsaI site), and a standard BsaI site that does not overlap with the dST1Cas9 recognition sequence (FIG. 33B). For the first design in which the guide RNA pairs with the bottom strand of the methylation-protectable BsaI site, strain DH10B-2W276R was constructed, which expresses guide RNA guide #498 for dST1Cas9, which targets the methylation-protectable BsaI site (GTCTAGTATGCGGATGCCAGTGAGAAGGTCTC (SEQ ID NO: 110), with the BsaI recognition sequence underlined) in test plasmid pMOP_testN24. For the second design in which guide RNA pairs with the top strand of the methylation-protectable BsaI site, strain DH10B-2W278R was constructed, which expresses guide RNA guide #500 for dST1Cas9, which targets methylation-protectable BsaI site (CTTCTCAAGCCCCAGAATGGTGGTCTC (SEQ ID NO: 111), with the BsaI recognition sequence underlined) in test plasmid pMOP_testN32. For both designs, the test plasmid prepared in the corresponding strain can be cut at the BsaI site that overlaps with the guide RNA target sequence, but not the standard BsaI site (FIG. 33C). This demonstrates that dST1Cas9 can selectively protect BsaI sites that overlap with the specific RNA-guided dST1Cas9 binding sequence from methylation by M2.Eco31I.

The dST1Cas9-based methylation protection systems can also be combined with methylation switching mechanism to build one-pot Universal assembly systems. For example, both designs of dST1Cas9-based methylation-protectable BsaI sites can be combined with M.Sen0738I-based methylation switching mechanism to build dST1Cas9-protectable and M.Sen0738I-switchable BsaI sites (FIG. 34). The methylation-protectable BsaI sites targeted by dST1Cas9-guide #498 in strain DH10B-2W276R and dST1Cas9-guide #500 in DH10B-2W278R have indeed incorporated designs to make them methylation switchable by M.Sen0738I. These can then be used to build Universal assembly systems for one-pot DNA assembly, whereas the insert plasmids were prepared in strains that deploy dST1Cas9-based methylation protection, and the assembly vectors were prepared in strains that deploy methylation switching (FIG. 35-38).

The designed Universal assembly systems using dST1Cas9/M2.Eco31I-based methylation protection and M.Sen0738I-based methylation switching were then used for proof-of-principle assembly of a 3.6 kb DNA from the human MICA locus (Genbank sequence KF724576.1:23-3633) from 4 fragments, each of which contains an internal BsaI site that does not overlap with the RNA-guided dST1Cas9 binding sequence (FIG. 39A, Table 4). The insert plasmids carry the starting fragments flanked by dST1Cas9-protectable and M.Sen0738I-switchable BsaI sites that generates compatible adhesive ends for DNA assembly. Preparation of insert plasmids in strains that express dST1Cas9, M2.Eco31I, and the corresponding guide RNA for dST1Cas9-based methylation protection results in selective methylation of the internal BsaI sites, whereas the flanking BsaI sites were not methylated due to methylation protection. In both dST1Cas9-based methylation protection strains (DH10B-2W276R and DH10B-2W278R), the internal BsaI sites were fully methylated, and the percentage of insert plasmids with both flanking BsaI sites protected by dST1Cas9 from M2.Eco31I methylation exceeds 50% (FIG. 39B and FIG. 39C). One pot assembly using these insert plasmids prepared in corresponding strains for dST1Cas9-based methylation protection (DH10B-2W276R or DH10B-2W278R), and compatible assembly vector prepared in strains for M.Sen0738I-based methylation switching (DH10B-2W148R), followed by direct transformation into corresponding strains for dST1Cas9-based methylation protection (DH10B-2W276R or DH10B-2W278R), results in successful assembly of the 3.6 kb fragment (FIG. 39D). The assembly efficiency is 75% (6/8) for the proof-of-principle assembly using the dST1Cas9-guide #498/M2.Eco31I/M.Sen0738I universal assembly system, and 100% (8/8) for the dST1Cas9-guide #500/M2.Eco31I/M.Sen0738I system (FIG. 39D). As the assembly was transformed into corresponding methylation protection strains, the internal BsaI sites of the assembled plasmids were fully methylated, whereas the flanking BsaI sites were protected from methylation (FIG. 39E). The 3.6 kb assembled fragment can thus be released intact from the assembled plasmid, which enables the assembled plasmid to be used as insert plasmid for next stage of Universal assembly. The percentage of the successfully assembled plasmids with both flanking BsaI sites protected from M2.Eco31I methylation is higher for plasmids assembled in dST1Cas9-guide #498 based methylation protection strain (DH10B-2W276R) than dST1Cas9-guide #500 based strain (DH10B-2W278R) (FIG. 39E), suggesting strain DH10B-2W276R is more efficient for methylation protection.

These data together demonstrate that Universal assembly system based on dST1Cas9-based methylation protection can assemble DNA with internal BsaI sites in a one-pot reaction.

### EXAMPLE 5—HIERARCHICAL ASSEMBLY OF ˜260 KB DNA

The set of scarless assembly vectors from the scarless assembly scheme described in Example 1 was also used to assemble a large piece of ˜260 kb DNA fragment.

Methods

Reagents

Enzymes for molecular biology are from NEB unless otherwise stated. A high-fidelity version of BsaI restriction enzyme named BsaI-HFv2 is used in place of BsaI for all the experiments.

Strains and Plasmids

E. coli strains DH10B-2W214R and DH10B-2W148R have been described in Example 1. The 10 insert plasmids carrying the starting fragments (pMOBK401_L3G1, pMOBK401_L3G2, pMOBK401_L3G3, pMOBK401_L3G4, pMOBK401_L3G5, pMOBK401_L3G6, pMOBK401_L3G7, pMOBK401_L3G8, pMOBK401_L3G9, pMOBK401_L3G10) are kanamycin-resistant plasmids with F replication origin. The assembly vectors (pMOBK401_VL, pMOBC401_VL, pMOBC401_VM, pMOBC401_VR) are the kanamycin (pMOBK401) or chloramphenicol (pMOBC401) resistant scarless assembly vectors with F replication origin from the scarless assembly scheme described in Example 1.

DNA Assembly

The 260 kb DNA was assembled in two stages using M.Sen0738I and dCas9-guide #401-based universal assembly system. In stage one, the insert fragments were assembled 2-4 fragments per group using 30 fmol of each insert plasmid prepared in DH10B-2W214R, 15 fmol of the recipient assembly vector prepared in DH10B-2W148R with 10 U BsaI-HFv2 (NEB) and 15 U T4 DNA ligase HC (Thermo Fisher) in a 20 ul assembly reaction in 1×T4 ligase buffer (NEB). The reaction condition was: 3TC 15 min, followed by 45 cycles of 3TC 2 min plus 16° C. 5 min, then 3TC 20 min, and 80° C. 5 min. 20 U BsaI-HFv2 was then added to the assembly reaction for incubation at 37° C. for 45 min followed by heat inactivation at 80° C. for 5 min. The assembly reaction was dialysed for 1 h with water, and then transformed into NEB10B competent cells by electroporation at 0.9 kV 100Ω 25 μF using 1 mm electroporation cuvettes a Gene Pulser electroporation device (Bio-Rad). 1 ml LB medium was then added to the cells, and cultured at 3TC for 1 h. Cells were then plated on LB plates supplemented with chloramphenicol 12.5 μg/ml, IPTG 100 μM, X-gal 50 μg/ml at 37° C. overnight. White colonies were screened by NotI restriction digest followed by pulsed-field electrophoresis. Plasmid DNA from positive clones were transformed into DH10B-2W214R, and then used as insert plasmids for the stage two assembly. In stage two, the assembly reaction set up and transformation procedure was the same as stage one. Transformed cells were plated onto LB plates supplemented with kanamycin 30 μg/ml, IPTG 100 μM, X-gal 50 μg/ml at 37° C. overnight. White colonies were screened by NotI digestion followed by pulsed-field electrophoresis.

Results

The set of scarless assembly vectors from the scarless assembly scheme (FIG. 22) was used to assemble a ˜260 kb DNA fragment from the human CLEC16A locus from 10 fragments in two stages (FIG. 40, Table 5 and Table 6). The 10 starting fragments cloned in insert plasmids with flanking dCas9/guide #401 methylation-protectable and M.Sen0738I-switchable BsaI sites carry suitable overhang sequences depending on the position the assembled fragment would take up in the next round of assembly, as designed based on the process illustrated in FIG. 26. In round one of the assembly, groups of 2-4 fragments were assembled into suitable assembly vectors depending on the position that the assembled fragment would take up in the next round of assembly. In round two, the three intermediate insert plasmids prepared in DH10B-2W214R were assembled in a single reaction into the final product. The success rate of the first round assembly exceeds 70%, and the success rate at the second round is around 17%. This demonstrates that the same set of scarless assembly vector from the scarless assembly scheme combined with Universal assembly can be used for scarless assembly of large DNA molecules up to ˜260 kb.

### EXAMPLE 6—UNIVERSAL ASSEMBLY BASED ON IN VITRO METHYLATION SWITCHING AND IN VITRO METHYLATION PROTECTION

Here we describe the Universal Assembly approach based on in vitro methylation switching and in vitro methylation protection, using M.Osp807II methylation switching, and dCas9 with M2.Eco31I or M2.BsaI for methylation protection. Compared with the in vivo approach, the in vitro approach uses recombinant methylases in protein form to perform methylation switching and methylation protection of DNA directly in a tube. This eliminates the need for bacteria in this part of the assembly process and so eliminates the need to develop bacterial strains expressing particular methylases.

Methods

Reagents

Molecular biology reagents were from NEB unless otherwise stated. A high-fidelity version of BsaI restriction enzyme named BsaI-HFv2 is used in place of BsaI for all the experiments.

Plasmid Construction

Plasmids for recombinant protein expression were constructed by standard cloning techniques with PCR and restriction enzymes. Briefly, pET-M.Osp807II plasmid for expression of M.Osp807II with N terminal His tag was constructed by cloning a PCR product carrying E. coli-codon optimized M.Osp807II into pET-15b vector (Novagen) using NdeI/BamHI. pET-M2.Eco31I plasmid for expression of M2.Eco31I with deletion of the first 7 amino acids and with C terminal His tag was constructed by cloning a PCR product carrying E. coli-codon optimized M2.Eco31I into pET-30b (+) vector (Novagen) using NdeI/HindIII. pET-M2.BsaI plasmid for expression of M2.BsaI with C terminal His tag was constructed by cloning a PCR product carrying E. coli-codon optimized M2.BsaI into pET-30b (+) vector (Novagen) using NdeI/HindIII. Plasmid sequences for pET-M.Osp807II, pET-M2.Eco31I and pET-M2.BsaI plasmids are listed in the supplementary information.

Recombinant Protein Purification

Recombinant proteins were produced using the following protocol. E. coli strain BL21(DE3)pLysS carrying the plasmids for recombinant protein expression were cultured in 500 ml LB medium at 3TC until OD600˜0.5 to 0.7. Protein expression was induced with 0.5 mM IPTG at 20° C. for 16 h. Cell pellets were frozen at −20° C., thawed, and resuspended in 15 ml 1× binding buffer (50 mM sodium phosphate pH 8.0, 300 mM NaCl, 1 mM imidazole, 10% glycerol). The cells were sonicated on ice, and lysed cells were pelleted at 14,000 g at 4° C. for 30 min. Cleared supernatants containing soluble proteins were mixed with 2 ml Ni-NTA His Bind Resin (Merck) pre-equilibrated with 1× binding buffer, and incubated on ice for 1 hour. The resin/soluble protein were loaded onto a column, and the flowthrough were collected and loaded onto the column that contains the resin a second time. The resin was washed once with 25 ml 1× binding buffer, once with 30 ml 1× binding buffer with 10 mM imidazole, and once with 20 ml 1× binding buffer with 50 mM imidazole. The purified protein was eluted from the column using 8 ml elution buffer (1× binding buffer with 150 mM imidazole). Eluted fractions were desalted using PD-10 column using the desalting buffer (50 mM sodium phosphate pH7.4, 200 mM NaCl, 10% glycerol), and proteins were aliquoted and stored at −80° C.

Assays for Testing Methylase Activity

Methylation reaction was set up using 200 ng plasmid DNA substrate and 1 μL enzyme (˜2 μM concentration) in 1×BamHI methyltransferase buffer (NEB) supplemented with 160 μM SAM in a 20 μL reaction. The reaction was incubated at 37° C. for 60 min and heat-inactivated at 80° C. for 20 min. Following methylation, 3 μL 10× CutSmart Buffer (NEB), 4 μL 50 mM MgCl2 and 0.5 μL restriction enzyme and 2.5 μL water was added to the 20 μL methylation reaction, and incubated at 37° C. for 1 h. Samples were analyzed by 1% agarose gel electrophoresis.

In Vitro Methylation Switching of DNA Assembly Vector

2 μg plasmid DNA were methylated using 2 μL 2.4 μM M.Osp807II in 1×BamHI methyltransferase buffer (NEB) supplemented with 160 μM SAM in a 20 μL reaction at 37° C. for 1 h followed by heat inactivation at 80° C. for 20 min. Methylated assembly vector plasmids were purified using Qiaquick PCR clean up kit (Qiagen).

In Vitro Methylation Protection of Insert DNA Plasmids

Cas9 sgRNA guide #360 were generated by in vitro transcription and purified using Precision gRNA Synthesis kit (ThermoFisher) following manufacture's protocol. For methylation protection reaction, 7.8 pmol dCas9 (NEB) was incubated with 7.8 pmol sgRNA and 3 μL 10×NEBuffer 3.1 (NEB) in a 15 μL reaction at 25° C. for 10 min, following which 780 fmol insert plasmids were added to the pre-formed dCas9/sgRNA reaction to a final volume 28 μL and incubated at 3TC for 15 min. 1 μL 2.4 μM M2.Eco31I and 1 μL 3.2 mM SAM were then added to the reaction, and incubated at 35° C. for 15 min, followed by heat inactivation at 80° C. for 20 min. Methylated insert DNA plasmids were purified using Qiaquick PCR clean up kit (Qiagen).

DNA Assembly

The DNA assembly reaction contains 60 fmol of each insert DNA plasmid (pMOK360_F3A, pMOK360_F3B, pMOK360_F3C and pMOK360_F3D) that has been subject to in vitro methylation protection using dCas9/sgRNA/M2.Eco31I or dCas9/sgRNA/M2.BsaI, 60 fmol DNA assembly vector pMOP360_F3V that has been subject to in vitro methylation switching using M.Osp807II, 1000 U NEB T4 DNA ligase, 5 U BsaI in 20 μL 1×T4 DNA ligase buffer (NEB). The reaction condition was 37° C. 15 min followed 45 cycles of 37° C. 2 min plus 16° C. 5 min, then 37° C. 20 min and 80° C. 5 min. 2 μL 10× CutSmart buffer and 10 U BsaI was added to the reaction and incubated at 37° C. for 3 h. The reaction was transformed into chemically competent DH10B cells and plated on LB agar plates with 100 μg/ml Ampicillin, 100 μM IPTG and 50 μg/ml X-Gal, and incubated at 37° C. overnight. White colonies were expanded and screened by restriction digestion using DraIII.

Results

Here we developed methods for Universal assembly by in vitro methylation using purified recombinant enzymes.

In Vitro Methylation Switching

The methylation switching step in Universal assembly can be carried out in vitro using recombinant switch methylase M.Osp807II, which selectively block M.Osp807II-methylation switchable BsaI sites from restriction by BsaI (FIG. 41).

In Vitro Methylation Protection

The methylation protection step can be carried out using recombinant dCas9 and recombinant methylase M2.Eco31I or M2.BsaI that methylates and blocks BsaI sites (FIG. 42 and FIG. 43) in a multi-step reaction (FIG. 44). DNA molecules were first incubated with dCas9/sgRNA, which selectively binds to the methylation-protectable BsaI sites, but not non-protectable standard BsaI sites (FIG. 44). Recombinant M2.Eco31I or M2.BsaI were then added to the reaction to methylate the BsaI sites in the DNA (FIG. 44). However, because the methylation-protectable BsaI sites were stably bound by dCas9/sgRNA in the previous step, this prevents the methylation-protectable BsaI sites from being methylated by M2.Eco31I or M2.BsaI in vitro, therefore results in selective methylation of non-protectable standard BsaI sites in the DNA. The reaction was then subject to heat inactivation and cleaned up using spin columns to remove stably bound dCas9/sgRNA and M2.Eco31I or M2.BsaI, generating purified DNA with selective methylation of non-protectable BsaI sites only (FIG. 44).

DNA Assembly Based on In Vitro Methylation Switching and Methylation Protection

The in vitro methylation switching and methylation protection methods can then be used for one pot assembly of DNA (FIG. 45). The assembly vector was subject to in vitro methylation switching by M.Osp807II to selectively methylate the pair of methylation-switchable BsaI sites located within the assembly vector backbone. The insert DNA plasmids were subject to in vitro methylation protection with dCas9/sgRNA and either M2.Eco31I or M2.BsaI to selectively methylate the internal BsaI sites located within the insert fragment, whereas the methylation-protectable BsaI sites that flanks the insert fragment remain unmethylated due to protection of dCas9/sgRNA. One pot assembly using methylated assembly vector and insert DNA plasmid generates plasmid carrying orderly assembled fragments in assembly vector backbone, in which all the BsaI sites were methylated. Transformation of the assembled plasmid into normal E. coli strains generates unmethylated assembled plasmids with the assembled fragment flanked by methylation-protectable BsaI sites ready to use as insert plasmid for next round of DNA assembly based on in vitro methylation protection.

Using M.Osp807II-based in vitro methylation switching, and dCas9/sgRNA/M2.Eco31I or M2.BsaI based in vitro methylation protection, proof-of-principle in vitro Universal assembly was then carried out for assembly of a ˜3.7 kb DNA from the MICA locus from 4 ˜1 kb insert fragments, each of which contain an internal BsaI sites (FIG. 46A). Over 99% of the resulting colonies after the transformation of the DNA assembly reaction were white. A selection of these were expanded and tested with digestion, with 80% (4/5) of the colonies verified by restriction digest for either M2.Eco31I or M2.BsaI-based system (FIG. 46B).

### Tables

Table 1. Practical Universal Assembly systems
Table 2. Feasible designs of excision-type overhang sequences for scarless assembly based on M.Osp807II and M.Sen0738I-based methylation switching of BsaI sites
Table 3. Scarless assembly of a ˜90 kb DNA using Universal assembly
Table 4. Practical Universal Assembly systems using dST1Cas9-based methylation protection
Table 5. Scarless assembly of a ˜260 kb DNA using Universal assembly
Table 6. Insert plasmids for ˜260 kb DNA assembly

TABLE 1
Practical Universal Assembly systems

| | Methylation | | | Methylation protection | | |
|---|---|---|---|---|---|---|
| | switching for | | | for insert | | |
| | vector preparation | | | plasmid preparation | | |
| | | | Test | | Protection | guide RNA |
| Systems | Methylase | Strain | plasmid | Methylase | mechanism | sequence |
| 1 | M.Osp | DH10B- | pMOP_BsaNC | M2.Eco311 | dCas9 | GTGCAGTAC |
| | 80711 | MOsp80 | | | | CTCTCACGA |
| | | 711 | | | | CT (SEQ |
| | | | | | | ID NO: |
| | | | | | | 105) |
| 2 | M.Sen | DH10B- | pMOP_testN10 | M2.Eco311 | dCas9 | GAGTAATCA |
| | 07381 | 2W148R | | | | CGCCAGTGC |
| | | | | | | AT (SEQ |
| | | | | | | ID NO: |
| | | | | | | 106) |

| | Proof of principle assembly | | | | |
|---|---|---|---|---|---|
| | | Test | Insert | | Assembled |
| Systems | Strain | plasmid | plasmids | Vector | plasmid |
| 1 | DH10B- | pMOPtestN8 | pMOK360_F3A, | pMOP360_F3V | pMOP360_F3W |
| | 2W213R | | pMOK360_F3B, | | |
| | | | pMOK360_F3C, | | |
| | | | pMOK360_F3D | | |
| 2 | DH10B- | pMOPtestN20 | pMOK401_F4A, | pMOP401_F4V | pMOP401_F4W |
| | 2W214R | | pMOK401_F4B, | | |
| | | | pMOK401_F4C, | | |
| | | | pMOK401_F4D | | |

TABLE 2
Designs of excision-type overhang sequences
for scarless assembly based on M.Osp807II
and M.Sen0738I-based methylation switching
of BsaI sites

| | Excision- | Pre-assembly | Excised |
|---|---|---|---|
| Bases | type | overhang | adaptor |
| excised | design* | sequence** | sequence*** |
| 4 bp | GGTCTCA | CTCA/TGAG | CTCA/TGAG |
| | NGAGACC | | |
| 4 bp | GGTCTCC | CTCC/GGAG | CTCC/GGAG |
| | NGAGACC | | |
| 4 bp | GGTCTCG | CTCG/CGAG | CTCG/CGAG |
| | NGAGACC | | |
| 4 bp | GGTCTCT | CTCT/AGAG | CTCT/AGAG |
| | NGAGACC | | |
| 5 bp | GGTCTCN | TCTC/GAGA | TCTCN/NGAGA |
| | GAGACC | | |
| 6 bp | GGTCTNG | GTCT/AGAC | GTCTCN/NGAGAC |
| | AGACC | | |

*Methylated base due to methylation switching methylation opposite to the base highlighted in bold
**Forward and reverse-complement overhang sequence forward and reverse complement orientations
***Adaptor sequence to be excised from the end of the insert DNA in

TABLE 3
Scarless assembly of a ~90 kb DNA using Universal assembly

| | | | | | | Size of | |
|---|---|---|---|---|---|---|---|
| Round of | | | Assembly | | Assembled | assembled | Success |
| assembly | Insert plasmids | | vector | | plasmid | fragment | rate |
| First | pMOLC401_F1, | pMOLC401_F2, | pMOLC401_F3, | pMOBK401_ | pMOBK401_G1 | 25.7 kb | 83% |
| | pMOLC401_F4 | | | VL | | | (5/6) |
| First | pMOLC401_F5, | pMOLC401_F6, | pMOLC401_F7, | pMOBK401_ | pMOBK401_G2 | 25.7 kb | 100% |
| | pMOLC401_F8 | | | VM | | | (6/6) |
| First | pMOLC401_F9, | pMOLC401_F10, | pMOLC401_F11, | pMOBK401_ | pMOBK401_G3 | 26.5 kb | 83% |
| | pMOLC401_F12 | | | VM | | | (5/6) |
| First | pMOLC401_F13, | pMOLC401_F14 | | pMOBK401_ | pMOBK401_G4 | 12.6 kb | 100% |
| | | | | VR | | | (6/6) |
| Second | pMOBK401_G1, | pMOBK401_G2, | pMOBK401_G3, | pMOBC401_ | pMOBC401_H1 | 90.2 kb | 100% |
| | pMOBK401_G4 | | | VM | | | (12/12) |

TABLE 4
Practical Universal Assembly systems using
dST1Cas9-based methylation protection

| | | | | Methylation protection | | |
|---|---|---|---|---|---|---|
| | Methylation switching | | | for insert plasmid | | |
| | for vector preparation | | | preparation | | |
| | | | Test | | Protection | guide RNA |
| Systems | Methylase | Strain | plasmid | Methylase | mechanism | sequence |
| 3 | M.Sen0738I | DH10B- | pMOPtest | M2.Eco31I | dST1Cas9 | GTCTAGTATG |
| | | 2W148R | N10 | | | CGGATGCCAG |
| 4 | M.Sen0738I | DH10B- | pMOPtest | M2.Eco31I | dST1Cas9 | GAGACCACCA |
| | | 2W148R | N10 | | | TTCTGGGGCT |

| | Methylation | | | | |
|---|---|---|---|---|---|
| | protection for | | | | |
| | insert plasmid | | | | |
| | preparation | | Proof of principle assembly | | |
| | | Test | Insert | | Assembled |
| Systems | Strain | plasmid | plasmids | Vector | plasmid |
| 3 | DH10B- | pMOP_test | pMOK498_F5A, | pMOP498_F5V | pMOP498_F5W |
| | 2W276R | N24 | pMOK498_F5B, | | |
| | | | pMOK498_F5C, | | |
| | | | pMOK498_F5D | | |
| 4 | DH10B- | pMOP_test | pMOK500_F6A, | PMOP500_F6V | pMOP500_F6W |
| | 2W278R | N32 | pMOK500_F6B, | | |
| | | | pMOK500_F6C, | | |
| | | | pMOK500_F6D | | |

TABLE 5
Scarless assembly of a ~260 kb DNA using Universal assembly

| Round of | | | | Size of assembled | Success |
|---|---|---|---|---|---|
| assembly | Insert plasmids | Assembly vector | Assembled plasmid | fragment | rate |
| First | pMOBK401_L3G1, pMOBK401_L3G2 | pMOBC401_VL | pMOBC401_L3H1 | 51.4 kb | 100% (6/6) |
| First | pMOBK401_L3G3, pMOBK401_L3G4, | pMOBC401_VM | pMOBC401_L3H2 | 104.8 kb | 100% (4/4) |
| | pMOBK401_L3G5, pMOBK401_L3G6 | | | | |
| First | pMOBK401_L3G7, pMOBK401_L3G8, | pMOBC401_VR | pMOBC401_L3H3 | 104.6 kb | 78% (7/9) |
| | pMOBK401_L3G9, pMOBK401_L3G10 | | | | |
| Second | pMOBC401_L3H1, pMOBC401_L3H2, | pMOBK401_VL | pMOBK401_L3I1 | 260.7 kb | 17% (2/12) |
| | pMOBC401_L3H3 | | | | |

TABLE 6
Insert plasmids for ~260 kb DNA assembly

| Plasmid | Insert fragment coordinates |
|---|---|
| pMOBK401_L3G1 | NC_000016.10:10923586-10952567 |
| pMOBK401_L3G2 | NC_000016.10:10952564-10974968 |
| pMOBK401_L3G3 | NC_000016.10:10974965-11004845 |
| pMOBK401_L3G4 | NC_000016.10:11004842-11027273 |
| pMOBK401_L3G5 | NC_000016.10:11027270-11057197 |
| pMOBK401_L3G6 | NC_000016.10:11057194-11079729 |
| pMOBK401_L3G7 | NC_000016.10:11079726-11102053 |
| pMOBK401_L3G8 | NC_000016.10:11102050-11132052 |
| pMOBK401_L3G9 | NC_000016.10:11132049-11161958 |
| pMOBK401_L3G10 | NC_000016.10:11161955-11184328 |
| pMOBC401_L3H1 | NC_000016.10:10923586-10974968 |
| pMOBC401_L3H2 | NC_000016.10:10974965-11079729 |
| pMOBC401_L3H3 | NC_000016.10:11079726-11184328 |
| pMOBK401_L3I1 | NC_000016.10:10923586-11184328 |

Sequences for Generation of Modified E. coli Strains:

The modified bacterial strain described herein may be modified/transformed with, or comprise, nucleic acid comprising one or more of the following sequences. It is understood that the sequences may or may not be limited with a specific guide sequences (as identified by underlining) used. In some embodiment, the specific guide RNA sequences (as identified by underlining) may be of any sequence. All polynucleotides and their sequences described herein, including functional variants thereof, may be considered to be an aspect or embodiment of the invention.

>2W148R (Linear DNA sequence for recombineering to generate the stable E. coli strain DH10B-2W148R) (SEQ ID NO: 116)

TTCTGTTACCCATCCAATTGTTCAAAATTCTTGCTGATGAAACCCGTCTG
GGCATCGTTTTACTGCTCAGCGAACTGGGAGAGTTATGCGTCTGCGATCT
CTGCACTGCTCTCGACCAGTCGCAGCCCAAGATCTCCCGCCACCTGGCAT
TGCTGCGTGAAAGCGGGCTATTGCTGGACCGCAAGCAAGGTAAGTGGGTT
CATTACCGCTTATCACCGCATATTCCAGCATGGGCGGCGAAAATTATTGA
TGAGGCCTGGCGATGTGAACAGGAAAAGGTTCAGGCGATTGTCCGCAACC
TGGCTCGACAAAACTGTTCCGGGGACAGTAAGAACATTTGCAGTTAAAAA
TTTAGCTAAACACATATGAATTTTCAGATGTGTTTTATCCGGGAGGCATT
ATGTTACTGGCAGGCGCTATCTTTGTCCTGACCATCGTATTGGTTATCTG
GCAGCCGAAAGGTTTAGGCATCGGCTGGAGTGCAACGCTCGGCGCAGTAC
TGGCGTTAGTTACGGGCGTGGTCCATCCGGGTGATATTCCGGTGGTGTGG
AATATCGTCTGGAACGCGACGGCTGCGTTTATCGCCGTCATTATCATCAG
CCTGCTGCTGGATGAGTCCGGCTTTTTTGAATGGGCGGCGCTGCACGTCT
CACGCTGGGGTAATGGTCGTGGTCGCTTGCTGTTTACCTGGATTGTCCTG
CTCGGTGCTGCCGTTGCCGCCCTGTTTGCCAATGATGGCGCGGCGCTTAT
TTTGACACCGATTGTCATCGCCATGCTGCTGGCTTTAGGGTTCAGTAAAG
GCACTACGCTGGCGTTCGTGATGGCGGCCGGATTCATTGCCGATACCGCC
AGCCTGCCGCTTATTGTCTCCAACCTGGTGAATATCGTTTCCGCTGATTT
CTTTGGCCTCGGCTTTCGCGAATACGCCTCGGTGATGGTGCCGGTGGATA
TCGCCGCGATTGTTGCCACGCTGGTGATGTTACATCTCTATTTTCGCAAA
GATATTCCGCAGAACTACGATATGGCGCTGCTGAAATCTCCCGCAGAAGC
GATCAAAGATCCTGCTACGTTCAAAACTGGGCAAGGAGTTGACGGCTAGC
TCAGTCCTAGGTACAGTGCTAGCTACTAGAGAAAGAGGAGAAATACAAAT
GTCAATCTCATCTGCTATTAAAAGCTTGCAAGACATCATGCGCAAAGATG
CCGGTGTTGATGGTGATGCCCAGCGTTTGGGCCAGCTTTCTTGGTTACTG
TTTTTAAAAATCTTTGATGCACAGGAACAGGCACTGGAGATTGAACAAGA
AAAGTATCGTCTGCCTATGCCAGAACGCTATTTATGGCGCAATTGGGCCG
CCGATAATGAAGGAATTACAGGGGATAAATTGTTGGCGTTCGTCAACGAT
GATCTTTTTCCCACCTTGAAGGACTTGCCCGCCCAGATCGACATCAACCC
CCGCGGGTATGTAGTAAAGCAGGCTTTCTCCGACGCTTATAACTACATGA
AAAACGGGACGTTGCTGCGCCAAGTTATCAACAAGTTAAATGAGATTGAC
TTTACACGTGCTTCAGAGCGCCATCTTTTTGGAGATATCTACGAGCAAAT
TTTGCGTGACTTGCAGGCTGCCGGTAATGCTGGTGAATTTTATACGCCTC
GCGCTGTTACTCGCTTCATGGTTGAACGTGTAGACCCTAAACTGGGTGAG
TCGATCATGGATCCGGCTTGTGGTACGGGGGGATTTTTGGCATGCGCCTT
CGACCACGTTAAAAACCATTATGCCCATACTGTGACTGACCATCAAATCT
TGCAGAAACAAATTCATGGGGTCGAAAAGAAGCAATTACCACACTTGTTG
TGTACGACCAACATGCTGCTTCACGGCATCGAAGTCCCCGTTCAAATCCG
CCACGACAATACTCTTAATAAACCACTTTCATCGTGGGACGAGCAAATGG
ATGTTATCATCACTAACCCCCCATTCGGGGGTACTGAGGAAGATGGCATT
GAAAAGAACTTCCCCTCTGACATGCAGACACGTGAGACGGCGGACTTATT
TCTTCAGCTTATTATTGAGGTCTTAGCTAAGAACGGTCGTGCCGCCGTGG
TGTTGCCCGATGGTACGCTTTTTGGTGAAGGTGTTAAAACTAAGATTAAA
AAGTTGCTGACCGAAGAGTGCAATTTGCACACAATTGTCCGTTTACCTAA
CGGCGTTTTTAACCCCTATACGGGGATCAAAACTAACCTGTTATTTTTCA
CGAAAGGGCAGCCGACCAAGGAGATCTGGTTTTATGAACACCCTTACCCC
GCAGGTGTTAAAAACTACAGCAAGACGAAACCAATGAAATTTGAGGAGTT
TCAGGCAGAAATTGATTGGTGGGGTAACGAAGCGGATGGGTTTGCAAGTC
GCGTCGAGAATGAGCAGGCCTGGAAAGTCTCTATTGATGAAGTTATCGCC
CGTAACTTCAATCTTGATATTAAGAACCCCCACCAGGCAGAAACCGTATC
CCATGACCCAGACGAGCTGTTAGCACAATACGCCAAACAGCAAGAGGCCA
TCCAAACTCTTCGCCATCAGCTTCGTGACATCTTAGGAACTGCGTTGAGT
GGTAAAGAGGCGAACTAAGCTTCTCGGTACCAAATTCCAGAAAAGAGGCC
TCCCGAAAGGGGGGCCTTTTTTCGTTTTGGTCCCGCTAGGTGGAGTTGAC
GGCTAGCTCAGTCCTAGGTACAGTGCTAGCTACTAGAGAAAGAGGAGAAA
TACAAATGGCGGTGGAAAAACTTATTGTAGATCATATCGACACCTGGACT
ACCGCCCTTCAGACGCGCTCCACGGCCGGTCGCGGGAGCAGCGGCAAAAT
CGACTTGTATGGAATTAAGAAGCTTCGCGAACTTATTTTAGAACTGGCCG
TACGTGGCAAGCTGGTCCCGCAAGACCCAAATGACAAGCCCGCTTCGGTG
CTTTTAGAACGCATCGCGACAGAAAAAGCGGAGCTGGTGAAACAAGGTAA
GATTAAAAAGCAGAAGCCCTTGCCAGAAATCTCTGAGGAGGAAAAGCCAT
TTGAACTGCCAGCGGGCTGGGAATGGGCTCGTTTAAATGAATTAGCTCCT
ATGGGAATCATTGACGGGGATTGGATCGAGTCAAAAGACCAGGACCCGTC
AGGCGCTTACCGTTTGATTCAGTTAGCTGATGTTGGCGTGGGAGATTTCA
AGGACAAGTCCGATCGCTATATCAACACTTCGACGTTTCATCGTTTGAAC
TGCCATCAACTGATGGAGGGTGATATCCTTATCGCGCGCTTACCGAACCC
GATCGGACGCGCGTGCATCTTTCCTAAGCTTTCCCAAAGCGCTATCACTG
TAGTAGATATTGCTACAATGCGCCCTTCAGGAAATTATAGTGCGGAATAT
ATTATTTCCGCCATTAACAGTTTAACATTTCGTCAGCAAGTAGAATCGTT
TGGAAAGGGCGCGACGCGCTTCCGCATCGCAACCGGGCACCTGAAAACAT
TGCTTTTGCCAATTCCACCCGTCCAGGAACAATATAGTATCTTCAAAAAA
ATTAAGGAGCTGATGTCCCTTTGCGATCAGTTAGAACAATATAGCCTGAC
TTCCCTTGATGCACATCAACAATTAGTGGAAACGTTGTTAACCACCTTGA
CGGACAGCCAGAATGCAGATGAGTTAGCTGAAAATTGGGCCCGTATCAGC
GAACACTTCGATACTCTTTTTACAACTGAAGTCAGTATCGACGCCCTGAA
GCAGACGATCCTGCAACTGGCTGTCATGGGTAAGCTGGTGCCACAGGACC
CAAACGATGAACCAGCATCTGAACTGCTGAAGCGTATTGCACAGGAAAAA
GCACAGTTGGTAAAGGATGGAAAGATGAAGAAGCAAAAGCCATTACCGCC
GATTAGTGATGAGGAAAAACCATTCGAGTTACCATCGGGTTGGGAATGGT
GTTTATTTGAGGACGTCGTAGATATTCAATCGGGCATTACCAAGGGCCGC
AACTTAGCAAATCGCAAGCTTATTTCTATCCCGTATTTGCGCGTAGCCAA
CGTGCAGCGCGGTTATTTGGACCTTTCAGAAGTTAAAGAAATCGACATTC
CCGAAGAAGAGAAGGATAAGTACCACGTGATCAAGGGCGACTTGTTAATC
ACGGAAGGCGGCGATTGGGACACAGTAGGGCGTACTACTGTTTGGTGCCA
CGACTGGTATATCGCCAATCAAAACCACGTGTTTAAGGGACGTATTATCG
GGCAGGACATCGATCCCTATTGGCTTGAGACGTACATGAACTCTCCTTAC
GCCCGTGATTACTTCGCGAGTGCCTCTAAACAAACTACCAACCTGGCGAG
TATCAACAAGACGCAACTTCGTGGATGTCCAGTGGCTATTCCACCTAGTA
GCGAAGCAGAAAAAATCATGCTTAAATTAAACGATTTTAATGAACTGTGT
GAAAAATTAAAGCTGCAGATTCAGAGCGCTCAACAGACGCAGCTTCATCT
TGCGGACGCCCTGACTGATGCCGCAATTAACTAAGCTTAAAAAAAAAAAA
GGCCTCCCAAATCGGGGGGCCTTTTTTATTGATAACAAAACGCTACTAAC
TGTCTATGCCTGGGAAAGGGTGGGCAGGAGATGGGGCAGTGCAGGAAAAG
TGGCACTATGAACCCTGCAGCCCTAGTTTGACAATTAATCATTGGCATAG
TATATCTGCATAGTATAATACAACTCACTATAGCAATTGTACTAACCTTC
TTCTCTTTCCTCTCCTGACAGGAGGAGCCATCATGGCCAAGTTGACCAGT
GCCGTTCCGGTGCTCACCGCGCGCGACGTCGCCGGAGCGGTCGAGTTCTG
GACCGACCGGCTCGGGTTCTCCCGGGACTTCGTGGAGGACGACTTCGCCG
GTGTGGTCCGGGACGACGTGACCCTGTTCATCAGCGCGGTCCAGGACCAG
GTGGTGCCGGACAACACCCTGGCCTGGGTGTGGGTGCGCGGCCTGGACGA
GCTGTACGCCGAGTGGTCGGAGGTCGTGTCCACGAACTTCCGGGACGCCT
CCGGGCCGGCCATGACCGAGATCGGCGAGCAGCCGTGGGGGCGGGAGTTC
GCCCTGCGCGACCCGGCCGGCAACTGCGTGCACTTTGTGGCAGAGGAGCA
GGACTGATTACCTGTAACAGAGCATTAGCGCAAGGTGATTTTTGTCTTCT
TGCGCTAATTTTTTCTGGGTTGTTTTACTGCTTCTGCTGGTGGGATTTTT
CGTCCTGGAACCGCTCGGCATTCCGGTGAGCGCCATTGCAGCTGTGGGCG
CGCTGATATTATTTGTCGTCGCTAAACGCGGTCATGCGATTAATACGGGT
AAAGTCCTGCGCGGTGCCCCCTGGCAGATTGTCATCTTCTCGCTCGGCAT
GTATCTGGTGGTTTATGGCCTGCGCAATGCCGGATTAACGGAATATCTTT
CTGGCGTACTCAACGTGCTGGCGGATAACGGCCTGTGGGCCGCGACGCTC
GGCACCGGATTCCTCACCGCCTTCCTCTCTTCTATTATGAACAATATGCC
GACGGTACTGGTTGGCGCGTTGTCCATTGATGGCAGCACGGCATCTGGCG
TTATCAAAGAAGCGATGGTTTATGCCAATGTGATTGGCTGCGATTTGGGA
CCGAAAATTACCCCAATTGGTAGCCTGGCTACGCTACTCTGGCTGCACGT
ACTTTCGCAGAAGAATATGACTATCAGCTGGGGATATTACTTCCGTACAG
GGATTATCATGACCCTGCCTGTGCTGTTTGTGACGCTGGCTGCGCTGGCG
CTACGTCTCTCTTTCACTTTGTAATGAGATACTGATATGAGCAACATTAC
CATTTATCACAACCCGGCCTGCGGCACGTCGCGTAATACGCTGGAGATGA
TCCGCAACAGCGGCACAGAACCGACTATTATCCATTATCTGGAAACTCCG
CCAACGCGCGATGAACTGGTCAAACTCATTGCCGATATGGGGATTTCCGT
ACGCGCGCTGCTGCGTAAAAACGTCGAACCGTATGAGGAGCTGGGCCTTG
CGGAAGATAAATTTACTGACGATCGGTTAATCGACTTTATGCTTCAGCAC
CCGATTCTGATTAATCGCCCGATTGTGGTGACGCCGCTGGGAACTCGCCT
GTGCCGCCCTTCAGAAGTGGTGCTGGAAATTCTGCCAGATGCGCAAAAAG
GCGCATTCTCCAAGGAAGATGGCGAGAAAGTGGTTGATGAAGCGGGTAAG
CGCCTG

>2W213R (Linear DNA sequence for recombineering to generate the stable E. coli strain DH10B-2W213R, with the 20 bp sequence corresponding to the guide RNA sequence guide #360 that specifies dCas9 specificity underlined). (SEQ ID NO: 117)

TTCTGTTACCCATCCAATTGTTCAAAATTCTTGCTGATGAAACCCGTCTG
GGCATCGTTTTACTGCTCAGCGAACTGGGAGAGTTATGCGTCTGCGATCT
CTGCACTGCTCTCGACCAGTCGCAGCCCAAGATCTCCCGCCACCTGGCAT
TGCTGCGTGAAAGCGGGCTATTGCTGGACCGCAAGCAAGGTAAGTGGGTT
CATTACCGCTTATCACCGCATATTCCAGCATGGGCGGCGAAAATTATTGA
TGAGGCCTGGCGATGTGAACAGGAAAAGGTTCAGGCGATTGTCCGCAACC
TGGCTCGACAAAACTGTTCCGGGGACAGTAAGAACATTTGCAGTTAAAAA
TTTAGCTAAACACATATGAATTTTCAGATGTGTTTTATCCGGGAGGCATT
ATGTTACTGGCAGGCGCTATCTTTGTCCTGACCATCGTATTGGTTATCTG
GCAGCCGAAAGGTTTAGGCATCGGCTGGAGTGCAACGCTCGGCGCAGTAC
TGGCGTTAGTTACGGGCGTGGTCCATCCGGGTGATATTCCGGTGGTGTGG
AATATCGTCTGGAACGCGACGGCTGCGTTTATCGCCGTCATTATCATCAG
CCTGCTGCTGGATGAGTCCGGCTTTTTTGAATGGGCGGCGCTGCACGTCT
CACGCTGGGGTAATGGTCGTGGTCGCTTGCTGTTTACCTGGATTGTCCTG
CTCGGTGCTGCCGTTGCCGCCCTGTTTGCCAATGATGGCGCGGCGCTTAT
TTTGACACCGATTGTCATCGCCATGCTGCTGGCTTTAGGGTTCAGTAAAG
GCACTACGCTGGCGTTCGTGATGGCGGCCGGATTCATTGCCGATACCGCC
AGCCTGCCGCTTATTGTCTCCAACCTGGTGAATATCGTTTCCGCTGATTT
CTTTGGCCTCGGCTTTCGCGAATACGCCTCGGTGATGGTGCCGGTGGATA
TCGCCGCGATTGTTGCCACGCTGGTGATGTTACATCTCTATTTTCGCAAA
GATATTCCGCAGAACTACGATATGGCGCTGCTGAAATCTCCCGCAGAAGC
GATCAAAGATCCTGCTACGTTCAAAACTGGGCAAGGAGTTGACAGCTAGC
TCAGTCCTAGGTATAATGCTAGCGTGCAGTACCTCTCACGACTGTTTTAG
AGCTAGAAATAGCAAGTTAAAATAAGGCTAGTCCGTTATCAACTTGAAAA
AGTGGCACCGAGTCGGTGCTTTTTTTGAAGCTTGGGCCCGAACAAAAAGC
TTCTCGGTACCAAATTCCAGAAAAGAGGCCTCCCGAAAGGGGGGCCTTTT
TTCGTTTTGGTCCCGCTAGGTTTGACAGCTAGCTCAGTCCTAGGTATAAT
GCTAGCAGAGAAAGAGGAGAAATACAAATGGATAAGAAATACTCAATAGG
CTTAGCCATCGGCACAAATAGCGTCGGATGGGCGGTGATCACTGATGAAT
ATAAGGTTCCGTCTAAAAAGTTCAAGGTTCTGGGAAATACAGACCGCCAC
AGTATCAAAAAAAATCTTATAGGGGCTCTTTTATTTGACAGTGGAGAGAC
AGCGGAAGCGACTCGTCTCAAACGGACAGCTCGTAGAAGGTATACACGTC
GGAAGAATCGTATTTGTTATCTACAGGAGATTTTTTCAAATGAGATGGCG
AAAGTAGATGATAGTTTCTTTCATCGACTTGAAGAGTCTTTTTTGGTGGA
AGAAGACAAGAAGCATGAACGTCATCCTATTTTTGGAAATATAGTAGATG
AAGTTGCTTATCATGAGAAATATCCAACTATCTATCATCTGCGAAAAAAA
TTGGTAGATTCTACTGATAAAGCGGATTTGCGCTTAATCTATTTGGCCTT
AGCGCATATGATTAAGTTTCGTGGTCATTTTTTGATTGAGGGAGATTTAA
ATCCTGATAATAGTGATGTGGACAAACTATTTATCCAGTTGGTACAAACC
TACAATCAATTATTTGAAGAAAACCCTATTAACGCAAGTGGAGTAGATGC
TAAAGCGATTCTTTCTGCACGATTGAGTAAATCAAGACGATTAGAAAATC
TCATTGCTCAGCTCCCCGGTGAGAAGAAAAATGGCTTATTTGGGAATCTC
ATTGCTTTGTCATTGGGTTTGACCCCTAATTTTAAATCAAATTTTGATTT
GGCAGAAGATGCTAAATTACAGCTTTCAAAAGATACTTACGATGATGATT
TAGATAATTTATTGGCGCAAATTGGAGATCAATATGCTGATTTGTTTTTG
GCAGCTAAGAATTTATCAGATGCTATTTTACTTTCAGATATCCTAAGAGT
AAATACTGAAATAACTAAGGCTCCCCTATCAGCTTCAATGATTAAACGCT
ACGATGAACATCATCAAGACTTGACTCTTTTAAAAGCTTTAGTTCGACAA
CAACTTCCAGAAAAGTATAAAGAAATCTTTTTTGATCAATCAAAAAACGG
ATATGCAGGTTATATTGATGGGGGAGCTAGCCAAGAAGAATTTTATAAAT
TTATCAAACCAATTTTAGAAAAAATGGATGGTACTGAGGAATTATTGGTG
AAACTAAATCGTGAAGATTTGCTGCGCAAGCAACGGACCTTTGACAACGG
CTCTATTCCCCATCAAATTCACTTGGGTGAGCTGCATGCTATTTTGAGAA
GACAAGAAGACTTTTATCCATTTTTAAAAGACAATCGTGAGAAGATTGAA
AAAATCTTGACTTTTCGAATTCCTTATTATGTTGGTCCATTGGCGCGTGG
CAATAGTCGTTTTGCATGGATGACTCGGAAGTCTGAAGAAACAATTACCC
CATGGAATTTTGAAGAAGTTGTCGATAAAGGTGCTTCAGCTCAATCATTT
ATTGAACGCATGACAAACTTTGATAAAAATCTTCCAAATGAAAAAGTACT
ACCAAAACATAGTTTGCTTTATGAGTATTTTACGGTTTATAACGAATTGA
CAAAGGTCAAATATGTTACTGAAGGAATGCGAAAACCAGCATTTCTTTCA
GGTGAACAGAAGAAAGCCATTGTTGATTTACTCTTCAAAACAAATCGAAA
AGTAACCGTTAAGCAATTAAAAGAAGATTATTTCAAAAAAATAGAATGTT
TTGATAGTGTTGAAATTTCAGGAGTTGAAGATAGATTTAATGCTTCATTA
GGTACCTACCATGATTTGCTAAAAATTATTAAAGATAAAGATTTTTTGGA
TAATGAAGAAAATGAAGATATCTTAGAGGATATTGTTTTAACATTGACCT
TATTTGAAGATAGGGAGATGATTGAGGAAAGACTTAAAACATATGCTCAC
CTCTTTGATGATAAGGTGATGAAACAGCTTAAACGTCGCCGTTATACTGG
TTGGGGACGTTTGTCTCGAAAATTGATTAATGGTATTAGGGATAAGCAAT
CTGGCAAAACAATATTAGATTTTTTGAAATCAGATGGTTTTGCCAATCGC
AATTTTATGCAGCTGATCCATGATGATAGTTTGACATTTAAAGAAGACAT
TCAAAAAGCACAAGTGTCTGGACAAGGCGATAGTTTACATGAACATATTG
CAAATTTAGCTGGTAGCCCTGCTATTAAAAAAGGTATTTTACAGACTGTA
AAAGTTGTTGATGAATTGGTCAAAGTAATGGGGCGGCATAAGCCAGAAAA
TATCGTTATTGAAATGGCACGTGAAAATCAGACAACTCAAAAGGGCCAGA
AAAATTCGCGAGAGCGTATGAAACGAATCGAAGAAGGTATCAAAGAATTA
GGAAGTCAGATTCTTAAAGAGCATCCTGTTGAAAATACTCAATTGCAAAA
TGAAAAGCTCTATCTCTATTATCTCCAAAATGGAAGAGACATGTATGTGG
ACCAAGAATTAGATATTAATCGTTTAAGTGATTATGATGTCGATGCCATT
GTTCCACAAAGTTTCCTTAAAGACGATTCAATAGACAATAAGGTCTTAAC
GCGTTCTGATAAAAATCGTGGTAAATCGGATAACGTTCCAAGTGAAGAAG
TAGTCAAAAAGATGAAAAACTATTGGAGACAACTTCTAAACGCCAAGTTA
ATCACTCAACGTAAGTTTGATAATTTAACGAAAGCTGAACGTGGAGGTTT
GAGTGAACTTGATAAAGCTGGTTTTATCAAACGCCAATTGGTTGAAACTC
GCCAAATCACTAAGCATGTGGCACAAATTTTGGATAGTCGCATGAATACT
AAATACGATGAAAATGATAAACTTATTCGAGAGGTTAAAGTGATTACCTT
AAAATCTAAATTAGTTTCTGACTTCCGAAAAGATTTCCAATTCTATAAAG
TACGTGAGATTAACAATTACCATCATGCCCATGATGCGTATCTAAATGCC
GTCGTTGGAACTGCTTTGATTAAGAAATATCCAAAACTTGAATCGGAGTT
TGTCTATGGTGATTATAAAGTTTATGATGTTCGTAAAATGATTGCTAAGT
CTGAGCAAGAAATAGGCAAAGCAACCGCAAAATATTTCTTTTACTCTAAT
ATCATGAACTTCTTCAAAACAGAAATTACACTTGCAAATGGAGAGATTCG
CAAACGCCCTCTAATCGAAACTAATGGGGAAACTGGAGAAATTGTCTGGG
ATAAAGGGCGAGATTTTGCCACAGTGCGCAAAGTATTGTCCATGCCCCAA
GTCAATATTGTCAAGAAAACAGAAGTACAGACAGGCGGATTCTCCAAGGA
GTCAATTTTACCAAAAAGAAATTCGGACAAGCTTATTGCTCGTAAAAAAG
ACTGGGATCCAAAAAAATATGGTGGTTTTGATAGTCCAACGGTAGCTTAT
TCAGTCCTAGTGGTTGCTAAGGTGGAAAAAGGGAAATCGAAGAAGTTAAA
ATCCGTTAAAGAGTTACTAGGGATCACAATTATGGAAAGAAGTTCCTTTG
AAAAAAATCCGATTGACTTTTTAGAAGCTAAAGGATATAAGGAAGTTAAA
AAAGACTTAATCATTAAACTACCTAAATATAGTCTTTTTGAGTTAGAAAA
CGGTCGTAAACGGATGCTGGCTAGTGCCGGAGAATTACAAAAAGGAAATG
AGCTGGCTCTGCCAAGCAAATATGTGAATTTTTTATATTTAGCTAGTCAT
TATGAAAAGTTGAAGGGTAGTCCAGAAGATAACGAACAAAAACAATTGTT
TGTGGAGCAGCATAAGCATTATTTAGATGAGATTATTGAGCAAATCAGTG
AATTTTCTAAGCGTGTTATTTTAGCAGATGCCAATTTAGATAAAGTTCTT
AGTGCATATAACAAACATAGAGACAAACCAATACGTGAACAAGCAGAAAA
TATTATTCATTTATTTACGTTGACGAATCTTGGAGCTCCCGCTGCTTTTA
AATATTTTGATACAACAATTGATCGTAAACGATATACGTCTACAAAAGAA
GTTTTAGATGCCACTCTTATCCATCAATCCATCACTGGTCTTTATGAAAC
ACGCATTGATTTGAGTCAGCTAGGAGGTGACTAAGCTTAAAAAAAAAAAA
GGCCTCCCAAATCGGGGGGCCTTTTTTATTGATAACAAAACGCTTGCCGG
AGCTGATAGCTAGCTCAGTCCTAGGGATTATGCTAGCTACTAGAGAAAGA
GGAGAAATACAAATGACCAAGTCTGAAACATTCATGATTCCAAACCACAA
GGCCGCCAAATTAAGTGAGCTGGATATGATGATCGTTAACTCTGTCCCGC
CTGGGGGAAACTGGAAGAATATTCCCTTGGATGTACCATCGAAACGTATC
GAACAGATTCGTGACAGCTATGCTCAAGGAAAAGGGTCGCGCAGCACATA
CTACGGGCGTTTATTGCCCGATATGCCAGCTTATACGATCAATACTTATT
TCAATCGTCCTGGCAACGGATGCCACATTCACTACGAGCAAGATCGCGTA
CTTTCACAACGCGAAGCTGCACGTCTGCAGTCGTTCCCTGATGACTTTAT
CTTTTTTGGAGGTCAAACGGCGATTAATACGCAAATCGGTAATGCCGTGC
CTCCCTTTCTTGCGTTTCTTATTGCAAAAGAAATTGAAAAAGCGATCGGT
AATACCGGCTACTACATTGACTTATTCAGTGGTGCAGGCGGATTGGGGTT
GGGCTTTAAGTGGGCCGGGTGGACTCCATTGTTAGCTAATGACATTGAGG
AAAAGTACTTACAGACATACTCGAACAACGTACACAAAGAAGTTTTGTGC
GGAAGCATTTCGGACAACGAAACTTTTTCTAAGATCGCAGACAAGATTTC
TGGCTTTAAGAAATTATATTTTGATAAACAGCTGTGGATTCTGGGCGGGC
CTCCGTGCCAGGGATTTAGCACGGCTGGCAACGCGCGTACAATGGACGAC
CCACGCAACAGTCTGTTTATGCACTACAAGTCGCTGCTTAACGAGATTAA
GCCGAATGGATTCATTTTCGAGAACGTCGCCGGCCTGTTGAACATGGAAA
AAGGAAAGGTCTTTGAACGTGTTAAGGAGGAATTCTCGTCCACAATGAAA
ACCATGAATGGTTGGATTTTAAATTCGGAACATTACGCAATTCCACAACG
CCGTAAGCGTGTAATTCTTGTGGGCAGCAATGATCCGCTGTTCTCGATCG
AACCACCTCAGAAGCTGACGGAAGATAAAGAGTCTTGGGTGTCAGTAAAA
GATGCGTTATCTGACCTTCCCCCATTACAACACGGCGAGGATGGATCTGG
TAAATACTATATCCACCACCCGGAAAATGATTACCAGTTGTTTATGCGTG
GAAACATTACACCCTCAGAGTATTATGAACGCAACATTAAGCCGTCGCTT
TAAGCTTGACGAACAATAAGGCCTCCCAAATCGGGGGGCCTTTTTATTTT
TCAACAAAACGCTACTAACTGTCTATGCCTGGGAAAGGGTGGGCAGGAGA
TGGGGCAGTGCAGGAAAAGTGGCACTATGAACCCTGCAGCCCTAGTTTGA
CAATTAATCATTGGCATAGTATATCTGCATAGTATAATACAACTCACTAT
AGCAATTGTACTAACCTTCTTCTCTTTCCTCTCCTGACAGGAGGAGCCAT
CATGGCCAAGTTGACCAGTGCCGTTCCGGTGCTCACCGCGCGCGACGTCG
CCGGAGCGGTCGAGTTCTGGACCGACCGGCTCGGGTTCTCCCGGGACTTC
GTGGAGGACGACTTCGCCGGTGTGGTCCGGGACGACGTGACCCTGTTCAT
CAGCGCGGTCCAGGACCAGGTGGTGCCGGACAACACCCTGGCCTGGGTGT
GGGTGCGCGGCCTGGACGAGCTGTACGCCGAGTGGTCGGAGGTCGTGTCC
ACGAACTTCCGGGACGCCTCCGGGCCGGCCATGACCGAGATCGGCGAGCA
GCCGTGGGGGCGGGAGTTCGCCCTGCGCGACCCGGCCGGCAACTGCGTGC
ACTTTGTGGCAGAGGAGCAGGACTGATTACCTGTAACAGAGCATTAGCGC
AAGGTGATTTTTGTCTTCTTGCGCTAATTTTTTCTGGGTTGTTTTACTGC
TTCTGCTGGTGGGATTTTTCGTCCTGGAACCGCTCGGCATTCCGGTGAGC
GCCATTGCAGCTGTGGGCGCGCTGATATTATTTGTCGTCGCTAAACGCGG
TCATGCGATTAATACGGGTAAAGTCCTGCGCGGTGCCCCCTGGCAGATTG
TCATCTTCTCGCTCGGCATGTATCTGGTGGTTTATGGCCTGCGCAATGCC
GGATTAACGGAATATCTTTCTGGCGTACTCAACGTGCTGGCGGATAACGG
CCTGTGGGCCGCGACGCTCGGCACCGGATTCCTCACCGCCTTCCTCTCTT
CTATTATGAACAATATGCCGACGGTACTGGTTGGCGCGTTGTCCATTGAT
GGCAGCACGGCATCTGGCGTTATCAAAGAAGCGATGGTTTATGCCAATGT
GATTGGCTGCGATTTGGGACCGAAAATTACCCCAATTGGTAGCCTGGCTA
CGCTACTCTGGCTGCACGTACTTTCGCAGAAGAATATGACTATCAGCTGG
GGATATTACTTCCGTACAGGGATTATCATGACCCTGCCTGTGCTGTTTGT
GACGCTGGCTGCGCTGGCGCTACGTCTCTCTTTCACTTTGTAATGAGATA
CTGATATGAGCAACATTACCATTTATCACAACCCGGCCTGCGGCACGTCG
CGTAATACGCTGGAGATGATCCGCAACAGCGGCACAGAACCGACTATTAT
CCATTATCTGGAAACTCCGCCAACGCGCGATGAACTGGTCAAACTCATTG

CCGATATGGGGATTTCCGTACGCGCGCTGCTGCGTAAAAACGTCGAACCG

TATGAGGAGCTGGGCCTTGCGGAAGATAAATTTACTGACGATCGGTTAAT
CGACTTTATGCTTCAGCACCCGATTCTGATTAATCGCCCGATTGTGGTGA
CGCCGCTGGGAACTCGCCTGTGCCGCCCTTCAGAAGTGGTGCTGGAAATT
CTGCCAGATGCGCAAAAAGGCGCATTCTCCAAGGAAGATGGCGAGAAAGT
GGTTGATGAAGCGGGTAAGCGCCTG

>2W214R (Linear DNA sequence for recombineering to generate the stable E. coli strain DH10B-2W214R, with the 20 bp sequence corresponding to the guide RNA sequence guide #401 that specifies dCas9 specificity underlined). (SEQ ID NO: 118)

TTCTGTTACCCATCCAATTGTTCAAAATTCTTGCTGATGAAACCCGTCTG
GGCATCGTTTTACTGCTCAGCGAACTGGGAGAGTTATGCGTCTGCGATCT
CTGCACTGCTCTCGACCAGTCGCAGCCCAAGATCTCCCGCCACCTGGCAT
TGCTGCGTGAAAGCGGGCTATTGCTGGACCGCAAGCAAGGTAAGTGGGTT
CATTACCGCTTATCACCGCATATTCCAGCATGGGCGGCGAAAATTATTGA
TGAGGCCTGGCGATGTGAACAGGAAAAGGTTCAGGCGATTGTCCGCAACC
TGGCTCGACAAAACTGTTCCGGGGACAGTAAGAACATTTGCAGTTAAAAA
TTTAGCTAAACACATATGAATTTTCAGATGTGTTTTATCCGGGAGGCATT
ATGTTACTGGCAGGCGCTATCTTTGTCCTGACCATCGTATTGGTTATCTG
GCAGCCGAAAGGTTTAGGCATCGGCTGGAGTGCAACGCTCGGCGCAGTAC
TGGCGTTAGTTACGGGCGTGGTCCATCCGGGTGATATTCCGGTGGTGTGG
AATATCGTCTGGAACGCGACGGCTGCGTTTATCGCCGTCATTATCATCAG
CCTGCTGCTGGATGAGTCCGGCTTTTTTGAATGGGCGGCGCTGCACGTCT
CACGCTGGGGTAATGGTCGTGGTCGCTTGCTGTTTACCTGGATTGTCCTG
CTCGGTGCTGCCGTTGCCGCCCTGTTTGCCAATGATGGCGCGGCGCTTAT
TTTGACACCGATTGTCATCGCCATGCTGCTGGCTTTAGGGTTCAGTAAAG
GCACTACGCTGGCGTTCGTGATGGCGGCCGGATTCATTGCCGATACCGCC
AGCCTGCCGCTTATTGTCTCCAACCTGGTGAATATCGTTTCCGCTGATTT
CTTTGGCCTCGGCTTTCGCGAATACGCCTCGGTGATGGTGCCGGTGGATA
TCGCCGCGATTGTTGCCACGCTGGTGATGTTACATCTCTATTTTCGCAAA
GATATTCCGCAGAACTACGATATGGCGCTGCTGAAATCTCCCGCAGAAGC
GATCAAAGATCCTGCTACGTTCAAAACTGGGCAAGGAGTTGACAGCTAGC
TCAGTCCTAGGTATAATGCTAGCGAGTAATCACGCCAGTGCATGTTTTAG
AGCTAGAAATAGCAAGTTAAAATAAGGCTAGTCCGTTATCAACTTGAAAA
AGTGGCACCGAGTCGGTGCTTTTTTTGAAGCTTGGGCCCGAACAAAAAGC
TTCTCGGTACCAAATTCCAGAAAAGAGGCCTCCCGAAAGGGGGGCCTTTT
TTCGTTTTGGTCCCGCTAGGTTTGACAGCTAGCTCAGTCCTAGGTATAAT
GCTAGCAGAGAAAGAGGAGAAATACAAATGGATAAGAAATACTCAATAGG
CTTAGCCATCGGCACAAATAGCGTCGGATGGGCGGTGATCACTGATGAAT
ATAAGGTTCCGTCTAAAAAGTTCAAGGTTCTGGGAAATACAGACCGCCAC
AGTATCAAAAAAAATCTTATAGGGGCTCTTTTATTTGACAGTGGAGAGAC
AGCGGAAGCGACTCGTCTCAAACGGACAGCTCGTAGAAGGTATACACGTC
GGAAGAATCGTATTTGTTATCTACAGGAGATTTTTTCAAATGAGATGGCG
AAAGTAGATGATAGTTTCTTTCATCGACTTGAAGAGTCTTTTTTGGTGGA
AGAAGACAAGAAGCATGAACGTCATCCTATTTTTGGAAATATAGTAGATG
AAGTTGCTTATCATGAGAAATATCCAACTATCTATCATCTGCGAAAAAAA
TTGGTAGATTCTACTGATAAAGCGGATTTGCGCTTAATCTATTTGGCCTT
AGCGCATATGATTAAGTTTCGTGGTCATTTTTTGATTGAGGGAGATTTAA
ATCCTGATAATAGTGATGTGGACAAACTATTTATCCAGTTGGTACAAACC
TACAATCAATTATTTGAAGAAAACCCTATTAACGCAAGTGGAGTAGATGC
TAAAGCGATTCTTTCTGCACGATTGAGTAAATCAAGACGATTAGAAAATC
TCATTGCTCAGCTCCCCGGTGAGAAGAAAAATGGCTTATTTGGGAATCTC
ATTGCTTTGTCATTGGGTTTGACCCCTAATTTTAAATCAAATTTTGATTT
GGCAGAAGATGCTAAATTACAGCTTTCAAAAGATACTTACGATGATGATT
TAGATAATTTATTGGCGCAAATTGGAGATCAATATGCTGATTTGTTTTTG
GCAGCTAAGAATTTATCAGATGCTATTTTACTTTCAGATATCCTAAGAGT
AAATACTGAAATAACTAAGGCTCCCCTATCAGCTTCAATGATTAAACGCT
ACGATGAACATCATCAAGACTTGACTCTTTTAAAAGCTTTAGTTCGACAA
CAACTTCCAGAAAAGTATAAAGAAATCTTTTTTGATCAATCAAAAAACGG
ATATGCAGGTTATATTGATGGGGGAGCTAGCCAAGAAGAATTTTATAAAT
TTATCAAACCAATTTTAGAAAAAATGGATGGTACTGAGGAATTATTGGTG
AAACTAAATCGTGAAGATTTGCTGCGCAAGCAACGGACCTTTGACAACGG
CTCTATTCCCCATCAAATTCACTTGGGTGAGCTGCATGCTATTTTGAGAA
GACAAGAAGACTTTTATCCATTTTTAAAAGACAATCGTGAGAAGATTGAA
AAAATCTTGACTTTTCGAATTCCTTATTATGTTGGTCCATTGGCGCGTGG
CAATAGTCGTTTTGCATGGATGACTCGGAAGTCTGAAGAAACAATTACCC
CATGGAATTTTGAAGAAGTTGTCGATAAAGGTGCTTCAGCTCAATCATTT
ATTGAACGCATGACAAACTTTGATAAAAATCTTCCAAATGAAAAAGTACT
ACCAAAACATAGTTTGCTTTATGAGTATTTTACGGTTTATAACGAATTGA
CAAAGGTCAAATATGTTACTGAAGGAATGCGAAAACCAGCATTTCTTTCA
GGTGAACAGAAGAAAGCCATTGTTGATTTACTCTTCAAAACAAATCGAAA
AGTAACCGTTAAGCAATTAAAAGAAGATTATTTCAAAAAAATAGAATGTT
TTGATAGTGTTGAAATTTCAGGAGTTGAAGATAGATTTAATGCTTCATTA
GGTACCTACCATGATTTGCTAAAAATTATTAAAGATAAAGATTTTTTGGA
TAATGAAGAAAATGAAGATATCTTAGAGGATATTGTTTTAACATTGACCT
TATTTGAAGATAGGGAGATGATTGAGGAAAGACTTAAAACATATGCTCAC
CTCTTTGATGATAAGGTGATGAAACAGCTTAAACGTCGCCGTTATACTGG
TTGGGGACGTTTGTCTCGAAAATTGATTAATGGTATTAGGGATAAGCAAT
CTGGCAAAACAATATTAGATTTTTTGAAATCAGATGGTTTTGCCAATCGC
AATTTTATGCAGCTGATCCATGATGATAGTTTGACATTTAAAGAAGACAT
TCAAAAAGCACAAGTGTCTGGACAAGGCGATAGTTTACATGAACATATTG
CAAATTTAGCTGGTAGCCCTGCTATTAAAAAAGGTATTTTACAGACTGTA
AAAGTTGTTGATGAATTGGTCAAAGTAATGGGGCGGCATAAGCCAGAAAA
TATCGTTATTGAAATGGCACGTGAAAATCAGACAACTCAAAAGGGCCAGA
AAAATTCGCGAGAGCGTATGAAACGAATCGAAGAAGGTATCAAAGAATTA
GGAAGTCAGATTCTTAAAGAGCATCCTGTTGAAAATACTCAATTGCAAAA
TGAAAAGCTCTATCTCTATTATCTCCAAAATGGAAGAGACATGTATGTGG
ACCAAGAATTAGATATTAATCGTTTAAGTGATTATGATGTCGATGCCATT
GTTCCACAAAGTTTCCTTAAAGACGATTCAATAGACAATAAGGTCTTAAC
GCGTTCTGATAAAAATCGTGGTAAATCGGATAACGTTCCAAGTGAAGAAG
TAGTCAAAAAGATGAAAAACTATTGGAGACAACTTCTAAACGCCAAGTTA
ATCACTCAACGTAAGTTTGATAATTTAACGAAAGCTGAACGTGGAGGTTT
GAGTGAACTTGATAAAGCTGGTTTTATCAAACGCCAATTGGTTGAAACTC
GCCAAATCACTAAGCATGTGGCACAAATTTTGGATAGTCGCATGAATACT
AAATACGATGAAAATGATAAACTTATTCGAGAGGTTAAAGTGATTACCTT
AAAATCTAAATTAGTTTCTGACTTCCGAAAAGATTTCCAATTCTATAAAG
TACGTGAGATTAACAATTACCATCATGCCCATGATGCGTATCTAAATGCC
GTCGTTGGAACTGCTTTGATTAAGAAATATCCAAAACTTGAATCGGAGTT
TGTCTATGGTGATTATAAAGTTTATGATGTTCGTAAAATGATTGCTAAGT
CTGAGCAAGAAATAGGCAAAGCAACCGCAAAATATTTCTTTTACTCTAAT
ATCATGAACTTCTTCAAAACAGAAATTACACTTGCAAATGGAGAGATTCG
CAAACGCCCTCTAATCGAAACTAATGGGGAAACTGGAGAAATTGTCTGGG
ATAAAGGGCGAGATTTTGCCACAGTGCGCAAAGTATTGTCCATGCCCCAA
GTCAATATTGTCAAGAAAACAGAAGTACAGACAGGCGGATTCTCCAAGGA
GTCAATTTTACCAAAAAGAAATTCGGACAAGCTTATTGCTCGTAAAAAAG
ACTGGGATCCAAAAAAATATGGTGGTTTTGATAGTCCAACGGTAGCTTAT
TCAGTCCTAGTGGTTGCTAAGGTGGAAAAAGGGAAATCGAAGAAGTTAAA
ATCCGTTAAAGAGTTACTAGGGATCACAATTATGGAAAGAAGTTCCTTTG
AAAAAAATCCGATTGACTTTTTAGAAGCTAAAGGATATAAGGAAGTTAAA
AAAGACTTAATCATTAAACTACCTAAATATAGTCTTTTTGAGTTAGAAAA
CGGTCGTAAACGGATGCTGGCTAGTGCCGGAGAATTACAAAAAGGAAATG
AGCTGGCTCTGCCAAGCAAATATGTGAATTTTTTATATTTAGCTAGTCAT
TATGAAAAGTTGAAGGGTAGTCCAGAAGATAACGAACAAAAACAATTGTT
TGTGGAGCAGCATAAGCATTATTTAGATGAGATTATTGAGCAAATCAGTG
AATTTTCTAAGCGTGTTATTTTAGCAGATGCCAATTTAGATAAAGTTCTT
AGTGCATATAACAAACATAGAGACAAACCAATACGTGAACAAGCAGAAAA
TATTATTCATTTATTTACGTTGACGAATCTTGGAGCTCCCGCTGCTTTTA
AATATTTTGATACAACAATTGATCGTAAACGATATACGTCTACAAAAGAA
GTTTTAGATGCCACTCTTATCCATCAATCCATCACTGGTCTTTATGAAAC
ACGCATTGATTTGAGTCAGCTAGGAGGTGACTAAGCTTAAAAAAAAAAAA
GGCCTCCCAAATCGGGGGGCCTTTTTTATTGATAACAAAACGCTTGCCGG
AGCTGATAGCTAGCTCAGTCCTAGGGATTATGCTAGCTACTAGAGAAAGA
GGAGAAATACAAATGACCAAGTCTGAAACATTCATGATTCCAAACCACAA
GGCCGCCAAATTAAGTGAGCTGGATATGATGATCGTTAACTCTGTCCCGC
CTGGGGGAAACTGGAAGAATATTCCCTTGGATGTACCATCGAAACGTATC
GAACAGATTCGTGACAGCTATGCTCAAGGAAAAGGGTCGCGCAGCACATA
CTACGGGCGTTTATTGCCCGATATGCCAGCTTATACGATCAATACTTATT
TCAATCGTCCTGGCAACGGATGCCACATTCACTACGAGCAAGATCGCGTA
CTTTCACAACGCGAAGCTGCACGTCTGCAGTCGTTCCCTGATGACTTTAT
CTTTTTTGGAGGTCAAACGGCGATTAATACGCAAATCGGTAATGCCGTGC
CTCCCTTTCTTGCGTTTCTTATTGCAAAAGAAATTGAAAAAGCGATCGGT
AATACCGGCTACTACATTGACTTATTCAGTGGTGCAGGCGGATTGGGGTT
GGGCTTTAAGTGGGCCGGGTGGACTCCATTGTTAGCTAATGACATTGAGG
AAAAGTACTTACAGACATACTCGAACAACGTACACAAAGAAGTTTTGTGC
GGAAGCATTTCGGACAACGAAACTTTTTCTAAGATCGCAGACAAGATTTC
TGGCTTTAAGAAATTATATTTTGATAAACAGCTGTGGATTCTGGGCGGGC
CTCCGTGCCAGGGATTTAGCACGGCTGGCAACGCGCGTACAATGGACGAC
CCACGCAACAGTCTGTTTATGCACTACAAGTCGCTGCTTAACGAGATTAA
GCCGAATGGATTCATTTTCGAGAACGTCGCCGGCCTGTTGAACATGGAAA
AAGGAAAGGTCTTTGAACGTGTTAAGGAGGAATTCTCGTCCACAATGAAA
ACCATGAATGGTTGGATTTTAAATTCGGAACATTACGCAATTCCACAACG
CCGTAAGCGTGTAATTCTTGTGGGCAGCAATGATCCGCTGTTCTCGATCG
AACCACCTCAGAAGCTGACGGAAGATAAAGAGTCTTGGGTGTCAGTAAAA
GATGCGTTATCTGACCTTCCCCCATTACAACACGGCGAGGATGGATCTGG
TAAATACTATATCCACCACCCGGAAAATGATTACCAGTTGTTTATGCGTG
GAAACATTACACCCTCAGAGTATTATGAACGCAACATTAAGCCGTCGCTT
TAAGCTTGACGAACAATAAGGCCTCCCAAATCGGGGGGCCTTTTTATTTT
TCAACAAAACGCTACTAACTGTCTATGCCTGGGAAAGGGTGGGCAGGAGA
TGGGGCAGTGCAGGAAAAGTGGCACTATGAACCCTGCAGCCCTAGTTTGA
CAATTAATCATTGGCATAGTATATCTGCATAGTATAATACAACTCACTAT
AGCAATTGTACTAACCTTCTTCTCTTTCCTCTCCTGACAGGAGGAGCCAT
CATGGCCAAGTTGACCAGTGCCGTTCCGGTGCTCACCGCGCGCGACGTCG
CCGGAGCGGTCGAGTTCTGGACCGACCGGCTCGGGTTCTCCCGGGACTTC
GTGGAGGACGACTTCGCCGGTGTGGTCCGGGACGACGTGACCCTGTTCAT
CAGCGCGGTCCAGGACCAGGTGGTGCCGGACAACACCCTGGCCTGGGTGT
GGGTGCGCGGCCTGGACGAGCTGTACGCCGAGTGGTCGGAGGTCGTGTCC
ACGAACTTCCGGGACGCCTCCGGGCCGGCCATGACCGAGATCGGCGAGCA
GCCGTGGGGGCGGGAGTTCGCCCTGCGCGACCCGGCCGGCAACTGCGTGC
ACTTTGTGGCAGAGGAGCAGGACTGATTACCTGTAACAGAGCATTAGCGC
AAGGTGATTTTTGTCTTCTTGCGCTAATTTTTTCTGGGTTGTTTTACTGC
TTCTGCTGGTGGGATTTTTCGTCCTGGAACCGCTCGGCATTCCGGTGAGC
GCCATTGCAGCTGTGGGCGCGCTGATATTATTTGTCGTCGCTAAACGCGG
TCATGCGATTAATACGGGTAAAGTCCTGCGCGGTGCCCCCTGGCAGATTG
TCATCTTCTCGCTCGGCATGTATCTGGTGGTTTATGGCCTGCGCAATGCC
GGATTAACGGAATATCTTTCTGGCGTACTCAACGTGCTGGCGGATAACGG
CCTGTGGGCCGCGACGCTCGGCACCGGATTCCTCACCGCCTTCCTCTCTT
CTATTATGAACAATATGCCGACGGTACTGGTTGGCGCGTTGTCCATTGAT
GGCAGCACGGCATCTGGCGTTATCAAAGAAGCGATGGTTTATGCCAATGT
GATTGGCTGCGATTTGGGACCGAAAATTACCCCAATTGGTAGCCTGGCTA
CGCTACTCTGGCTGCACGTACTTTCGCAGAAGAATATGACTATCAGCTGG
GGATATTACTTCCGTACAGGGATTATCATGACCCTGCCTGTGCTGTTTGT
GACGCTGGCTGCGCTGGCGCTACGTCTCTCTTTCACTTTGTAATGAGATA
CTGATATGAGCAACATTACCATTTATCACAACCCGGCCTGCGGCACGTCG
CGTAATACGCTGGAGATGATCCGCAACAGCGGCACAGAACCGACTATTAT
CCATTATCTGGAAACTCCGCCAACGCGCGATGAACTGGTCAAACTCATTG
CCGATATGGGGATTTCCGTACGCGCGCTGCTGCGTAAAAACGTCGAACCG
TATGAGGAGCTGGGCCTTGCGGAAGATAAATTTACTGACGATCGGTTAAT
CGACTTTATGCTTCAGCACCCGATTCTGATTAATCGCCCGATTGTGGTGA
CGCCGCTGGGAACTCGCCTGTGCCGCCCTTCAGAAGTGGTGCTGGAAATT
CTGCCAGATGCGCAAAAAGGCGCATTCTCCAAGGAAGATGGCGAGAAAGT
GGTTGATGAAGCGGGTAAGCGCCTG

>2W89R (Linear DNA sequence for recombineering to generate the stable E. coli strain DH10B-2W89R) (SEQ ID NO: 119)

TTCTGTTACCCATCCAATTGTTCAAAATTC
TTGCTGATGAAACCCGTCTGGGCATCGTTTTACTGCTCAGCGAACTGGGA
GAGTTATGCGTCTGCGATCTCTGCACTGCTCTCGACCAGTCGCAGCCCAA
GATCTCCCGCCACCTGGCATTGCTGCGTGAAAGCGGGCTATTGCTGGACC
GCAAGCAAGGTAAGTGGGTTCATTACCGCTTATCACCGCATATTCCAGCA
TGGGCGGCGAAAATTATTGATGAGGCCTGGCGATGTGAACAGGAAAAGGT
TCAGGCGATTGTCCGCAACCTGGCTCGACAAAACTGTTCCGGGGACAGTA
AGAACATTTGCAGTTAAAAATTTAGCTAAACACATATGAATTTTCAGATG
TGTTTTATCCGGGAGGCATTATGTTACTGGCAGGCGCTATCTTTGTCCTG
ACCATCGTATTGGTTATCTGGCAGCCGAAAGGTTTAGGCATCGGCTGGAG
TGCAACGCTCGGCGCAGTACTGGCGTTAGTTACGGGCGTGGTCCATCCGG
GTGATATTCCGGTGGTGTGGAATATCGTCTGGAACGCGACGGCTGCGTTT
ATCGCCGTCATTATCATCAGCCTGCTGCTGGATGAGTCCGGCTTTTTTGA
ATGGGCGGCGCTGCACGTCTCACGCTGGGGTAATGGTCGTGGTCGCTTGC
TGTTTACCTGGATTGTCCTGCTCGGTGCTGCCGTTGCCGCCCTGTTTGCC
AATGATGGCGCGGCGCTTATTTTGACACCGATTGTCATCGCCATGCTGCT
GGCTTTAGGGTTCAGTAAAGGCACTACGCTGGCGTTCGTGATGGCGGCCG
GATTCATTGCCGATACCGCCAGCCTGCCGCTTATTGTCTCCAACCTGGTG
AATATCGTTTCCGCTGATTTCTTTGGCCTCGGCTTTCGCGAATACGCCTC
GGTGATGGTGCCGGTGGATATCGCCGCGATTGTTGCCACGCTGGTGATGT
TACATCTCTATTTTCGCAAAGATATTCCGCAGAACTACGATATGGCGCTG
CTGAAATCTCCCGCAGAAGCGATCAAAGATCCTGCTACGTTCAAAACTGG
GCAAGGAG
CTGATAGCTAGCTCAGTCCTAGGGATTATGCTAGCTACTAG
AGAAAGAGGAGAAATACAAATGGAGGAGATCTTCTATATGAAACACATCC
ATTTAATCAACAGCCTGTCGCTGGACGAAACTACTAAGTTCACGAAAAAA
GCTACCGGTAAATACTACACCGACCCCAAAATCGCGCTGTTAATGATCGA
GAAGTTACTTCCGCTGATTAACTCCTGTGATAAAAAGAGCTATAACGTTG
CTGATCCCTTTTCAGGTGACGGTCGTCTGATCACTCTTTTGATCAAGCAG
TGGATGATTAACGGCTTCCCCGATGTCGAATGGAACGTCTACCTGTTTGA
CATTGAGAACACCGGTCTGACTTACGCCAAAAACGCTCTGTCGGAATTGA
AGTTAGCCGGCGCTAATATCAATATTACAATTAAGAACTCGGATGTATTT
TACGAATTTAAGAAGTATGTAGACTACTTCGACTGTGTGATCACAAATCC
TCCCTGGGAGAACATTAAACCTGATTCTCGTGAGCTTGATTTTTTCGAAC
CAAGCATGAAATCCATGTATATTGACAGCCTTCGTGAATTTGATGATTAC
CTTTCACGTGTGCTTCCCTATAGTCAGCCGAAGCGCAAGTTCGCTGGCTG
GGGTACGAACTTAAGTCGTGTTGGTGCAGAGTTATCTCTTGAGATTTGCA
ACAAAAATGGGTTAGTAGCGATCGTCATGCCCGCCAGCTTTTTTGCGGAC
GAGCAATCCTATATTTTACGTGAGAAGTTTTTCAATTCCGGACGTATCGA
CTGTATTAACTATTATCCAGCCGAAGCAAAACTTTTCGGTGGAGCAGATG
TGAGCTCCTGCTCCTTCATTTTTAACAAGGGAGAATCCTTGAATGATAAT
ATTCAATTAAGCGTCTACGATAAAAATTTGAACATCAAGTCATTGGGTTT
TTTCGATTTGTCAAGCATCGATTCCCAATATCTGTCGATCCCCGTGTCTC
AAGGCGTGCATGCGGTCCATCTTTTGCGCAAACTTCAAGAGGGTTACCCA
ACGTGGGGTAGTTTAGAAAAGAACGGCGAGATTTGGGCGGGCCGTGAAAT
CGACGAAACTGGCTCGTCGGATTGGACTCAGAAGTCGGGTGGGGGGCTGT
TGTTTATTAAGGGCAAAATGATTGGCCGCTACAATTTCCACAATGAAAAG
TCTCTGCGCATTACGAAAAAGATTGACAAGGTGCTTTCCAATAGCAACTT
CGTCCGCATCGCCTGGCGCGATATTTCGCGCCCAAGCCAAAAACGCCGCA
TGATCGCAACTATCATTCCGCCTAACTCGTTGGCTGGTAACTCATTGGGT
GTAGTATACTACAAATCCGGGTCCCAGGATTCCCTGTTTTCCTTGCTTGG
AATTATTAACTCTCTGTGCTTCGAATTTCAATTGCGCTCCTTTTTGGCTA
CTGGGCATGTTAGTCTGTCTGCTCTTCGTAAAACCGCGATCCCTAGCGAA
AAGATCTTACTGCAACACAGTGAGTTGAAACAGCTGGTAATTAGCTGCAT
TGAGGGGTGCTGCGATGCGGAATTAAAGATTGAAGCATATGTGGCGAAGA
ACATTTACAAACTTGACCTGAATGAGTTCAACAAATTGCTTAGTAGCTTC
GACAAAATCGAGTTGGCAGAAAAAGAGTCTTTGTTACGCATCTTCCAGCA
CTACGATTAAGCTTCTCGGTACCAAATTCCAGAAAAGAGGCCTCCCGAAA
GGGGGGCCTTTTTTCGTTTTGGTCCCGCTAGGTGGAGTTGACGGCTAGCT
CAGTCCTAGGTACAGTGCTAGCTACTAGAGAAAGAGGAGAAATACAAATG
GCTATCACTAACTTCGTAAAGCGCATCCAAGATGTGATGCGTAATGACGC
TGGAGTTAATGGGGATGCACAACGCATCGAGCAAATCGTATGGATCTTAT
TTTTAAAGATTTATGATGCAAAGGAAGAGGAGTGGGAGTTGGTAAACGAA
AAGTATTCCTCGATTATCCCGGAGGAATGTAAATGGCGTAATTGGGCCGT
AGATGAAAAGGACGGTGAAGCTCTGACAGGCGACGCGATTTTAAACTTTG
TCAATAACACACTGTTCCCTATCCTGAAAAATTTAGAGATTGATGACGAA
ACCCCGATGAATCAGGTCATCGTGAAGTACGCTTTCGAGGACGCCAACAA
TTATATGAAAGATGGAGTTCTTCTTCGCCAGGTAGTTAATATCATTGATG
AGATTGATTTCACTGAATATGAAGAGCGCCATGCATTTGGTACGATTTAT
GAGAGCTTTTTAAAAGATCTTCAAAGTGCGGGTAACGCTGGTGAGTTCTA
CACGCCCCGCGCCGTGACCGACTTTATGGTCGAAGTGATCAAACCAAAGC
TTGGAGAGAAAGTAGCCGACTTTGCTTGCGGCACAGGGGGGTTCTTAGTG
TCTGCGTTGAATGCTCTTGAGAAACAGGTTGGGAATTCACTTGAGAATCG
CGAGATCTATAATAATTCTATTTACGGTGTGGAAAAGAAAGGGCTGCCCC
ATATTCTGTGTGTGACAAATATGTTGTTGCATGACATCGACAACCCGGAC
ATCATTCACGGGAACACACTGGAAACAGATTACAAGGAGTACCGCCGTAT
GGAACAATTCGACATTGTACTGATGAACCCGCCATACGGTGGGAATGAAA
AGGAGAGTGTTAAGGCTAACTTCCCTTCGGATCTTCGCTCCTCCGAGACA
GCTGACTTATTTATCGATATTATTATGTTTCGTTTGAAGAAAAATGGGCG
TTGTGCGATCATTCTTCCTGACGGCTTTCTGTTTGGTACTGATAACGCTA
AGGTCAACATTAAGAAGAAGTTACTTGGGGAATTTAACTTGCACACAATC
GTCCGCCTTCCCCACTCTGTGTTCGCGCCTTACACCTCGATTACGACTAA
TATTCTTTTTTTCGACAATACGCACCCTACCGAAGAAACATGGTTTTATC
GCCTGGACATGCCCGATGGGTATAAGAATTTTTCTAAAACAAAACCGATG
AAGTTAGAGCACTTTGGGTCGGCAATTGAATGGTGGGACAACCGTGAGGA
GATCGAAGTTGATGGATTTCCGAAAGCGAAGAAATACACCGTCGAGGACA
TTGAAAACTTAGGGTACAACTTGGACTTGTGTGGCTTTCCCCACGAGGAA
GAGGAGATTTTGGACCCCATGGACTTAATTCGCGAGTACCAGGAGAAGCG
CGCCTCCTTAAACGCAGAGATTGACCATGTGCTGGAGAAGATTACCTCTA
TGTTAGGAGGGAACTAAGCTTAAAAAAAAAAAAGGCCTCCCAAATCGGGG
GGCCTTTTTTATTGATAACAAAACGCTTGCCGGAGTTGACGGCTAGCTCA
GTCCTAGGTACAGTGCTAGCTACTAGAGAAAGAGGAGAAATACAAATGAC
AGCACAAGATTTAAAGAACAGTATCTTGCAGCTTGCGATTCAAGGAAAAT
TAGTCGAACAGCGTGCCGAAGAAGGCACCGCGAAGGAGCTTTTAGAGCAG
ATCAAGACCGAAAAGGAACGTTTAATCAATGAAAAAAAGATCAAAAAAGA
AAAGCCGCTTAGTGAAATTACGGAAGATGAGATTCCGTTTGAGATCCCGG
AGTCGTGGGAATGGGTCCGCTTGAATGAGATCTACAATTTTATTGATTAT
CGTGGGAAAACACCCAATAAAATCGATGAAGGAGTCGTTTTGATCACGGC
GTCGAACGTGCGTAAGGGCTACTTAGACTTTAGTAAGTCGGATTTTATTT
CCGAAAATGAGTATAAGGAGCGTATGACGCGCGGGATTACGCGCAAGGGT
GACCTTCTGTTCACCACCGAGGCACCACTTGGTTACGTGGCGGTGAACAC
GATTGAAATTGCTTCTTGCGGACAACGCGTGATCACATTTCAACAGTACG
GCGTTAACGTATTTTGTAACGAGCTGATGTGTTATTTCATCCAGTCACCA
TTCTTCCAGAACGAGCTGTTAGTCAAAGCTACAGGGACTACGGCGAAAGG
CATCAAGGCTGAAAAATTAAAGTATTTGCTGATCCCTGTGCCGCCATTAG
AAGAGCAACAACGCATTATTGCTAAAATCGAGGACATTTTACCTTACATT
GAACAATACGACAAAGCCTATTCAAAACTGGAGGTATTTAACAAGAAATT
CCCTGAGGATATGCAAAAGAGTATTCTGCAATACGCGATTCAGGGGAAGC
TGGTCGAACAGCGTCAGGAAGAAGGAACGGCGGAAGAGCTGTACGAACAG
ATCCAGGCAGAAAAGGAGCGCCTTATCAAAGAGGGGAAAATCAAGAAAGA
AAAACCATTAAGCGAGTTTACGGAGGAGGAAATTCCTTATGAGATTCCTG
ACTCTTGGAAATGGGTTAAGCTTGGCGATGTATTCCAAATTAACCCTCGT
AATTCGATCGAGGATAAGATCGAAGTCTCGTTTATTCCAATGACGTTGTT
GCAGGAAGGGTATGTATCTAAGTTCACATTTGAAATCAAAAAATGGGGCG
ATGTCAAGAAAGGTTTCACACATTTTAAGGATAACGACATCATCTTCGCT
AAAATCACCCCTTGTTTCCAGAATTTAAAATCAGCCATTATGGAGAACCT
GAAAAATGGATATGGCGCAGGCACGACAGAATTGCACGTCTTACGCTGTT
ACAAAATGCTTAGTTTAGAATATTTTCTGTGGTTCGTAAAGAGCCCTTAC
TTCATGTCGTTCTGCGAAGCCAACATGAGCGGTACAGCCGGACAACAACG
TGTGGGTACGGACATTGTCAAGAATGTTCTTTTACCATTACCCCCGTTAG
AGGAGCAAAAACGTATCGTATATGTAATTGAAAAGTATTTCCCATTTTGT
CAGCAATTGCGTAAATAAGCTTGACGAACAATAAGGCCTCCCAAATCGGG
GGGCCTTTTTATTTTTCAACAAAACGCT
ACTAACTGTCTATGCCTGGGA
AAGGGTGGGCAGGAGATGGGGCAGTGCAGGAAAAGTGGCACTATGAACCC
TGCAGCCCTAGTTTGACAATTAATCATTGGCATAGTATATCTGCATAGTA
TAATACAACTCACTATAGCAATTGTACTAACCTTCTTCTCTTTCCTCTCC
TGACAGGAGGAGCCATCATGGCCAAGTTGACCAGTGCCGTTCCGGTGCTC
ACCGCGCGCGACGTCGCCGGAGCGGTCGAGTTCTGGACCGACCGGCTCGG
GTTCTCCCGGGACTTCGTGGAGGACGACTTCGCCGGTGTGGTCCGGGACG
ACGTGACCCTGTTCATCAGCGCGGTCCAGGACCAGGTGGTGCCGGACAAC
ACCCTGGCCTGGGTGTGGGTGCGCGGCCTGGACGAGCTGTACGCCGAGTG
GTCGGAGGTCGTGTCCACGAACTTCCGGGACGCCTCCGGGCCGGCCATGA
CCGAGATCGGCGAGCAGCCGTGGGGGCGGGAGTTCGCCCTGCGCGACCCG
GCCGGCAACTGCGTGCACTTTGTGGCAGAGGAGCAGGACTGATTACCTGT
AACAGAGCATTAGCGCAAGGTGATTTTTGTCTTCTTGCGCTAATTTTTTC
TGGGTTGTTTTACTGCTTCTGCTGGTGGGATTTTTCGTCCTGGAACCGCT
CGGCATTCCGGTGAGCGCCATTGCAGCTGTGGGCGCGCTGATATTATTTG
TCGTCGCTAAACGCGGTCATGCGATTAATACGGGTAAAGTCCTGCGCGGT
GCCCCCTGGCAGATTGTCATCTTCTCGCTCGGCATGTATCTGGTGGTTTA
TGGCCTGCGCAATGCCGGATTAACGGAATATCTTTCTGGCGTACTCAACG
TGCTGGCGGATAACGGCCTGTGGGCCGCGACGCTCGGCACCGGATTCCTC
ACCGCCTTCCTCTCTTCTATTATGAACAATATGCCGACGGTACTGGTTGG
CGCGTTGTCCATTGATGGCAGCACGGCATCTGGCGTTATCAAAGAAGCGA
TGGTTTATGCCAATGTGATTGGCTGCGATTTGGGACCGAAAATTACCCCA
ATTGGTAGCCTGGCTACGCTACTCTGGCTGCACGTACTTTCGCAGAAGAA
TATGACTATCAGCTGGGGATATTACTTCCGTACAGGGATTATCATGACCC
TGCCTGTGCTGTTTGTGACGCTGGCTGCGCTGGCGCTACGTCTCTCTTTC
ACTTTGTAATGAGATACTGATATGAGCAACATTACCATTTATCACAACCC
GGCCTGCGGCACGTCGCGTAATACGCTGGAGATGATCCGCAACAGCGGCA
CAGAACCGACTATTATCCATTATCTGGAAACTCCGCCAACGCGCGATGAA
CTGGTCAAACTCATTGCCGATATGGGGATTTCCGTACGCGCGCTGCTGCG
TAAAAACGTCGAACCGTATGAGGAGCTGGGCCTTGCGGAAGATAAATTTA
CTGACGATCGGTTAATCGACTTTATGCTTCAGCACCCGATTCTGATTAAT
CGCCCGATTGTGGTGACGCCGCTGGGAACTCGCCTGTGCCGCCCTTCAGA
AGTGGTGCTGGAAATTCTGCCAGATGCGCAAAAAGGCGCATTCTCCAAGG
AAGATGGCGAGAAAGTGGTTGATGAAGCGGGTAAGCGCCTG

>2W91R (Linear DNA sequence for recombineering to generate the stable E. coli strain DH10B-2W91R) (SEQ ID NO: 120)

TTCTGTTACCCATCCAATTGTTCAAAATTC
TTGCTGATGAAACCCGTCTGGGCATCGTTTTACTGCTCAGCGAACTGGGA
GAGTTATGCGTCTGCGATCTCTGCACTGCTCTCGACCAGTCGCAGCCCAA
GATCTCCCGCCACCTGGCATTGCTGCGTGAAAGCGGGCTATTGCTGGACC
GCAAGCAAGGTAAGTGGGTTCATTACCGCTTATCACCGCATATTCCAGCA
TGGGCGGCGAAAATTATTGATGAGGCCTGGCGATGTGAACAGGAAAAGGT
TCAGGCGATTGTCCGCAACCTGGCTCGACAAAACTGTTCCGGGGACAGTA
AGAACATTTGCAGTTAAAAATTTAGCTAAACACATATGAATTTTCAGATG
TGTTTTATCCGGGAGGCATTATGTTACTGGCAGGCGCTATCTTTGTCCTG
ACCATCGTATTGGTTATCTGGCAGCCGAAAGGTTTAGGCATCGGCTGGAG
TGCAACGCTCGGCGCAGTACTGGCGTTAGTTACGGGCGTGGTCCATCCGG
GTGATATTCCGGTGGTGTGGAATATCGTCTGGAACGCGACGGCTGCGTTT
ATCGCCGTCATTATCATCAGCCTGCTGCTGGATGAGTCCGGCTTTTTTGA
ATGGGCGGCGCTGCACGTCTCACGCTGGGGTAATGGTCGTGGTCGCTTGC
TGTTTACCTGGATTGTCCTGCTCGGTGCTGCCGTTGCCGCCCTGTTTGCC
AATGATGGCGCGGCGCTTATTTTGACACCGATTGTCATCGCCATGCTGCT
GGCTTTAGGGTTCAGTAAAGGCACTACGCTGGCGTTCGTGATGGCGGCCG
GATTCATTGCCGATACCGCCAGCCTGCCGCTTATTGTCTCCAACCTGGTG
AATATCGTTTCCGCTGATTTCTTTGGCCTCGGCTTTCGCGAATACGCCTC
GGTGATGGTGCCGGTGGATATCGCCGCGATTGTTGCCACGCTGGTGATGT
TACATCTCTATTTTCGCAAAGATATTCCGCAGAACTACGATATGGCGCTG
CTGAAATCTCCCGCAGAAGCGATCAAAGATCCTGCTACGTTCAAAACTGG
GCAAGGAG
TTGACGGCTAGCTCAGTCCTAGGTACAGTGCTAGCTACTAG
AGAAAGAGGAGAAATACAAATGACCAAGTCTGAAACATTCATGATTCCAA
ACCACAAGGCCGCCAAATTAAGTGAGCTGGATATGATGATCGTTAACTCT
GTCCCGCCTGGGGGAAACTGGAAGAATATTCCCTTGGATGTACCATCGAA
ACGTATCGAACAGATTCGTGACAGCTATGCTCAAGGAAAAGGGTCGCGCA
GCACATACTACGGGCGTTTATTGCCCGATATGCCAGCTTATACGATCAAT
ACTTATTTCAATCGTCCTGGCAACGGATGCCACATTCACTACGAGCAAGA
TCGCGTACTTTCACAACGCGAAGCTGCACGTCTGCAGTCGTTCCCTGATG
ACTTTATCTTTTTTGGAGGTCAAACGGCGATTAATACGCAAATCGGTAAT
GCCGTGCCTCCCTTTCTTGCGTTTCTTATTGCAAAAGAAATTGAAAAAGC
GATCGGTAATACCGGCTACTACATTGACTTATTCAGTGGTGCAGGCGGAT
TGGGGTTGGGCTTTAAGTGGGCCGGGTGGACTCCATTGTTAGCTAATGAC
ATTGAGGAAAAGTACTTACAGACATACTCGAACAACGTACACAAAGAAGT
TTTGTGCGGAAGCATTTCGGACAACGAAACTTTTTCTAAGATCGCAGACA
AGATTTCTGGCTTTAAGAAATTATATTTTGATAAACAGCTGTGGATTCTG
GGCGGGCCTCCGTGCCAGGGATTTAGCACGGCTGGCAACGCGCGTACAAT
GGACGACCCACGCAACAGTCTGTTTATGCACTACAAGTCGCTGCTTAACG
AGATTAAGCCGAATGGATTCATTTTCGAGAACGTCGCCGGCCTGTTGAAC
ATGGAAAAAGGAAAGGTCTTTGAACGTGTTAAGGAGGAATTCTCGTCCAC
AATGAAAACCATGAATGGTTGGATTTTAAATTCGGAACATTACGCAATTC
CACAACGCCGTAAGCGTGTAATTCTTGTGGGCAGCAATGATCCGCTGTTC
TCGATCGAACCACCTCAGAAGCTGACGGAAGATAAAGAGTCTTGGGTGTC
AGTAAAAGATGCGTTATCTGACCTTCCCCCATTACAACACGGCGAGGATG
GATCTGGTAAATACTATATCCACCACCCGGAAAATGATTACCAGTTGTTT
ATGCGTGGAAACATTACACCCTCAGAGTATTATGAACGCAACATTAAGCC
GTCGCTTTAAGCTTCTCGGTACCAAATTCCAGAAAAGAGGCCTCCCGAAA
GGGGGGCCTTTTTTCGTTTTGGTCCCGCTAGGTGGAGTTGACGGCTAGCT
CAGTCCTAGGTACAGTGCTAGCTACTAGAGAAAGAGGAGAAATACAAATG
GCTATCACTAACTTCGTAAAGCGCATCCAAGATGTGATGCGTAATGACGC
TGGAGTTAATGGGGATGCACAACGCATCGAGCAAATCGTATGGATCTTAT
TTTTAAAGATTTATGATGCAAAGGAAGAGGAGTGGGAGTTGGTAAACGAA
AAGTATTCCTCGATTATCCCGGAGGAATGTAAATGGCGTAATTGGGCCGT
AGATGAAAAGGACGGTGAAGCTCTGACAGGCGACGCGATTTTAAACTTTG
TCAATAACACACTGTTCCCTATCCTGAAAAATTTAGAGATTGATGACGAA
ACCCCGATGAATCAGGTCATCGTGAAGTACGCTTTCGAGGACGCCAACAA
TTATATGAAAGATGGAGTTCTTCTTCGCCAGGTAGTTAATATCATTGATG
AGATTGATTTCACTGAATATGAAGAGCGCCATGCATTTGGTACGATTTAT
GAGAGCTTTTTAAAAGATCTTCAAAGTGCGGGTAACGCTGGTGAGTTCTA
CACGCCCCGCGCCGTGACCGACTTTATGGTCGAAGTGATCAAACCAAAGC
TTGGAGAGAAAGTAGCCGACTTTGCTTGCGGCACAGGGGGGTTCTTAGTG
TCTGCGTTGAATGCTCTTGAGAAACAGGTTGGGAATTCACTTGAGAATCG
CGAGATCTATAATAATTCTATTTACGGTGTGGAAAAGAAAGGGCTGCCCC
ATATTCTGTGTGTGACAAATATGTTGTTGCATGACATCGACAACCCGGAC
ATCATTCACGGGAACACACTGGAAACAGATTACAAGGAGTACCGCCGTAT
GGAACAATTCGACATTGTACTGATGAACCCGCCATACGGTGGGAATGAAA
AGGAGAGTGTTAAGGCTAACTTCCCTTCGGATCTTCGCTCCTCCGAGACA
GCTGACTTATTTATCGATATTATTATGTTTCGTTTGAAGAAAAATGGGCG
TTGTGCGATCATTCTTCCTGACGGCTTTCTGTTTGGTACTGATAACGCTA
AGGTCAACATTAAGAAGAAGTTACTTGGGGAATTTAACTTGCACACAATC
GTCCGCCTTCCCCACTCTGTGTTCGCGCCTTACACCTCGATTACGACTAA
TATTCTTTTTTTCGACAATACGCACCCTACCGAAGAAACATGGTTTTATC
GCCTGGACATGCCCGATGGGTATAAGAATTTTTCTAAAACAAAACCGATG
AAGTTAGAGCACTTTGGGTCGGCAATTGAATGGTGGGACAACCGTGAGGA
GATCGAAGTTGATGGATTTCCGAAAGCGAAGAAATACACCGTCGAGGACA
TTGAAAACTTAGGGTACAACTTGGACTTGTGTGGCTTTCCCCACGAGGAA
GAGGAGATTTTGGACCCCATGGACTTAATTCGCGAGTACCAGGAGAAGCG
CGCCTCCTTAAACGCAGAGATTGACCATGTGCTGGAGAAGATTACCTCTA
TGTTAGGAGGGAACTAAGCTTAAAAAAAAAAAAGGCCTCCCAAATCGGGG
GGCCTTTTTTATTGATAACAAAACGCTTGCCGGAGTTGACGGCTAGCTCA
GTCCTAGGTACAGTGCTAGCTACTAGAGAAAGAGGAGAAATACAAATGAC
AGCACAAGATTTAAAGAACAGTATCTTGCAGCTTGCGATTCAAGGAAAAT
TAGTCGAACAGCGTGCCGAAGAAGGCACCGCGAAGGAGCTTTTAGAGCAG
ATCAAGACCGAAAAGGAACGTTTAATCAATGAAAAAAAGATCAAAAAAGA
AAAGCCGCTTAGTGAAATTACGGAAGATGAGATTCCGTTTGAGATCCCGG
AGTCGTGGGAATGGGTCCGCTTGAATGAGATCTACAATTTTATTGATTAT
CGTGGGAAAACACCCAATAAAATCGATGAAGGAGTCGTTTTGATCACGGC
GTCGAACGTGCGTAAGGGCTACTTAGACTTTAGTAAGTCGGATTTTATTT
CCGAAAATGAGTATAAGGAGCGTATGACGCGCGGGATTACGCGCAAGGGT
GACCTTCTGTTCACCACCGAGGCACCACTTGGTTACGTGGCGGTGAACAC
GATTGAAATTGCTTCTTGCGGACAACGCGTGATCACATTTCAACAGTACG
GCGTTAACGTATTTTGTAACGAGCTGATGTGTTATTTCATCCAGTCACCA
TTCTTCCAGAACGAGCTGTTAGTCAAAGCTACAGGGACTACGGCGAAAGG
CATCAAGGCTGAAAAATTAAAGTATTTGCTGATCCCTGTGCCGCCATTAG
AAGAGCAACAACGCATTATTGCTAAAATCGAGGACATTTTACCTTACATT
GAACAATACGACAAAGCCTATTCAAAACTGGAGGTATTTAACAAGAAATT
CCCTGAGGATATGCAAAAGAGTATTCTGCAATACGCGATTCAGGGGAAGC
TGGTCGAACAGCGTCAGGAAGAAGGAACGGCGGAAGAGCTGTACGAACAG
ATCCAGGCAGAAAAGGAGCGCCTTATCAAAGAGGGGAAAATCAAGAAAGA
AAAACCATTAAGCGAGTTTACGGAGGAGGAAATTCCTTATGAGATTCCTG
ACTCTTGGAAATGGGTTAAGCTTGGCGATGTATTCCAAATTAACCCTCGT
AATTCGATCGAGGATAAGATCGAAGTCTCGTTTATTCCAATGACGTTGTT
GCAGGAAGGGTATGTATCTAAGTTCACATTTGAAATCAAAAAATGGGGCG
ATGTCAAGAAAGGTTTCACACATTTTAAGGATAACGACATCATCTTCGCT
AAAATCACCCCTTGTTTCCAGAATTTAAAATCAGCCATTATGGAGAACCT
GAAAAATGGATATGGCGCAGGCACGACAGAATTGCACGTCTTACGCTGTT
ACAAAATGCTTAGTTTAGAATATTTTCTGTGGTTCGTAAAGAGCCCTTAC
TTCATGTCGTTCTGCGAAGCCAACATGAGCGGTACAGCCGGACAACAACG
TGTGGGTACGGACATTGTCAAGAATGTTCTTTTACCATTACCCCCGTTAG
AGGAGCAAAAACGTATCGTATATGTAATTGAAAAGTATTTCCCATTTTGT
CAGCAATTGCGTAAATAAGCTTGACGAACAATAAGGCCTCCCAAATCGGG
GGGCCTTTTTATTTTTCAACAAAACGCT
ACTAACTGTCTATGCCTGGGA
AAGGGTGGGCAGGAGATGGGGCAGTGCAGGAAAAGTGGCACTATGAACCC
TGCAGCCCTAGTTTGACAATTAATCATTGGCATAGTATATCTGCATAGTA
TAATACAACTCACTATAGCAATTGTACTAACCTTCTTCTCTTTCCTCTCC
TGACAGGAGGAGCCATCATGGCCAAGTTGACCAGTGCCGTTCCGGTGCTC
ACCGCGCGCGACGTCGCCGGAGCGGTCGAGTTCTGGACCGACCGGCTCGG
GTTCTCCCGGGACTTCGTGGAGGACGACTTCGCCGGTGTGGTCCGGGACG
ACGTGACCCTGTTCATCAGCGCGGTCCAGGACCAGGTGGTGCCGGACAAC
ACCCTGGCCTGGGTGTGGGTGCGCGGCCTGGACGAGCTGTACGCCGAGTG
GTCGGAGGTCGTGTCCACGAACTTCCGGGACGCCTCCGGGCCGGCCATGA
CCGAGATCGGCGAGCAGCCGTGGGGGCGGGAGTTCGCCCTGCGCGACCCG
GCCGGCAACTGCGTGCACTTTGTGGCAGAGGAGCAGGACTGATTACCTGT
AACAGAGCATTAGCGCAAGGTGATTTTTGTCTTCTTGCGCTAATTTTTTC
TGGGTTGTTTTACTGCTTCTGCTGGTGGGATTTTTCGTCCTGGAACCGCT
CGGCATTCCGGTGAGCGCCATTGCAGCTGTGGGCGCGCTGATATTATTTG
TCGTCGCTAAACGCGGTCATGCGATTAATACGGGTAAAGTCCTGCGCGGT
GCCCCCTGGCAGATTGTCATCTTCTCGCTCGGCATGTATCTGGTGGTTTA
TGGCCTGCGCAATGCCGGATTAACGGAATATCTTTCTGGCGTACTCAACG
TGCTGGCGGATAACGGCCTGTGGGCCGCGACGCTCGGCACCGGATTCCTC
ACCGCCTTCCTCTCTTCTATTATGAACAATATGCCGACGGTACTGGTTGG
CGCGTTGTCCATTGATGGCAGCACGGCATCTGGCGTTATCAAAGAAGCGA
TGGTTTATGCCAATGTGATTGGCTGCGATTTGGGACCGAAAATTACCCCA
ATTGGTAGCCTGGCTACGCTACTCTGGCTGCACGTACTTTCGCAGAAGAA
TATGACTATCAGCTGGGGATATTACTTCCGTACAGGGATTATCATGACCC
TGCCTGTGCTGTTTGTGACGCTGGCTGCGCTGGCGCTACGTCTCTCTTTC
ACTTTGTAATGAGATACTGATATGAGCAACATTACCATTTATCACAACCC
GGCCTGCGGCACGTCGCGTAATACGCTGGAGATGATCCGCAACAGCGGCA
CAGAACCGACTATTATCCATTATCTGGAAACTCCGCCAACGCGCGATGAA
CTGGTCAAACTCATTGCCGATATGGGGATTTCCGTACGCGCGCTGCTGCG
TAAAAACGTCGAACCGTATGAGGAGCTGGGCCTTGCGGAAGATAAATTTA
CTGACGATCGGTTAATCGACTTTATGCTTCAGCACCCGATTCTGATTAAT
CGCCCGATTGTGGTGACGCCGCTGGGAACTCGCCTGTGCCGCCCTTCAGA
AGTGGTGCTGGAAATTCTGCCAGATGCGCAAAAAGGCGCATTCTCCAAGG
AAGATGGCGAGAAAGTGGTTGATGAAGCGGGTAAGCGCCTG

>2W92R (Linear DNA sequence for recombineering to generate the stable E. coli strain DH10B-2W92R) (SEQ ID NO: 121)

TTCTGTTACCCATCCAATTGTTCAAAATTC
TTGCTGATGAAACCCGTCTGGGCATCGTTTTACTGCTCAGCGAACTGGGA
GAGTTATGCGTCTGCGATCTCTGCACTGCTCTCGACCAGTCGCAGCCCAA
GATCTCCCGCCACCTGGCATTGCTGCGTGAAAGCGGGCTATTGCTGGACC
GCAAGCAAGGTAAGTGGGTTCATTACCGCTTATCACCGCATATTCCAGCA
TGGGCGGCGAAAATTATTGATGAGGCCTGGCGATGTGAACAGGAAAAGGT
TCAGGCGATTGTCCGCAACCTGGCTCGACAAAACTGTTCCGGGGACAGTA
AGAACATTTGCAGTTAAAAATTTAGCTAAACACATATGAATTTTCAGATG
TGTTTTATCCGGGAGGCATTATGTTACTGGCAGGCGCTATCTTTGTCCTG
ACCATCGTATTGGTTATCTGGCAGCCGAAAGGTTTAGGCATCGGCTGGAG
TGCAACGCTCGGCGCAGTACTGGCGTTAGTTACGGGCGTGGTCCATCCGG
GTGATATTCCGGTGGTGTGGAATATCGTCTGGAACGCGACGGCTGCGTTT
ATCGCCGTCATTATCATCAGCCTGCTGCTGGATGAGTCCGGCTTTTTTGA
ATGGGCGGCGCTGCACGTCTCACGCTGGGGTAATGGTCGTGGTCGCTTGC
TGTTTACCTGGATTGTCCTGCTCGGTGCTGCCGTTGCCGCCCTGTTTGCC
AATGATGGCGCGGCGCTTATTTTGACACCGATTGTCATCGCCATGCTGCT
GGCTTTAGGGTTCAGTAAAGGCACTACGCTGGCGTTCGTGATGGCGGCCG
GATTCATTGCCGATACCGCCAGCCTGCCGCTTATTGTCTCCAACCTGGTG
AATATCGTTTCCGCTGATTTCTTTGGCCTCGGCTTTCGCGAATACGCCTC
GGTGATGGTGCCGGTGGATATCGCCGCGATTGTTGCCACGCTGGTGATGT
TACATCTCTATTTTCGCAAAGATATTCCGCAGAACTACGATATGGCGCTG
CTGAAATCTCCCGCAGAAGCGATCAAAGATCCTGCTACGTTCAAAACTGG
GCAAGGAG
TTTATGGCTAGCTCAGTCCTAGGTACAATGCTAGCTACTAG
AGAAAGAGGAGAAATACAAATGACCAAGTCTGAAACATTCATGATTCCAA
ACCACAAGGCCGCCAAATTAAGTGAGCTGGATATGATGATCGTTAACTCT
GTCCCGCCTGGGGGAAACTGGAAGAATATTCCCTTGGATGTACCATCGAA
ACGTATCGAACAGATTCGTGACAGCTATGCTCAAGGAAAAGGGTCGCGCA
GCACATACTACGGGCGTTTATTGCCCGATATGCCAGCTTATACGATCAAT
ACTTATTTCAATCGTCCTGGCAACGGATGCCACATTCACTACGAGCAAGA
TCGCGTACTTTCACAACGCGAAGCTGCACGTCTGCAGTCGTTCCCTGATG
ACTTTATCTTTTTTGGAGGTCAAACGGCGATTAATACGCAAATCGGTAAT
GCCGTGCCTCCCTTTCTTGCGTTTCTTATTGCAAAAGAAATTGAAAAAGC
GATCGGTAATACCGGCTACTACATTGACTTATTCAGTGGTGCAGGCGGAT
TGGGGTTGGGCTTTAAGTGGGCCGGGTGGACTCCATTGTTAGCTAATGAC
ATTGAGGAAAAGTACTTACAGACATACTCGAACAACGTACACAAAGAAGT
TTTGTGCGGAAGCATTTCGGACAACGAAACTTTTTCTAAGATCGCAGACA
AGATTTCTGGCTTTAAGAAATTATATTTTGATAAACAGCTGTGGATTCTG
GGCGGGCCTCCGTGCCAGGGATTTAGCACGGCTGGCAACGCGCGTACAAT
GGACGACCCACGCAACAGTCTGTTTATGCACTACAAGTCGCTGCTTAACG
AGATTAAGCCGAATGGATTCATTTTCGAGAACGTCGCCGGCCTGTTGAAC
ATGGAAAAAGGAAAGGTCTTTGAACGTGTTAAGGAGGAATTCTCGTCCAC
AATGAAAACCATGAATGGTTGGATTTTAAATTCGGAACATTACGCAATTC
CACAACGCCGTAAGCGTGTAATTCTTGTGGGCAGCAATGATCCGCTGTTC
TCGATCGAACCACCTCAGAAGCTGACGGAAGATAAAGAGTCTTGGGTGTC
AGTAAAAGATGCGTTATCTGACCTTCCCCCATTACAACACGGCGAGGATG
GATCTGGTAAATACTATATCCACCACCCGGAAAATGATTACCAGTTGTTT
ATGCGTGGAAACATTACACCCTCAGAGTATTATGAACGCAACATTAAGCC
GTCGCTTTAAGCTTCTCGGTACCAAATTCCAGAAAAGAGGCCTCCCGAAA
GGGGGGCCTTTTTTCGTTTTGGTCCCGCTAGGTGGAGTTGACGGCTAGCT
CAGTCCTAGGTACAGTGCTAGCTACTAGAGAAAGAGGAGAAATACAAATG
GCTATCACTAACTTCGTAAAGCGCATCCAAGATGTGATGCGTAATGACGC
TGGAGTTAATGGGGATGCACAACGCATCGAGCAAATCGTATGGATCTTAT
TTTTAAAGATTTATGATGCAAAGGAAGAGGAGTGGGAGTTGGTAAACGAA
AAGTATTCCTCGATTATCCCGGAGGAATGTAAATGGCGTAATTGGGCCGT
AGATGAAAAGGACGGTGAAGCTCTGACAGGCGACGCGATTTTAAACTTTG
TCAATAACACACTGTTCCCTATCCTGAAAAATTTAGAGATTGATGACGAA
ACCCCGATGAATCAGGTCATCGTGAAGTACGCTTTCGAGGACGCCAACAA
TTATATGAAAGATGGAGTTCTTCTTCGCCAGGTAGTTAATATCATTGATG
AGATTGATTTCACTGAATATGAAGAGCGCCATGCATTTGGTACGATTTAT
GAGAGCTTTTTAAAAGATCTTCAAAGTGCGGGTAACGCTGGTGAGTTCTA
CACGCCCCGCGCCGTGACCGACTTTATGGTCGAAGTGATCAAACCAAAGC
TTGGAGAGAAAGTAGCCGACTTTGCTTGCGGCACAGGGGGGTTCTTAGTG
TCTGCGTTGAATGCTCTTGAGAAACAGGTTGGGAATTCACTTGAGAATCG
CGAGATCTATAATAATTCTATTTACGGTGTGGAAAAGAAAGGGCTGCCCC
ATATTCTGTGTGTGACAAATATGTTGTTGCATGACATCGACAACCCGGAC
ATCATTCACGGGAACACACTGGAAACAGATTACAAGGAGTACCGCCGTAT
GGAACAATTCGACATTGTACTGATGAACCCGCCATACGGTGGGAATGAAA
AGGAGAGTGTTAAGGCTAACTTCCCTTCGGATCTTCGCTCCTCCGAGACA
GCTGACTTATTTATCGATATTATTATGTTTCGTTTGAAGAAAAATGGGCG
TTGTGCGATCATTCTTCCTGACGGCTTTCTGTTTGGTACTGATAACGCTA
AGGTCAACATTAAGAAGAAGTTACTTGGGGAATTTAACTTGCACACAATC
GTCCGCCTTCCCCACTCTGTGTTCGCGCCTTACACCTCGATTACGACTAA
TATTCTTTTTTTCGACAATACGCACCCTACCGAAGAAACATGGTTTTATC
GCCTGGACATGCCCGATGGGTATAAGAATTTTTCTAAAACAAAACCGATG
AAGTTAGAGCACTTTGGGTCGGCAATTGAATGGTGGGACAACCGTGAGGA
GATCGAAGTTGATGGATTTCCGAAAGCGAAGAAATACACCGTCGAGGACA
TTGAAAACTTAGGGTACAACTTGGACTTGTGTGGCTTTCCCCACGAGGAA
GAGGAGATTTTGGACCCCATGGACTTAATTCGCGAGTACCAGGAGAAGCG
CGCCTCCTTAAACGCAGAGATTGACCATGTGCTGGAGAAGATTACCTCTA
TGTTAGGAGGGAACTAAGCTTAAAAAAAAAAAAGGCCTCCCAAATCGGGG
GGCCTTTTTTATTGATAACAAAACGCTTGCCGGAGTTGACGGCTAGCTCA
GTCCTAGGTACAGTGCTAGCTACTAGAGAAAGAGGAGAAATACAAATGAC
AGCACAAGATTTAAAGAACAGTATCTTGCAGCTTGCGATTCAAGGAAAAT
TAGTCGAACAGCGTGCCGAAGAAGGCACCGCGAAGGAGCTTTTAGAGCAG
ATCAAGACCGAAAAGGAACGTTTAATCAATGAAAAAAAGATCAAAAAAGA
AAAGCCGCTTAGTGAAATTACGGAAGATGAGATTCCGTTTGAGATCCCGG
AGTCGTGGGAATGGGTCCGCTTGAATGAGATCTACAATTTTATTGATTAT
CGTGGGAAAACACCCAATAAAATCGATGAAGGAGTCGTTTTGATCACGGC
GTCGAACGTGCGTAAGGGCTACTTAGACTTTAGTAAGTCGGATTTTATTT
CCGAAAATGAGTATAAGGAGCGTATGACGCGCGGGATTACGCGCAAGGGT
GACCTTCTGTTCACCACCGAGGCACCACTTGGTTACGTGGCGGTGAACAC
GATTGAAATTGCTTCTTGCGGACAACGCGTGATCACATTTCAACAGTACG
GCGTTAACGTATTTTGTAACGAGCTGATGTGTTATTTCATCCAGTCACCA
TTCTTCCAGAACGAGCTGTTAGTCAAAGCTACAGGGACTACGGCGAAAGG
CATCAAGGCTGAAAAATTAAAGTATTTGCTGATCCCTGTGCCGCCATTAG
AAGAGCAACAACGCATTATTGCTAAAATCGAGGACATTTTACCTTACATT
GAACAATACGACAAAGCCTATTCAAAACTGGAGGTATTTAACAAGAAATT
CCCTGAGGATATGCAAAAGAGTATTCTGCAATACGCGATTCAGGGGAAGC
TGGTCGAACAGCGTCAGGAAGAAGGAACGGCGGAAGAGCTGTACGAACAG
ATCCAGGCAGAAAAGGAGCGCCTTATCAAAGAGGGGAAAATCAAGAAAGA
AAAACCATTAAGCGAGTTTACGGAGGAGGAAATTCCTTATGAGATTCCTG
ACTCTTGGAAATGGGTTAAGCTTGGCGATGTATTCCAAATTAACCCTCGT
AATTCGATCGAGGATAAGATCGAAGTCTCGTTTATTCCAATGACGTTGTT
GCAGGAAGGGTATGTATCTAAGTTCACATTTGAAATCAAAAAATGGGGCG
ATGTCAAGAAAGGTTTCACACATTTTAAGGATAACGACATCATCTTCGCT
AAAATCACCCCTTGTTTCCAGAATTTAAAATCAGCCATTATGGAGAACCT
GAAAAATGGATATGGCGCAGGCACGACAGAATTGCACGTCTTACGCTGTT
ACAAAATGCTTAGTTTAGAATATTTTCTGTGGTTCGTAAAGAGCCCTTAC
TTCATGTCGTTCTGCGAAGCCAACATGAGCGGTACAGCCGGACAACAACG
TGTGGGTACGGACATTGTCAAGAATGTTCTTTTACCATTACCCCCGTTAG
AGGAGCAAAAACGTATCGTATATGTAATTGAAAAGTATTTCCCATTTTGT
CAGCAATTGCGTAAATAAGCTTGACGAACAATAAGGCCTCCCAAATCGGG
GGGCCTTTTTATTTTTCAACAAAACGCT
ACTAACTGTCTATGCCTGGGA
AAGGGTGGGCAGGAGATGGGGCAGTGCAGGAAAAGTGGCACTATGAACCC
TGCAGCCCTAGTTTGACAATTAATCATTGGCATAGTATATCTGCATAGTA
TAATACAACTCACTATAGCAATTGTACTAACCTTCTTCTCTTTCCTCTCC
TGACAGGAGGAGCCATCATGGCCAAGTTGACCAGTGCCGTTCCGGTGCTC
ACCGCGCGCGACGTCGCCGGAGCGGTCGAGTTCTGGACCGACCGGCTCGG
GTTCTCCCGGGACTTCGTGGAGGACGACTTCGCCGGTGTGGTCCGGGACG
ACGTGACCCTGTTCATCAGCGCGGTCCAGGACCAGGTGGTGCCGGACAAC
ACCCTGGCCTGGGTGTGGGTGCGCGGCCTGGACGAGCTGTACGCCGAGTG
GTCGGAGGTCGTGTCCACGAACTTCCGGGACGCCTCCGGGCCGGCCATGA
CCGAGATCGGCGAGCAGCCGTGGGGGCGGGAGTTCGCCCTGCGCGACCCG
GCCGGCAACTGCGTGCACTTTGTGGCAGAGGAGCAGGACTGATTACCTGT
AACAGAGCATTAGCGCAAGGTGATTTTTGTCTTCTTGCGCTAATTTTTTC
TGGGTTGTTTTACTGCTTCTGCTGGTGGGATTTTTCGTCCTGGAACCGCT
CGGCATTCCGGTGAGCGCCATTGCAGCTGTGGGCGCGCTGATATTATTTG
TCGTCGCTAAACGCGGTCATGCGATTAATACGGGTAAAGTCCTGCGCGGT
GCCCCCTGGCAGATTGTCATCTTCTCGCTCGGCATGTATCTGGTGGTTTA
TGGCCTGCGCAATGCCGGATTAACGGAATATCTTTCTGGCGTACTCAACG
TGCTGGCGGATAACGGCCTGTGGGCCGCGACGCTCGGCACCGGATTCCTC
ACCGCCTTCCTCTCTTCTATTATGAACAATATGCCGACGGTACTGGTTGG
CGCGTTGTCCATTGATGGCAGCACGGCATCTGGCGTTATCAAAGAAGCGA
TGGTTTATGCCAATGTGATTGGCTGCGATTTGGGACCGAAAATTACCCCA
ATTGGTAGCCTGGCTACGCTACTCTGGCTGCACGTACTTTCGCAGAAGAA
TATGACTATCAGCTGGGGATATTACTTCCGTACAGGGATTATCATGACCC
TGCCTGTGCTGTTTGTGACGCTGGCTGCGCTGGCGCTACGTCTCTCTTTC
ACTTTGTAATGAGATACTGATATGAGCAACATTACCATTTATCACAACCC
GGCCTGCGGCACGTCGCGTAATACGCTGGAGATGATCCGCAACAGCGGCA
CAGAACCGACTATTATCCATTATCTGGAAACTCCGCCAACGCGCGATGAA
CTGGTCAAACTCATTGCCGATATGGGGATTTCCGTACGCGCGCTGCTGCG
TAAAAACGTCGAACCGTATGAGGAGCTGGGCCTTGCGGAAGATAAATTTA
CTGACGATCGGTTAATCGACTTTATGCTTCAGCACCCGATTCTGATTAAT
CGCCCGATTGTGGTGACGCCGCTGGGAACTCGCCTGTGCCGCCCTTCAGA
AGTGGTGCTGGAAATTCTGCCAGATGCGCAAAAAGGCGCATTCTCCAAGG
AAGATGGCGAGAAAGTGGTTGATGAAGCGGGTAAGCGCCTG

>2W94R (Linear DNA sequence for recombineering to generate the stable E. coli strain DH10B-2W94R) (SEQ ID NO: 122)

TTCTGTTACCCATCCAATTGTTCAAAATTC
TTGCTGATGAAACCCGTCTGGGCATCGTTTTACTGCTCAGCGAACTGGGA
GAGTTATGCGTCTGCGATCTCTGCACTGCTCTCGACCAGTCGCAGCCCAA
GATCTCCCGCCACCTGGCATTGCTGCGTGAAAGCGGGCTATTGCTGGACC
GCAAGCAAGGTAAGTGGGTTCATTACCGCTTATCACCGCATATTCCAGCA
TGGGCGGCGAAAATTATTGATGAGGCCTGGCGATGTGAACAGGAAAAGGT
TCAGGCGATTGTCCGCAACCTGGCTCGACAAAACTGTTCCGGGGACAGTA
AGAACATTTGCAGTTAAAAATTTAGCTAAACACATATGAATTTTCAGATG
TGTTTTATCCGGGAGGCATTATGTTACTGGCAGGCGCTATCTTTGTCCTG
ACCATCGTATTGGTTATCTGGCAGCCGAAAGGTTTAGGCATCGGCTGGAG
TGCAACGCTCGGCGCAGTACTGGCGTTAGTTACGGGCGTGGTCCATCCGG
GTGATATTCCGGTGGTGTGGAATATCGTCTGGAACGCGACGGCTGCGTTT
ATCGCCGTCATTATCATCAGCCTGCTGCTGGATGAGTCCGGCTTTTTTGA
ATGGGCGGCGCTGCACGTCTCACGCTGGGGTAATGGTCGTGGTCGCTTGC
TGTTTACCTGGATTGTCCTGCTCGGTGCTGCCGTTGCCGCCCTGTTTGCC
AATGATGGCGCGGCGCTTATTTTGACACCGATTGTCATCGCCATGCTGCT
GGCTTTAGGGTTCAGTAAAGGCACTACGCTGGCGTTCGTGATGGCGGCCG
GATTCATTGCCGATACCGCCAGCCTGCCGCTTATTGTCTCCAACCTGGTG
AATATCGTTTCCGCTGATTTCTTTGGCCTCGGCTTTCGCGAATACGCCTC
GGTGATGGTGCCGGTGGATATCGCCGCGATTGTTGCCACGCTGGTGATGT
TACATCTCTATTTTCGCAAAGATATTCCGCAGAACTACGATATGGCGCTG
CTGAAATCTCCCGCAGAAGCGATCAAAGATCCTGCTACGTTCAAAACTGG
GCAAGGAG
CTGATAGCTAGCTCAGTCCTAGGGATTATGCTAGCTACTAG
AGAAAGAGGAGAAATACAAATGACCAAGTCTGAAACATTCATGATTCCAA
ACCACAAGGCCGCCAAATTAAGTGAGCTGGATATGATGATCGTTAACTCT
GTCCCGCCTGGGGGAAACTGGAAGAATATTCCCTTGGATGTACCATCGAA
ACGTATCGAACAGATTCGTGACAGCTATGCTCAAGGAAAAGGGTCGCGCA
GCACATACTACGGGCGTTTATTGCCCGATATGCCAGCTTATACGATCAAT
ACTTATTTCAATCGTCCTGGCAACGGATGCCACATTCACTACGAGCAAGA
TCGCGTACTTTCACAACGCGAAGCTGCACGTCTGCAGTCGTTCCCTGATG
ACTTTATCTTTTTTGGAGGTCAAACGGCGATTAATACGCAAATCGGTAAT
GCCGTGCCTCCCTTTCTTGCGTTTCTTATTGCAAAAGAAATTGAAAAAGC
GATCGGTAATACCGGCTACTACATTGACTTATTCAGTGGTGCAGGCGGAT
TGGGGTTGGGCTTTAAGTGGGCCGGGTGGACTCCATTGTTAGCTAATGAC
ATTGAGGAAAAGTACTTACAGACATACTCGAACAACGTACACAAAGAAGT
TTTGTGCGGAAGCATTTCGGACAACGAAACTTTTTCTAAGATCGCAGACA
AGATTTCTGGCTTTAAGAAATTATATTTTGATAAACAGCTGTGGATTCTG
GGCGGGCCTCCGTGCCAGGGATTTAGCACGGCTGGCAACGCGCGTACAAT
GGACGACCCACGCAACAGTCTGTTTATGCACTACAAGTCGCTGCTTAACG
AGATTAAGCCGAATGGATTCATTTTCGAGAACGTCGCCGGCCTGTTGAAC
ATGGAAAAAGGAAAGGTCTTTGAACGTGTTAAGGAGGAATTCTCGTCCAC
AATGAAAACCATGAATGGTTGGATTTTAAATTCGGAACATTACGCAATTC
CACAACGCCGTAAGCGTGTAATTCTTGTGGGCAGCAATGATCCGCTGTTC
TCGATCGAACCACCTCAGAAGCTGACGGAAGATAAAGAGTCTTGGGTGTC
AGTAAAAGATGCGTTATCTGACCTTCCCCCATTACAACACGGCGAGGATG
GATCTGGTAAATACTATATCCACCACCCGGAAAATGATTACCAGTTGTTT
ATGCGTGGAAACATTACACCCTCAGAGTATTATGAACGCAACATTAAGCC
GTCGCTTTAAGCTTCTCGGTACCAAATTCCAGAAAAGAGGCCTCCCGAAA
GGGGGGCCTTTTTTCGTTTTGGTCCCGCTAGGTGGAGTTGACGGCTAGCT
CAGTCCTAGGTACAGTGCTAGCTACTAGAGAAAGAGGAGAAATACAAATG
GCTATCACTAACTTCGTAAAGCGCATCCAAGATGTGATGCGTAATGACGC
TGGAGTTAATGGGGATGCACAACGCATCGAGCAAATCGTATGGATCTTAT
TTTTAAAGATTTATGATGCAAAGGAAGAGGAGTGGGAGTTGGTAAACGAA
AAGTATTCCTCGATTATCCCGGAGGAATGTAAATGGCGTAATTGGGCCGT
AGATGAAAAGGACGGTGAAGCTCTGACAGGCGACGCGATTTTAAACTTTG
TCAATAACACACTGTTCCCTATCCTGAAAAATTTAGAGATTGATGACGAA
ACCCCGATGAATCAGGTCATCGTGAAGTACGCTTTCGAGGACGCCAACAA
TTATATGAAAGATGGAGTTCTTCTTCGCCAGGTAGTTAATATCATTGATG
AGATTGATTTCACTGAATATGAAGAGCGCCATGCATTTGGTACGATTTAT
GAGAGCTTTTTAAAAGATCTTCAAAGTGCGGGTAACGCTGGTGAGTTCTA
CACGCCCCGCGCCGTGACCGACTTTATGGTCGAAGTGATCAAACCAAAGC
TTGGAGAGAAAGTAGCCGACTTTGCTTGCGGCACAGGGGGGTTCTTAGTG
TCTGCGTTGAATGCTCTTGAGAAACAGGTTGGGAATTCACTTGAGAATCG
CGAGATCTATAATAATTCTATTTACGGTGTGGAAAAGAAAGGGCTGCCCC
ATATTCTGTGTGTGACAAATATGTTGTTGCATGACATCGACAACCCGGAC
ATCATTCACGGGAACACACTGGAAACAGATTACAAGGAGTACCGCCGTAT
GGAACAATTCGACATTGTACTGATGAACCCGCCATACGGTGGGAATGAAA
AGGAGAGTGTTAAGGCTAACTTCCCTTCGGATCTTCGCTCCTCCGAGACA
GCTGACTTATTTATCGATATTATTATGTTTCGTTTGAAGAAAAATGGGCG
TTGTGCGATCATTCTTCCTGACGGCTTTCTGTTTGGTACTGATAACGCTA
AGGTCAACATTAAGAAGAAGTTACTTGGGGAATTTAACTTGCACACAATC
GTCCGCCTTCCCCACTCTGTGTTCGCGCCTTACACCTCGATTACGACTAA
TATTCTTTTTTTCGACAATACGCACCCTACCGAAGAAACATGGTTTTATC
GCCTGGACATGCCCGATGGGTATAAGAATTTTTCTAAAACAAAACCGATG
AAGTTAGAGCACTTTGGGTCGGCAATTGAATGGTGGGACAACCGTGAGGA
GATCGAAGTTGATGGATTTCCGAAAGCGAAGAAATACACCGTCGAGGACA
TTGAAAACTTAGGGTACAACTTGGACTTGTGTGGCTTTCCCCACGAGGAA
GAGGAGATTTTGGACCCCATGGACTTAATTCGCGAGTACCAGGAGAAGCG
CGCCTCCTTAAACGCAGAGATTGACCATGTGCTGGAGAAGATTACCTCTA
TGTTAGGAGGGAACTAAGCTTAAAAAAAAAAAAGGCCTCCCAAATCGGGG
GGCCTTTTTTATTGATAACAAAACGCTTGCCGGAGTTGACGGCTAGCTCA
GTCCTAGGTACAGTGCTAGCTACTAGAGAAAGAGGAGAAATACAAATGAC
AGCACAAGATTTAAAGAACAGTATCTTGCAGCTTGCGATTCAAGGAAAAT
TAGTCGAACAGCGTGCCGAAGAAGGCACCGCGAAGGAGCTTTTAGAGCAG
ATCAAGACCGAAAAGGAACGTTTAATCAATGAAAAAAAGATCAAAAAAGA
AAAGCCGCTTAGTGAAATTACGGAAGATGAGATTCCGTTTGAGATCCCGG
AGTCGTGGGAATGGGTCCGCTTGAATGAGATCTACAATTTTATTGATTAT
CGTGGGAAAACACCCAATAAAATCGATGAAGGAGTCGTTTTGATCACGGC
GTCGAACGTGCGTAAGGGCTACTTAGACTTTAGTAAGTCGGATTTTATTT
CCGAAAATGAGTATAAGGAGCGTATGACGCGCGGGATTACGCGCAAGGGT
GACCTTCTGTTCACCACCGAGGCACCACTTGGTTACGTGGCGGTGAACAC
GATTGAAATTGCTTCTTGCGGACAACGCGTGATCACATTTCAACAGTACG
GCGTTAACGTATTTTGTAACGAGCTGATGTGTTATTTCATCCAGTCACCA
TTCTTCCAGAACGAGCTGTTAGTCAAAGCTACAGGGACTACGGCGAAAGG
CATCAAGGCTGAAAAATTAAAGTATTTGCTGATCCCTGTGCCGCCATTAG
AAGAGCAACAACGCATTATTGCTAAAATCGAGGACATTTTACCTTACATT
GAACAATACGACAAAGCCTATTCAAAACTGGAGGTATTTAACAAGAAATT
CCCTGAGGATATGCAAAAGAGTATTCTGCAATACGCGATTCAGGGGAAGC
TGGTCGAACAGCGTCAGGAAGAAGGAACGGCGGAAGAGCTGTACGAACAG
ATCCAGGCAGAAAAGGAGCGCCTTATCAAAGAGGGGAAAATCAAGAAAGA
AAAACCATTAAGCGAGTTTACGGAGGAGGAAATTCCTTATGAGATTCCTG
ACTCTTGGAAATGGGTTAAGCTTGGCGATGTATTCCAAATTAACCCTCGT
AATTCGATCGAGGATAAGATCGAAGTCTCGTTTATTCCAATGACGTTGTT
GCAGGAAGGGTATGTATCTAAGTTCACATTTGAAATCAAAAAATGGGGCG
ATGTCAAGAAAGGTTTCACACATTTTAAGGATAACGACATCATCTTCGCT
AAAATCACCCCTTGTTTCCAGAATTTAAAATCAGCCATTATGGAGAACCT
GAAAAATGGATATGGCGCAGGCACGACAGAATTGCACGTCTTACGCTGTT
ACAAAATGCTTAGTTTAGAATATTTTCTGTGGTTCGTAAAGAGCCCTTAC
TTCATGTCGTTCTGCGAAGCCAACATGAGCGGTACAGCCGGACAACAACG
TGTGGGTACGGACATTGTCAAGAATGTTCTTTTACCATTACCCCCGTTAG
AGGAGCAAAAACGTATCGTATATGTAATTGAAAAGTATTTCCCATTTTGT
CAGCAATTGCGTAAATAAGCTTGACGAACAATAAGGCCTCCCAAATCGGG
GGGCCTTTTTATTTTTCAACAAAACGCT
ACTAACTGTCTATGCCTGGGA
AAGGGTGGGCAGGAGATGGGGCAGTGCAGGAAAAGTGGCACTATGAACCC
TGCAGCCCTAGTTTGACAATTAATCATTGGCATAGTATATCTGCATAGTA
TAATACAACTCACTATAGCAATTGTACTAACCTTCTTCTCTTTCCTCTCC
TGACAGGAGGAGCCATCATGGCCAAGTTGACCAGTGCCGTTCCGGTGCTC
ACCGCGCGCGACGTCGCCGGAGCGGTCGAGTTCTGGACCGACCGGCTCGG
GTTCTCCCGGGACTTCGTGGAGGACGACTTCGCCGGTGTGGTCCGGGACG
ACGTGACCCTGTTCATCAGCGCGGTCCAGGACCAGGTGGTGCCGGACAAC
ACCCTGGCCTGGGTGTGGGTGCGCGGCCTGGACGAGCTGTACGCCGAGTG
GTCGGAGGTCGTGTCCACGAACTTCCGGGACGCCTCCGGGCCGGCCATGA
CCGAGATCGGCGAGCAGCCGTGGGGGCGGGAGTTCGCCCTGCGCGACCCG
GCCGGCAACTGCGTGCACTTTGTGGCAGAGGAGCAGGACTGATTACCTGT
AACAGAGCATTAGCGCAAGGTGATTTTTGTCTTCTTGCGCTAATTTTTTC
TGGGTTGTTTTACTGCTTCTGCTGGTGGGATTTTTCGTCCTGGAACCGCT
CGGCATTCCGGTGAGCGCCATTGCAGCTGTGGGCGCGCTGATATTATTTG
TCGTCGCTAAACGCGGTCATGCGATTAATACGGGTAAAGTCCTGCGCGGT
GCCCCCTGGCAGATTGTCATCTTCTCGCTCGGCATGTATCTGGTGGTTTA
TGGCCTGCGCAATGCCGGATTAACGGAATATCTTTCTGGCGTACTCAACG
TGCTGGCGGATAACGGCCTGTGGGCCGCGACGCTCGGCACCGGATTCCTC
ACCGCCTTCCTCTCTTCTATTATGAACAATATGCCGACGGTACTGGTTGG
CGCGTTGTCCATTGATGGCAGCACGGCATCTGGCGTTATCAAAGAAGCGA
TGGTTTATGCCAATGTGATTGGCTGCGATTTGGGACCGAAAATTACCCCA
ATTGGTAGCCTGGCTACGCTACTCTGGCTGCACGTACTTTCGCAGAAGAA
TATGACTATCAGCTGGGGATATTACTTCCGTACAGGGATTATCATGACCC
TGCCTGTGCTGTTTGTGACGCTGGCTGCGCTGGCGCTACGTCTCTCTTTC
ACTTTGTAATGAGATACTGATATGAGCAACATTACCATTTATCACAACCC
GGCCTGCGGCACGTCGCGTAATACGCTGGAGATGATCCGCAACAGCGGCA
CAGAACCGACTATTATCCATTATCTGGAAACTCCGCCAACGCGCGATGAA
CTGGTCAAACTCATTGCCGATATGGGGATTTCCGTACGCGCGCTGCTGCG
TAAAAACGTCGAACCGTATGAGGAGCTGGGCCTTGCGGAAGATAAATTTA
CTGACGATCGGTTAATCGACTTTATGCTTCAGCACCCGATTCTGATTAAT
CGCCCGATTGTGGTGACGCCGCTGGGAACTCGCCTGTGCCGCCCTTCAGA
AGTGGTGCTGGAAATTCTGCCAGATGCGCAAAAAGGCGCATTCTCCAAGG
AAGATGGCGAGAAAGTGGTTGATGAAGCGGGTAAGCGCCTG

>2W276R (Linear DNA sequence for recombineering to generate the stable E. coli strain DH10B-2W276R, with the 20 bp sequence corresponding to the guide RNA sequence guide #498 that specifies dST1Cas9 specificity underlined). (SEQ ID NO: 123)

TTCTGTTACCCATCCAATTGTTCAAAATTC
TTGCTGATGAAACCCGTCTGGGCATCGTTTTACTGCTCAGCGAACTGGGA
GAGTTATGCGTCTGCGATCTCTGCACTGCTCTCGACCAGTCGCAGCCCAA
GATCTCCCGCCACCTGGCATTGCTGCGTGAAAGCGGGCTATTGCTGGACC
GCAAGCAAGGTAAGTGGGTTCATTACCGCTTATCACCGCATATTCCAGCA
TGGGCGGCGAAAATTATTGATGAGGCCTGGCGATGTGAACAGGAAAAGGT
TCAGGCGATTGTCCGCAACCTGGCTCGACAAAACTGTTCCGGGGACAGTA
AGAACATTTGCAGTTAAAAATTTAGCTAAACACATATGAATTTTCAGATG
TGTTTTATCCGGGAGGCATTATGTTACTGGCAGGCGCTATCTTTGTCCTG
ACCATCGTATTGGTTATCTGGCAGCCGAAAGGTTTAGGCATCGGCTGGAG
TGCAACGCTCGGCGCAGTACTGGCGTTAGTTACGGGCGTGGTCCATCCGG
GTGATATTCCGGTGGTGTGGAATATCGTCTGGAACGCGACGGCTGCGTTT
ATCGCCGTCATTATCATCAGCCTGCTGCTGGATGAGTCCGGCTTTTTTGA
ATGGGCGGCGCTGCACGTCTCACGCTGGGGTAATGGTCGTGGTCGCTTGC
TGTTTACCTGGATTGTCCTGCTCGGTGCTGCCGTTGCCGCCCTGTTTGCC
AATGATGGCGCGGCGCTTATTTTGACACCGATTGTCATCGCCATGCTGCT
GGCTTTAGGGTTCAGTAAAGGCACTACGCTGGCGTTCGTGATGGCGGCCG
GATTCATTGCCGATACCGCCAGCCTGCCGCTTATTGTCTCCAACCTGGTG
AATATCGTTTCCGCTGATTTCTTTGGCCTCGGCTTTCGCGAATACGCCTC
GGTGATGGTGCCGGTGGATATCGCCGCGATTGTTGCCACGCTGGTGATGT
TACATCTCTATTTTCGCAAAGATATTCCGCAGAACTACGATATGGCGCTG
CTGAAATCTCCCGCAGAAGCGATCAAAGATCCTGCTACGTTCAAAACTGG
GCAAGGAGTTGACAGCTAGCTCAGTCCTAGGTATAATGCTAGCGTCTAG
TATGCGGATGCCAGGTTTTTGTACTCGAAAGAAGCTACAAAGATAAGGC
TTCATGCCGAAATCAACACCCTGTCATTTTATGGCAGGGTGTTTTTGAAG
CTTGGGCCCGAACAAAAAGCTTCTCGGTACCAAATTCCAGAAAAGAGGCC
TCCCGAAAGGGGGGCCTTTTTTCGTTTTGGTCCCGCTAGGTTTGACAGCT
AGCTCAGTCCTAGGTATAATGCTAGCAGAGAAAGAGGAGAAATACAAATG
AGTGACCTGGTATTAGGTCTGGCGATTGGCATTGGGTCTGTTGGCGTCGG
AATTTTGAACAAAGTTACCGGAGAGATTATCCACAAAAACTCGCGTATTT
TCCCAGCTGCCCAAGCTGAAAATAACTTGGTTCGCCGTACAAATCGCCAG
GGTCGTCGTTTGGCTCGCCGCAAGAAACACCGCCGCGTGCGCCTTAATCG
CCTGTTCGAAGAATCCGGTTTAATCACCGACTTCACGAAGATCTCGATTA
ATCTTAATCCATACCAATTACGCGTAAAGGGGCTTACGGATGAGTTAAGC
AACGAGGAACTGTTCATTGCGTTGAAAAATATGGTCAAACATCGCGGTAT
TAGCTATTTAGATGACGCCAGCGACGACGGTAATTCATCGGTGGGCGATT
ATGCCCAAATCGTAAAAGAAAACTCGAAGCAACTTGAGACAAAAACGCCT
GGCCAAATTCAACTTGAGCGCTATCAGACCTATGGACAGTTGCGCGGTGA
TTTCACTGTCGAAAAAGACGGAAAAAAACACCGCCTGATTAATGTATTTC
CCACCTCGGCTTACCGCAGTGAAGCTTTGCGCATTCTTCAGACCCAACAA
GAGTTTAATCCCCAGATCACAGACGAGTTCATCAATCGTTATCTGGAAAT
CCTGACTGGCAAACGTAAATATTATCATGGACCCGGCAACGAGAAGTCCC
GCACTGACTATGGCCGTTATCGCACCAGTGGTGAGACTCTTGATAATATT
TTTGGCATTTTAATTGGGAAGTGTACTTTCTATCCGGATGAGTTTCGTGC
TGCCAAGGCCTCTTATACGGCGCAAGAATTTAATTTATTGAACGATCTTA
ATAATCTTACGGTCCCTACTGAAACTAAGAAATTATCAAAGGAGCAGAAG
AATCAAATCATCAATTACGTAAAAAATGAAAAGGCTATGGGGCCGGCTAA
ATTGTTCAAATACATTGCGAAATTACTGAGTTGCGACGTTGCCGATATTA
AGGGGTACCGCATTGATAAAAGCGGGAAGGCGGAGATTCACACTTTCGAA
GCCTATCGCAAAATGAAAACGTTGGAAACTTTAGACATCGAACAAATGGA
TCGTGAAACGTTGGACAAGTTGGCTTACGTCCTTACTCTGAATACAGAAC
GCGAAGGCATTCAGGAAGCCTTGGAGCATGAATTCGCAGATGGAAGTTTC
TCTCAAAAGCAAGTAGACGAGTTAGTTCAATTCCGCAAAGCGAACTCGTC
AATTTTTGGAAAGGGCTGGCACAATTTCAGTGTGAAGTTAATGATGGAAC
TTATTCCAGAATTGTATGAGACGAGTGAGGAGCAAATGACGATCTTAACG
CGCCTTGGTAAGCAAAAAACGACATCTTCCAGCAATAAAACAAAATACAT
CGATGAGAAACTTTTGACCGAGGAAATTTATAATCCGGTCGTTGCAAAGA
GTGTTCGCCAAGCAATCAAGATTGTCAATGCCGCCATCAAAGAATATGGA
GATTTTGATAATATCGTTATCGAGATGGCACGCGAGACAAACGAGGACGA
CGAGAAGAAAGCGATTCAGAAGATTCAGAAGGCAAACAAAGACGAAAAGG
ATGCAGCCATGCTGAAAGCTGCAAATCAATATAATGGTAAGGCTGAGCTT
CCTCATAGTGTGTTTCACGGGCATAAACAGTTGGCGACGAAGATCCGTCT
GTGGCATCAGCAGGGAGAGCGTTGCTTATACACCGGCAAGACTATCAGTA
TTCACGACTTGATTAACAATAGTAACCAATTCGAAGTTGATGCTATTCTT
CCTTTATCAATCACATTCGACGACTCTCTGGCTAATAAAGTCTTGGTATA
CGCGACAGCCAACCAAGAAAAAGGACAGCGTACCCCATATCAAGCTTTGG
ATTCAATGGATGACGCCTGGTCCTTCCGTGAGCTGAAGGCTTTCGTGCGC
GAGTCCAAAACCTTGTCTAACAAAAAAAAGGAGTACTTATTGACGGAGGA
GGACATTAGTAAATTCGACGTACGCAAAAAGTTTATTGAGCGCAACCTTG
TTGATACTCGTTACGCATCTCGTGTTGTCCTTAACGCCTTACAGGAGCAT
TTTCGCGCACACAAGATCGACACCAAGGTGTCTGTCGTACGTGGACAATT
CACCAGCCAACTTCGCCGCCACTGGGGGATTGAAAAGACCCGTGACACAT
ATCACCACCATGCCGTTGACGCTCTTATCATTGCTGCGAGTTCCCAGTTG
AATTTATGGAAGAAACAAAAGAACACACTGGTCAGTTATTCTGAAGATCA
ACTTCTGGACATTGAAACTGGAGAGCTGATTTCTGACGATGAATATAAGG
AGTCAGTTTTCAAAGCACCATATCAGCATTTTGTAGATACGCTGAAATCT
AAAGAGTTCGAGGACTCAATCTTGTTCTCGTATCAGGTTGACTCTAAATT
CAATCGCAAGATCAGTGATGCCACTATCTACGCCACTCGCCAAGCGAAGG
TGGGGAAAGATAAAGCCGACGAGACATATGTCTTAGGGAAAATCAAAGAT
ATCTACACACAGGATGGGTACGATGCTTTCATGAAAATTTATAAGAAAGA
CAAAAGTAAATTCTTAATGTATCGCCACGATCCTCAGACTTTCGAGAAGG
TGATCGAGCCTATTCTTGAAAATTATCCTAATAAACAGATCAACGACAAG
GGTAAGGAAGTGCCATGCAATCCTTTTCTGAAGTATAAAGAAGAACACGG
TTACATCCGTAAGTATTCAAAAAAGGGGAATGGACCGGAGATCAAGAGTC
TGAAATACTACGACAGTAAACTTGGGAATCACATTGATATCACCCCGAAG
GATTCCAACAACAAGGTGGTCCTTCAGTCTGTTTCGCCTTGGCGCGCTGA
CGTTTATTTCAATAAGACGACGGGCAAATATGAGATTTTGGGTCTTAAAT
ATGCAGATTTGCAATTTGATAAAGGAACTGGGACATACAAAATTTCACAA
GAAAAATACAATGACATTAAGAAAAAGGAGGGGGTAGACAGTGATTCAGA
GTTTAAGTTTACCTTATATAAAAATGATCTTTTGTTAGTCAAGGACACCG
AGACGAAGGAACAACAGCTGTTTCGCTTCTTATCGCGCACAATGCCTAAA
CAAAAACATTATGTTGAACTGAAGCCCTACGACAAACAAAAATTTGAGGG
AGGTGAAGCATTAATCAAGGTCCTTGGGAATGTAGCTAACTCGGGCCAGT
GTAAGAAGGGTCTTGGCAAATCTAACATCTCTATCTATAAGGTTCGCACG
GATGTCTTGGGAAACCAACATATCATTAAGAACGAAGGAGACAAACCAAA
GTTGGACTTCTAAGCTTAAAAAAAAAAAAGGCCTCCCAAATCGGGGGGCC
TTTTTTATTGATAACAAAACGCTTGCCGGAGCTGATAGCTAGCTCAGTCC
TAGGGATTATGCTAGCTACTAGAGAAAGAGGAGAAATACAAATGACCAAG
TCTGAAACATTCATGATTCCAAACCACAAGGCCGCCAAATTAAGTGAGCT
GGATATGATGATCGTTAACTCTGTCCCGCCTGGGGGAAACTGGAAGAATA
TTCCCTTGGATGTACCATCGAAACGTATCGAACAGATTCGTGACAGCTAT
GCTCAAGGAAAAGGGTCGCGCAGCACATACTACGGGCGTTTATTGCCCGA
TATGCCAGCTTATACGATCAATACTTATTTCAATCGTCCTGGCAACGGAT
GCCACATTCACTACGAGCAAGATCGCGTACTTTCACAACGCGAAGCTGCA
CGTCTGCAGTCGTTCCCTGATGACTTTATCTTTTTTGGAGGTCAAACGGC
GATTAATACGCAAATCGGTAATGCCGTGCCTCCCTTTCTTGCGTTTCTTA
TTGCAAAAGAAATTGAAAAAGCGATCGGTAATACCGGCTACTACATTGAC
TTATTCAGTGGTGCAGGCGGATTGGGGTTGGGCTTTAAGTGGGCCGGGTG
GACTCCATTGTTAGCTAATGACATTGAGGAAAAGTACTTACAGACATACT
CGAACAACGTACACAAAGAAGTTTTGTGCGGAAGCATTTCGGACAACGAA
ACTTTTTCTAAGATCGCAGACAAGATTTCTGGCTTTAAGAAATTATATTT
TGATAAACAGCTGTGGATTCTGGGCGGGCCTCCGTGCCAGGGATTTAGCA
CGGCTGGCAACGCGCGTACAATGGACGACCCACGCAACAGTCTGTTTATG
CACTACAAGTCGCTGCTTAACGAGATTAAGCCGAATGGATTCATTTTCGA
GAACGTCGCCGGCCTGTTGAACATGGAAAAAGGAAAGGTCTTTGAACGTG
TTAAGGAGGAATTCTCGTCCACAATGAAAACCATGAATGGTTGGATTTTA
AATTCGGAACATTACGCAATTCCACAACGCCGTAAGCGTGTAATTCTTGT
GGGCAGCAATGATCCGCTGTTCTCGATCGAACCACCTCAGAAGCTGACGG
AAGATAAAGAGTCTTGGGTGTCAGTAAAAGATGCGTTATCTGACCTTCCC
CCATTACAACACGGCGAGGATGGATCTGGTAAATACTATATCCACCACCC
GGAAAATGATTACCAGTTGTTTATGCGTGGAAACATTACACCCTCAGAGT
ATTATGAACGCAACATTAAGCCGTCGCTTTAAGCTTGACGAACAATAAGG
CCTCCCAAATCGGGGGGCCTTTTTATTTTTCAACAAAACGCTACTAACTG
TCTATGCCTGGGAAAGGGTGGGCAGGAGATGGGGCAGTGCAGGAAAAGTG
GCACTATGAACCCTGCAGCCCTAGTTTGACAATTAATCATTGGCATAGTA
TATCTGCATAGTATAATACAACTCACTATAGCAATTGTACTAACCTTCTT
CTCTTTCCTCTCCTGACAGGAGGAGCCATCATGGCCAAGTTGACCAGTGC
CGTTCCGGTGCTCACCGCGCGCGACGTCGCCGGAGCGGTCGAGTTCTGGA
CCGACCGGCTCGGGTTCTCCCGGGACTTCGTGGAGGACGACTTCGCCGGT
GTGGTCCGGGACGACGTGACCCTGTTCATCAGCGCGGTCCAGGACCAGGT
GGTGCCGGACAACACCCTGGCCTGGGTGTGGGTGCGCGGCCTGGACGAGC
TGTACGCCGAGTGGTCGGAGGTCGTGTCCACGAACTTCCGGGACGCCTCC
GGGCCGGCCATGACCGAGATCGGCGAGCAGCCGTGGGGGCGGGAGTTCGC
CCTGCGCGACCCGGCCGGCAACTGCGTGCACTTTGTGGCAGAGGAGCAGG
ACTGATTACCTGTAACAGAGCATTAGCGCAAGGTGATTTTTGTCTTCTTG
CGCTAATTTTTTCTGGGTTGTTTTACTGCTTCTGCTGGTGGGATTTTTCG
TCCTGGAACCGCTCGGCATTCCGGTGAGCGCCATTGCAGCTGTGGGCGCG
CTGATATTATTTGTCGTCGCTAAACGCGGTCATGCGATTAATACGGGTAA
AGTCCTGCGCGGTGCCCCCTGGCAGATTGTCATCTTCTCGCTCGGCATGT
ATCTGGTGGTTTATGGCCTGCGCAATGCCGGATTAACGGAATATCTTTCT
GGCGTACTCAACGTGCTGGCGGATAACGGCCTGTGGGCCGCGACGCTCGG
CACCGGATTCCTCACCGCCTTCCTCTCTTCTATTATGAACAATATGCCGA
CGGTACTGGTTGGCGCGTTGTCCATTGATGGCAGCACGGCATCTGGCGTT
ATCAAAGAAGCGATGGTTTATGCCAATGTGATTGGCTGCGATTTGGGACC
GAAAATTACCCCAATTGGTAGCCTGGCTACGCTACTCTGGCTGCACGTAC
TTTCGCAGAAGAATATGACTATCAGCTGGGGATATTACTTCCGTACAGGG
ATTATCATGACCCTGCCTGTGCTGTTTGTGACGCTGGCTGCGCTGGCGCT
ACGTCTCTCTTTCACTTTGTAATGAGATACTGATATGAGCAACATTACCA
TTTATCACAACCCGGCCTGCGGCACGTCGCGTAATACGCTGGAGATGATC
CGCAACAGCGGCACAGAACCGACTATTATCCATTATCTGGAAACTCCGCC
AACGCGCGATGAACTGGTCAAACTCATTGCCGATATGGGGATTTCCGTAC
GCGCGCTGCTGCGTAAAAACGTCGAACCGTATGAGGAGCTGGGCCTTGCG
GAAGATAAATTTACTGACGATCGGTTAATCGACTTTATGCTTCAGCACCC
GATTCTGATTAATCGCCCGATTGTGGTGACGCCGCTGGGAACTCGCCTGT
GCCGCCCTTCAGAAGTGGTGCTGGAAATTCTGCCAGATGCGCAAAAAGGC
GCATTCTCCAAGGAAGATGGCGAGAAAGTGGTTGATGAAGCGGGTAAGCG
CCTG

>2W278R (Linear DNA sequence for recombineering to generate the stable E. coli strain DH10B-2W278R, with the 20 bp sequence corresponding to the guide RNA sequence guide #500 that specifies dST1Cas9 specificity underlined). (SEQ ID NO: 124)

TTCTGTTACCCATCCAATTGTTCAAAATTC
TTGCTGATGAAACCCGTCTGGGCATCGTTTTACTGCTCAGCGAACTGGGA
GAGTTATGCGTCTGCGATCTCTGCACTGCTCTCGACCAGTCGCAGCCCAA
GATCTCCCGCCACCTGGCATTGCTGCGTGAAAGCGGGCTATTGCTGGACC
GCAAGCAAGGTAAGTGGGTTCATTACCGCTTATCACCGCATATTCCAGCA
TGGGCGGCGAAAATTATTGATGAGGCCTGGCGATGTGAACAGGAAAAGGT
TCAGGCGATTGTCCGCAACCTGGCTCGACAAAACTGTTCCGGGGACAGTA
AGAACATTTGCAGTTAAAAATTTAGCTAAACACATATGAATTTTCAGATG
TGTTTTATCCGGGAGGCATTATGTTACTGGCAGGCGCTATCTTTGTCCTG
ACCATCGTATTGGTTATCTGGCAGCCGAAAGGTTTAGGCATCGGCTGGAG
TGCAACGCTCGGCGCAGTACTGGCGTTAGTTACGGGCGTGGTCCATCCGG
GTGATATTCCGGTGGTGTGGAATATCGTCTGGAACGCGACGGCTGCGTTT
ATCGCCGTCATTATCATCAGCCTGCTGCTGGATGAGTCCGGCTTTTTTGA
ATGGGCGGCGCTGCACGTCTCACGCTGGGGTAATGGTCGTGGTCGCTTGC
TGTTTACCTGGATTGTCCTGCTCGGTGCTGCCGTTGCCGCCCTGTTTGCC
AATGATGGCGCGGCGCTTATTTTGACACCGATTGTCATCGCCATGCTGCT
GGCTTTAGGGTTCAGTAAAGGCACTACGCTGGCGTTCGTGATGGCGGCCG
GATTCATTGCCGATACCGCCAGCCTGCCGCTTATTGTCTCCAACCTGGTG
AATATCGTTTCCGCTGATTTCTTTGGCCTCGGCTTTCGCGAATACGCCTC
GGTGATGGTGCCGGTGGATATCGCCGCGATTGTTGCCACGCTGGTGATGT
TACATCTCTATTTTCGCAAAGATATTCCGCAGAACTACGATATGGCGCTG
CTGAAATCTCCCGCAGAAGCGATCAAAGATCCTGCTACGTTCAAAACTGG
GCAAGGAGTTGACAGCTAGCTCAGTCCTAGGTATAATGCTAGCGAGACC
ACCATTCTGGGGCTGTTTTTGTACTCGAAAGAAGCTACAAAGATAAGGC
TTCATGCCGAAATCAACACCCTGTCATTTTATGGCAGGGTGTTTTTGAAG
CTTGGGCCCGAACAAAAAGCTTCTCGGTACCAAATTCCAGAAAAGAGGCC
TCCCGAAAGGGGGGCCTTTTTTCGTTTTGGTCCCGCTAGGTTTGACAGCT
AGCTCAGTCCTAGGTATAATGCTAGCAGAGAAAGAGGAGAAATACAAATG
AGTGACCTGGTATTAGGTCTGGCGATTGGCATTGGGTCTGTTGGCGTCGG
AATTTTGAACAAAGTTACCGGAGAGATTATCCACAAAAACTCGCGTATTT
TCCCAGCTGCCCAAGCTGAAAATAACTTGGTTCGCCGTACAAATCGCCAG
GGTCGTCGTTTGGCTCGCCGCAAGAAACACCGCCGCGTGCGCCTTAATCG
CCTGTTCGAAGAATCCGGTTTAATCACCGACTTCACGAAGATCTCGATTA
ATCTTAATCCATACCAATTACGCGTAAAGGGGCTTACGGATGAGTTAAGC
AACGAGGAACTGTTCATTGCGTTGAAAAATATGGTCAAACATCGCGGTAT
TAGCTATTTAGATGACGCCAGCGACGACGGTAATTCATCGGTGGGCGATT
ATGCCCAAATCGTAAAAGAAAACTCGAAGCAACTTGAGACAAAAACGCCT
GGCCAAATTCAACTTGAGCGCTATCAGACCTATGGACAGTTGCGCGGTGA
TTTCACTGTCGAAAAAGACGGAAAAAAACACCGCCTGATTAATGTATTTC
CCACCTCGGCTTACCGCAGTGAAGCTTTGCGCATTCTTCAGACCCAACAA
GAGTTTAATCCCCAGATCACAGACGAGTTCATCAATCGTTATCTGGAAAT
CCTGACTGGCAAACGTAAATATTATCATGGACCCGGCAACGAGAAGTCCC
GCACTGACTATGGCCGTTATCGCACCAGTGGTGAGACTCTTGATAATATT
TTTGGCATTTTAATTGGGAAGTGTACTTTCTATCCGGATGAGTTTCGTGC
TGCCAAGGCCTCTTATACGGCGCAAGAATTTAATTTATTGAACGATCTTA
ATAATCTTACGGTCCCTACTGAAACTAAGAAATTATCAAAGGAGCAGAAG
AATCAAATCATCAATTACGTAAAAAATGAAAAGGCTATGGGGCCGGCTAA
ATTGTTCAAATACATTGCGAAATTACTGAGTTGCGACGTTGCCGATATTA
AGGGGTACCGCATTGATAAAAGCGGGAAGGCGGAGATTCACACTTTCGAA
GCCTATCGCAAAATGAAAACGTTGGAAACTTTAGACATCGAACAAATGGA
TCGTGAAACGTTGGACAAGTTGGCTTACGTCCTTACTCTGAATACAGAAC
GCGAAGGCATTCAGGAAGCCTTGGAGCATGAATTCGCAGATGGAAGTTTC
TCTCAAAAGCAAGTAGACGAGTTAGTTCAATTCCGCAAAGCGAACTCGTC
AATTTTTGGAAAGGGCTGGCACAATTTCAGTGTGAAGTTAATGATGGAAC
TTATTCCAGAATTGTATGAGACGAGTGAGGAGCAAATGACGATCTTAACG
CGCCTTGGTAAGCAAAAAACGACATCTTCCAGCAATAAAACAAAATACAT
CGATGAGAAACTTTTGACCGAGGAAATTTATAATCCGGTCGTTGCAAAGA
GTGTTCGCCAAGCAATCAAGATTGTCAATGCCGCCATCAAAGAATATGGA
GATTTTGATAATATCGTTATCGAGATGGCACGCGAGACAAACGAGGACGA
CGAGAAGAAAGCGATTCAGAAGATTCAGAAGGCAAACAAAGACGAAAAGG
ATGCAGCCATGCTGAAAGCTGCAAATCAATATAATGGTAAGGCTGAGCTT
CCTCATAGTGTGTTTCACGGGCATAAACAGTTGGCGACGAAGATCCGTCT
GTGGCATCAGCAGGGAGAGCGTTGCTTATACACCGGCAAGACTATCAGTA
TTCACGACTTGATTAACAATAGTAACCAATTCGAAGTTGATGCTATTCTT
CCTTTATCAATCACATTCGACGACTCTCTGGCTAATAAAGTCTTGGTATA
CGCGACAGCCAACCAAGAAAAAGGACAGCGTACCCCATATCAAGCTTTGG
ATTCAATGGATGACGCCTGGTCCTTCCGTGAGCTGAAGGCTTTCGTGCGC
GAGTCCAAAACCTTGTCTAACAAAAAAAAGGAGTACTTATTGACGGAGGA
GGACATTAGTAAATTCGACGTACGCAAAAAGTTTATTGAGCGCAACCTTG
TTGATACTCGTTACGCATCTCGTGTTGTCCTTAACGCCTTACAGGAGCAT
TTTCGCGCACACAAGATCGACACCAAGGTGTCTGTCGTACGTGGACAATT
CACCAGCCAACTTCGCCGCCACTGGGGGATTGAAAAGACCCGTGACACAT
ATCACCACCATGCCGTTGACGCTCTTATCATTGCTGCGAGTTCCCAGTTG
AATTTATGGAAGAAACAAAAGAACACACTGGTCAGTTATTCTGAAGATCA
ACTTCTGGACATTGAAACTGGAGAGCTGATTTCTGACGATGAATATAAGG
AGTCAGTTTTCAAAGCACCATATCAGCATTTTGTAGATACGCTGAAATCT
AAAGAGTTCGAGGACTCAATCTTGTTCTCGTATCAGGTTGACTCTAAATT
CAATCGCAAGATCAGTGATGCCACTATCTACGCCACTCGCCAAGCGAAGG
TGGGGAAAGATAAAGCCGACGAGACATATGTCTTAGGGAAAATCAAAGAT
ATCTACACACAGGATGGGTACGATGCTTTCATGAAAATTTATAAGAAAGA
CAAAAGTAAATTCTTAATGTATCGCCACGATCCTCAGACTTTCGAGAAGG
TGATCGAGCCTATTCTTGAAAATTATCCTAATAAACAGATCAACGACAAG
GGTAAGGAAGTGCCATGCAATCCTTTTCTGAAGTATAAAGAAGAACACGG
TTACATCCGTAAGTATTCAAAAAAGGGGAATGGACCGGAGATCAAGAGTC
TGAAATACTACGACAGTAAACTTGGGAATCACATTGATATCACCCCGAAG
GATTCCAACAACAAGGTGGTCCTTCAGTCTGTTTCGCCTTGGCGCGCTGA
CGTTTATTTCAATAAGACGACGGGCAAATATGAGATTTTGGGTCTTAAAT
ATGCAGATTTGCAATTTGATAAAGGAACTGGGACATACAAAATTTCACAA
GAAAAATACAATGACATTAAGAAAAAGGAGGGGGTAGACAGTGATTCAGA
GTTTAAGTTTACCTTATATAAAAATGATCTTTTGTTAGTCAAGGACACCG
AGACGAAGGAACAACAGCTGTTTCGCTTCTTATCGCGCACAATGCCTAAA
CAAAAACATTATGTTGAACTGAAGCCCTACGACAAACAAAAATTTGAGGG
AGGTGAAGCATTAATCAAGGTCCTTGGGAATGTAGCTAACTCGGGCCAGT
GTAAGAAGGGTCTTGGCAAATCTAACATCTCTATCTATAAGGTTCGCACG
GATGTCTTGGGAAACCAACATATCATTAAGAACGAAGGAGACAAACCAAA
GTTGGACTTCTAAGCTTAAAAAAAAAAAAGGCCTCCCAAATCGGGGGGCC
TTTTTTATTGATAACAAAACGCTTGCCGGAGCTGATAGCTAGCTCAGTCC
TAGGGATTATGCTAGCTACTAGAGAAAGAGGAGAAATACAAATGACCAAG
TCTGAAACATTCATGATTCCAAACCACAAGGCCGCCAAATTAAGTGAGCT
GGATATGATGATCGTTAACTCTGTCCCGCCTGGGGGAAACTGGAAGAATA
TTCCCTTGGATGTACCATCGAAACGTATCGAACAGATTCGTGACAGCTAT
GCTCAAGGAAAAGGGTCGCGCAGCACATACTACGGGCGTTTATTGCCCGA
TATGCCAGCTTATACGATCAATACTTATTTCAATCGTCCTGGCAACGGAT
GCCACATTCACTACGAGCAAGATCGCGTACTTTCACAACGCGAAGCTGCA
CGTCTGCAGTCGTTCCCTGATGACTTTATCTTTTTTGGAGGTCAAACGGC
GATTAATACGCAAATCGGTAATGCCGTGCCTCCCTTTCTTGCGTTTCTTA
TTGCAAAAGAAATTGAAAAAGCGATCGGTAATACCGGCTACTACATTGAC
TTATTCAGTGGTGCAGGCGGATTGGGGTTGGGCTTTAAGTGGGCCGGGTG
GACTCCATTGTTAGCTAATGACATTGAGGAAAAGTACTTACAGACATACT
CGAACAACGTACACAAAGAAGTTTTGTGCGGAAGCATTTCGGACAACGAA
ACTTTTTCTAAGATCGCAGACAAGATTTCTGGCTTTAAGAAATTATATTT
TGATAAACAGCTGTGGATTCTGGGCGGGCCTCCGTGCCAGGGATTTAGCA
CGGCTGGCAACGCGCGTACAATGGACGACCCACGCAACAGTCTGTTTATG
CACTACAAGTCGCTGCTTAACGAGATTAAGCCGAATGGATTCATTTTCGA
GAACGTCGCCGGCCTGTTGAACATGGAAAAAGGAAAGGTCTTTGAACGTG
TTAAGGAGGAATTCTCGTCCACAATGAAAACCATGAATGGTTGGATTTTA
AATTCGGAACATTACGCAATTCCACAACGCCGTAAGCGTGTAATTCTTGT
GGGCAGCAATGATCCGCTGTTCTCGATCGAACCACCTCAGAAGCTGACGG
AAGATAAAGAGTCTTGGGTGTCAGTAAAAGATGCGTTATCTGACCTTCCC
CCATTACAACACGGCGAGGATGGATCTGGTAAATACTATATCCACCACCC
GGAAAATGATTACCAGTTGTTTATGCGTGGAAACATTACACCCTCAGAGT
ATTATGAACGCAACATTAAGCCGTCGCTTTAAGCTTGACGAACAATAAGG
CCTCCCAAATCGGGGGGCCTTTTTATTTTTCAACAAAACGCTACTAACTG
TCTATGCCTGGGAAAGGGTGGGCAGGAGATGGGGCAGTGCAGGAAAAGTG
GCACTATGAACCCTGCAGCCCTAGTTTGACAATTAATCATTGGCATAGTA
TATCTGCATAGTATAATACAACTCACTATAGCAATTGTACTAACCTTCTT
CTCTTTCCTCTCCTGACAGGAGGAGCCATCATGGCCAAGTTGACCAGTGC
CGTTCCGGTGCTCACCGCGCGCGACGTCGCCGGAGCGGTCGAGTTCTGGA
CCGACCGGCTCGGGTTCTCCCGGGACTTCGTGGAGGACGACTTCGCCGGT
GTGGTCCGGGACGACGTGACCCTGTTCATCAGCGCGGTCCAGGACCAGGT
GGTGCCGGACAACACCCTGGCCTGGGTGTGGGTGCGCGGCCTGGACGAGC
TGTACGCCGAGTGGTCGGAGGTCGTGTCCACGAACTTCCGGGACGCCTCC
GGGCCGGCCATGACCGAGATCGGCGAGCAGCCGTGGGGGCGGGAGTTCGC
CCTGCGCGACCCGGCCGGCAACTGCGTGCACTTTGTGGCAGAGGAGCAGG
ACTGATTACCTGTAACAGAGCATTAGCGCAAGGTGATTTTTGTCTTCTTG
CGCTAATTTTTTCTGGGTTGTTTTACTGCTTCTGCTGGTGGGATTTTTCG
TCCTGGAACCGCTCGGCATTCCGGTGAGCGCCATTGCAGCTGTGGGCGCG
CTGATATTATTTGTCGTCGCTAAACGCGGTCATGCGATTAATACGGGTAA
AGTCCTGCGCGGTGCCCCCTGGCAGATTGTCATCTTCTCGCTCGGCATGT
ATCTGGTGGTTTATGGCCTGCGCAATGCCGGATTAACGGAATATCTTTCT
GGCGTACTCAACGTGCTGGCGGATAACGGCCTGTGGGCCGCGACGCTCGG
CACCGGATTCCTCACCGCCTTCCTCTCTTCTATTATGAACAATATGCCGA
CGGTACTGGTTGGCGCGTTGTCCATTGATGGCAGCACGGCATCTGGCGTT
ATCAAAGAAGCGATGGTTTATGCCAATGTGATTGGCTGCGATTTGGGACC
GAAAATTACCCCAATTGGTAGCCTGGCTACGCTACTCTGGCTGCACGTAC
TTTCGCAGAAGAATATGACTATCAGCTGGGGATATTACTTCCGTACAGGG
ATTATCATGACCCTGCCTGTGCTGTTTGTGACGCTGGCTGCGCTGGCGCT
ACGTCTCTCTTTCACTTTGTAATGAGATACTGATATGAGCAACATTACCA
TTTATCACAACCCGGCCTGCGGCACGTCGCGTAATACGCTGGAGATGATC
CGCAACAGCGGCACAGAACCGACTATTATCCATTATCTGGAAACTCCGCC
AACGCGCGATGAACTGGTCAAACTCATTGCCGATATGGGGATTTCCGTAC
GCGCGCTGCTGCGTAAAAACGTCGAACCGTATGAGGAGCTGGGCCTTGCG
GAAGATAAATTTACTGACGATCGGTTAATCGACTTTATGCTTCAGCACCC
GATTCTGATTAATCGCCCGATTGTGGTGACGCCGCTGGGAACTCGCCTGT
GCCGCCCTTCAGAAGTGGTGCTGGAAATTCTGCCAGATGCGCAAAAAGGC
GCATTCTCCAAGGAAGATGGCGAGAAAGTGGTTGATGAAGCGGGTAAGCG
CCTG

Further Plasmid Sequences

>pET-M.Osp807II (for expression of recombinant M.Osp807II with N terminal His tag) (SEQ ID NO: 127)

TTCTTGAAGACGAAAGGGCCTCGTGATACGCCTATTTTTATAGGTTAATG
TCATGATAATAATGGTTTCTTAGACGTCAGGTGGCACTTTTCGGGGAAAT
GTGCGCGGAACCCCTATTTGTTTATTTTTCTAAATACATTCAAATATGTA
TCCGCTCATGAGACAATAACCCTGATAAATGCTTCAATAATATTGAAAAA
GGAAGAGTATGAGTATTCAACATTTCCGTGTCGCCCTTATTCCCTTTTTT
GCGGCATTTTGCCTTCCTGTTTTTGCTCACCCAGAAACGCTGGTGAAAGT
AAAAGATGCTGAAGATCAGTTGGGTGCACGAGTGGGTTACATCGAACTGG
ATCTCAACAGCGGTAAGATCCTTGAGAGTTTTCGCCCCGAAGAACGTTTT
CCAATGATGAGCACTTTTAAAGTTCTGCTATGTGGCGCGGTATTATCCCG
TGTTGACGCCGGGCAAGAGCAACTCGGTCGCCGCATACACTATTCTCAGA
ATGACTTGGTTGAGTACTCACCAGTCACAGAAAAGCATCTTACGGATGGC
ATGACAGTAAGAGAATTATGCAGTGCTGCCATAACCATGAGTGATAACAC
TGCGGCCAACTTACTTCTGACAACGATCGGAGGACCGAAGGAGCTAACCG
CTTTTTTGCACAACATGGGGGATCATGTAACTCGCCTTGATCGTTGGGAA
CCGGAGCTGAATGAAGCCATACCAAACGACGAGCGTGACACCACGATGCC
TGCAGCAATGGCAACAACGTTGCGCAAACTATTAACTGGCGAACTACTTA
CTCTAGCTTCCCGGCAACAATTAATAGACTGGATGGAGGCGGATAAAGTT
GCAGGACCACTTCTGCGCTCGGCCCTTCCGGCTGGCTGGTTTATTGCTGA
TAAATCTGGAGCCGGTGAGCGTGGGTCTCGCGGTATCATTGCAGCACTGG
GGCCAGATGGTAAGCCCTCCCGTATCGTAGTTATCTACACGACGGGGAGT
CAGGCAACTATGGATGAACGAAATAGACAGATCGCTGAGATAGGTGCCTC
ACTGATTAAGCATTGGTAACTGTCAGACCAAGTTTACTCATATATACTTT
AGATTGATTTAAAACTTCATTTTTAATTTAAAAGGATCTAGGTGAAGATC
CTTTTTGATAATCTCATGACCAAAATCCCTTAACGTGAGTTTTCGTTCCA
CTGAGCGTCAGACCCCGTAGAAAAGATCAAAGGATCTTCTTGAGATCCTT
TTTTTCTGCGCGTAATCTGCTGCTTGCAAACAAAAAAACCACCGCTACCA
GCGGTGGTTTGTTTGCCGGATCAAGAGCTACCAACTCTTTTTCCGAAGGT
AACTGGCTTCAGCAGAGCGCAGATACCAAATACTGTCCTTCTAGTGTAGC
CGTAGTTAGGCCACCACTTCAAGAACTCTGTAGCACCGCCTACATACCTC
GCTCTGCTAATCCTGTTACCAGTGGCTGCTGCCAGTGGCGATAAGTCGTG
TCTTACCGGGTTGGACTCAAGACGATAGTTACCGGATAAGGCGCAGCGGT
CGGGCTGAACGGGGGGTTCGTGCACACAGCCCAGCTTGGAGCGAACGACC
TACACCGAACTGAGATACCTACAGCGTGAGCTATGAGAAAGCGCCACGCT
TCCCGAAGGGAGAAAGGCGGACAGGTATCCGGTAAGCGGCAGGGTCGGAA
CAGGAGAGCGCACGAGGGAGCTTCCAGGGGGAAACGCCTGGTATCTTTAT
AGTCCTGTCGGGTTTCGCCACCTCTGACTTGAGCGTCGATTTTTGTGATG
CTCGTCAGGGGGGCGGAGCCTATGGAAAAACGCCAGCAACGCGGCCTTTT
TACGGTTCCTGGCCTTTTGCTGGCCTTTTGCTCACATGTTCTTTCCTGCG
TTATCCCCTGATTCTGTGGATAACCGTATTACCGCCTTTGAGTGAGCTGA
TACCGCTCGCCGCAGCCGAACGACCGAGCGCAGCGAGTCAGTGAGCGAGG
AAGCGGAAGAGCGCCTGATGCGGTATTTTCTCCTTACGCATCTGTGCGGT
ATTTCACACCGCATATATGGTGCACTCTCAGTACAATCTGCTCTGATGCC
GCATAGTTAAGCCAGTATACACTCCGCTATCGCTACGTGACTGGGTCATG
GCTGCGCCCCGACACCCGCCAACACCCGCTGACGCGCCCTGACGGGCTTG
TCTGCTCCCGGCATCCGCTTACAGACAAGCTGTGACCGTCTCCGGGAGCT
GCATGTGTCAGAGGTTTTCACCGTCATCACCGAAACGCGCGAGGCAGCTG
CGGTAAAGCTCATCAGCGTGGTCGTGAAGCGATTCACAGATGTCTGCCTG
TTCATCCGCGTCCAGCTCGTTGAGTTTCTCCAGAAGCGTTAATGTCTGGC
TTCTGATAAAGCGGGCCATGTTAAGGGCGGTTTTTTCCTGTTTGGTCACT
GATGCCTCCGTGTAAGGGGGATTTCTGTTCATGGGGGTAATGATACCGAT
GAAACGAGAGAGGATGCTCACGATACGGGTTACTGATGATGAACATGCCC
GGTTACTGGAACGTTGTGAGGGTAAACAACTGGCGGTATGGATGCGGCGG
GACCAGAGAAAAATCACTCAGGGTCAATGCCAGCGCTTCGTTAATACAGA
TGTAGGTGTTCCACAGGGTAGCCAGCAGCATCCTGCGATGCAGATCCGGA
ACATAATGGTGCAGGGCGCTGACTTCCGCGTTTCCAGACTTTACGAAACA
CGGAAACCGAAGACCATTCATGTTGTTGCTCAGGTCGCAGACGTTTTGCA
GCAGCAGTCGCTTCACGTTCGCTCGCGTATCGGTGATTCATTCTGCTAAC
CAGTAAGGCAACCCCGCCAGCCTAGCCGGGTCCTCAACGACAGGAGCACG
ATCATGCGCACCCGTGGCCAGGACCCAACGCTGCCCGAGATGCGCCGCGT
GCGGCTGCTGGAGATGGCGGACGCGATGGATATGTTCTGCCAAGGGTTGG
TTTGCGCATTCACAGTTCTCCGCAAGAATTGATTGGCTCCAATTCTTGGA
GTGGTGAATCCGTTAGCGAGGTGCCGCCGGCTTCCATTCAGGTCGAGGTG
GCCCGGCTCCATGCACCGCGACGCAACGCGGGGAGGCAGACAAGGTATAG
GGCGGCGCCTACAATCCATGCCAACCCGTTCCATGTGCTCGCCGAGGCGG
CATAAATCGCCGTGACGATCAGCGGTCCAGTGATCGAAGTTAGGCTGGTA
AGAGCCGCGAGCGATCCTTGAAGCTGTCCCTGATGGTCGTCATCTACCTG
CCTGGACAGCATGGCCTGCAACGCGGGCATCCCGATGCCGCCGGAAGCGA
GAAGAATCATAATGGGGAAGGCCATCCAGCCTCGCGTCGCGAACGCCAGC
AAGACGTAGCCCAGCGCGTCGGCCGCCATGCCGGCGATAATGGCCTGCTT
CTCGCCGAAACGTTTGGTGGCGGGACCAGTGACGAAGGCTTGAGCGAGGG
CGTGCAAGATTCCGAATACCGCAAGCGACAGGCCGATCATCGTCGCGCTC
CAGCGAAAGCGGTCCTCGCCGAAAATGACCCAGAGCGCTGCCGGCACCTG
TCCTACGAGTTGCATGATAAAGAAGACAGTCATAAGTGCGGCGACGATAG
TCATGCCCCGCGCCCACCGGAAGGAGCTGACTGGGTTGAAGGCTCTCAAG
GGCATCGGTCGAGATCCCGGTGCCTAATGAGTGAGCTAACTTACATTAAT
TGCGTTGCGCTCACTGCCCGCTTTCCAGTCGGGAAACCTGTCGTGCCAGC
TGCATTAATGAATCGGCCAACGCGCGGGGAGAGGCGGTTTGCGTATTGGG
CGCCAGGGTGGTTTTTCTTTTCACCAGTGAGACGGGCAACAGCTGATTGC
CCTTCACCGCCTGGCCCTGAGAGAGTTGCAGCAAGCGGTCCACGCTGGTT
TGCCCCAGCAGGCGAAAATCCTGTTTGATGGTGGTTAACGGCGGGATATA
ACATGAGCTGTCTTCGGTATCGTCGTATCCCACTACCGAGATATCCGCAC
CAACGCGCAGCCCGGACTCGGTAATGGCGCGCATTGCGCCCAGCGCCATC
TGATCGTTGGCAACCAGCATCGCAGTGGGAACGATGCCCTCATTCAGCAT
TTGCATGGTTTGTTGAAAACCGGACATGGCACTCCAGTCGCCTTCCCGTT
CCGCTATCGGCTGAATTTGATTGCGAGTGAGATATTTATGCCAGCCAGCC
AGACGCAGACGCGCCGAGACAGAACTTAATGGGCCCGCTAACAGCGCGAT
TTGCTGGTGACCCAATGCGACCAGATGCTCCACGCCCAGTCGCGTACCGT
CTTCATGGGAGAAAATAATACTGTTGATGGGTGTCTGGTCAGAGACATCA
AGAAATAACGCCGGAACATTAGTGCAGGCAGCTTCCACAGCAATGGCATC
CTGGTCATCCAGCGGATAGTTAATGATCAGCCCACTGACGCGTTGCGCGA
GAAGATTGTGCACCGCCGCTTTACAGGCTTCGACGCCGCTTCGTTCTACC
ATCGACACCACCACGCTGGCACCCAGTTGATCGGCGCGAGATTTAATCGC
CGCGACAATTTGCGACGGCGCGTGCAGGGCCAGACTGGAGGTGGCAACGC
CAATCAGCAACGACTGTTTGCCCGCCAGTTGTTGTGCCACGCGGTTGGGA
ATGTAATTCAGCTCCGCCATCGCCGCTTCCACTTTTTCCCGCGTTTTCGC
AGAAACGTGGCTGGCCTGGTTCACCACGCGGGAAACGGTCTGATAAGAGA
CACCGGCATACTCTGCGACATCGTATAACGTTACTGGTTTCACATTCACC
ACCCTGAATTGACTCTCTTCCGGGCGCTATCATGCCATACCGCGAAAGGT
TTTGCGCCATTCGATGGTGTCCGGGATCTCGACGCTCTCCCTTATGCGAC
TCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGG
CCACGGGGCCTGCCACCATACCCACGCCGAAACAAGCGCTCATGAGCCCG
AAGTGGCGAGCCCGATCTTCCCCATCGGTGATGTCGGCGATATAGGCGCC
AGCAACCGCACCTGTGGCGCCGGTGATGCCGGCCACGATGCGTCCGGCGT
AGAGGATCGAGATCTCGATCCCGCGAAATTAATACGACTCACTATAGGGG
AATTGTGAGCGGATAACAATTCCCCTCTAGAAATAATTTTGTTTAACTTT
AAGAAGGAGATATACCATGGGCAGCAGCCATCATCATCATCATCACAGCA
GCGGCCTGGTGCCGCGCGGCAGCCATATGGCAAAGAAGGACACAAACCTT
AAGCTTTCCCACCTTTATAAGGGTAACGTATCGGAAGTCTACGGGCGCTG
GCCTAGTCCGGACTTAATTGTATCGGATGGGGCATACGGTGTTCGTGGAT
TTCGCGGGGACACCGTTGACGCTGCCGGGTTGACGGACTGGTATAAGCCA
CACGTGCTGGCGTGGGCCAAAGCGGCCAAGCCTTCCACTTCTTTGTGGTT
TTGGAACACAGAAGTAGGCTGGGCCACGGTCCACCCTTTATTATTGTCCA
CCGGATGGGAATACGTGCAGTTAGCTGTCTGGGATAAAGGACTGGCGCAC
ATTGCCGGCAATGTTAACGGGAAAACAATCCGTCAATTGCCCGTGGTAAC
AGAGGTGGCGGCCCTTTATCGCCGTACGGTGTACCTGGAAACTGGAGACG
GACTTACATTAAACGCAAAGTCATGGTTGCGCGCAGAGTGGCGTCGCTCG
GGCCTGTCCCTTTCAAAGTCGAATGAAGCGTGTGGTGTGAAGAACGCTGC
GACCCGTAAATATTTGACAGCAGACTGGCTGTGGTACTGGCCGCCTGGTG
ACGCGGTCCAAAAGATGGCGGAGTACTGCATGCAATATGGTAAAAAAACT
TCTTGGCCCTATTTCAGTCTGGATGGCAAAACTATGATTTCAGCTCATGA
TTGGGATAGTTTACGCACTACATGGAATCACCGCAATGGAGTCACAAACG
TGTGGAGCCGTCCGCCGTTGGCCGATAGTGAGCGTTTGAAAGGGACAATG
GAACGCTCCGCACCTCGCACGTATAAACCCACCAAACAGTCAGCAGCACA
TCTGAACCAAAAGCCCCTGGACCTGATGCTTACTCAGGTAGCAGCAGCCA
GTAATGTCGGCGATACAGTCTGGGAACCATTTGGAGGTCTGTGCTCGGCT
AGTGTGGCATCGTCATTACTTGGTCGCCGCAGTTACGCTGCAGAAATTGA
CGACACATTTTACAAGTTAGCAGCGGCCCGCCTGAATGAGGCAAATGCCT
ATTTTGAATCGAACGGTGTTTACGAGTTTAAAGAAGGGGAATAAGGATCC
GGCTGCTAACAAAGCCCGAAAGGAAGCTGAGTTGGCTGCTGCCACCGCTG
AGCAATAACTAGCATAACCCCTTGGGGCCTCTAAACGGGTCTTGAGGGGT
TTTTTGCTGAAAGGAGGAACTATATCCGGATATCCCGCAAGAGGCCCGGC
AGTACCGGCATAACCAAGCCTATGCCTACAGCATCCAGGGTGACGGTGCC
GAGGATGACGATGAGCGCATTGTTAGATTTCATACACGGTGCCTGACTGC
GTTAGCAATTTAACTGTGATAAACTACCGCATTAAAGCTTATCGATGATA
AGCTGTCAAACATGAGAA

>pET-M2.Eco31I (for expression of recombinant M2.Eco31I with 7 amino acid N terminal truncation and with C terminal His tag) (SEQ ID NO: 128)

TGGCGAATGGGACGCGCCCTGTAGCGGCGCATTAAGCGCGGCGGGTGTGG
TGGTTACGCGCAGCGTGACCGCTACACTTGCCAGCGCCCTAGCGCCCGCT
CCTTTCGCTTTCTTCCCTTCCTTTCTCGCCACGTTCGCCGGCTTTCCCCG
TCAAGCTCTAAATCGGGGGCTCCCTTTAGGGTTCCGATTTAGTGCTTTAC
GGCACCTCGACCCCAAAAAACTTGATTAGGGTGATGGTTCACGTAGTGGG
CCATCGCCCTGATAGACGGTTTTTCGCCCTTTGACGTTGGAGTCCACGTT
CTTTAATAGTGGACTCTTGTTCCAAACTGGAACAACACTCAACCCTATCT
CGGTCTATTCTTTTGATTTATAAGGGATTTTGCCGATTTCGGCCTATTGG
TTAAAAAATGAGCTGATTTAACAAAAATTTAACGCGAATTTTAACAAAAT
ATTAACGTTTACAATTTCAGGTGGCACTTTTCGGGGAAATGTGCGCGGAA
CCCCTATTTGTTTATTTTTCTAAATACATTCAAATATGTATCCGCTCATG
AATTAATTCTTAGAAAAACTCATCGAGCATCAAATGAAACTGCAATTTAT
TCATATCAGGATTATCAATACCATATTTTTGAAAAAGCCGTTTCTGTAAT
GAAGGAGAAAACTCACCGAGGCAGTTCCATAGGATGGCAAGATCCTGGTA
TCGGTCTGCGATTCCGACTCGTCCAACATCAATACAACCTATTAATTTCC
CCTCGTCAAAAATAAGGTTATCAAGTGAGAAATCACCATGAGTGACGACT
GAATCCGGTGAGAATGGCAAAAGTTTATGCATTTCTTTCCAGACTTGTTC
AACAGGCCAGCCATTACGCTCGTCATCAAAATCACTCGCATCAACCAAAC
CGTTATTCATTCGTGATTGCGCCTGAGCGAGACGAAATACGCGATCGCTG
TTAAAAGGACAATTACAAACAGGAATCGAATGCAACCGGCGCAGGAACAC
TGCCAGCGCATCAACAATATTTTCACCTGAATCAGGATATTCTTCTAATA
CCTGGAATGCTGTTTTCCCGGGGATCGCAGTGGTGAGTAACCATGCATCA
TCAGGAGTACGGATAAAATGCTTGATGGTCGGAAGAGGCATAAATTCCGT
CAGCCAGTTTAGTCTGACCATCTCATCTGTAACATCATTGGCAACGCTAC
CTTTGCCATGTTTCAGAAACAACTCTGGCGCATCGGGCTTCCCATACAAT
CGATAGATTGTCGCACCTGATTGCCCGACATTATCGCGAGCCCATTTATA
CCCATATAAATCAGCATCCATGTTGGAATTTAATCGCGGCCTAGAGCAAG
ACGTTTCCCGTTGAATATGGCTCATAACACCCCTTGTATTACTGTTTATG
TAAGCAGACAGTTTTATTGTTCATGACCAAAATCCCTTAACGTGAGTTTT
CGTTCCACTGAGCGTCAGACCCCGTAGAAAAGATCAAAGGATCTTCTTGA
GATCCTTTTTTTCTGCGCGTAATCTGCTGCTTGCAAACAAAAAAACCACC
GCTACCAGCGGTGGTTTGTTTGCCGGATCAAGAGCTACCAACTCTTTTTC
CGAAGGTAACTGGCTTCAGCAGAGCGCAGATACCAAATACTGTCCTTCTA
GTGTAGCCGTAGTTAGGCCACCACTTCAAGAACTCTGTAGCACCGCCTAC
ATACCTCGCTCTGCTAATCCTGTTACCAGTGGCTGCTGCCAGTGGCGATA
AGTCGTGTCTTACCGGGTTGGACTCAAGACGATAGTTACCGGATAAGGCG
CAGCGGTCGGGCTGAACGGGGGGTTCGTGCACACAGCCCAGCTTGGAGCG
AACGACCTACACCGAACTGAGATACCTACAGCGTGAGCTATGAGAAAGCG
CCACGCTTCCCGAAGGGAGAAAGGCGGACAGGTATCCGGTAAGCGGCAGG
GTCGGAACAGGAGAGCGCACGAGGGAGCTTCCAGGGGGAAACGCCTGGTA
TCTTTATAGTCCTGTCGGGTTTCGCCACCTCTGACTTGAGCGTCGATTTT
TGTGATGCTCGTCAGGGGGGCGGAGCCTATGGAAAAACGCCAGCAACGCG
GCCTTTTTACGGTTCCTGGCCTTTTGCTGGCCTTTTGCTCACATGTTCTT
TCCTGCGTTATCCCCTGATTCTGTGGATAACCGTATTACCGCCTTTGAGT
GAGCTGATACCGCTCGCCGCAGCCGAACGACCGAGCGCAGCGAGTCAGTG
AGCGAGGAAGCGGAAGAGCGCCTGATGCGGTATTTTCTCCTTACGCATCT
GTGCGGTATTTCACACCGCATATATGGTGCACTCTCAGTACAATCTGCTC
TGATGCCGCATAGTTAAGCCAGTATACACTCCGCTATCGCTACGTGACTG
GGTCATGGCTGCGCCCCGACACCCGCCAACACCCGCTGACGCGCCCTGAC
GGGCTTGTCTGCTCCCGGCATCCGCTTACAGACAAGCTGTGACCGTCTCC
GGGAGCTGCATGTGTCAGAGGTTTTCACCGTCATCACCGAAACGCGCGAG
GCAGCTGCGGTAAAGCTCATCAGCGTGGTCGTGAAGCGATTCACAGATGT
CTGCCTGTTCATCCGCGTCCAGCTCGTTGAGTTTCTCCAGAAGCGTTAAT
GTCTGGCTTCTGATAAAGCGGGCCATGTTAAGGGCGGTTTTTTCCTGTTT
GGTCACTGATGCCTCCGTGTAAGGGGGATTTCTGTTCATGGGGGTAATGA
TACCGATGAAACGAGAGAGGATGCTCACGATACGGGTTACTGATGATGAA
CATGCCCGGTTACTGGAACGTTGTGAGGGTAAACAACTGGCGGTATGGAT
GCGGCGGGACCAGAGAAAAATCACTCAGGGTCAATGCCAGCGCTTCGTTA
ATACAGATGTAGGTGTTCCACAGGGTAGCCAGCAGCATCCTGCGATGCAG
ATCCGGAACATAATGGTGCAGGGCGCTGACTTCCGCGTTTCCAGACTTTA
CGAAACACGGAAACCGAAGACCATTCATGTTGTTGCTCAGGTCGCAGACG
TTTTGCAGCAGCAGTCGCTTCACGTTCGCTCGCGTATCGGTGATTCATTC
TGCTAACCAGTAAGGCAACCCCGCCAGCCTAGCCGGGTCCTCAACGACAG
GAGCACGATCATGCGCACCCGTGGGGCCGCCATGCCGGCGATAATGGCCT
GCTTCTCGCCGAAACGTTTGGTGGCGGGACCAGTGACGAAGGCTTGAGCG
AGGGCGTGCAAGATTCCGAATACCGCAAGCGACAGGCCGATCATCGTCGC
GCTCCAGCGAAAGCGGTCCTCGCCGAAAATGACCCAGAGCGCTGCCGGCA
CCTGTCCTACGAGTTGCATGATAAAGAAGACAGTCATAAGTGCGGCGACG
ATAGTCATGCCCCGCGCCCACCGGAAGGAGCTGACTGGGTTGAAGGCTCT
CAAGGGCATCGGTCGAGATCCCGGTGCCTAATGAGTGAGCTAACTTACAT
TAATTGCGTTGCGCTCACTGCCCGCTTTCCAGTCGGGAAACCTGTCGTGC
CAGCTGCATTAATGAATCGGCCAACGCGCGGGGAGAGGCGGTTTGCGTAT
TGGGCGCCAGGGTGGTTTTTCTTTTCACCAGTGAGACGGGCAACAGCTGA
TTGCCCTTCACCGCCTGGCCCTGAGAGAGTTGCAGCAAGCGGTCCACGCT
GGTTTGCCCCAGCAGGCGAAAATCCTGTTTGATGGTGGTTAACGGCGGGA
TATAACATGAGCTGTCTTCGGTATCGTCGTATCCCACTACCGAGATGTCC
GCACCAACGCGCAGCCCGGACTCGGTAATGGCGCGCATTGCGCCCAGCGC
CATCTGATCGTTGGCAACCAGCATCGCAGTGGGAACGATGCCCTCATTCA
GCATTTGCATGGTTTGTTGAAAACCGGACATGGCACTCCAGTCGCCTTCC
CGTTCCGCTATCGGCTGAATTTGATTGCGAGTGAGATATTTATGCCAGCC
AGCCAGACGCAGACGCGCCGAGACAGAACTTAATGGGCCCGCTAACAGCG
CGATTTGCTGGTGACCCAATGCGACCAGATGCTCCACGCCCAGTCGCGTA
CCGTCTTCATGGGAGAAAATAATACTGTTGATGGGTGTCTGGTCAGAGAC
ATCAAGAAATAACGCCGGAACATTAGTGCAGGCAGCTTCCACAGCAATGG
CATCCTGGTCATCCAGCGGATAGTTAATGATCAGCCCACTGACGCGTTGC
GCGAGAAGATTGTGCACCGCCGCTTTACAGGCTTCGACGCCGCTTCGTTC
TACCATCGACACCACCACGCTGGCACCCAGTTGATCGGCGCGAGATTTAA
TCGCCGCGACAATTTGCGACGGCGCGTGCAGGGCCAGACTGGAGGTGGCA
ACGCCAATCAGCAACGACTGTTTGCCCGCCAGTTGTTGTGCCACGCGGTT
GGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAA
GAGACACCGGCATACTCTGCGACATCGTATAACGTTACTGGTTTCACATT
CACCACCCTGAATTGACTCTCTTCCGGGCGCTATCATGCCATACCGCGAA
AGGTTTTGCGCCATTCGATGGTGTCCGGGATCTCGACGCTCTCCCTTATG
CGACTCCTGCATTAGGAAGCAGCCCAGTAGTAGGTTGAGGCCGTTGAGCA
CCGCCGCCGCAAGGAATGGTGCATGCAAGGAGATGGCGCCCAACAGTCCC
CCGGCCACGGGGCCTGCCACCATACCCACGCCGAAACAAGCGCTCATGAG
CCCGAAGTGGCGAGCCCGATCTTCCCCATCGGTGATGTCGGCGATATAGG
CGCCAGCAACCGCACCTGTGGCGCCGGTGATGCCGGCCACGATGCGTCCG
GCGTAGAGGATCGAGATCGATCTCGATCCCGCGAAATTAATACGACTCAC
TATAGGGGAATTGTGAGCGGATAACAATTCCCCTCTAGAAATAATTTTGT
TTAACTTTAAGAAGGAGATATACATATGATTCCAAACCACAAGGCCGCCA
AATTAAGTGAGCTGGATATGATGATCGTTAACTCTGTCCCGCCTGGGGGA
AACTGGAAGAATATTCCCTTGGATGTACCATCGAAACGTATCGAACAGAT
TCGTGACAGCTATGCTCAAGGAAAAGGGTCGCGCAGCACATACTACGGGC
GTTTATTGCCCGATATGCCAGCTTATACGATCAATACTTATTTCAATCGT
CCTGGCAACGGATGCCACATTCACTACGAGCAAGATCGCGTACTTTCACA
ACGCGAAGCTGCACGTCTGCAGTCGTTCCCTGATGACTTTATCTTTTTTG
GAGGTCAAACGGCGATTAATACGCAAATCGGTAATGCCGTGCCTCCCTTT
CTTGCGTTTCTTATTGCAAAAGAAATTGAAAAAGCGATCGGTAATACCGG
CTACTACATTGACTTATTCAGTGGTGCAGGCGGATTGGGGTTGGGCTTTA
AGTGGGCCGGGTGGACTCCATTGTTAGCTAATGACATTGAGGAAAAGTAC
TTACAGACATACTCGAACAACGTACACAAAGAAGTTTTGTGCGGAAGCAT
TTCGGACAACGAAACTTTTTCTAAGATCGCAGACAAGATTTCTGGCTTTA
AGAAATTATATTTTGATAAACAGCTGTGGATTCTGGGCGGGCCTCCGTGC
CAGGGATTTAGCACGGCTGGCAACGCGCGTACAATGGACGACCCACGCAA
CAGTCTGTTTATGCACTACAAGTCGCTGCTTAACGAGATTAAGCCGAATG
GATTCATTTTCGAGAACGTCGCCGGCCTGTTGAACATGGAAAAAGGAAAG
GTCTTTGAACGTGTTAAGGAGGAATTCTCGTCCACAATGAAAACCATGAA
TGGTTGGATTTTAAATTCGGAACATTACGCAATTCCACAACGCCGTAAGC
GTGTAATTCTTGTGGGCAGCAATGATCCGCTGTTCTCGATCGAACCACCT
CAGAAGCTGACGGAAGATAAAGAGTCTTGGGTGTCAGTAAAAGATGCGTT
ATCTGACCTTCCCCCATTACAACACGGCGAGGATGGATCTGGTAAATACT
ATATCCACCACCCGGAAAATGATTACCAGTTGTTTATGCGTGGAAACATT
ACACCCTCAGAGTATTATGAACGCAACATTAAGCCGTCGCTTAAGCTTGC
GGCCGCACTCGAGCACCACCACCACCACCACTGAGATCCGGCTGCTAACA
AAGCCCGAAAGGAAGCTGAGTTGGCTGCTGCCACCGCTGAGCAATAACTA
GCATAACCCCTTGGGGCCTCTAAACGGGTCTTGAGGGGTTTTTTGCTGAA
AGGAGGAACTATATCCGGAT

>pET-M2.BsaI (for expression of M2.BsaI with C terminal His tag) (SEQ ID NO: 129)

TGGCGAATGGGACGCGCCCTGTAGCGGCGCATTAAGCGCGGCGGGTGTGG
TGGTTACGCGCAGCGTGACCGCTACACTTGCCAGCGCCCTAGCGCCCGCT
CCTTTCGCTTTCTTCCCTTCCTTTCTCGCCACGTTCGCCGGCTTTCCCCG
TCAAGCTCTAAATCGGGGGCTCCCTTTAGGGTTCCGATTTAGTGCTTTAC
GGCACCTCGACCCCAAAAAACTTGATTAGGGTGATGGTTCACGTAGTGGG
CCATCGCCCTGATAGACGGTTTTTCGCCCTTTGACGTTGGAGTCCACGTT
CTTTAATAGTGGACTCTTGTTCCAAACTGGAACAACACTCAACCCTATCT
CGGTCTATTCTTTTGATTTATAAGGGATTTTGCCGATTTCGGCCTATTGG
TTAAAAAATGAGCTGATTTAACAAAAATTTAACGCGAATTTTAACAAAAT
ATTAACGTTTACAATTTCAGGTGGCACTTTTCGGGGAAATGTGCGCGGAA
CCCCTATTTGTTTATTTTTCTAAATACATTCAAATATGTATCCGCTCATG
AATTAATTCTTAGAAAAACTCATCGAGCATCAAATGAAACTGCAATTTAT
TCATATCAGGATTATCAATACCATATTTTTGAAAAAGCCGTTTCTGTAAT
GAAGGAGAAAACTCACCGAGGCAGTTCCATAGGATGGCAAGATCCTGGTA
TCGGTCTGCGATTCCGACTCGTCCAACATCAATACAACCTATTAATTTCC
CCTCGTCAAAAATAAGGTTATCAAGTGAGAAATCACCATGAGTGACGACT
GAATCCGGTGAGAATGGCAAAAGTTTATGCATTTCTTTCCAGACTTGTTC
AACAGGCCAGCCATTACGCTCGTCATCAAAATCACTCGCATCAACCAAAC
CGTTATTCATTCGTGATTGCGCCTGAGCGAGACGAAATACGCGATCGCTG
TTAAAAGGACAATTACAAACAGGAATCGAATGCAACCGGCGCAGGAACAC
TGCCAGCGCATCAACAATATTTTCACCTGAATCAGGATATTCTTCTAATA
CCTGGAATGCTGTTTTCCCGGGGATCGCAGTGGTGAGTAACCATGCATCA
TCAGGAGTACGGATAAAATGCTTGATGGTCGGAAGAGGCATAAATTCCGT
CAGCCAGTTTAGTCTGACCATCTCATCTGTAACATCATTGGCAACGCTAC
CTTTGCCATGTTTCAGAAACAACTCTGGCGCATCGGGCTTCCCATACAAT
CGATAGATTGTCGCACCTGATTGCCCGACATTATCGCGAGCCCATTTATA
CCCATATAAATCAGCATCCATGTTGGAATTTAATCGCGGCCTAGAGCAAG
ACGTTTCCCGTTGAATATGGCTCATAACACCCCTTGTATTACTGTTTATG
TAAGCAGACAGTTTTATTGTTCATGACCAAAATCCCTTAACGTGAGTTTT
CGTTCCACTGAGCGTCAGACCCCGTAGAAAAGATCAAAGGATCTTCTTGA
GATCCTTTTTTTCTGCGCGTAATCTGCTGCTTGCAAACAAAAAAACCACC
GCTACCAGCGGTGGTTTGTTTGCCGGATCAAGAGCTACCAACTCTTTTTC
CGAAGGTAACTGGCTTCAGCAGAGCGCAGATACCAAATACTGTCCTTCTA
GTGTAGCCGTAGTTAGGCCACCACTTCAAGAACTCTGTAGCACCGCCTAC
ATACCTCGCTCTGCTAATCCTGTTACCAGTGGCTGCTGCCAGTGGCGATA
AGTCGTGTCTTACCGGGTTGGACTCAAGACGATAGTTACCGGATAAGGCG
CAGCGGTCGGGCTGAACGGGGGGTTCGTGCACACAGCCCAGCTTGGAGCG
AACGACCTACACCGAACTGAGATACCTACAGCGTGAGCTATGAGAAAGCG
CCACGCTTCCCGAAGGGAGAAAGGCGGACAGGTATCCGGTAAGCGGCAGG
GTCGGAACAGGAGAGCGCACGAGGGAGCTTCCAGGGGGAAACGCCTGGTA
TCTTTATAGTCCTGTCGGGTTTCGCCACCTCTGACTTGAGCGTCGATTTT
TGTGATGCTCGTCAGGGGGGCGGAGCCTATGGAAAAACGCCAGCAACGCG
GCCTTTTTACGGTTCCTGGCCTTTTGCTGGCCTTTTGCTCACATGTTCTT
TCCTGCGTTATCCCCTGATTCTGTGGATAACCGTATTACCGCCTTTGAGT
GAGCTGATACCGCTCGCCGCAGCCGAACGACCGAGCGCAGCGAGTCAGTG
AGCGAGGAAGCGGAAGAGCGCCTGATGCGGTATTTTCTCCTTACGCATCT
GTGCGGTATTTCACACCGCATATATGGTGCACTCTCAGTACAATCTGCTC
TGATGCCGCATAGTTAAGCCAGTATACACTCCGCTATCGCTACGTGACTG
GGTCATGGCTGCGCCCCGACACCCGCCAACACCCGCTGACGCGCCCTGAC
GGGCTTGTCTGCTCCCGGCATCCGCTTACAGACAAGCTGTGACCGTCTCC
GGGAGCTGCATGTGTCAGAGGTTTTCACCGTCATCACCGAAACGCGCGAG
GCAGCTGCGGTAAAGCTCATCAGCGTGGTCGTGAAGCGATTCACAGATGT
CTGCCTGTTCATCCGCGTCCAGCTCGTTGAGTTTCTCCAGAAGCGTTAAT
GTCTGGCTTCTGATAAAGCGGGCCATGTTAAGGGCGGTTTTTTCCTGTTT
GGTCACTGATGCCTCCGTGTAAGGGGGATTTCTGTTCATGGGGGTAATGA
TACCGATGAAACGAGAGAGGATGCTCACGATACGGGTTACTGATGATGAA
CATGCCCGGTTACTGGAACGTTGTGAGGGTAAACAACTGGCGGTATGGAT
GCGGCGGGACCAGAGAAAAATCACTCAGGGTCAATGCCAGCGCTTCGTTA
ATACAGATGTAGGTGTTCCACAGGGTAGCCAGCAGCATCCTGCGATGCAG
ATCCGGAACATAATGGTGCAGGGCGCTGACTTCCGCGTTTCCAGACTTTA
CGAAACACGGAAACCGAAGACCATTCATGTTGTTGCTCAGGTCGCAGACG
TTTTGCAGCAGCAGTCGCTTCACGTTCGCTCGCGTATCGGTGATTCATTC
TGCTAACCAGTAAGGCAACCCCGCCAGCCTAGCCGGGTCCTCAACGACAG
GAGCACGATCATGCGCACCCGTGGGGCCGCCATGCCGGCGATAATGGCCT
GCTTCTCGCCGAAACGTTTGGTGGCGGGACCAGTGACGAAGGCTTGAGCG
AGGGCGTGCAAGATTCCGAATACCGCAAGCGACAGGCCGATCATCGTCGC
GCTCCAGCGAAAGCGGTCCTCGCCGAAAATGACCCAGAGCGCTGCCGGCA
CCTGTCCTACGAGTTGCATGATAAAGAAGACAGTCATAAGTGCGGCGACG
ATAGTCATGCCCCGCGCCCACCGGAAGGAGCTGACTGGGTTGAAGGCTCT
CAAGGGCATCGGTCGAGATCCCGGTGCCTAATGAGTGAGCTAACTTACAT
TAATTGCGTTGCGCTCACTGCCCGCTTTCCAGTCGGGAAACCTGTCGTGC
CAGCTGCATTAATGAATCGGCCAACGCGCGGGGAGAGGCGGTTTGCGTAT
TGGGCGCCAGGGTGGTTTTTCTTTTCACCAGTGAGACGGGCAACAGCTGA
TTGCCCTTCACCGCCTGGCCCTGAGAGAGTTGCAGCAAGCGGTCCACGCT
GGTTTGCCCCAGCAGGCGAAAATCCTGTTTGATGGTGGTTAACGGCGGGA
TATAACATGAGCTGTCTTCGGTATCGTCGTATCCCACTACCGAGATGTCC
GCACCAACGCGCAGCCCGGACTCGGTAATGGCGCGCATTGCGCCCAGCGC
CATCTGATCGTTGGCAACCAGCATCGCAGTGGGAACGATGCCCTCATTCA
GCATTTGCATGGTTTGTTGAAAACCGGACATGGCACTCCAGTCGCCTTCC
CGTTCCGCTATCGGCTGAATTTGATTGCGAGTGAGATATTTATGCCAGCC
AGCCAGACGCAGACGCGCCGAGACAGAACTTAATGGGCCCGCTAACAGCG
CGATTTGCTGGTGACCCAATGCGACCAGATGCTCCACGCCCAGTCGCGTA
CCGTCTTCATGGGAGAAAATAATACTGTTGATGGGTGTCTGGTCAGAGAC
ATCAAGAAATAACGCCGGAACATTAGTGCAGGCAGCTTCCACAGCAATGG
CATCCTGGTCATCCAGCGGATAGTTAATGATCAGCCCACTGACGCGTTGC
GCGAGAAGATTGTGCACCGCCGCTTTACAGGCTTCGACGCCGCTTCGTTC
TACCATCGACACCACCACGCTGGCACCCAGTTGATCGGCGCGAGATTTAA
TCGCCGCGACAATTTGCGACGGCGCGTGCAGGGCCAGACTGGAGGTGGCA
ACGCCAATCAGCAACGACTGTTTGCCCGCCAGTTGTTGTGCCACGCGGTT
GGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAA
GAGACACCGGCATACTCTGCGACATCGTATAACGTTACTGGTTTCACATT
CACCACCCTGAATTGACTCTCTTCCGGGCGCTATCATGCCATACCGCGAA
AGGTTTTGCGCCATTCGATGGTGTCCGGGATCTCGACGCTCTCCCTTATG
CGACTCCTGCATTAGGAAGCAGCCCAGTAGTAGGTTGAGGCCGTTGAGCA
CCGCCGCCGCAAGGAATGGTGCATGCAAGGAGATGGCGCCCAACAGTCCC
CCGGCCACGGGGCCTGCCACCATACCCACGCCGAAACAAGCGCTCATGAG
CCCGAAGTGGCGAGCCCGATCTTCCCCATCGGTGATGTCGGCGATATAGG
CGCCAGCAACCGCACCTGTGGCGCCGGTGATGCCGGCCACGATGCGTCCG
GCGTAGAGGATCGAGATCGATCTCGATCCCGCGAAATTAATACGACTCAC
TATAGGGGAATTGTGAGCGGATAACAATTCCCCTCTAGAAATAATTTTGT
TTAACTTTAAGAAGGAGATATACATATGATTCCCAACCACGTATCTTCCA
AGTTAAGTGAACTTGACATGCTTATCATTAAACACGTGCCGCCCGGAGGA
AATTGGAAAGACATCCCCGAATGGGTGCCGTCTAAGCGTTTGGAGCAAAT
TCGTAAGTCTTATGCTGAGGGAAAAGGATCACGCTCAACTTACTATGGCC
GCCTGTTACCAGATATGCCAAGCTACACTATCAACACGTACTTCAACCGT
CCAGGTAATGGGTGCCACATCCATTATGAACAGGATCGCACTCTGTCACA
GCGTGAGGCCGCGCGCTTGCAGTCTTTCCCTGACGATTTCATCTTCTACG
GTAGCAAGACGGCCATCAACAACCAGATCGGCAACGCTGTTCCGCCCCTT
CTTGCTTATCAGATCGCCAAGGCATTTCCCTTCAAAGGCCAATTTGTCGA
TCTTTTTAGTGGGGCGGGGGGCCTTTCCTTAGGTTTCCTTTGGGCCGGGT
GGAAACCGATCATCGCAAATGACATCGATAAATGGGCGCTGACCACCTAC
ATGAATAATATTCACAATGAAGTAGTGCTTGGGGATATCCGCGACGAAAA
GGTCAGTGAGACTATCATTCAAAAGTGCCTGATCGCAAAGAAGTCGAACC
CGGACCGTCCATTGTTTGTTCTTGGGGGACCACCCTGCCAAGGGTTTTCG
ACTGCAGGAAAAAAACGTTCAATCGTGGACGAGCGCAACTGGCTGTTCGA
AAGTTACGTGAGTATCCTGAAAGAAGTCAAGCCAGACGGTTTTATTTTTG
AAAACGTGACTGGCCTGTTGTCGATGGAGAAGGGAGCATTCTTTGAGATG
GTTAAATCGGAATTGTCTAAGACGGTGTCGAATCTTTTCGTATATAAACT
TAATTCGGTGGATTACGGTGTGCCCCAACGCCGCAACCGCGTAGTCATCA
TCGGGGACTCCACCGGAACGAAGAACAGCGAGCCCCCTATCCCAATTACG
TCTCTTAAAGGAGAAAAGACCCTGTTTGACGCCTTAAGCAGTGCCATTTC
CGTAAAGGAGGCCCTTTCAGACTTACCCCTTTTGTCGCCTAATGAGGACG
GCTCTTGGAAGAACTACGTTTGTGAGCCACAAAATATTTATCAGTCATTC
ATGCGCAAGAAGATCACAGCCCAGCAGTATATCGAGATGCTGTCCTCCTT
AGCTATCATTAAGCTTGCGGCCGCACTCGAGCACCACCACCACCACCACT
GAGATCCGGCTGCTAACAAAGCCCGAAAGGAAGCTGAGTTGGCTGCTGCC
ACCGCTGAGCAATAACTAGCATAACCCCTTGGGGCCTCTAAACGGGTCTT
GAGGGGTTTTTTGCTGAAAGGAGGAACTATATCCGGAT

1. A nucleic acid comprising at least one methylation-protectable restriction element, the methylation-protectable restriction element comprising:
(i) a type IIS restriction enzyme recognition sequence, or a partial type IIS restriction enzyme recognition sequence, that is recognised by a type IIS restriction enzyme that cleaves outside of the recognition sequence;
(ii) a DNA methylase recognition sequence that is recognised and methylated by a DNA methylase,
wherein the DNA methylase recognition sequence is identical to, or is encompassed within, the type IIS restriction recognition sequence, such that methylation of the nucleic acid by the DNA methylase methylates the type IIS restriction enzyme recognition sequence and protects the nucleic acid from cleavage by the type IIS restriction enzyme; and
(iii) a recognition sequence for a sequence-specific DNA-binding protein, wherein the recognition sequence is positioned such that the binding of the sequence-specific DNA-binding protein overlaps with the DNA methylase recognition sequence such that binding of the sequence-specific DNA-binding protein is capable of preventing methylation of the type IIS restriction enzyme recognition sequence by the DNA methylase such that it is not protected from cleavage by the type IIS restriction enzyme.

2. The nucleic acid according to claim 1, wherein the type IIS restriction enzyme recognition sequence of the methylation-protectable restriction element comprises or consists of the sequence GGTCTC, or a partial sequence thereof, and the type IIS restriction enzyme is BsaI, or a variant thereof.

3. The nucleic acid according to claim 1, wherein the restriction enzyme that recognizes the restriction enzyme recognition sequence of the methylation-protectable restriction element is capable of cutting nucleic acid to leave at least a 2 bp overhang/sticky end.

4. The nucleic acid according to any preceding claim, wherein the sequence specific DNA binding protein is selected from:
a nucleic acid-guided DNA binding protein;
a second DNA methylase, such that the recognition sequence of the DNA binding protein is a second DNA methylase recognition sequence relative to the first DNA methylase recognition sequence of ii), and said sequences are different;
a transcription activator-like effector;
a deactivated endodeoxyribonuclease; and
a sequence specific zinc finger protein.

5. The nucleic acid according to any preceding claim, wherein the sequence specific DNA binding protein is a deactivated RNA-guided DNA endonuclease enzyme.

6. The nucleic acid according to any preceding claim, wherein the methylation-protectable restriction element further comprises a methylase-switch element, wherein the methylase-switch element comprises a recognition sequence for a switch DNA methylase,
wherein the methylase-switch element comprises the said type IIS restriction enzyme recognition sequence (i) of the methylation-protectable restriction element, and the switch DNA methylase recognition sequence is different to the DNA methylase recognition sequence (ii) of the methylation-protectable restriction element.

7. The nucleic acid according to claim 6, wherein the type IIS restriction enzyme recognition sequence (i) and the switch DNA methylase recognition sequence of the methylase-switch element overlap such that the base modified by the switch DNA methylase of the methylase-switch element lies within the type IIS restriction enzyme recognition sequence (i) such that methylation by the switch DNA methylase blocks the overlapping type IIS restriction enzyme recognition sequence (i).

8. The nucleic acid according to claim 6 or 7, wherein the switch DNA methylase for the methylase-switch element comprises or consists of M.Osp807II or M.Sen0738I.

9. The nucleic acid according to any preceding claim, wherein the nucleic acid further comprises a non-switchable type IIS restriction enzyme recognition sequence opposing the type IIS restriction enzyme recognition sequence (i) of the methylation-protectable restriction element.

10. The nucleic acid according to claim 9, wherein the opposing non-switchable restriction enzyme recognition sequence comprises the same sequence as the type IIS restriction enzyme recognition sequence (i) of the methylation-protectable restriction element, and is recognised by the same type IIS restriction enzyme that recognises the type IIS restriction enzyme recognition sequence (i) of the methylation-protectable restriction element.

11. The nucleic acid according to any one of claims 1 to 8, wherein the nucleic acid comprises an opposing methylation-protectable restriction element.

12. The nucleic acid according to claim 11, wherein the type IIS restriction enzyme recognition sequences of the first methylation-protectable restriction element and opposing methylation-protectable restriction element are the same.

13. The nucleic acid according to any of claims 9 to 12, wherein the nucleic acid comprises a maintenance-type design element wherein the opposing type IIS restriction enzyme recognition sequence opposing the methylation-protectable restriction element is arranged to direct the restriction enzyme to cut the nucleic acid at the same site as the type IIS restriction enzyme directed by the type IIS restriction enzyme recognition sequence (i) of the methylation-protectable restriction element, such that the same overhangs are produced regardless of which type IIS restriction enzyme recognition sequence of the maintenance-type design element direct the cutting.

14. The nucleic acid according to any of claims 9 to 13, wherein the nucleic acid comprises an excision-type design element, wherein the type IIS restriction enzyme recognition sequence (i) of the methylation-protectable restriction element and the opposing type IIS restriction enzyme recognition sequence are positioned close enough together such that the cut site of the opposing type IIS restriction enzyme recognition sequence is at least partially within the sequence of the type IIS restriction enzyme recognition sequence (i) of the methylation-protectable restriction element or the cut site is within the sequence that is in between the sequence of the type IIS restriction enzyme recognition sequence (i) of the methylation-protectable restriction element and the start of its cut site; or
wherein the nucleic acid comprises an excision-type design element where the opposing non-switchable type IIS restriction enzyme cuts x bases from the opposing type IIS restriction enzyme recognition sequence and generates y bases adhesive end, the distance (d) for the number of bases between the type IIS restriction enzyme recognition sequence (i) of the methylation-protectable restriction element and the opposing restriction enzyme recognition sequence is provided by the following equation: d≤2*x.

15. The nucleic acid according to any of claims 9 to 14, wherein the nucleic acid comprises an insertional-type design element, wherein a sequence-insert, comprising a comprise a functional sequence, is provided in between the methylation-protectable restriction element and the opposing type IIS restriction enzyme recognition sequence.

16. The nucleic acid according to any preceding claim, wherein the nucleic acid is a vector.

17. The nucleic acid according to any preceding claim, wherein the nucleic acid comprises at least two methylation-protectable restriction elements.

18. The nucleic acid according to claim 17, wherein the nucleic acid sequence between the cut sites of the two methylation-protectable restriction elements comprises two type IIS restriction enzyme recognition sequences, which respectively oppose the two methylation-protectable restriction elements.

19. The nucleic acid according to claim 17 or 18, wherein the nucleic acid sequence between the cut sites of the two methylation-protectable restriction elements is a discard sequence, optionally wherein the discard sequence comprises a selectable marker.

20. The nucleic acid according to claim 17, wherein the nucleic acid is a pre-cut linearized vector comprising a methylation-protectable restriction element at each end.

21. The nucleic acid according to any preceding claim, wherein the nucleic acid is:
(a) isolated or derived from a bacterial strain that expresses a DNA methylase that recognises (and methylates) the DNA methylase recognition sequence (ii) of the methylation-protectable restriction element; or
(b) methylated by a DNA methylase that recognises (and methylates) the DNA methylase recognition sequence.

22. The nucleic acid according to any preceding claim, wherein the nucleic acid is:
(a) isolated or derived from a bacterial strain that expresses a switch DNA methylase that recognises (and methylates) the switch DNA methylase recognition sequence of the methylation-protectable restriction element; or
(b) methylated by a switch DNA methylase that recognises (and methylates) the switch DNA methylase recognition sequence of the methylation-protectable restriction element.

23. The nucleic acid according to any preceding claim, wherein the nucleic acid is:
(a) isolated or derived from a bacterial strain that expresses the sequence specific DNA binding protein, and associated guide nucleic acid where necessary; or
(b) bound by (i.e. complexed with) the sequence specific DNA binding protein, and associated guide nucleic acid where necessary.

24. A method of assembling DNA comprising:
providing a linearised methylated nucleic acid according to any preceding claim, wherein the methylation-protectable restriction element comprises a methylation-switch element that is switched OFF by methylation of the type IIS restriction enzyme recognition sequence with the switch DNA methylase;
providing two or more DNA/insert fragments of interest for assembly with the linearised methylated nucleic acid, wherein a first DNA fragment comprises a complementary overhang for ligation with a first end of the linearised methylated nucleic acid, and a second DNA fragment comprises a complementary overhang for ligation with the other/second end of the linearised methylated nucleic acid; and
(i) the first and second DNA/insert fragments further comprise complementary overhangs for ligation with each other; or
(ii) the first and second DNA/insert fragments further comprise complementary overhangs for ligation with one or more further DNA/insert fragments having complementary overhangs, such that ligating the DNA fragments and the linearised methylated nucleic acid with a DNA ligase would result in a single assembled DNA molecule; and
ligating the DNA fragments and linearised methylated nucleic acid with a ligase to form a single assembled DNA molecule comprising the sequence of the assembled DNA fragments flanked by the restriction enzyme recognition sequences of the methylation-protectable restriction elements.

25. A method of assembling DNA comprising:
providing a linearised methylated nucleic acid according to any of claims 1 to 23, wherein the methylation-protectable restriction element comprises a methylation-switch element that is switched OFF by methylation of the type IIS restriction enzyme recognition sequence with the switch DNA methylase;
providing a DNA/insert fragment of interest for assembly with the linearised methylated nucleic acid, wherein the DNA/insert fragment of interest comprises complementary overhangs for ligation with the linearised methylated nucleic acid; and
ligating the DNA fragments and linearised methylated nucleic acid with a ligase to form a single assembled DNA molecule comprising the sequence of the assembled DNA fragments flanked by the restriction enzyme recognition sequences of the methylation-protectable restriction elements.

26. The method according to any of claim 24 or 25 wherein, the linearised nucleic acid is provided by providing a nucleic acid according to any of claims 1 to 23 in the form of a circular destination vector comprising two methylated methylation-protectable restriction elements and a discard sequence therebetween,
wherein each methylated methylation-protectable restriction element is opposed by an opposing non-switchable restriction enzyme recognition sequence in the discard sequence, and
further comprise the step of cutting the circular destination vector with restriction enzymes that recognise the opposing non-switchable restriction enzyme recognition sequences in the discard sequence, thereby leaving a linearised nucleic acid having overhangs defined by the restriction enzymes.

27. A method of assembling DNA comprising:
providing a linearised methylated nucleic acid according to any of claims 1 to 23, wherein the type IIS restriction enzyme recognition sequence of the methylation-protectable restriction element is protected from cutting by methylation with the DNA methylase that recognises the DNA methylase recognition sequence (ii) of the methylation-protectable restriction element;
providing two or more DNA/insert fragments of interest for assembly with the linearised methylated nucleic acid, wherein a first DNA fragment comprises a complementary overhang for ligation with a first end of the linearised methylated nucleic acid, and a second DNA fragment comprises a complementary overhang for ligation with the other/second end of the linearised methylated nucleic acid; and
(i) the first and second DNA/insert fragments further comprise complementary overhangs for ligation with each other; or
(ii) the first and second DNA/insert fragments further comprise complementary overhangs for ligation with one or more further DNA/insert fragments having complementary overhangs, such that ligating the DNA fragments and the linearised methylated nucleic acid with a DNA ligase would result in a single assembled DNA molecule; and
ligating the DNA fragments and linearised methylated nucleic acid with a ligase to form a single assembled DNA molecule comprising the sequence of the assembled DNA fragments flanked by the restriction enzyme recognition sequences of the methylation-protectable restriction elements.

28. A method of assembling DNA comprising:
providing a linearised methylated nucleic acid according to any of claims 1 to 23, wherein the type IIS restriction enzyme recognition sequence of the methylation-protectable restriction element is protected from cutting by methylation with the DNA methylase that recognises the DNA methylase recognition sequence (ii) of the methylation-protectable restriction element;
providing a DNA/insert fragment of interest for assembly with the linearised methylated nucleic acid, wherein the DNA/insert fragment of interest comprises complementary overhangs for ligation with the linearised methylated nucleic acid; and
ligating the DNA fragments and linearised methylated nucleic acid with a ligase to form a single assembled DNA molecule comprising the sequence of the assembled DNA fragments flanked by the restriction enzyme recognition sequences of the methylation-protectable restriction elements.

29. The method according to any of claim 27 or 28 wherein, the linearised nucleic acid is provided by providing a nucleic acid according to any one of claims 1 to 23 in the form of a circular destination vector comprising two pairs of opposing methylation-protectable restriction elements and a discard sequence therebetween,
wherein one pair of opposing methylation-protectable restriction elements comprises an outside methylation-protectable restriction element that will remain in the vector after linearization and an opposing inside methylation-protectable restriction element that is in the discard sequence, and wherein the second pair of opposing methylation-protectable restriction elements also comprise an outside methylation-protectable restriction element that will remain in the vector after linearization and an opposing inside methylation-protectable restriction element that is in the discard sequence,
wherein the opposing methylation-protectable restriction elements of a pair comprise different sequence specific DNA binding recognition sequences,
wherein the outside methylation-protectable restriction elements are methylated and thereby protected from cutting by the type IIS restriction enzyme that recognises the type IIS recognition sequences of the outside methylation-protectable restriction elements, and the inside methylation-protectable restriction elements are not methylated and thereby not protected from cutting by the type IIS restriction enzyme that recognises the type IIS recognition sequences of the inside methylation-protectable restriction elements; and
cutting the circular destination vector with the type IIS restriction enzyme that recognises the type IIS recognition sequences of the inside methylation-protectable restriction elements, thereby producing the linearised nucleic acid.

30. The method according to claim 29, wherein the outside methylation-protectable restriction elements are methylated and thereby protected from cutting by preparing/isolating the vector in a strain that expresses the DNA methylase that recognises the DNA methylase recognition sequence (ii) of the methylation-protectable restriction elements, but the strain does not express a functional sequence specific DNA binding protein that recognises the sequence specific DNA binding protein recognition sequence of the outside methylation-protectable restriction elements, and
the inside methylation-protectable restriction elements are not methylated and thereby not protected from cutting as the strain expresses the DNA methylase that recognises the DNA methylase recognition sequence (ii) of the methylation-protectable restriction elements, and expresses a functional sequence specific DNA binding protein that recognises the sequence specific DNA binding protein recognition sequence of the inside methylation-protectable restriction elements.

31. The method according to any of claims 24 to 30, wherein the DNA fragment(s) of interest for assembly with the nucleic acid are provided in a circular donation vector, wherein the method comprises the step of cutting the circular donation vector to release the DNA fragment(s) of interest.

32. The method according to claim 31, wherein the circular donation vector comprises two methylation-protectable restriction elements with a DNA fragment of interest therebetween.

33. The method according to claim 32, wherein the circular donation vector is methylated or at least exposed to methylation by the DNA methylase that recognises the DNA methylase recognition sequence (ii) in the presence of the sequence specific DNA binding protein.

34. The method according to any one of claims 31 to 33, wherein the circular donor vector(s) is purified/isolated from a bacterial strain that expresses the sequence-specific DNA-binding protein that recognises the sequence-specific DNA-binding protein recognition sequence of the methylation-protectable restriction element and the DNA methylase that recognises the DNA methylase recognition sequence (ii) of the methylation-protectable restriction element; or
wherein the circular donor vector(s) is methylated in vitro in the presence of the sequence-specific DNA-binding protein that recognises the sequence-specific DNA-binding protein recognition sequence of the methylation-protectable restriction element and the DNA methylase that recognises the DNA methylase recognition sequence (ii) of the methylation-protectable restriction element.

35. The method according to any one of claims 31 to 34, wherein the steps of restricting and ligating are combined, such that the circular destination vector, the donor vector(s), the restriction enzyme and the ligase are provided in the same composition.

36. A method of scarless DNA assembly of DNA fragments comprising the steps of:
(A) providing a first intermediate vector, comprising the steps of:
providing a first linearised methylated nucleic acid by providing an assembly vector comprising a nucleic acid according to any of claims 1 to 23, wherein the assembly vector comprises a maintenance-type design element and an excision-type design element flanking a discard sequence,
wherein the type IIS restriction enzyme recognition sequences (i) of the maintenance-type design element and the excision-type design element are selectively methylated, such that the outside type IIS restriction enzyme recognition sequences (i) of the maintenance-type design element and excision-type design element in the vector backbone are methylated, and the inside type IIS restriction enzyme recognition sequences of the maintenance-type design element and excision-type design element in the discard sequence are not methylated, and
cutting the assembly vector with the type IIS restriction enzyme that recognises the opposing type IIS restriction enzyme recognition sequences of the maintenance-type design element and excision-type design element that are in the discard sequence, and which are not methylated,
further providing a first DNA/insert fragment for assembly with the first linearised methylated nucleic acid, the first DNA/insert fragment having overhang ends that are adapted to ligate to the overhang ends of the first linearised methylated nucleic acid, wherein any DNA methylase recognition sequences in the DNA fragment have been methylated with the DNA methylase that recognises the DNA methylase recognition sequence (i);
ligating the first DNA/insert fragment for assembly and first linearised methylated nucleic acid with a ligase to form a first methylated intermediate vector comprising a first DNA/insert fragment for assembly flanked by methylation-protectable restriction elements;
transforming the first methylated intermediate vector into a bacterial strain that expresses the DNA methylase that recognises the DNA methylase recognition sequence (i) and the sequence-specific DNA binding protein that recognises the sequence-specific DNA binding protein recognition sequence of the methylation-protectable restriction elements in the first methylated intermediate vector, such that any type IIS restriction enzyme recognition sequences in the first DNA/insert fragment are methylated and the type IIS restriction enzyme recognition sequence of the methylation-protectable restriction elements are not protected;
isolating the first intermediate vector;
(B) providing a second intermediate vector, comprising the steps of:
providing a second linearised methylated nucleic acid by providing an assembly vector comprising a nucleic acid according to any of claims 1 to 23, wherein the assembly vector comprises a maintenance-type design element and an excision-type design element flanking a discard sequence,
wherein the type IIS restriction enzyme recognition sequences (i) of the maintenance-type design element and the excision-type design element are selectively methylated, such that the outside type IIS restriction enzyme recognition sequences (i) of the maintenance-type design element and excision-type design element in the vector backbone are methylated, and the inside type IIS restriction enzyme recognition sequences of the maintenance-type design element and excision-type design element in the discard sequence are not methylated, and
cutting the assembly vector with the type IIS restriction enzyme that recognises the opposing type IIS restriction enzyme recognition sequences of the maintenance-type design element and excision-type design element that are in the discard sequence, and which are not methylated,
further providing a second DNA/insert fragment for assembly with the second linearised methylated nucleic acid, the second DNA/insert fragment having overhang ends that are adapted to ligate to the overhang ends of the second linearised methylated nucleic acid, wherein any DNA methylase recognition sequences in the second DNA/insert fragment have been methylated with the DNA methylase with the DNA methylase that recognises the DNA methylase recognition sequence (i);
ligating the second DNA/insert fragment for assembly and second linearised methylated nucleic acid with a ligase to form a second methylated intermediate vector comprising a second DNA/insert fragment for assembly flanked by methylation-protectable restriction elements;
transforming the second methylated intermediate vector into a bacterial strain that expresses the DNA methylase that recognises the DNA methylase recognition sequence (i), and the sequence-specific DNA binding protein that recognises the sequence-specific DNA binding protein recognition sequence of the methylation-protectable restriction elements in the second methylated intermediate vector, such that any type IIS restriction enzyme recognition sequences in the second DNA/insert fragment are methylated and the type IIS restriction enzyme recognition sequence of the methylation-protectable restriction elements are not protected;
isolating the second intermediate vector;
(C) cutting the first intermediate vector with a type IIS restriction enzyme that recognises the type IIS restriction enzyme recognition sequences of the methylation-protectable restriction elements, thereby forming a first adapted DNA/insert fragment that comprises a maintained-overhang sequence that is determined by the maintenance-type design element and an opposing native-overhang sequence that is determined by the native sequence of the first DNA/insert fragment for assembly;
(D) cutting the second intermediate vector with a type IIS restriction enzyme that recognises the type IIS restriction enzyme recognition sequences of the methylation-protectable restriction elements, thereby forming a second adapted DNA fragment insert that comprises a maintained-overhang sequence that is determined by the maintenance-type design element and an opposing native-overhang sequence that is determined by the native sequence of the second DNA fragment for assembly;
wherein
(i) the first and second adapted DNA/insert fragments are end fragments wherein their native-overhang sequences are complementary, such that they are arranged to ligate together; or
(ii) one or more middle DNA/insert fragments for assembly are provided wherein the first and second adapted DNA/insert fragments are respective end fragments in the assembly, and the one or more middle DNA fragments are arranged to be ligated between the first and second adapted DNA/insert fragments via complementary native-overhang sequences;
further comprising the step of ligating together, with a ligase, the first and second adapted DNA/insert fragments, or the first and second adapted DNA/insert fragments and one or more middle DNA/insert fragments, to form an assembled DNA fragment having maintained-overhangs at each end, and
optionally ligating the assembled DNA fragment into a linearised destination vector.

37. The method according to claim 36, wherein a middle DNA/insert fragment for assembly is provided by
providing a further intermediate vector, comprising the steps of:
providing a further linearised methylated nucleic acid by providing an assembly vector comprising a nucleic acid according to any of claims 1 to 23, wherein the assembly vector comprises a pair of excision-type design elements flanking a discard sequence,
wherein the type IIS restriction enzyme recognition sequences (i) of the excision-type design elements are selectively methylated, such that the outside type IIS restriction enzyme recognition sequences (i) of the excision-type design elements in the vector backbone are methylated, and the inside type IIS restriction enzyme recognition sequences of the excision-type design elements in the discard sequence are not methylated, and
cutting the assembly vector with the type IIS restriction enzyme that recognises the opposing type IIS restriction enzyme recognition sequences of the excision-type design elements that are in the discard sequence, and which are not methylated,
further providing a middle DNA/insert fragment for assembly with the further linearised methylated nucleic acid, the middle DNA/insert fragment having overhang ends that are adapted to ligate to the overhang ends of the further linearised methylated nucleic acid, wherein any DNA methylase recognition sequences in the second DNA/insert fragment have been methylated with a DNA methylase that recognises the DNA methylase recognition sequence (ii) of the methylation-protectable restriction element;
ligating the middle DNA/insert fragment for assembly and further linearised methylated nucleic acid with a ligase to form a further methylated intermediate vector comprising a middle DNA/insert fragment for assembly flanked by methylation-protectable restriction elements;
transforming the further methylated intermediate vector into a bacterial strain that expresses the DNA methylase that recognises the DNA methylase recognition sequence (ii) of the methylation-protectable restriction element, and expresses the sequence-specific DNA binding protein that recognises the sequence-specific DNA binding protein recognition sequences of the methylation-protectable restriction element, such that any type IIS restriction enzyme recognition sequences in the second DNA/insert fragment are methylated and the type IIS restriction enzyme recognition sequence of the methylation-protectable restriction elements are not protected;
isolating the further intermediate vector;
cutting the further intermediate vector with a type IIS restriction enzyme that recognises the type IIS restriction enzyme recognition sequences of the methylation-protectable restriction elements, thereby forming a middle adapted DNA/insert fragment that comprises opposing native-overhang sequences that are determined by the native sequences of the middle DNA/insert fragment for assembly.

38. The method according to any of claim 36 or 37, wherein the first, second and/or middle DNA/insert fragment(s) for assembly are provided in one or more circular donation vectors, wherein the method comprises the step of cutting the circular donation vectors to release the DNA/insert fragment(s) for assembly.

39. The method according to claim 38, wherein the circular donation vectors comprise two methylation-protectable restriction elements with the DNA/insert fragment(s) for assembly therebetween.

40. The method according to claim 39, wherein the circular donation vectors are methylated or at least exposed to methylation by the DNA methylase that recognises the DNA methylase recognition sequence (ii) in the presence of the sequence specific DNA binding protein.

41. The method according to any one of claims 38 to 40, wherein the circular donor vectors are purified/isolated from a bacterial strain that expresses the sequence-specific DNA-binding protein that recognises the sequence-specific DNA-binding protein recognition sequence of the methylation-protectable restriction element and the DNA methylase that recognises the DNA methylase recognition sequence (ii) of the methylation-protectable restriction element; or
wherein the circular donor vectors are methylated in vitro in the presence of the sequence-specific DNA-binding protein that recognises the sequence-specific DNA-binding protein recognition sequence of the methylation-protectable restriction element and the DNA methylase that recognises the DNA methylase recognition sequence (ii) of the methylation-protectable restriction element.

42. Use of a sequence-specific DNA binding protein for controlling the methylation and/or restriction of the methylation-protectable restriction element of the nucleic acid according to any of claims 1 to 23.

43. The use according to claim 42, wherein the sequence-specific DNA binding protein is used to sterically prevent the binding of the DNA methylase that recognises the DNA methylase recognition sequence (ii) of the methylation-protectable restriction element.

44. Use of a nucleic acid comprising opposing BsaI/M2.Eco31I recognition sequences in combination with a sequence-specific DNA binding protein.

45. A method of methylation protecting BsaI recognition sequences in a vector comprising the nucleic acid according to any of claims 1 to 23, wherein the vector comprises at least one BsaI recognition sequence that is not part of the methylation-protectable restriction element of the nucleic acid,
wherein the methylation comprises methylating the vector with M2.Eco31I in the presence of the sequence-specific DNA binding protein which recognises and binds to the sequence-specific DNA binding protein recognition sequence of the methylation-protectable restriction element.

46. A modified bacterial strain that is modified to express the DNA methylase that recognises the DNA methylase recognition sequence (ii) of the methylation-protectable restriction element of the nucleic acid according to any of claims 1 to 23, and the sequence-specific DNA binding protein.

47. Use of a modified bacterial strain that is modified to express a DNA methylase that recognises the DNA methylase recognition sequence (ii) of the methylation-protectable restriction element of the nucleic acid according to any of claims 1 to 23, for manufacturing a nucleic acid molecule according to any of claims 1 to 23, and wherein the modified bacterial strain is further be modified to express a sequence-specific DNA-binding protein.

48. A composition comprising the nucleic acid according to any of claims 1 to 23, wherein the composition further comprises one or more of:
a) a DNA methylase that recognises the DNA methylase recognition sequence (ii) of the methylation-protectable restriction element;
b) a sequence-specific DNA-binding protein; and
c) a switch DNA methylase; and optionally a DNA ligase.

49. A kit comprising the nucleic acid according to any of claims 1 to 23, wherein the composition further comprises one or more of:
a) a DNA methylase that recognises the DNA methylase recognition sequence (ii) of the methylation-protectable restriction element;
b) a sequence-specific DNA-binding protein; and
c) a switch DNA methylase; and optionally a DNA ligase.

50. The kit according to claim 49, further comprising a modified bacterial strain that is modified to express one or more DNA methylases and/or a sequence-specific DNA binding protein, optionally with any guide nucleic acid as necessary.

51. A host cell comprising nucleic acid according to any of claims 1 to 23, wherein the host cell further comprises nucleic acid for the expression of one or more of:
a) a DNA methylase that recognises the DNA methylase recognition sequence (ii) of the methylation-protectable restriction element;
b) a sequence-specific DNA-binding protein; and
c) a switch DNA methylase.

52. Use of the nucleic acid according to any of claims 1 to 23 for assembling DNA fragments of interest, wherein the use of the nucleic acid is with one or more of:
a) a DNA methylase that recognises the DNA methylase recognition sequence (ii) of the methylation-protectable restriction element;
b) a sequence-specific DNA-binding protein described herein; and
c) a switch DNA methylase described herein.

53. Use of a sequence-specific DNA binding protein to protect a type IIS restriction enzyme recognition site from methylation by a DNA methylase that is capable of recognition and methylation of the type IIS restriction enzyme recognition site.

54. Use of a sequence-specific DNA binding protein for steric hindrance of methylation and/or restriction of a first type IIS restriction enzyme recognition sequence in a nucleic acid, wherein the sterically blocking is effected by binding to or near the first type IIS restriction enzyme recognition sequence in the nucleic acid, wherein the nucleic acid comprises a second type IIS restriction enzyme recognition sequence that is the same sequence as the first type IIS restriction enzyme recognition sequence and wherein the second type IIS restriction enzyme recognition sequence is not arranged to be bound by or sterically hindered by the sequence-specific DNA binding protein

55. A method of producing a nucleic acid in the form of a vector according to any of claims 16 to 23, the method comprising transforming a nucleic acid in the form of a vector according to claims 16 to 23 into a bacterial strain that is capable of replicating the vector, and wherein the bacterial strain is modified to express:
a) a DNA methylase that recognises the DNA methylase recognition sequence (ii) of the methylation-protectable restriction element, and
b) a sequence-specific DNA-binding protein described herein, for example dCas9 (optionally with the guide nucleic acid); and
optionally growing the bacteria, such that the nucleic acid is replicated and/or isolating the nucleic acid from the bacteria.

56. A nucleic acid-protein complex comprising the nucleic acid according to any one of claims 1-23 and a sequence specific DNA binding protein that is arranged to bind to the associated sequence specific DNA binding protein recognition sequence in the nucleic acid.
